(19) United States
(12) Patent Application Publication
(10) Pub. No.: US 2010/0113291 A1
(43) Pub. Date: May 6, 2010

(54) COMPLEXES OF NADP WITH THE PROTEIN MABA OF MYCOBACTERIUM TUBERCULOSIS OR WITH MUTANTS THEREOF, AND THEIR USES FOR DESIGNING AND SCREENING ANTIBIOTICS

(75) Inventors: Gilles Labesse, Montpellier (FR); Annaïk Quemard, Montgiscard (FR); Martin Gonsaud, Montpellier (FR); Stéphanie Ducasse-Cabanot, Lucq De Bearn (FR); Mamadou Daffe, Toulouse (FR)

Correspondence Address: YOUNG & THOMPSON, 209 Madison Street, Suite 500, Alexandria, VA, 22314, US

(73) Assignee: CENTRE NATIONAL DE LA RECHERCHE SCIENTIFIQUE, PARIS (FR)

(21) Appl. No.: 12/293,825

(22) PCT Filed: Mar. 20, 2007
(86) PCT No.: PCT/EP2007/002462

§ 371 Date: Jun. 22, 2009

Related U.S. Application Data
(60) Provisional application No. 60/784,914, filed on Mar. 23, 2006.

(51) Int. Cl. C40B 30/02; C07K 14/00; G06G 7/48

(52) U.S. Cl. 506/8; 530/395; 703/11

(57) ABSTRACT

The main subject of the present invention is the use of complexes of NADP with the protein MabA, or with a derived protein, and more particularly the crystallographic coordinates of these proteins in the frame of said complexes, within the framework of the implementation of methods for designing and screening ligands of these proteins, and advantageously ligands inhibiting the enzymatic activity of these proteins, namely antibiotics capable of being used within the framework of the treatment of mycobacteriosis.

The main subject of the present invention is the use of complexes of NADP with the protein MabA, or with a derived protein, and more particularly the crystallographic coordinates of these proteins in the frame of said complexes, within the framework of the implementation of methods for designing and screening ligands of these proteins, and advantageously ligands inhibiting the enzymatic activity of these proteins, namely antibiotics capable of being used within the framework of the treatment of mycobacteriosis.

Tuberculosis is one of the major causes of mortality by a single infectious agent, Mycobacterium tuberculosis. Moreover, for about fifteen years, there has been a recrudescence of this disease in industrialized countries. This phenomenon is linked in part to the appearance of antiobiotic-resistant strains of Mycobacterium tuberculosis. Thus, the design of new antituberculous medicaments has become a declared priority of the Word Health Organization.

The targets of the antituberculous antibiotics currently used in clinics form part of biosynthesis metabolisms of components of the envelope of Mycobacterium tuberculosis. In particular, the target of isoniazid (INH), a 1st-line antituberculous agent, is involved in the synthesis of very long-chain fatty acids (C60-C90), the mycolic acids. Isoniazid inhibits the activity of the protein InhA, which forms part of an enzyme complex, FAS-II, the function of which is to produce, by successive elongation cycles, long-chain fatty acids (C18-C32), precursors of the mycolic acids. InhA, a 2-trans-enoyl-ACP reductase, catalyzes the 4th stage of an elongation cycle, which comprises 4 stages. INH is a pro-drug which forms, with the coenzyme of InhA, NADH, an inhibiting adduct, INH-NAD(H). FAS-II comprises at least 3 other main enzymes, the latter therefore representing potential targets for novel antibiotics. The protein MabA catalyzes the 2nd stage of the cycle.

The present invention provides methods for the design of antibiotics for the treatment of mycobacterial infections, in particular tuberculosis.

This invention deals with the determination of the three-dimensional structure of the protein MabA and its C(60)V/S(144)L mutant when complexed with the oxidized form NADP of the native ligand of MabA, i.e. NADPH, on the atomic scale and the study of interactions of said complexes with different ligands or their effect on its enzymatic activity. The invention is based on the use of said complexes as a target for antibiotics. In particular, the study of the interaction of said complexes with different ligands or their effect on their enzymatic activity, by different methods, combined with methods using the crystallographic atomic coordinates of these proteins in the frame of said complexes integrated in a appropriate computer software, are used in order to design inhibitors of the enzymatic activity of MabA.

The present invention provides the methodological tools and material necessary for designing molecules representing potential anti-mycobacterial and antituberculous antibiotics.

The production and purification, in large quantities, of the protein MabA can be carried out very rapidly thanks to the overproduction of MabA provided with a poly-Histidine tag in to Escherichia coli and its purification in a single stage by metal chelation chromatography, producing a protein with a high degree of purity. The quantity and quality of the purified protein make it possible to obtain reliable results during studies of enzymatic activity or binding of ligands, but also allow the crystallization of the protein in order to resolve its three-dimensional structure. The development of conditions allowing the freezing of the MabA crystals in liquid nitrogen has made it possible to obtain sets of atomic resolution data (2.05 Å compared with 2.6 Å at ambient temperature) and opens the way to better data thanks to the use of synchrotron radiation. The frozen crystalline structure has revealed the role of compounds (in particular caesium) necessary for the growth of the MabA crystals and makes it possible to envisage rational optimization of crystal growth. The screening in aystallo of “pools” of ligands can also be carried out. The quantity of protein purified is also an important criterion for carrying out high through-put screenings of combinatorial libraries.

The protein MabA activity tests, and as a result the tests on inhibition by potential inhibitors, can be followed easily and rapidly by spectrophotometry, by monitoring the oxidizing of the reduction coenzyme, NADPH, at 340 nm. The inhibition constants (IC50 and Ki) and the inhibition mechanism (competitive, non-competitive, uncompetitive inhibition) for each molecule can be deduced from this. Moreover, tests on specific binding of ligands to the active site of MabA can be also carried out easily and rapidly, by spectrofluorimetry. The presence of the single Trp residue of the protein in the active site makes it possible, by excitation at 303 nm, to detect, from the variation in the fluorescence emission intensity at the emission maximum, the binding of a ligand and to deduce from this the disassociation constant (Kd). Similarly, FRET (Fluorescence Resonance Energy Transfer) experiments can be carried out in the presence of the coenzyme, NADPH, making it possible to conclude from this whether or not the ligand occupies the binding site of the NADPH (binding competition). The simplicity of these measurement methods, and the relatively low volumes that they require, will allow a miniaturization of the inhibition or ligand-binding tests, for the automatic high through-put screening of combinatorial libraries, thanks to an automatic device provided with a spectrophotometer or spectrofluorimeter.

Comparison of the structure of MabA with that of the protein InhA, a protein of the same structural super-family (RED) and belonging to the same enzyme complex (see above), suggested an inhibiting effect of isoniazid on MabA activity and detection of the active form of isoniazid (antituberculous), the INH-NADP(H) adduct. The binding of the adduct and inhibition of the MabA activity was then confirmed experimentally. Similarly, thanks to a strong structural similarity with other proteins, which have been or will be crystallized with ligands (for example, steroid derivatives, co-Crystallized with steroid dehydrogenases), the rational design of potential MabA inhibitors can be carried out rapidly.

The protein MabA is of particular interest as a target of anti-mycobacterial antibiotics. In fact, it forms part of the same enzymatic system as the protein InhA, target of the 1st-line antituberculous medicament, isoniazid. On the other hand, up to now, no protein homologous to MabA has been detected in animal cells. Moreover, comparison with the homologous proteins found in bacteria or plants has highlighted particular properties of MabA, which are linked to its function, since it uses long chain substrates. These characteristics are reflected in the structure of its active site, which makes it possible to envisage the design of inhibitors specific to MabA (in particular, in terms of size and hydrophobic character), and therefore of narrow-spectrum antibiotics. These different points provide MabA with criteria for pharmacological credibility.

Thus, the main object of the present invention is:

research into and design of medicaments effective against opportunist mycobacterial infections (M. avium, M. kansasii, M. fortuitum, M. chelonae etc.) presenting problems in hospitals (sterilization of medical instruments), and in the case of human immuno-deficiency (AIDS, administration of immunosuppressors during a graft, in the case of cancers etc.).

research into and design of medicaments effective against tuberculous infections, in particular medicaments which are effective on the strains of M. tuberculosis resistant to the antibiotics currently used in antituberculous therapy, and which are propagated in populations at risk (prison environment, economically disadvantaged environments etc.).

research into and design of medicaments effective against other bacterial infections, by taking proteins homologous to MabA as molecular targets.

A main subject of the present invention is the complex between NADP and:

the protein MabA, also called protein FabG1, recombinant in the purified form, said protein being a protein of mycobacteria, such as Mycobacterium tuberculosis, and more particularly M. tuberculosis strain H37Rv,

the recombinant proteins derived from the protein MabA, i.e. the MabA C(60)V/S(144)L, the MabA C(60)V, or the MabA S(144)L, said derived proteins being in purified form, and having an NADPH-dependent-ketoacyl reductase activity.

The recombinant protein MabA or the abovementioned derived recombinant proteins, in purified form, are already described in WO 03/082911. Briefly, they are obtained by transformation of strains of E. coli with a plasmid containing a sequence comprising the gene coding for the protein MabA, or comprising a sequence coding for said derived protein, followed by a purification stage during which:

the abovementioned recombinant E. coli bacteria are washed in a washing buffer, then taken up in a lysis buffer, and lysed by a freeze/thaw cycle in the presence of protease inhibitors and lysozyme,

after treatment by DNAse I and RNAse A, in the presence of MgCl₂, and centrifugation, the lysis supernatant of the bacteria obtained in the preceding stage, to which 10% (v/v) of glycerol, or 400 μM of NADP⁺ is added, is applied to an Ni-NTA agarose resin column,

after several washings with 5 mM buffer then 50 mM imidazole, the protein MabA, or the derived proteins, are eluted with the elution buffer.

According to an embodiment, the recombinant protein MabA or the abovementioned derived recombinant proteins, in purified form, are obtained according to the process described above in which the different bacteria washing, lysis, washing, and elution buffers are the following:

bacteria washing buffer: 10 mM potassium phosphate, pH 7.8,

lysis buffer: 50 mM potassium phosphate, pH 7.8 containing 500 mM of NaCl, 5 mM of imidazole,

washing buffer: 50 mM potassium phosphate, pH 7.8 containing 500 mM of NaCl, 5 and 50 mM of imidazole,

elution buffer: 50 mM potassium phosphate, pH 7.8 containing 500 mM of NaCl, and 175 mM of imidazole.

According to another embodiment of the invention, the recombinant protein MabA or the abovementioned derived recombinant proteins, in purified form, are obtained according to the process described above in which the different bacteria washing, lysis, washing, and elution buffers are the following:

bacteria washing buffer: Tris 10 mM, pH 8.0,

lysis buffer:
50 mM Tris buffer, pH 8.0, supplemented with 300 mM LiSO4 and 5 mM imidazole;
or 50 mM Tris buffer, pH 8.0, supplemented with 300 mM KCl and 5 mM imidazole,
washing buffer:
50 mM Tris buffer, pH 8.0, supplemented with 300 mM LiSO4 and 5 or 50 mM imidazole,
or 50 mM Tris buffer, pH 8.0, supplemented with 300 mM KCl and 5 or 50 mM imidazole.
elution buffer:
20 mM MES buffer, pH 6.4, LiSO4 300 mM and 175-750 mM imidazole;
or 20 mM PIPES buffer, pH 8.0, supplemented with 300 mM KCl and 175-750 mM imidazole,

1 mM DTT being added to these buffers in the case of the wild-type protein MabA.

The invention relates to binary complexes between the nicotinamide adenine dinucleotide phosphate of formula:

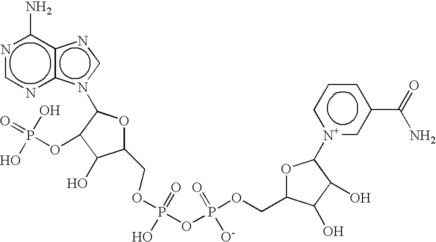

and:

the protein MabA of Mycobacterium tuberculosis, MabA having the following amino acid sequence SEQ ID NO: 1:

MTATATEGAK PPFVSRSVLV TGGNRGIGLA IAQRLAADGH
KVAVTHRGSG APKGLFGVEC DVTDSDAVDR AFTAVEEHQG
PVEVLVSNAG LSADAFLMRM TEEKFEKVIN ANLTGAFRVA
QRASRSMQRN KFGRMIFIGS VSGSWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH

or the proteins derived from the protein MabA mentioned above, and selected from the followings:
the MabA derived protein corresponding to the protein MabA in which the cysteine in position 60 is replaced by a valine residue, and the serine in position 144 is replaced by a leucine residue, said derived protein, also called C(60)V/S(144)L, corresponding to the following sequence SEQ ID NO 2:

MTATATEGAK PPFVSRSVLV TGGNRGIGLA IAQRLAADGH
KVAVTHRGSG APKGLFGVEV DVTDSDAVDR AFTAVEEHQG
PVEVLVSNAG LSADAFLMRM TEEKFEKVIN ANLTGAFRVA
QRASRSMQRN KFGRMIFIGS VSGLWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH

the MabA derived protein corresponding to the protein MabA in which the cysteine in position 60 is replaced by a valine residue, said derived protein, also called C(60)V, corresponding to the following sequence SEQ ID NO 3:

MTATATEGAK PPFVSRSVLV TGGNRGIGLA IAQRLAADGH
KVAVTHRGSG APKGLFGVEV DVTDSDAVDR AFTAVEEHQG
PVEVLVSNAG LSADAFLMRM TEEKFEKVIN ANLTGAFRVA
QRASRSMQRN KFGRMIFIGS VSGSWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH

the MabA derived protein corresponding to the protein MabA in which the serine in position 144 is replaced by a leucine residue, said derived protein, also called S(144)L, corresponding to the following sequence SEQ ID NO 4:

MTATATEGAK PPFVSRSVLV TGGNRGIGLA IAQRLAADGH
KVAVTHRGSG APKGLFGVEC DVTDSDAVDR AFTAVEEHQG
PVEVLVSNAG LSADAFLMRM TEEKFEKVIN ANLTGAFRVA
QRASRSMQRN KFGRMIFIGS VSGLWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH

The invention relates more particularly to ternary complexes of a binary complex as defined above, and a ligand of the protein MabA, or of a recombinant protein derived from the protein MabA, and more particularly a molecule ligand capable of binding specifically at the level of the active site of the protein MabA, or proteins similar in structure to the protein MabA, and inhibiting the enzymatic activity of the latter.

The invention concerns more particularly complexes as defined above, in crystallized form.

The invention also relates to crystals of complexes as defined above, as obtained by the hanging-drop vapour diffusion method, by mixing said protein (1 μl of a 10 mg/ml solution) with a solution (1 μl) of NADP (50-100 mM), polyethylene glycol 3000 (6-12%), CsCl (150-450 mM), and optionally glycerol (10%), in PIPES buffer (50 mM) at pH 6.6.

The invention relates more particularly to crystals of the complex between NADP and the recombinant protein MabA corresponding to the sequence SEQ ID NO: 1 as defined above (and modified by insertion, on the N-terminal side, of a poly-histidine tag of the following sequence SEQ ID NO: 5: MGSSHHHHHH SSGLVPRGSH), the atomic coordinates of the three-dimensional structure of protein MabA in said complex being represented in FIG. 7.

The invention also relates more particularly to crystals of the complex between NADP and the recombinant protein MabA C(60)V/S(144)L corresponding to the sequence SEQ ID NO: 2 as defined above (and modified by insertion, on the N-terminal side, of a poly-histidine tag of the following sequence SEQ ID NO: 5: MGSSHHHHHH SSGLVPRGSH), the atomic coordinates of the three-dimensional structure of protein MabA C(60)V/S(144)L in said complex being represented in FIG. 8.

The invention also relates more particularly to crystals of the recombinant protein MabA C(60)V/S(144)L corresponding to the sequence SEQ ID NO: 2 as defined above (and modified by insertion, on the N-terminal side, of a poly-histidine tag of the following sequence SEQ ID NO: 5: MGSSHHHHHH SSGLVPRGSH), the atomic coordinates of the three-dimensional structure of protein MabA C(60)V/S(144)L being represented in FIG. 9.

The invention also concerns a method for screening ligands of the protein MabA, or of protein MabA C(60)V/S(144)L, or of protein MabA C(60)V, or of protein MabA S(144)L, in crystallo, said method comprising:

either the co-crystallization of the purified recombinant protein MabA, or MabA C(60)V/S(144)L, or MabA C(60)V, or MabA S(144)L, in the presence of NADP and of the potential ligand (or a mixture of potential ligands),

or the soaking of the crystals of the complexes as defined above of NADP with MabA, or with MabA C(60)V/S(144)L, or with MabA C(60)V, or with MabA S(144)L, in a potential ligand solution (or a mixture of potential ligands),

and the determination by crystallography of the three-dimensional structure of the crystals of the ternary complexes of protein MabA, or MabA C(60)V/S(144)L, or MabA C(60)V, or MabA S(144)L, with a potential ligand and NADP.

The invention also concerns a method for designing or screening ligands of the protein MabA, said method comprising the use of the coordinates of the three-dimensional structure of crystals of protein MabA, or of protein MabA C(60)V/S(144)L, or of protein MabA C(60)V, or of protein MabA S(144)L, in complexes of said proteins with NADP, and more particularly of the coordinates of the three-dimensional structure of crystals of protein MabA, or of protein MabA C(60)V/S(144)L, represented in FIGS. 7 and 8 respectively, for screening in silico of the virtual combinatorial libraries of potential ligands, advantageously using appropriate computer softwares, and the detection and rational structural optimization of the ligands capable of binding to said protein.

The invention also relates to a method of rational design of ligands of the protein MabA, said method being carried out starting with known inhibitors of MabA for which the fine three-dimensional structure of the complex between said inhibitor and the recombinant protein MabA in purified form was determined, and rational structural optimization of said inhibitors by using an appropriate computer software in which the coordinates of the three-dimensional structure of protein MabA, or of protein MabA C(60)V/S(144)L, or of protein MabA C(60)V, or of protein MabA S(144)L, in crystals of complexes of said proteins with NADP, and more particularly the coordinates of the three-dimensional structure of protein MabA or of protein MabA C(60)V/S(144)L represented in FIGS. 7 and 8 respectively, have been entered.

The invention concerns more particularly a method as defined above, for designing or screening ligands of the protein MabA, or a recombinant protein derived from the protein MabA, and more particularly molecules capable of binding specifically at the level of the active site of the protein MabA, or proteins similar in structure to the protein MabA, and inhibiting the enzymatic activity of the latter.

The invention relates more particularly to a method as defined above, for designing or screening ligands acting as inhibitors of the protein MabA, or a recombinant protein derived from the protein MabA, these inhibitors being chosen in particular from:

the steroid derivatives,

the derivatives of the antituberculous antibiotic isoniazid (isonicotinic acid hydrazide), such as the derivatives of the isonicotinoyl-NAD(P) adduct,

the derivatives of N-acetyl cysteamine or other simplified types of derivatives of the coenzyme A, comprising a grafted fluorophore making it possible to use the fluorescence spectroscopy method, in particular time-resolved, for the detection of protein-ligand interactions,

the inhibiting derivatives of the protein InhA of Mycobacterium tuberculosis.

The invention also relates more particularly to a method as defined above, for designing or screening ligands of the protein MabA, or a recombinant protein derived from the protein MabA, that can be used in pharmaceutical compositions, in particular within the framework of the treatment of pathologies linked to mycobacterial infections, such as tuberculosis due to infection by Mycobacterium tuberculosis, or by Mycobacterium africanium, or leprosy due to infection by Mycobacterium leprae, or mycobacteriosis due to infection by opportunist mycobacteria, such as Mycobacterium avium, Mycobacterium fortuitum, Mycobacterium kansasii, Mycobacterium chelonae.

The invention is further illustrated by means of the following detailed description.

### LEGEND OF FIGURES

FIG. 1. Sequences alignment of MabA (FabG1) from M. tuberculosis with various orthologs, paralogs, and homologs. MabA_tub: MabA from M tuberculosis; MabA_smegm: MabA from M. smegmatis; MabA_lepre: MabA from M. leprae; FabG2, FabG4c. FabG3 (or PDB1NFG) and FabG5: sequence of the four paralogs annotated in the M. tuberculosis genome; KARbn (or PDB1EDO): KAR fram B. napus; KARec (or PDB1 Q7B): KAR tram E. coli. Secondary structure elements assigned fram the crystal structure of MabA-GSOV/S144L (PDB1UZN) are drawn on the top of the alignment. The figure was drawn with ESPript 2.2 (http://prodes.toulouse.inra.tr/ESPrip/ESPrip/index.php).

FIG. 2. The “open-Iorm” of MabA. Overview of the crystallographic dimer of holo MabA-C60V/S144L The monomer named A is colored in green and the monomer named B in red. In monomer A, the visible part of the NADP (the adenosine and the three phosphates) is represented in pink, orange, and blue. The nonvisible nicotinamide moiety and the corresponding ribose in A and the entire NADP in B were modeled and are shown in gray. The presence of the NADP leads to some conlormational changes in and around the catalytic site. Consequently, the loops β4/α4; β5/α5 and the C-terminus of both monomers are structured. These regions of the protein are shown in blue. All figures (except FIG. 4) were produced using PyMol (http://pymol.sourcelorge.net/).

FIG. 3. NADP electron density for holo MabA-C60V/S144L. Clear density is present for the adenosine and the phosphates, but no density is visible for the nicotinamide moiety after the oxygen A05*linked to the phosphate AP of the cofactor. Residues in hydrogen bound with the cofactor are represented in green. The threonine T21 hydrogen bonded to the asparagine N88 of the β4 strand, are both represented in blue.

FIG. 4. Closed and open structures of MabA. Stereoview of the superposed closed (in red) and open (in green) form of MabA. The structures are shown as traces with catalytic residues serine S140 and tyrosine Y153 as CPK, the incomplete NADP is illustrated as a blue

CPK figure. The regions of major conformational change are emphasized with bold. The figure was produced using VMD 1.8.2 (http://www.ks.uiuc.edu/Research/vmd/).

FIG. 5. Ligand-induced fit. (A) Overview of the “open-form” of the MabA tetramer. The monomers were named A (in red), B (in blue), and the crystallographic symmetric named A′ (in orange) and B′ (in-green). The four C-terminus (motif: “GGMGMGH”) of each monomer lay down at the common buried interface of the tetramer. (B) Detail of the common buried interface of the tetramer. The histidine side chains from a monomer points at hydrogen-bonding distance toward the main-chain carboxyl group of the histidine of the crystallographic symmetric monomer. The region is surrounded by the loop β5/α5 of the four monomers. (C) Element of the hydrogen-bound network between the C-terminus, the loops β5/α5, helix α5, water molecules (not showed) of the four monomers created by the binding of the cofactor, the loop β5/α5, and the C-terminus.

FIG. 6. Ligand docking and substrate specilicity, details of the substrate-binding pocket. (A) Model of the ternary complex MabA/cofactor/substrate. The nicotinamide moiety of NADP points into the substrate-binding site. In the docking the two keto groups of the acyl-CoA (C16) substrate are oriented in a similar manner as observed for InhA and postulated for KARbn. (B) Manual fitting of the C16 acyl substrate based on the comparison with the ternary complex InhA/NAD⁺/C16-substrate. The substrate lay down on a hydrophobic pocket composed of the residues W145, F205, 1198, and I147. This latest residue is substituted by an hydrophilic one in KARbn and KARec and could be responsible for the long/short chain specificity of the KAR. The fitting leads to propose a L shape for the acyl chain of the substrate in MabA (instead of a U shape in InhA). This is due to the presence of hydrophilic residues (S92, D94, and Q150) in the neighborhood of the reactional center. (C) These three hydrophilic residues S92, D94, and Q150 in MabA are conserved between the KAR and the distantly related KR domain of the polyketide synthase. Those residues could interact with the keto groups of the substrate and they form a “binding triad” of the β-keto acyl substrate in the KAR protein family.

Abbreviations: ACP, acyl carrier protein; ENR, enoyl-ACP reductase; FAS, fatty acid synthase; INH, isoniazid; KAR, β-ketoacyl-ACP reductase; KARbn, β-ketoacyl-ACP reductase from B. napus; KARec, β-ketoacyl-ACP reductase from E. coli; PDB, Protein Data Bank; SDR, short-chain dehydrogenases/reductases.

Mycobacterium tuberculosis, the agent of tuberculosis, is the leading cause of death from a single infectious agent. Diverse factors, including the AIDS epidemics, have provoked a resurgence of this disease in industrialized countries, while the disease is still a major problem in a lot of “emergent” countries. In 1996, WHO declared tuberculosis to be a global emergency after the emergence of multidrug resistant strains (1-3). Since then, the search for new targets to develop more effective drugs to control the spread of tuberculosis has become a priority (4).

Mycolic acids are very long chain fatty acids (C60-C90) are essential in the architecture and permeability of the envelope of mycobacteria (5). The front-line antituberculous drug isoniazid (INH) has been shown to impair the biosynthesis of these α-branched and β-hydroxylated molecules by inhibition of the fatty acid elongation type II system called FAS-II (6-8). This complex of several monofunctional enzymes catalyses the elongation of palmitoyl-CoA (C16) into C18-C30 saturated fatty acids, using malonyl-CoA as an elongation unit (9). In comparison, the other known bacterial type II systems perform de novo biosynthesis (10) rather than elongation. The 2-trans-enoyl-acyl carrier protein reductase (ENR) (6) catalyses the fourth and last step of the biosynthesis rounds monitored by the type II systems. INH and/or triclosan (11-13) inhibit ENR, also known as InhA in mycobacteria and FabI in other organisms, mainly bacteria and plants. InhA has been shown to be an essential enzyme for mycobacterial viability (8) suggesting that the production of long-chain fatty acids including mycolic acids may be targeted to inhibit mycobacterial growth.

The MabA protein has been shown to be part of the mycobacterial FAS-II system and to catalyze the second step of an elongation round, that is, the β-ketoacyl reduction (14). MabA is related in sequence to the KARs (sequence identity; 29-43% over 240 amino acids) including those of Escherichia coli (KARec) and of Brassica napus (KARbn) whose crystal structures have also been solved (17-19) (PDB1IO1 and PDB1EDO, respectively). Cloning, overexpression, purification, biochemical, and biophysical characterizations of MabA have been previously described (14, 15).

This enzyme exhibits particular biochemical properties such as a specificity for long-chain substrates, an optimal activity in mild acidic conditions, and has specific sequence motifs that are probably linked to the particular function of the mycobacterial FAS-II system (14). Furthermore, we recently showed that it was also a target of the isoniazid drug (16). The crystal structures of the apo-form of the wild-type (2.0 Å resolution) and of a C60V mutant (2.6 Å resolution) of MabA were also reported recently (15). However, this form does not allow the entrance of any ligand including the known cofactor, NADPH. This precludes the assessment of the potential binding of new chemical compounds.

Here, we report the design and the crystal structures of a double-mutant protein, MabA-C60V/S144L in the presence and the absence of a cofactor. This protein retained 84% of the wild-type enzyme activity, and has an affinity for the cofactor similar to that of the wild type (while the single mutant C60V was 60% active compared to the wild-type enzyme). Nonnative interchain disufides are observed during the purification of the wild-type enzyme, but are absent in the mutant isozymes.

The 1.5-Å structure of MabA-C60/S144L apo-form allowed a refined description of the particular movement features of MabA and more extensively of the rearranged loops. Cocrystallization with a high concentration (50-100 mM) of oxidized cofactor (NADP) revealed the structure of the “open” form (at 1.9-Å resolution). It showed that the active site loops adopt a conformation similar to that observed in holo-KARbn and holo-KARec, while the cofactor is not fully ordered. It also revealed the unique conformation of the C-terminus, which contains the specific sequence signature of mycobacterial MabA proteins: GMGMGH. Finally, a new structure of the wild-type enzyme in the presence of 50 mM of NADP has been solved, and has been shown to be a mixture of two “closed” and “open” conformations. The observed rearrangements are consistent with previous studies by spectrofluorimetry and comparative modeling studies on the wild-type MabA (14, 15). The specific functional and structural features of MabA are discussed in view of the observed cofactor induced conformational change of the active site.

Experimental Procedures

Cloning, directed mutagenesis, purifications, crystallization, and steady-state kinetic experiments were performed as previously described (15, 16). All the data sets were collected at cryogenic temperature using synchrotron radiation (ESRF, beam line BM14) with crystals stabilized using mineral oil as a cryoprotection agent.

Isomorphic crystals were obtained and the structure refinement was initiated by a rigid body minimization with the crystal structure of the MabA-C60V and manual rebuilding with the graphics program O (20) Structure refinement using REFMAC 5.0 in the CCP4 package (21) was performed and after a few steps of rigid-body minimization, restrained refinement using maximum likelihood. Water molecules were added automatically using ARP/Warp.22 TLS parameters (23) were then applied to refine further both apo and holo-forms. In the holo-form of the double mutant, the use of TLS revealed some additional electron density. This was modeled as a truncated NADP. Partial refinement of the wild-type MabA cocrystallized in the presence of NADP (50 mM) revealed the presence of an equal mixture of both the apo and the holo-forms. The trace of NADP was also visible but the cofactor was not modeled.

Analysis of the Ramachandran plot using PROCHECK (24) showed that all modeled residues for the “closed” apo-form and “open” holo-form were found in the allowed regions. But, for the “mixed” form few residues are found in the switching regions (A89, L91, A93, S142, and W145). The details of the refinement statistics and model accuracy are listed in Table 1.

Coordinates and structure factors have been deposited in the Protein Data Bank (25) under the code PDB1UZL (mixed form of MabA), PSB1UZM (“closed” form of MabAC60V/S144L) and PDB1UZN (“open form” of MabA-C60V/S144L).

Results

Sequence Comparison of MabA

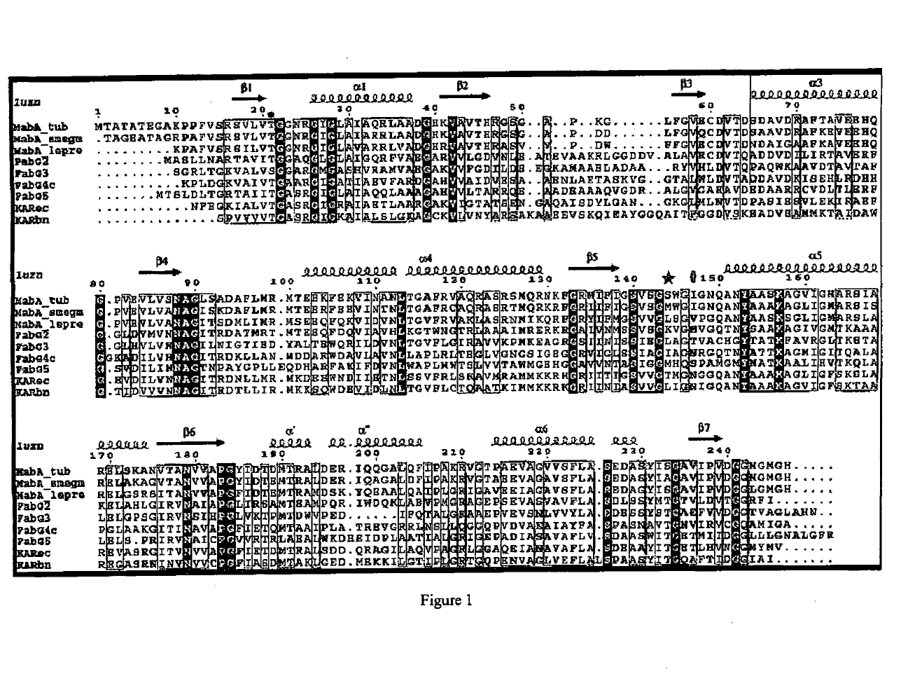

Through PSI-BLAS˜6 searches in sequence databanks, MabA appeared highly conserved among mycobacterial species (M. tuberculosis, M. bovis, M. avium, and M. smegmatis: 81-84% identical) and to a lesser extent in M. leprae (62% identical). These particular mycobacterial KARs form a specific subfamily whose signature is their specific sequence at the very end of C-terminus “GMGMGH” (FIG. 1). This “GH” motif is unique among the SDRs.

Identity scores with other KARs (16 sequences) from various organisms, bacteria, or plants, ranged from 29 to 43% over the whole sequence. Searching only in the genome of M. tuberculosis H37Rv revealed up to 100 SDR-like sequences sharing between 20 and 40% sequence identity with MabA. Among them, five were annotated as KARs (or FabGs with MabA as FabG1 ; FIG. 1). The critical residues for cofactor binding and catalysis in the SDR superfamily (27, 28) are well conserved in the MabA sequence as well as in most of the other similar sequences found in mycobacteria. The specific role of each mycobacterial SDR and especially that of the five putative KARs remains an open question. The crystal structure of FabG3 was recently solved, and the protein has been shown to be a steroid dehydrogenase instead of a ketoacyl reductase. This result suggests that a careful analysis will be necessary to properly annotate the function of numerous sequences belonging to the same superfamily. Solving more structure of the family should help in refining the tremendous task of a fine genome annotation.

Mutant Design and Characterization

The crystal structure of the apo-form of the wild-type MabA was previously reported (15). However, the active site of MabA is partially occupied by two disordered loops preventing the entrance of any ligand including the known cofactor NADPH. This phenomenon was also observed in the case of the apo-form of KARec, but not of the holo-form of KARbn. Thus, the enhanced flexibility does not appear to correlate with the substrate specificity of KARs since KARec and KARbn work on short acyl derivatives (C4-C8), while MabA from mycobacteria preferentially uses longer acyl chains (C12 and C16) (14).

The sequences of MabA and KARec in the two flexible regions of the active site (loops connecting two secondary elements (β4-α4 and β5-α5) were compared to the corresponding regions of KARbn. Sequence changes were analyzed in view of the structure of holo-KARbn and related SDRs (15). In the crystallized holo-forms of SDRs both connecting loops are ordered. The catalytic triad (equivalent to 8140, Y153, and K157 in MabA) surrounds the second loop (β5-α5). In a subset of eight SDRs sharing the same sequence length between the catalytic serine and the catalytic tyrosine, a similar conformation was previously observed (15). No particular sequence change could be correlated to the observed flexibility of the segment β4-α4. In contrast, MabA and KARec possess various amino acids that could enhance the main-chain flexibility between the residues S140 and Q150 (FIG. 1).

In eight SDRs analyzed previously (see above) a large and usually hydrophobie amino acid anchor the β5-α5 loop in its active conformation. In MabA and KARec a small polar amino acid is present at this position (S144 in MabA, T142 in KARec). In the predicted structure of holo-MabA, the serine S144 would be surrounded by three apolar residues (I161, A180, and P238, and respectively, I159, A180, and H236 in KARec). In contrast, the equivalent hydrophobic residue in holo-KARbn, leucine L158 interacts through van der Waals contacts with three residues (I175, C196, and T253). Furthermore, this serine S144 in MabA would be located at the C-terminus of a helical conserved turn in the connecting loop β5-α5 of the SDRs. As a result, we predict that the presence of a leucine, with an enhancement of the hydrophobicity at this position 144 in MabA, has a stabilizing effect. Also, a leucine residue is expected to stabilize the predicted α-helical conformation.

An additional residue is expected to play a major role in the local flexibility of MabA and KARec. A glycine is present at the end of β5 in both MabA (G139) and KARec (G137), while residues with larger side chains are present in the other SDR (A153 in KARbn). A substitution of glycine 139 with alanine was predicted to lower the rearrangement observed in the apo-form of the wild-type enzyme.

Thus, the serine S144 was mutated into leucine in MabA-C60V. In steady-state kinetic experiments, in the presence of acetoacetyl-CoA and NADPH (both at 100 μM), MabA-C60V/S144L displayed a slightly lower activity than the wild-type enzyme (Vi of 0.50±0.03 μmol/min/mg compared to 0.60±0.01 μmol/min/mg for MabA-wt), and a very slight decrease in affinity for the cofactor as shown by spectrofluorimetry (K_{D} of 11.2 μM compared to 8.7 μM for MabA-wt). This double mutant showed an enzymatic activity level and an affinity for its cofactor closer to that of the wild-type enzyme than the single mutant form MabA-C60V. On the contrary, a triple mutant bearing an alanine instead of the glycine at position 139 (MabA-C60V/G139A/S144L) appeared totally inactive and was not studied further.

Refined Structures of Active and Inactive Forms of MabA

Crystallogenesis of MabA-C60V/S144L in the presence or in the absence of NADP gave two sets of crystals.

In the absence of a cofactor, the structure of MabA-C60V/S144L apo-form was solved at a resolution of 1.49 Å. This structure revealed a “closed” form of the protein similar to for the apo-forms of MabA iso-enzymes previously described (15). The mutations C60V or S144L did not affect the “closed” conformation of MabA monomers (RMSD approx 0.1 Å), and the common core is very similar to the conformation of the two other KAR structures (PDB1I01 and PDB1EDO). The RMSD between this “closed” form of MabA-C60V/S144L and, respectively, KARec and KARbn is 0.7 and 0.9 Å over a common core of 112 Cα carbons (corresponding to 40% of the structure). Once again, no electron density was observed for the residues 94 to 99 (loop β4-α4), 142 to 149 (loop β5-α5) and 245 to 247 (the very C-terminus) of both monomers. In the second monomer of the crystal structure (labeled B) the region between strand β6 and helix α6 (residues 189-202) is also not visible, like are several SDRs in the absence of substrate (PDB2AE1, PDB1FKS, and PDB1BDB). In monomer A, this region (residues 190-210) adopts a conformation stabilized by the crystal packing, and is composed of two short helices (a 3₁₀-helix and an α-helix). Similar helical segments have been described in several other SDRs, including InhA, and were shown to be involved in substrate recognition (29).

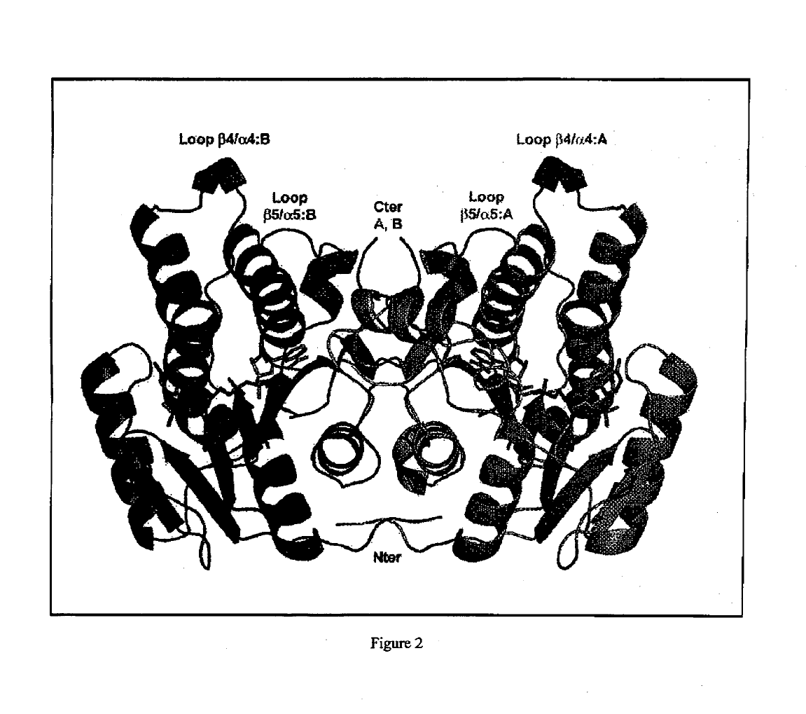

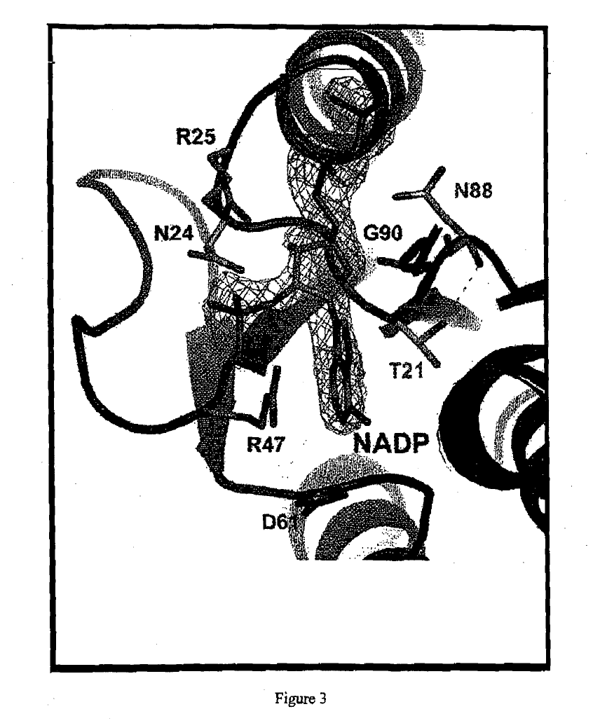

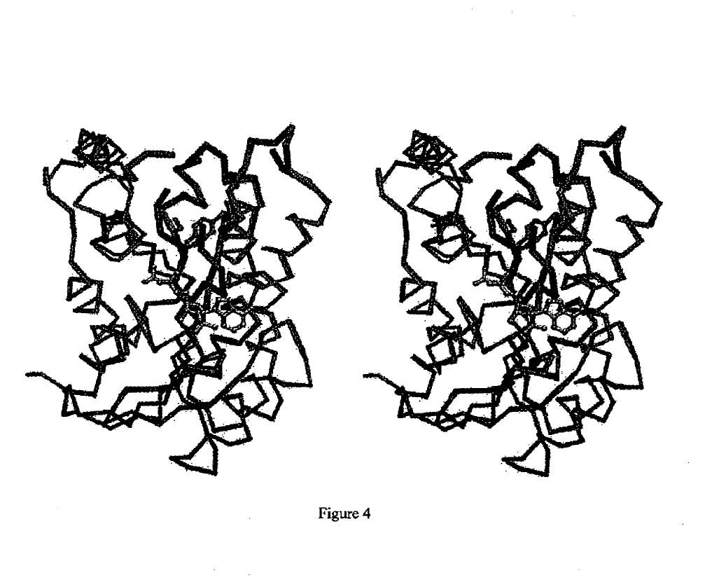

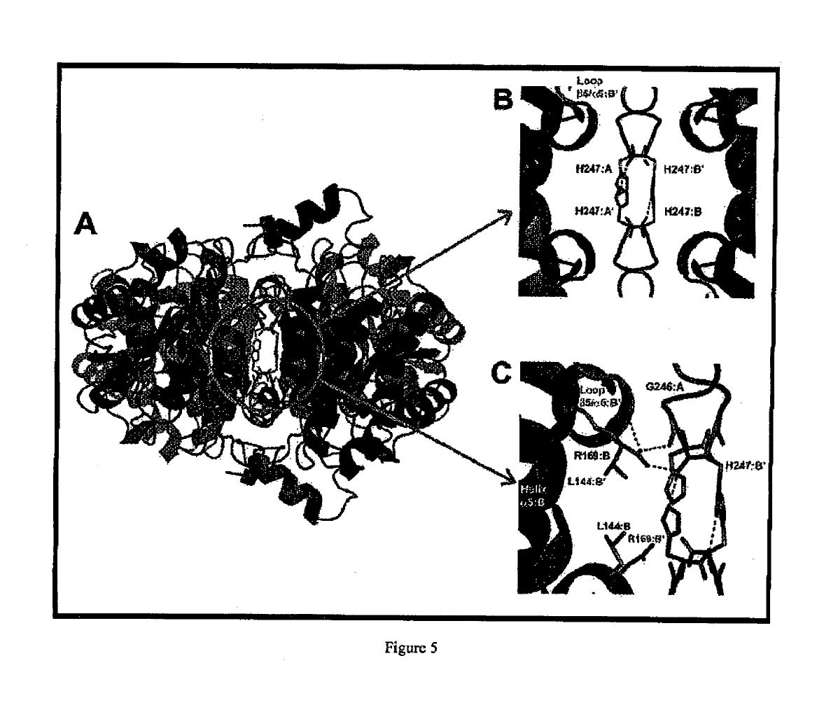

The structure of the crystals obtained in the presence of a high concentration (50-100 mM) of oxidized cofactor (NADP) was solved at a 1.91 Å resolution and displayed an “open” form of MabA-C60V/S144L (FIG. 2). This structure is similar to the structure of holo-KARbn and the structure recently solved of holo-KARec (19). Here, the region between strand β6 and helix α6 (residues 190-210 in MabA) showed little change compared to the “closed” conformation. On the contrary, in both monomers clear density appeared to trace both the backbone of residues 94 to 99, 142 to 149, and the very end of the C-terminus (245-247). During the last steps of refinement, the use of TLS (23) improved the overall agreement of the model with the crystallographic data. Meanwhile, clear but only partial density appeared for the NADP Molecule (FIG. 3). Although the adenosine part and the three phosphate groups are visible, the nicotinamide moiety and the corresponding ribose appeared disordered. Some rearrangements of the nicotinamide moiety have been recently described in several SDRs (e.g., PDB1IY8), while in the present case local and extreme mobility seemed to occur. The visible part of the cofactor appeared to adopt a conformation observed in other SDRs. This conformation is stabilized by various interactions including a highly conserved hydrogen-bonding network involving the aspartate D61, the glycine-rich loop β4-α4, and asparagine N88. The additional 3′ phosphate group appeared to interact with the side chains of two arginines R25 and R47.

The knowledge of the two distinct conformations (“closed” and “open”) of MabA-C60V/S144L were then used to solve the structure of MabA-wt at a resolution of 2.00 Å cocrystallized in presence of 50 mM of NADP. The two self-excluding conformations of both loops β4-α4 and β5-α5 were observed simultaneously in both monomers, revealing the coexistence of both open and closed forms within the same crystal. Compared to the double mutant, weaker density corresponding to the cofactor appeared at the end of the refinement of the structure, so the cofactor was not modeled.

During structural analysis of the various forms of MabA-C60V/S144L, as in the previous structures of MabA, sharp peaks appeared on the electron density map at the interface of the two symmetry-related monomers. The peaks were attributed to cesium ion with full occupancy. The positively charged mono cations were found in two different environments. In the first type of site, the cesium ion showed interactions with an aromatic ring (side chain of phenylalanine FI3), a backbone carbonyl group and water molecules as already observed. In the second environment, only main-chain carbonyl groups and water molecules stabilize the cesium cations (15). For both forms, the N-terminus of the protein (residues 1-8), as well as the fusion peptide bearing the polyhistidine tag, were not visible in the electronic density.

Common Core Analysis

As observed in solution for KARs from plants (30), MabA, KARec, and KARbn all form a tetrameric structure in their crystal forms (15, 17-19). The tetrameric structure is well conserved in both the “open” and “closed” conformations of MabA. This asymmetric-unit dimer of MabA is stabilized by a large interface comprising the β7 strand. The second dimer interface is composed of the two helices α4 and α5. Relatively small RMSD values were measured between the “open” conformation of MabA (including the two active site loops β4-α4 and β5-α5) and the crystal structures of KARbn (RMSD of 0.5 Å over 180 Cα) and various other reductases (e.g., PDB1YBV). The superposition over a common core of 112 Cα carbons (40% of the structure), of MabA and InhA (PDBIBVR) showed a RMSD of 1.4 Å, despite the low sequence conservation (20%). The conformational changes induced upon cofactor binding seemed to be restricted to the active site and the neighbouring C-terminus (see below) rather than involving the overall structure of MabA. This result is in agreement with our crosslinking data in the presence and in the absence of cofactor (15).

Ligand-Induced Fit

In the “closed” apo-form of MabA, several rearrangements affect the structure of the active site compared to the “open” holo-forms of SDRs. Some residues could not be seen in the electron density of the “closed” conformation, even at high resolution (see above). This suggested that regions β4-α4 (residues 94-99) and β5-α5 (residues 144-147) are highly disordered. A similar situation was observed in the crystal structure of KARec. On the contrary, in the holo-form of the related KARbn and KARec, the corresponding residues are well ordered in the crystal. In other SDRs, such as the apo-form of the dimeric 3α-hydroxysteroid dehydrogenase from Comamonas testosterone, these regions remain in the same conformation whether the cofactor is present or not (31).

In the “open” form the loop (α5-β5) adopts the conformation predicted by similarity with the holo-form of KARbn (see above, FIGS. 2 and 4). The strand β4 extends to residue D94, whose side chain is in close contact with glutamine Q150. In agreement with previous modeling studies (15), the side chain of the leucine at position 144 is brought to close contact with isoleucine I161 (distance Cγ-Cδ1 4.2 Å). Furthermore, the hydrophobic side chain of residue 144 is also surrounded by two hydrophobic side chains (A158 and V236) while lying in the vicinity of the C-terminal histidine H247 from a second monomer (distance C82-C82 5.9 Å).

The switch from the “closed” to the “open” conformation also makes the phenol ring of the catalytic tyrosine Y153 rotate by 90° , and it points into the active site like in KARbn (15, 19). This reorientation is induced by the rearrangement of the catalytic serine (S140 in MabA). Serine S140 and valine V141 are moved by roughly 5 and 8 &Arin; (Cα-Cα distance), respectively. In the apo-form, residues S142 and G143 are in contact with the tyrosine Y185 closing the active site. This particular rearrangement prevents entrance of the ribose and the nicotinamide ring of NADP (15). This suggested that the overall rearrangement is induced by the empty space normally filled by the cofactor. Such a structural rearrangement is consistent with our fluorescence study that showed significant changes of the tryptophan W145 environment upon cofactor and ligand binding (14). In the “open” form, tryptophan W145 and isoleucine I147 are brought into the substrate-binding site. The tryptophan side chain is sandwiched by the side chains of methionine M243 and arginine R169 from the crystallographically related another monomer. A hydrogen-bonding network involving two water molecules connects the same residues through their side-chain amino group (Ne1 for W145 and Nz1 for R169) or main-chain carbonyl group (e.g., M243 and G246) with the symmetry-related equivalents (FIG. 5). The hydrophobic environment in the active site cavity made up by the tryptophan indol ring and the neighboring methionine M243, and isoleucine I147 might correlate with the unusual specificity of MabA for long-chain substrates (14). Unexpectedly, the two C-terminal residues (G246 and H247) clearly appeared in the electron density of the “open” conformation of both the double mutant and the wild-type enzymes. These two amino acids are not visible in the “closed” conformation. The C-terminus of MabA (motif“GGMGMGH”) is buried at the tetrameric interface in a unique way [FIG. 5(A)]. Although the C-terminal tails in related SDRs are solvent exposed, the four C-terminal segments are in close contacts (distance G246-G246′ approx 4.6 Å). The histidine (H247) side chain from one monomer points toward the main-chain carboxyl group of a histidine of another monomer at hydrogen-bonding distance [FIG. 5(B)]. This conformation is also stabilized by favorable interactions with neighboring residues including R169 via a salt bridge with the C-terminal carboxyl group [FIG. 5(C)]. Each arginine R169 side chain formed a hydrogen bond with the carbonyl of residue 144 of a symmetry-related monomer [a serine in wild-type MabA, substituted in MabA-C60V/S144L; FIG. 5(C)]. In the apo-form, this C-terminal conformation is no longer stabilized due to the flexibility of the loop.

If the stabilization of the segments α5-β5 and the C-terminus in the holo-form of MabA seems to be concomitant, the orientation of the very end of the C-terminus of MabA suggests that this segment is not involved in substrate recognition, although it comprises a sequence motif specific to mycobacterial MabA.

Ligand Docking and Substrate Specificity

The structure of MabA holo-form with a complete cofactor was modeled, using the structures of complexed “open”MabA and of holo-KARbn in binary complex with NADP (17). In the holo-forms, the nicotinamide moiety of NADP, involved in hydride transfer during catalysis, points deeply into the substrate binding site. The entry of the cofactor requires only little rearrangements of the segment T188-M190. The threonine residue belongs to a sequence motif (PGxxxT) specific to the SDRs and it is expected to be hydrogen bonded to the amide group of the nicotinamide through its side-chain hydroxyl. In NADP-bound MabA, the segment P184-M190 adopts a conformation similar to that observed in holo-KARbn and other SDRs (21). However, the hydroxyl group of threonine T188 in MabA appeared slightly too far away (approx 3.8 Å) from the modeled cofactor amide group, in agreement with the absence of ordered nicotinamide.

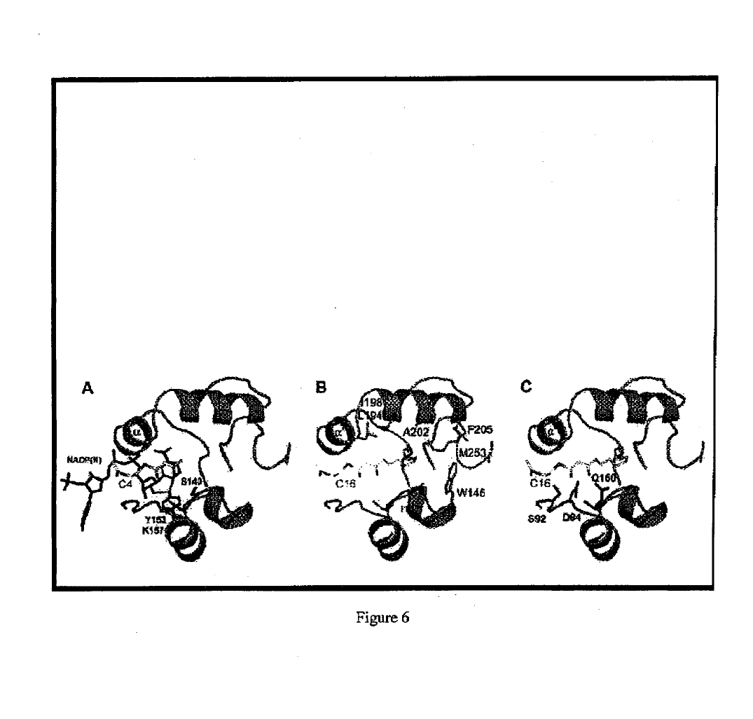

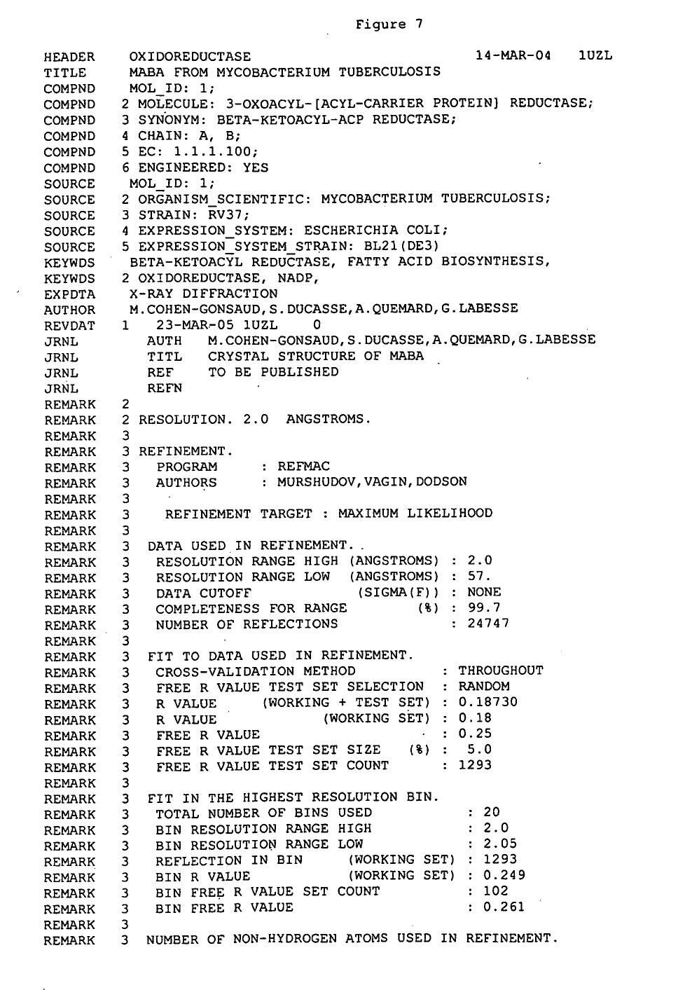

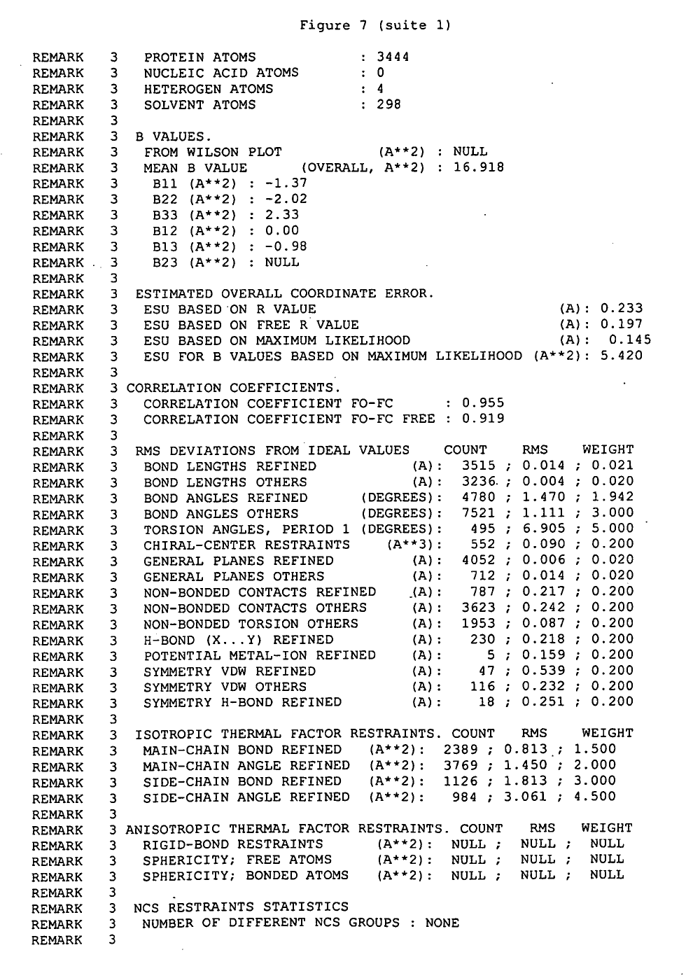

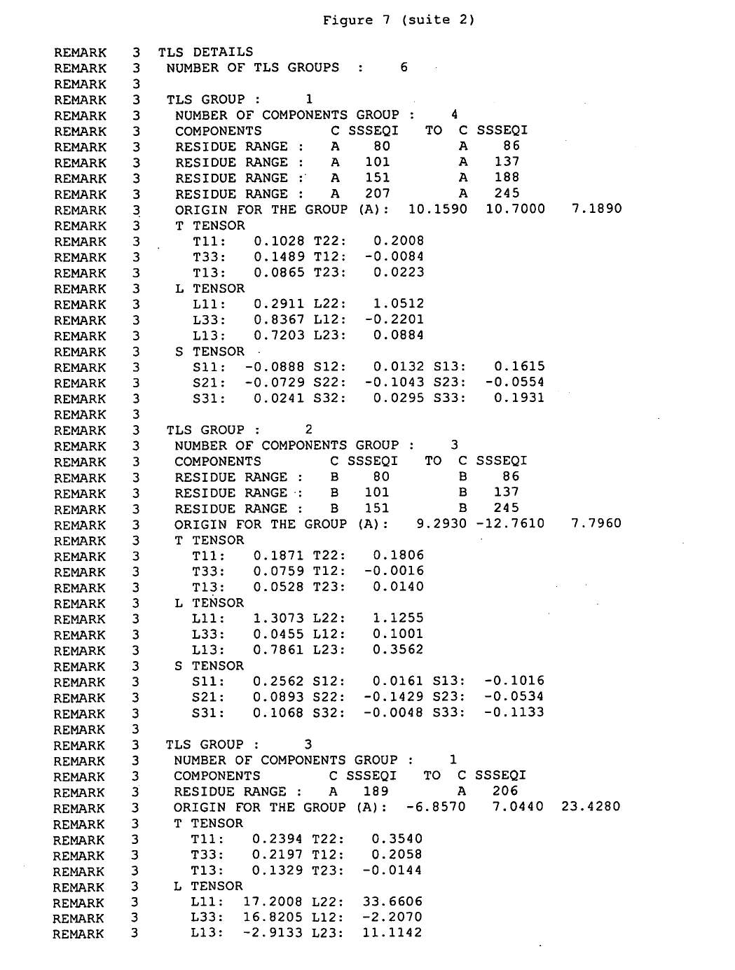

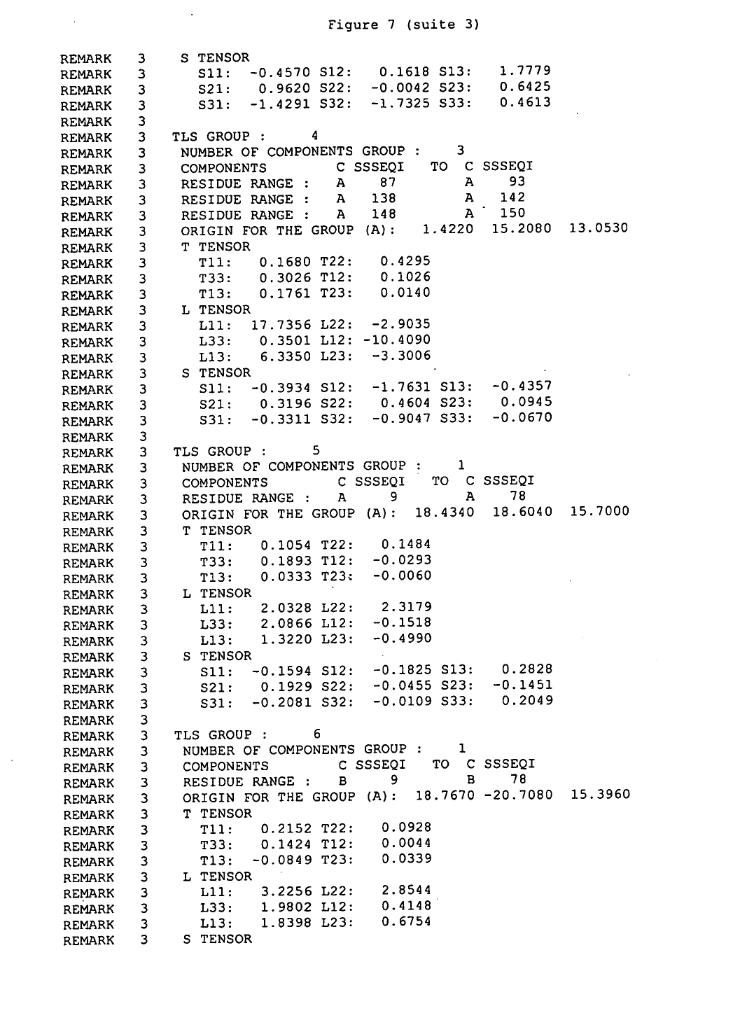

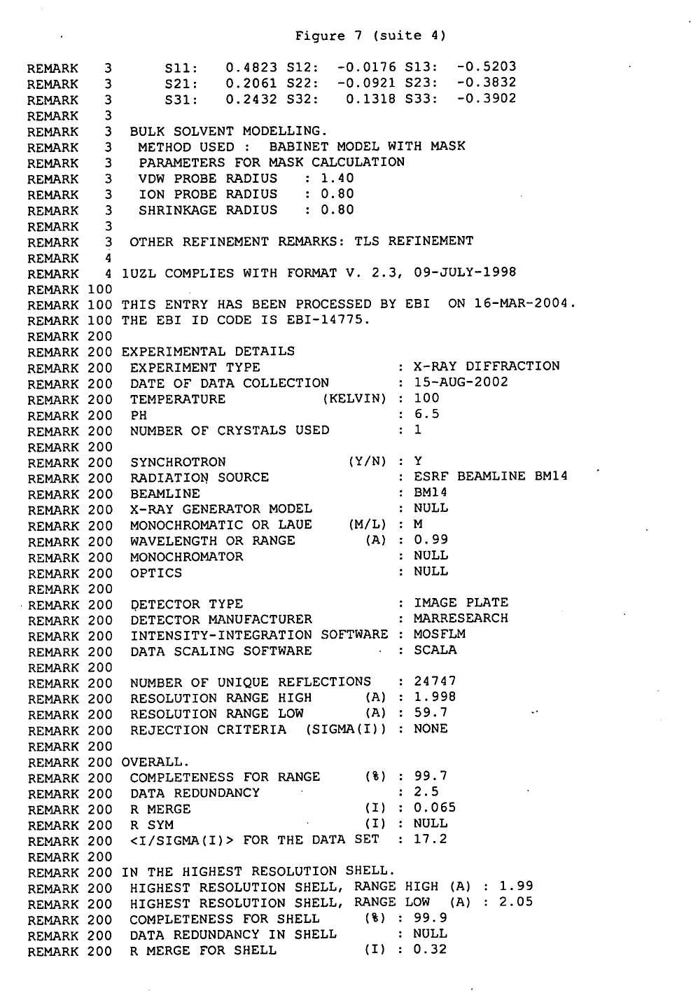

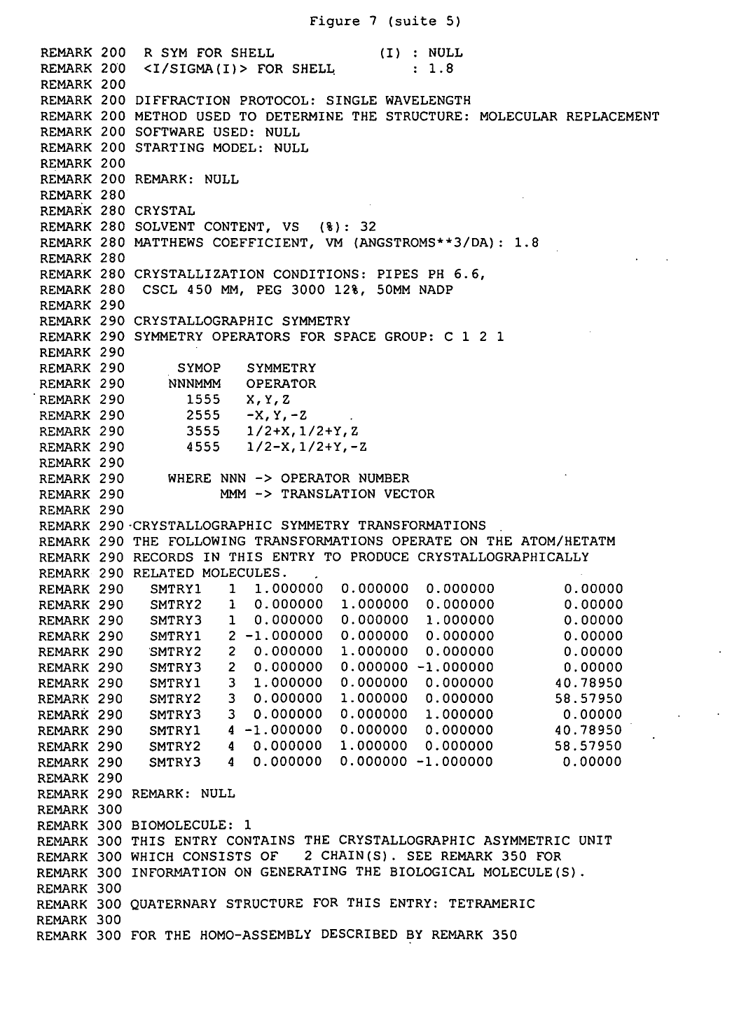

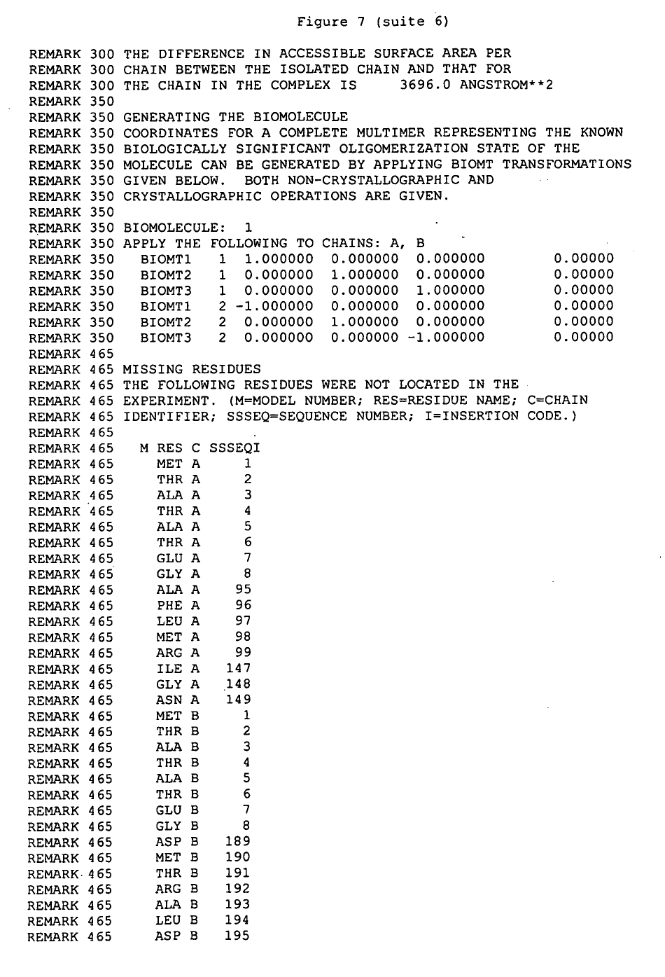

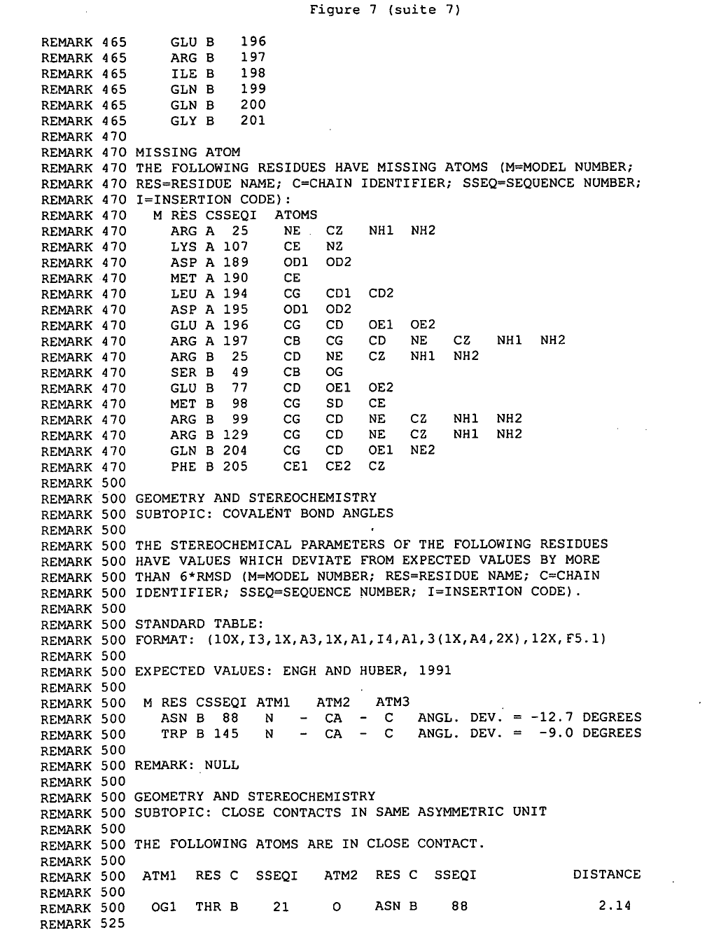

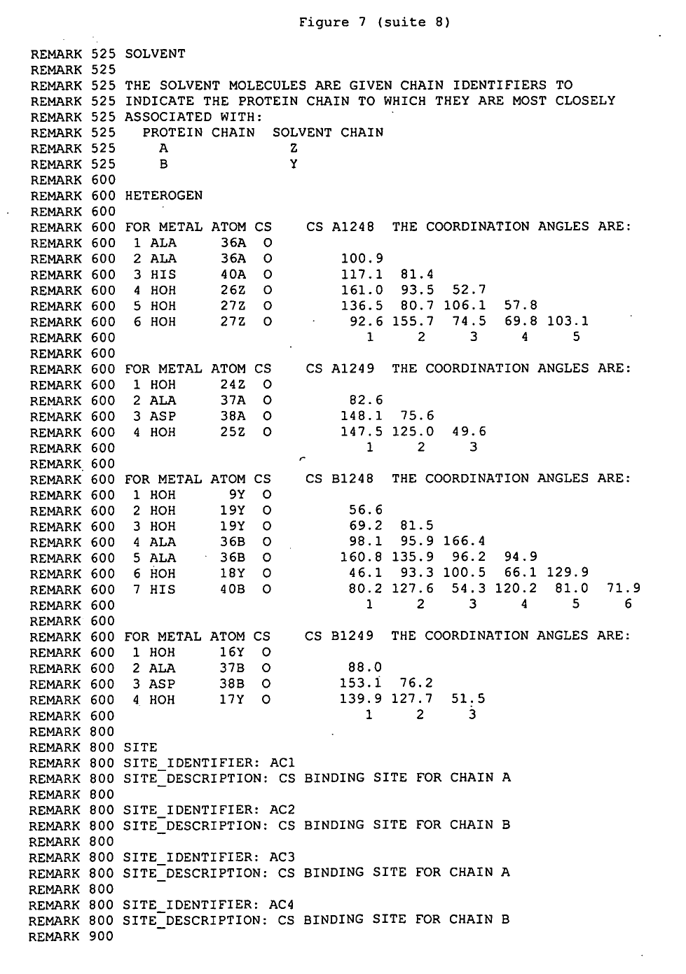

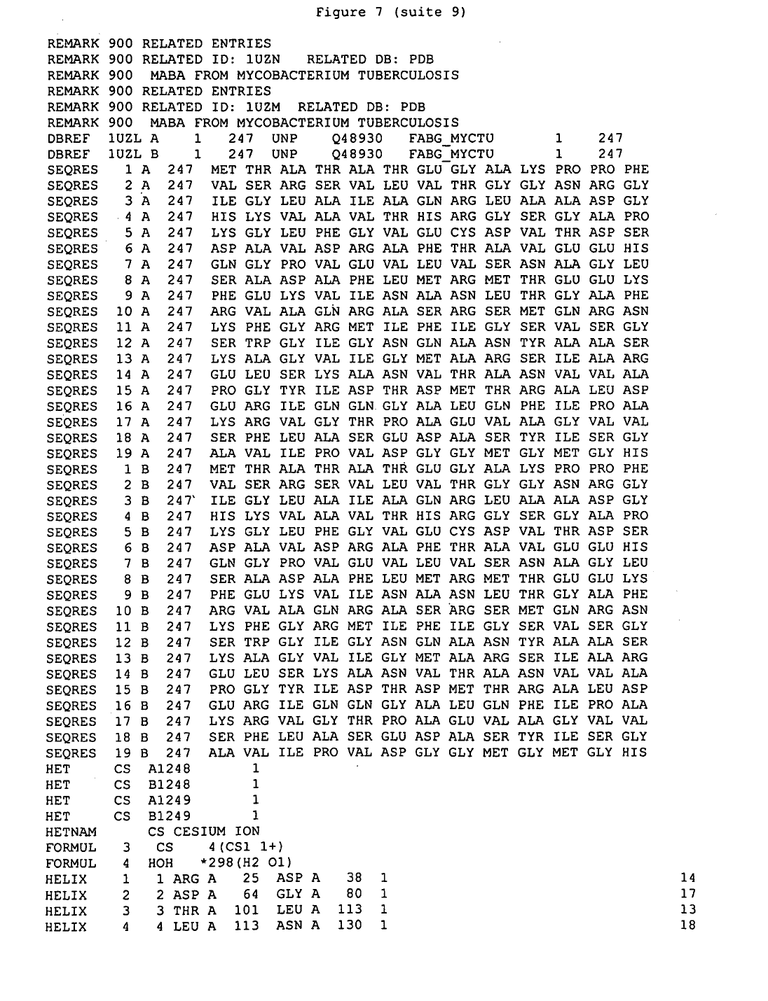

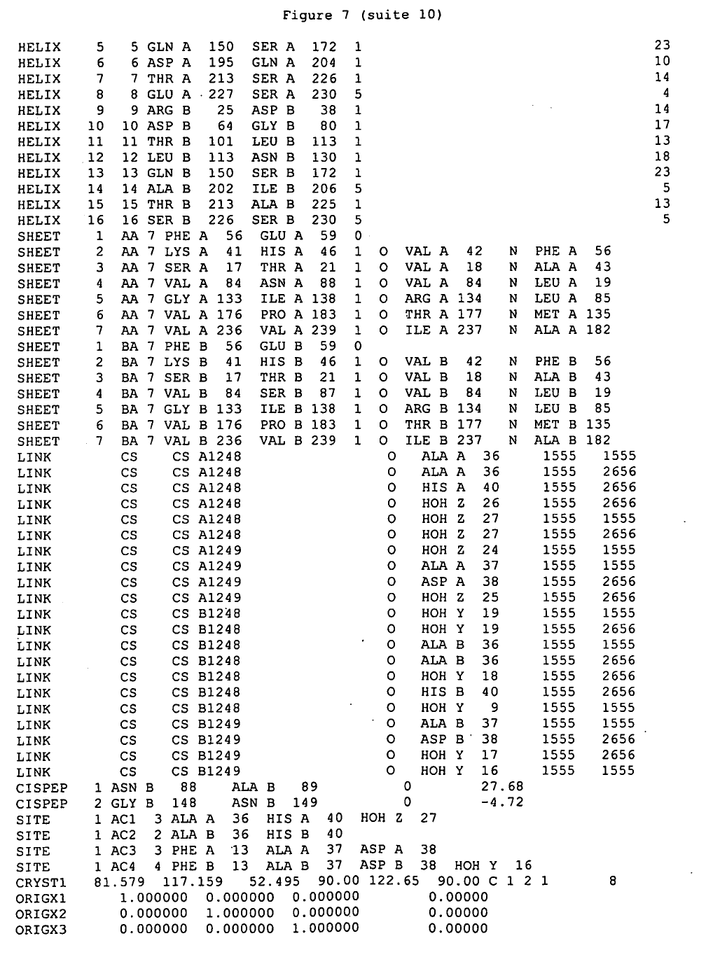

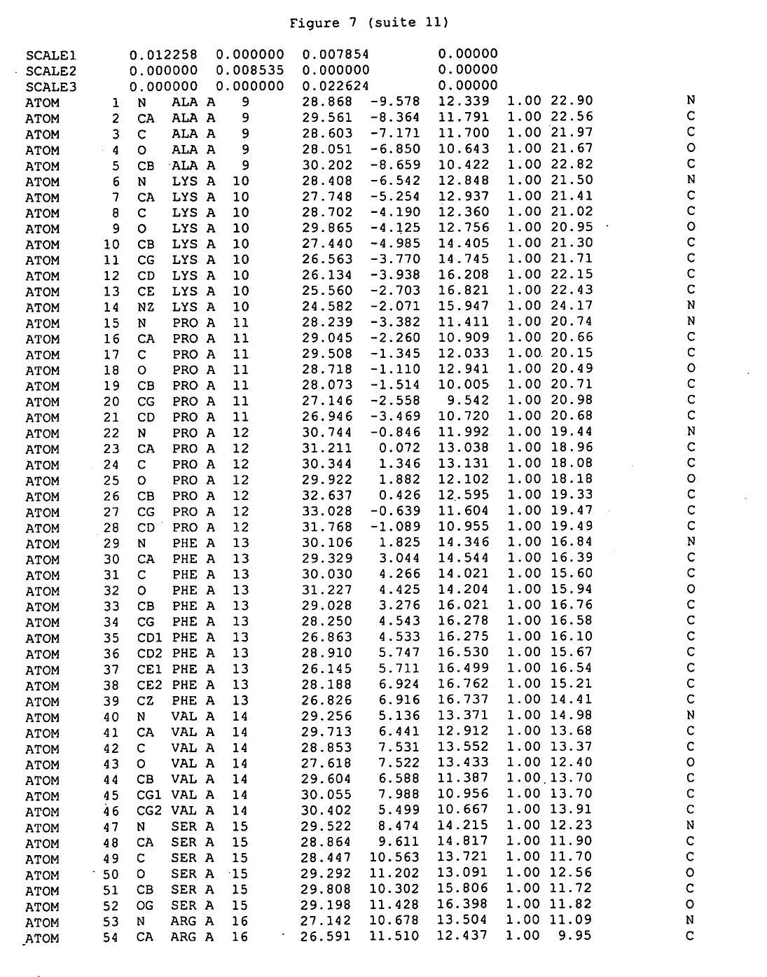

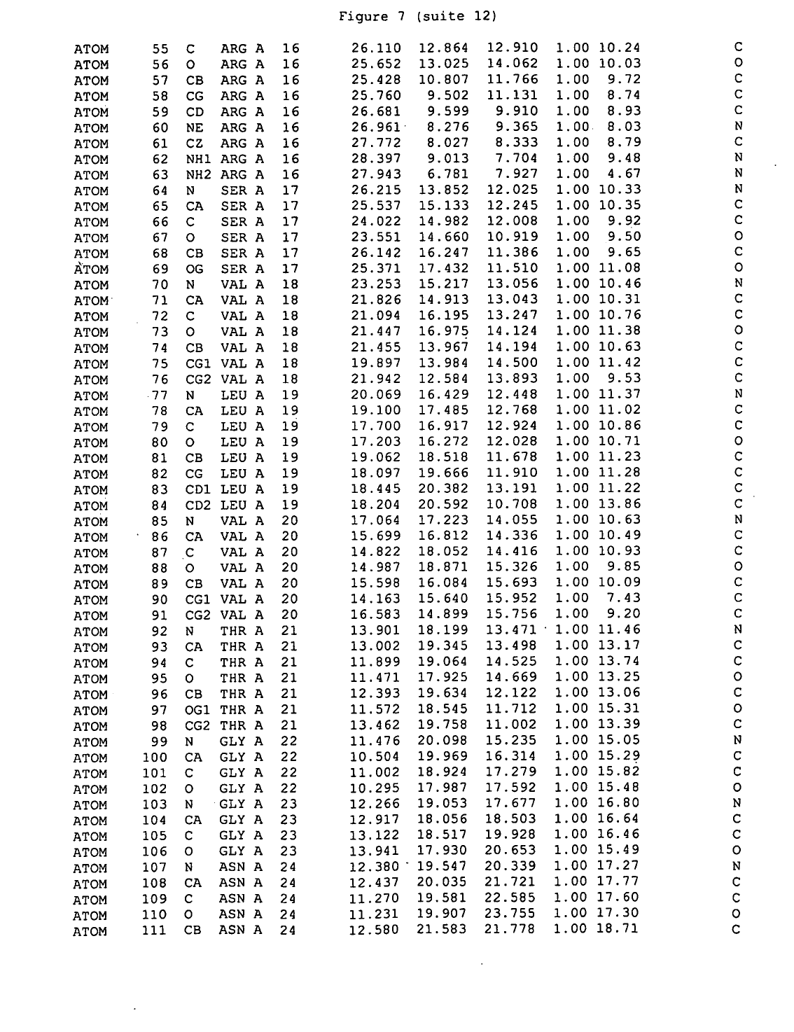

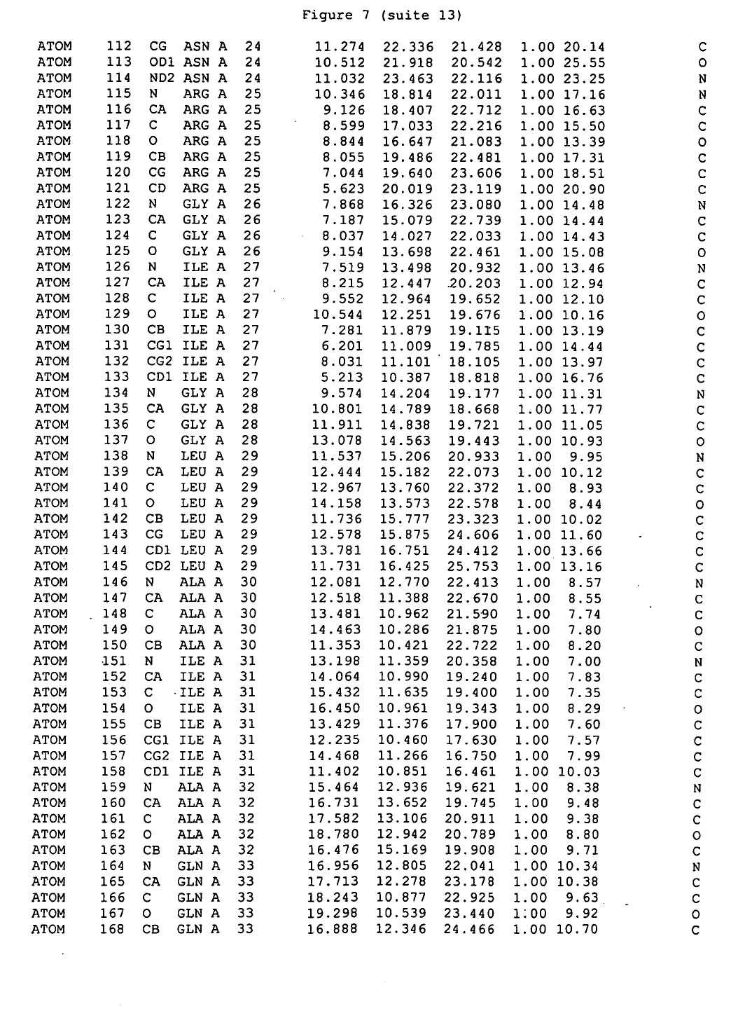

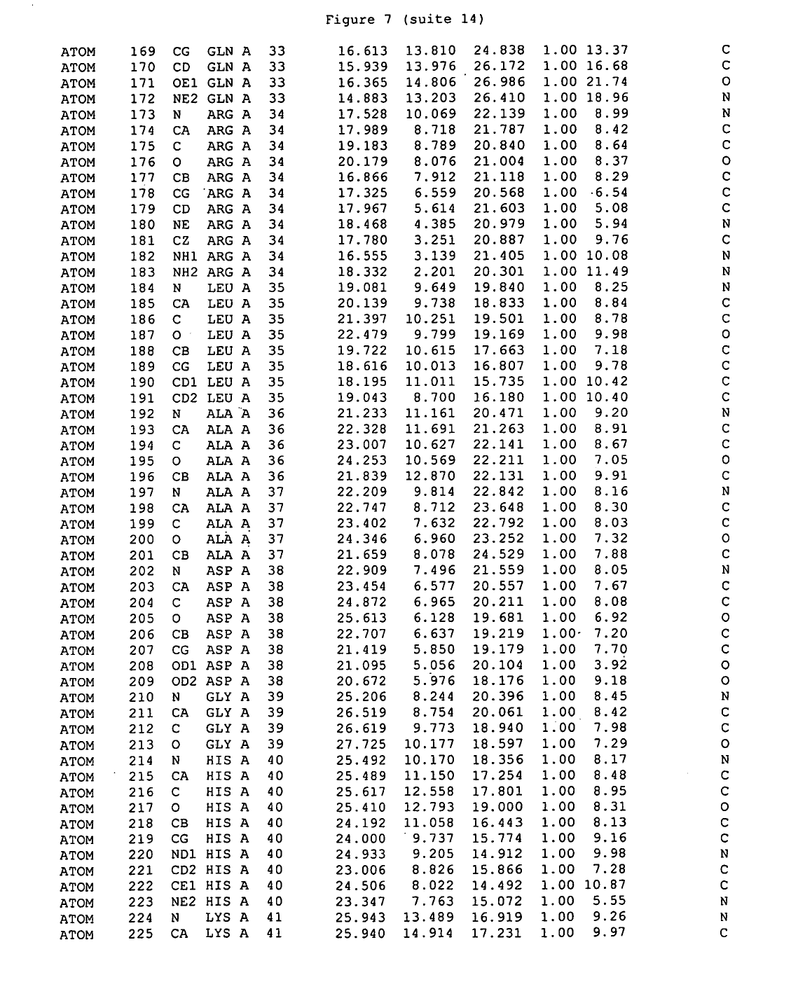

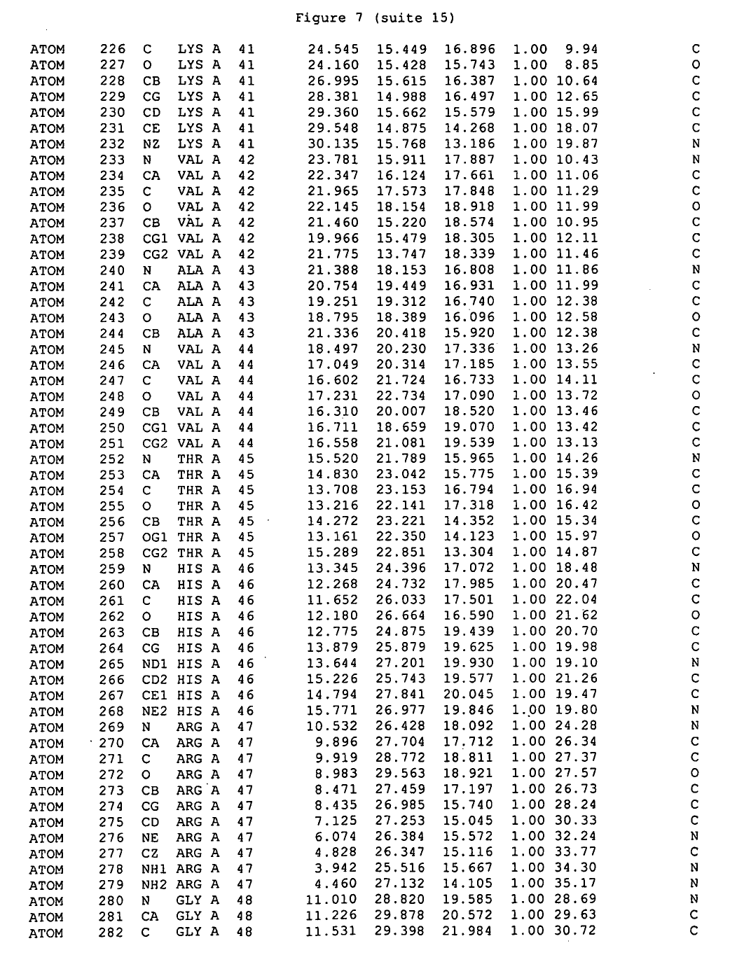

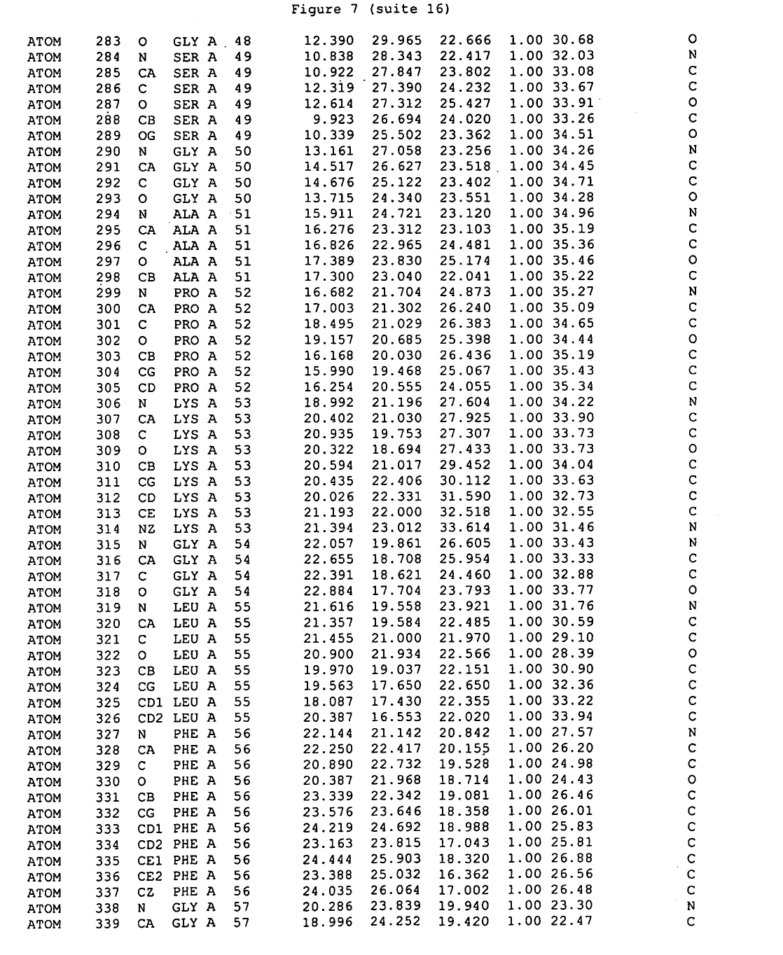

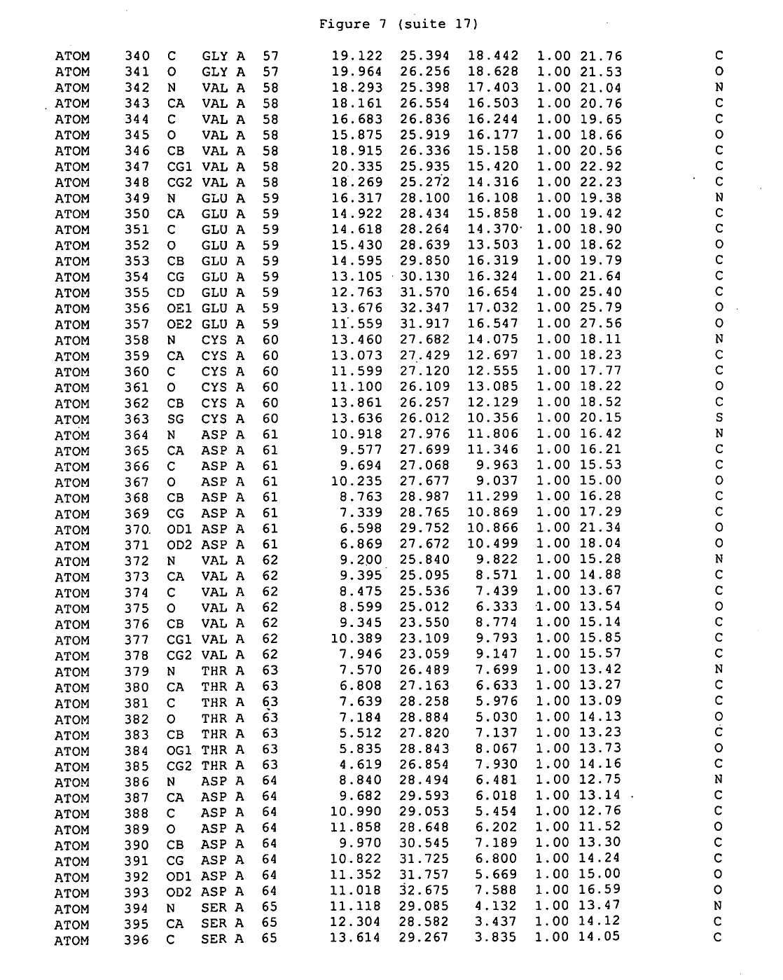

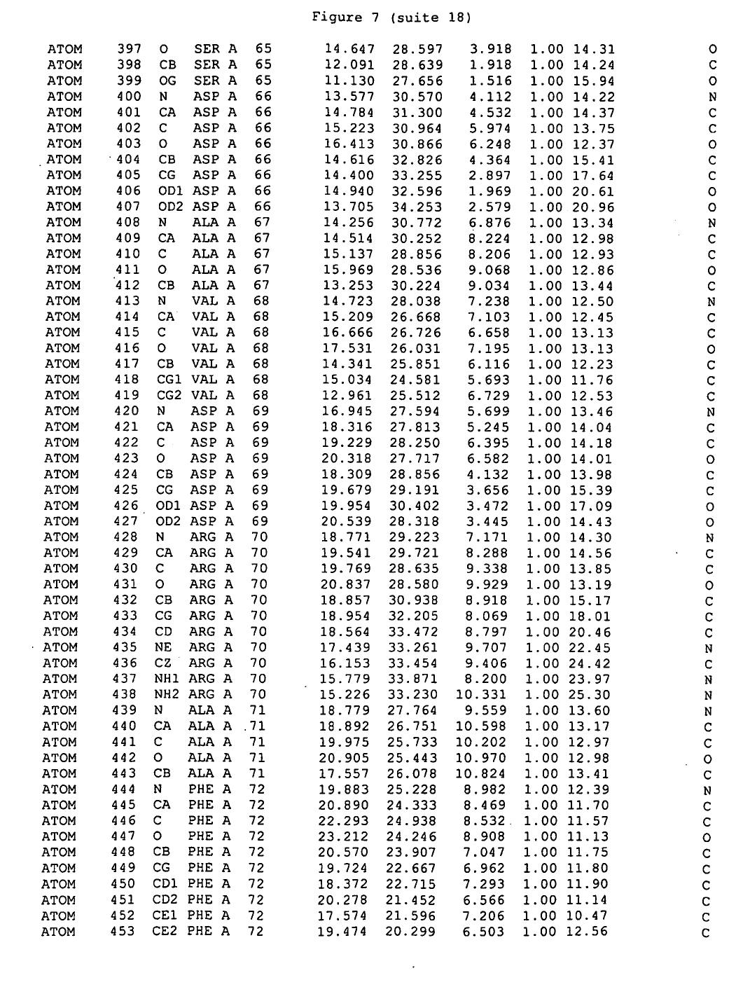

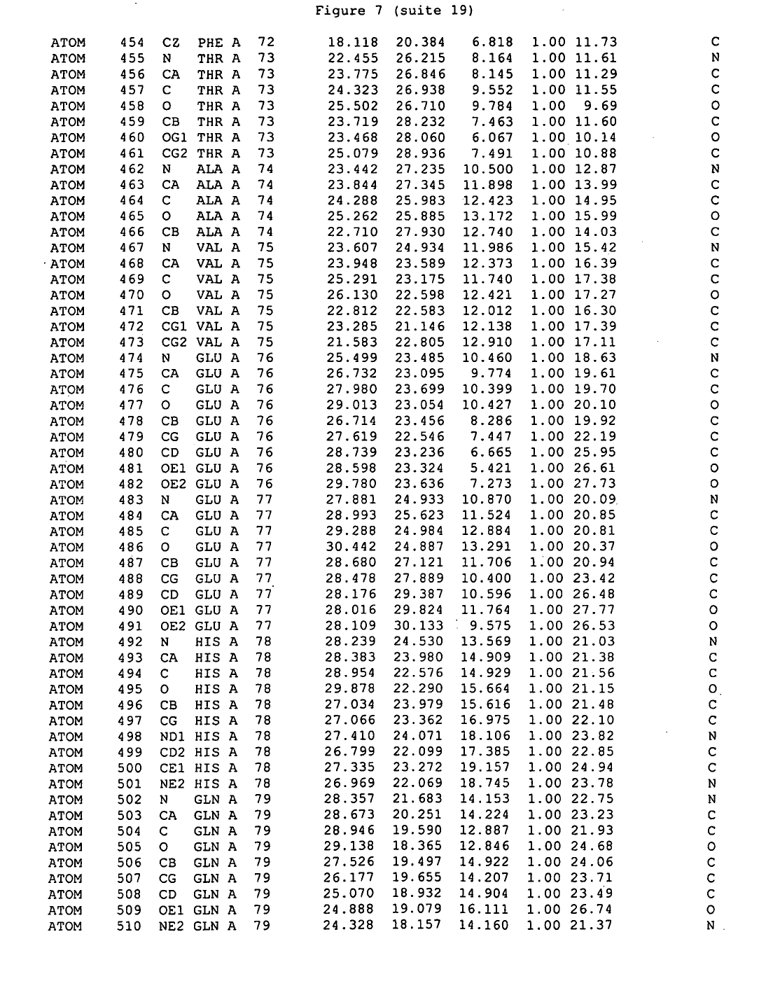

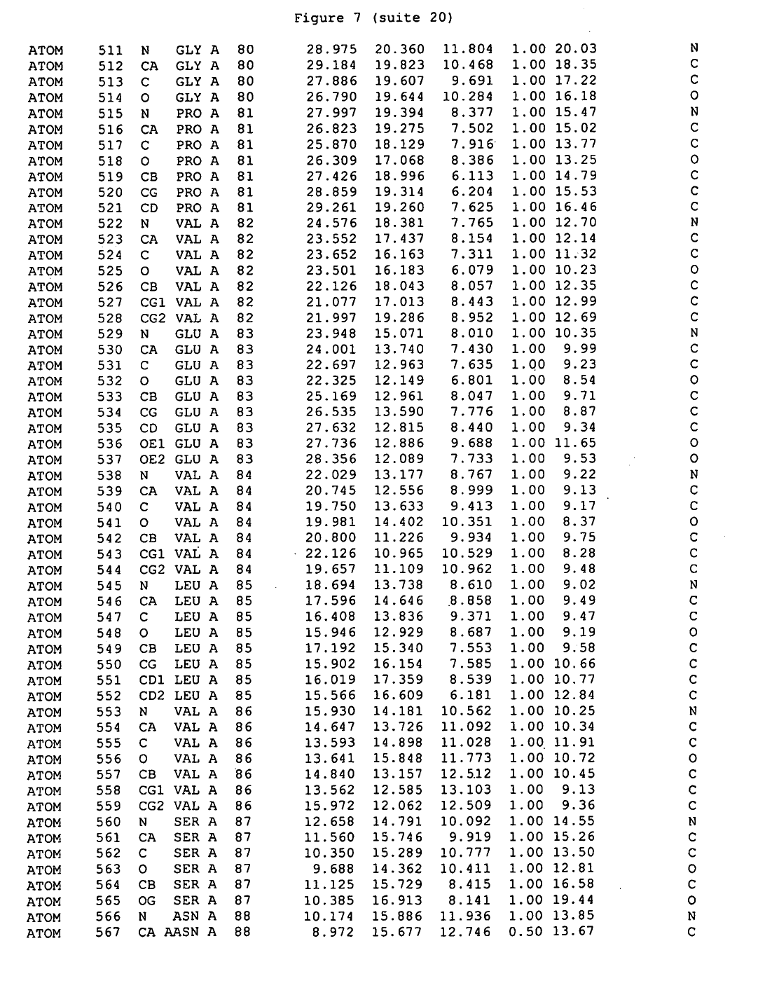

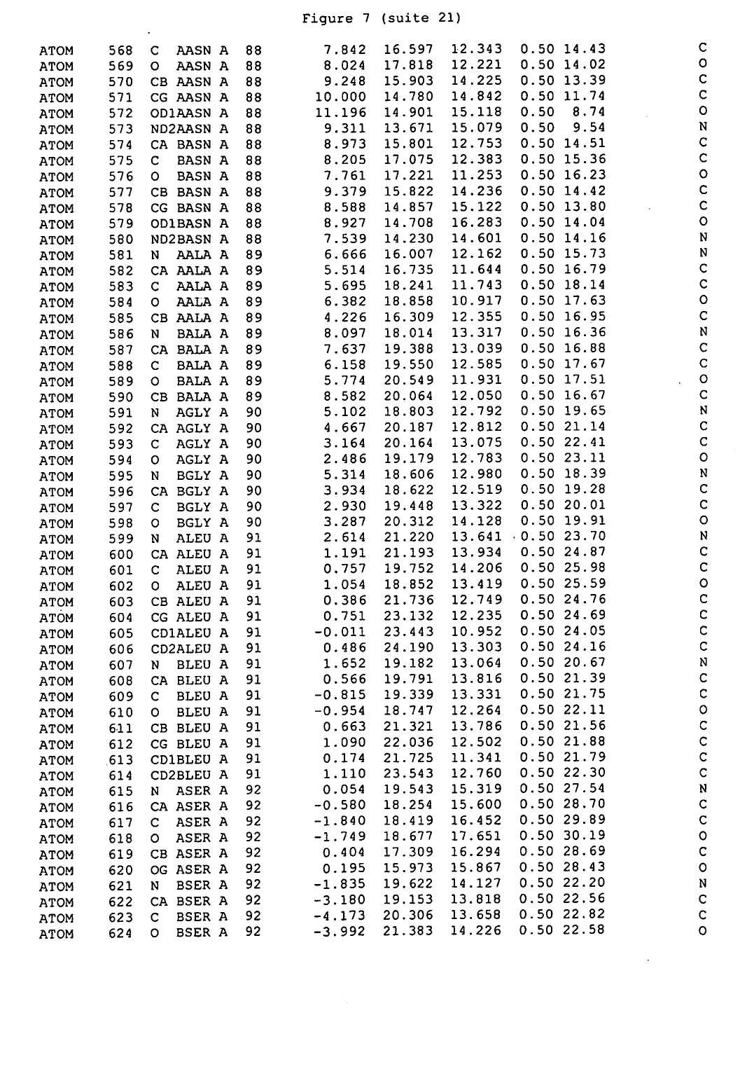

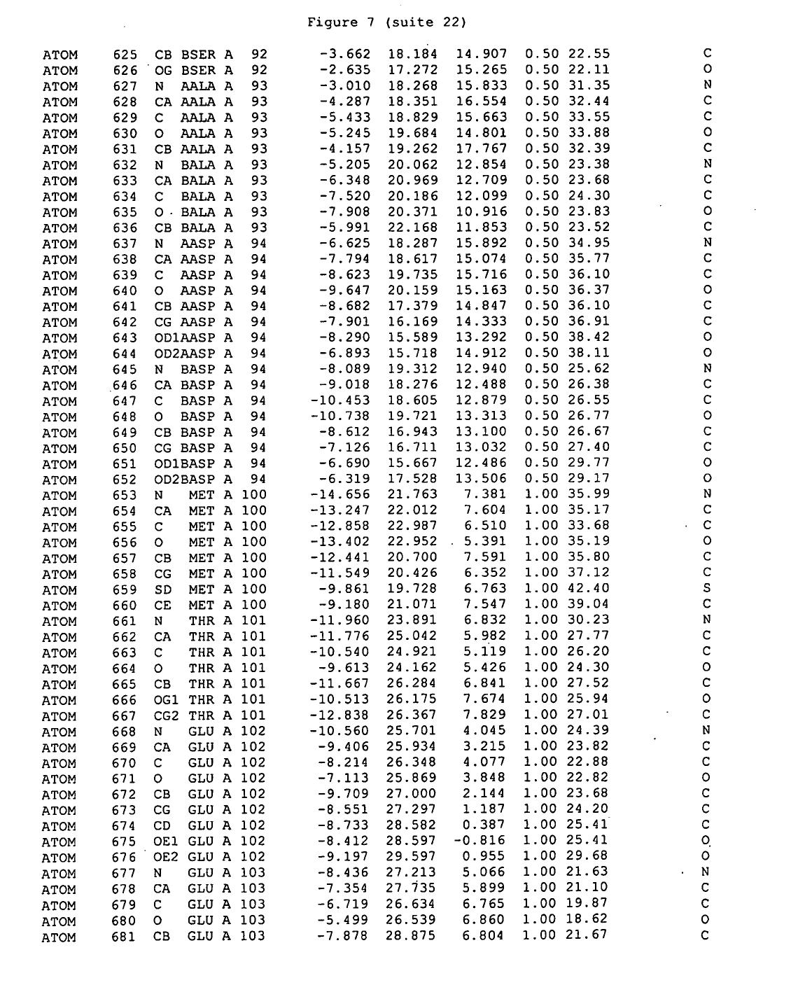

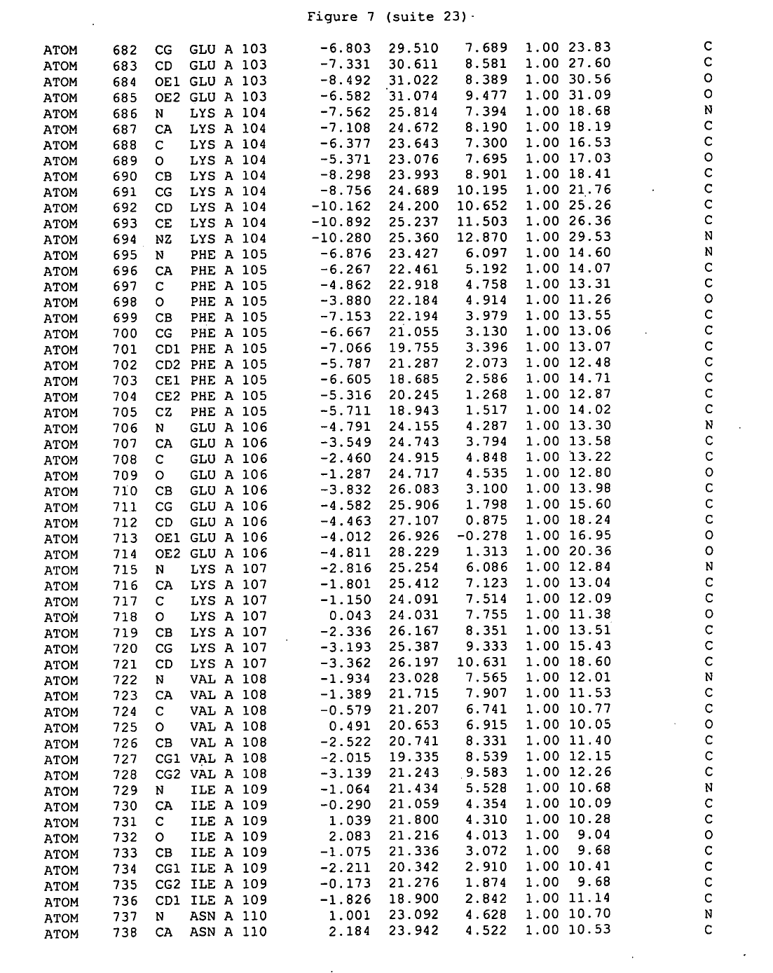

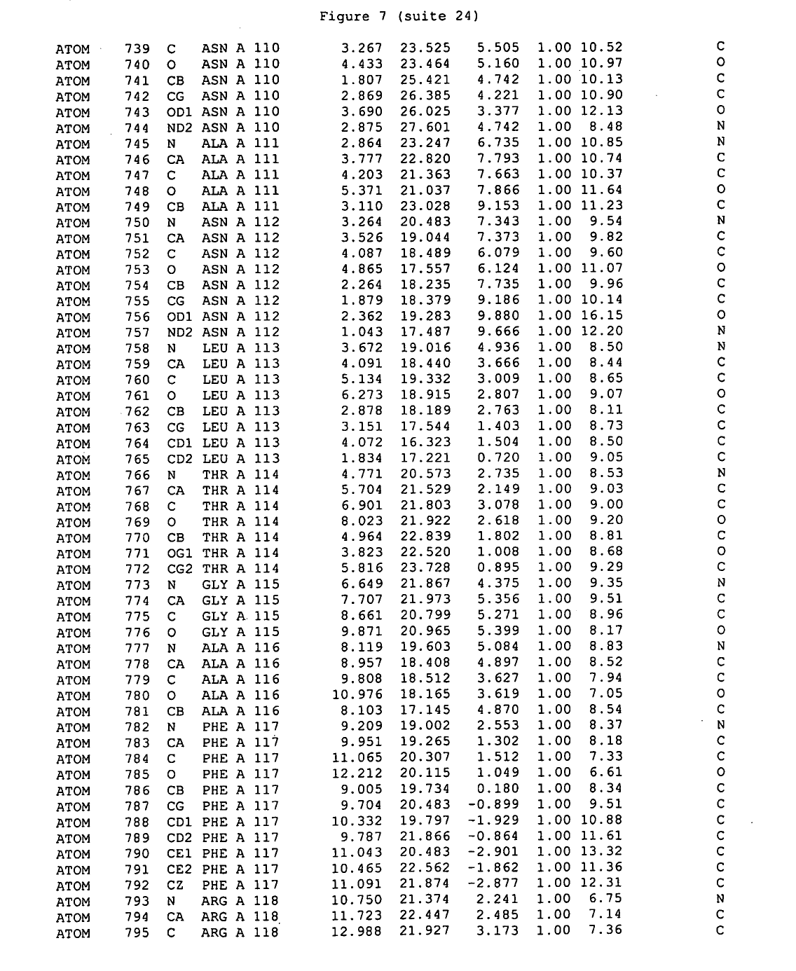

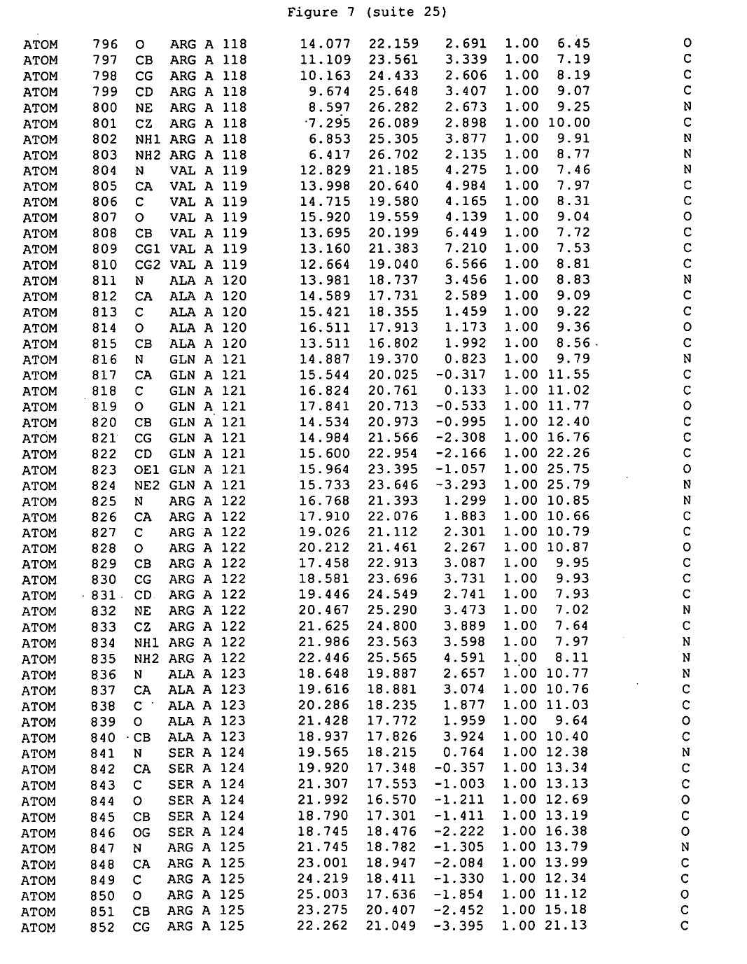

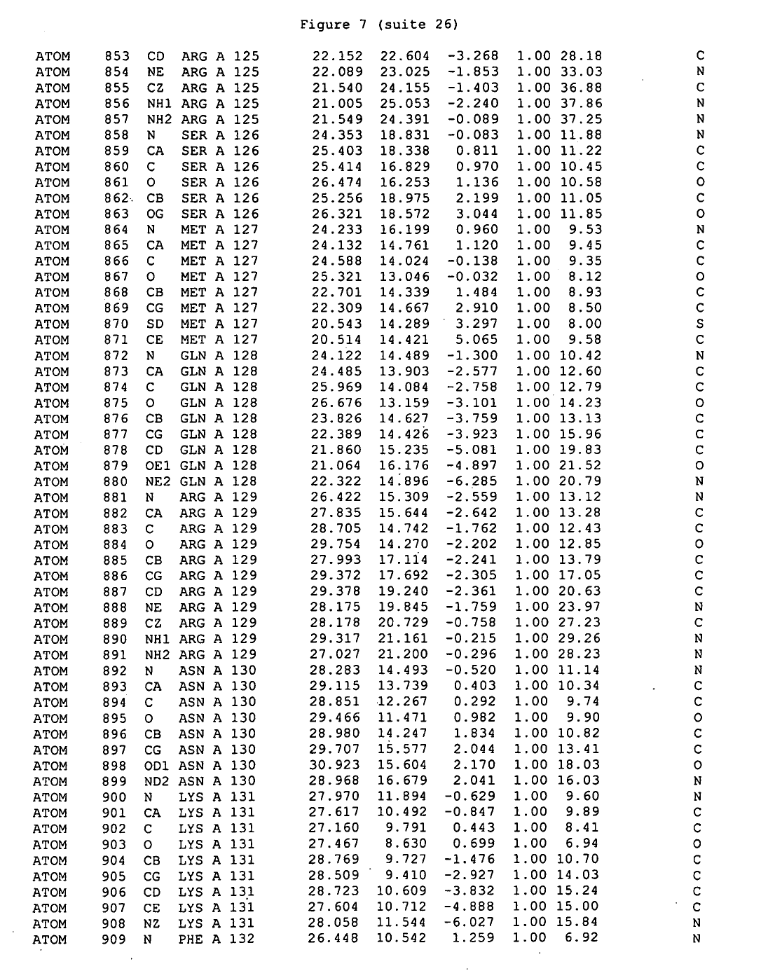

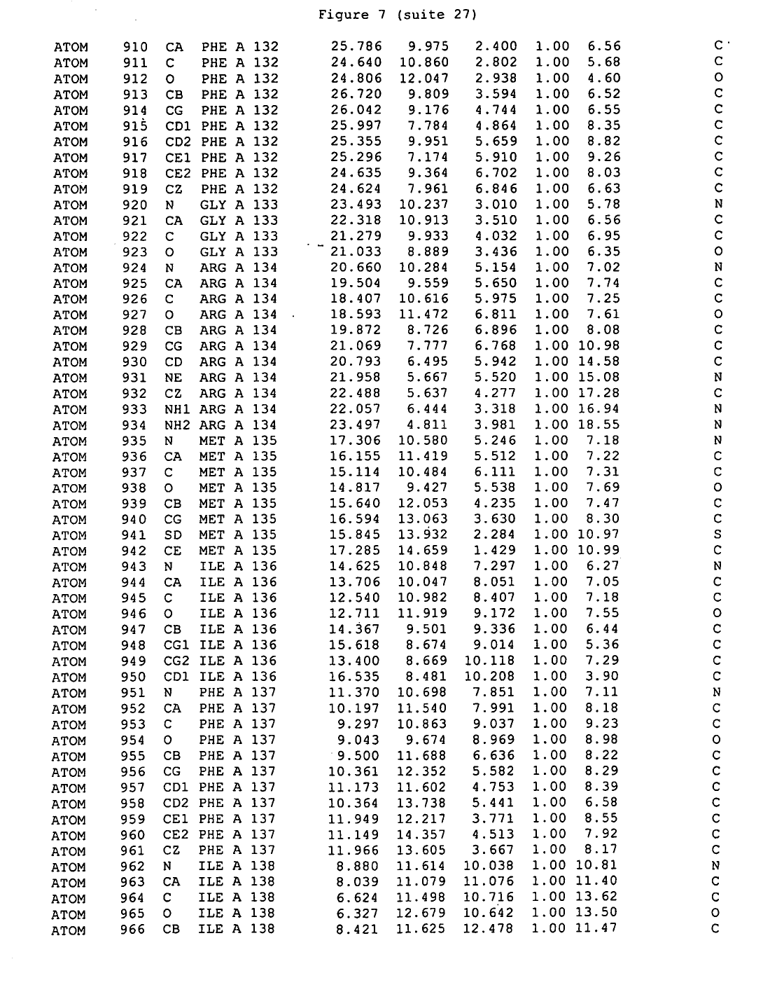

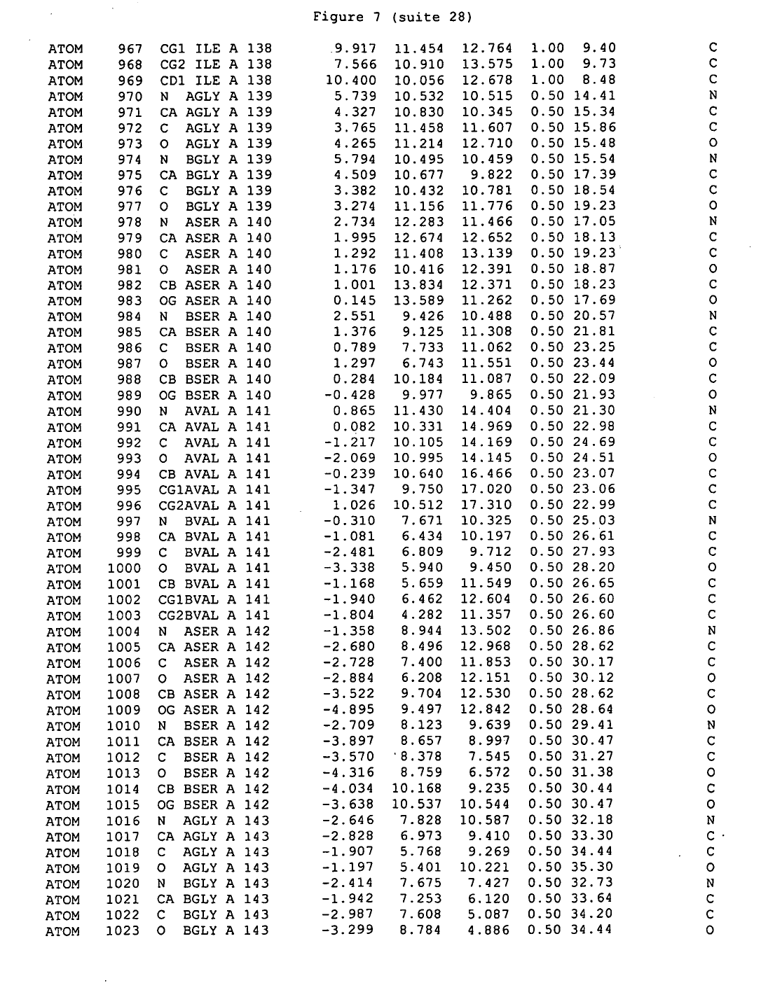

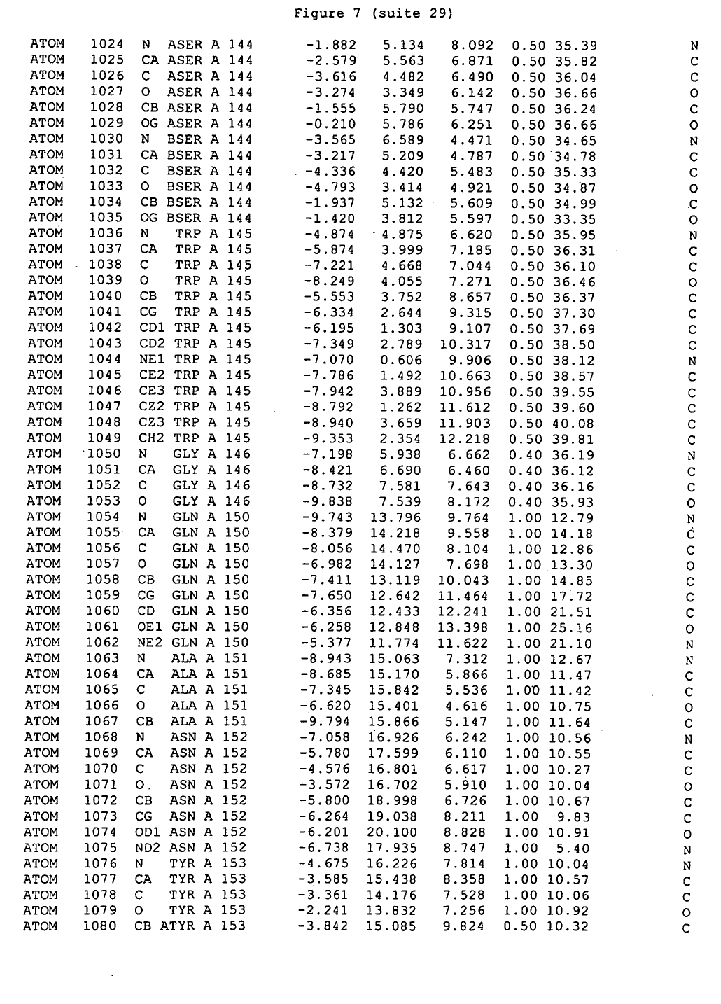

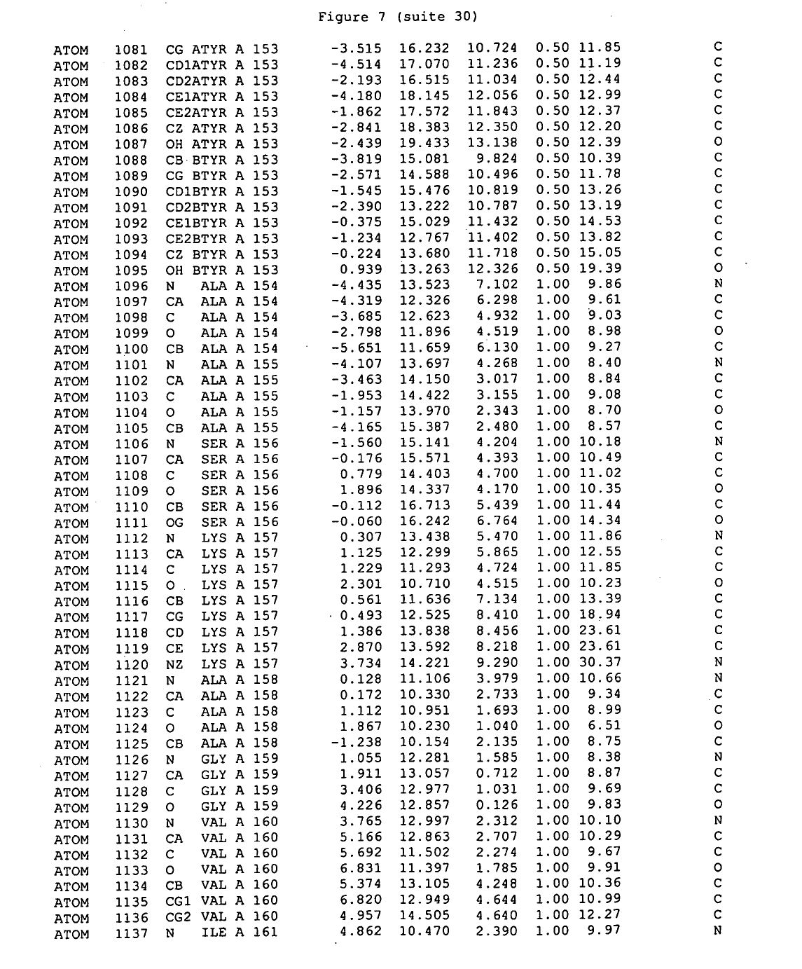

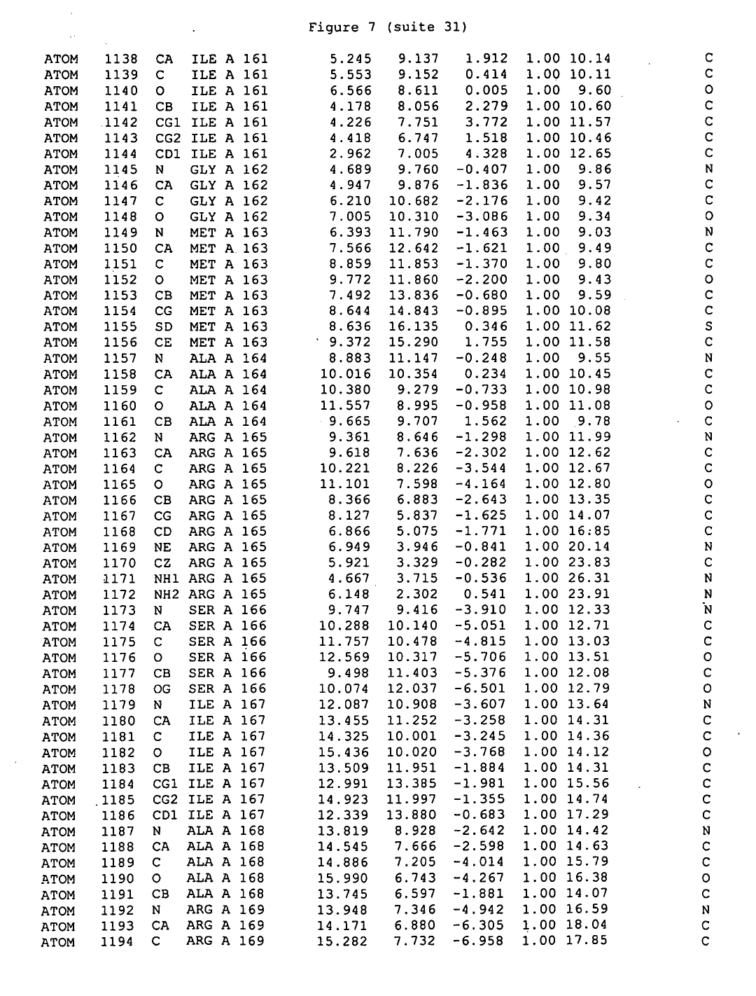

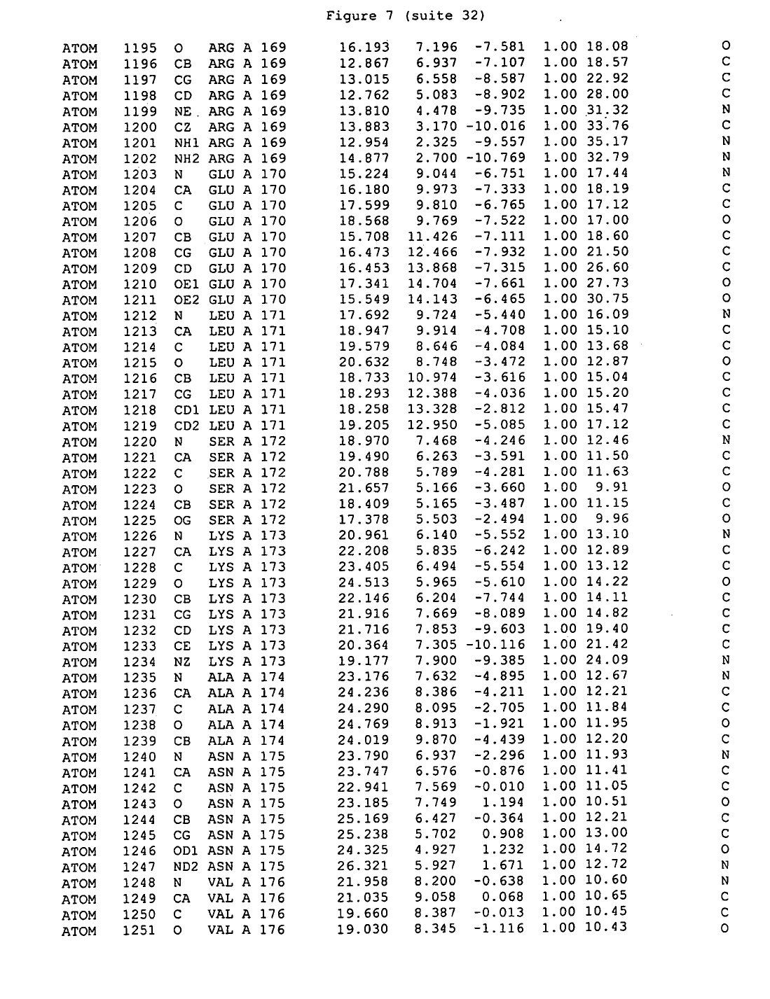

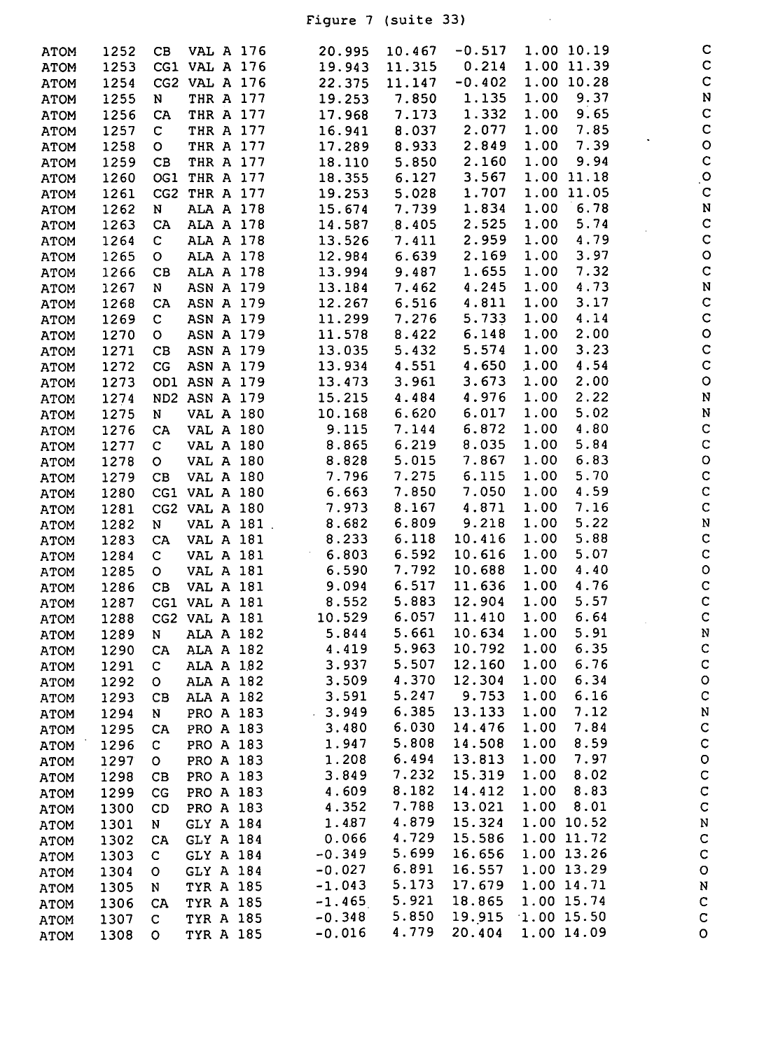

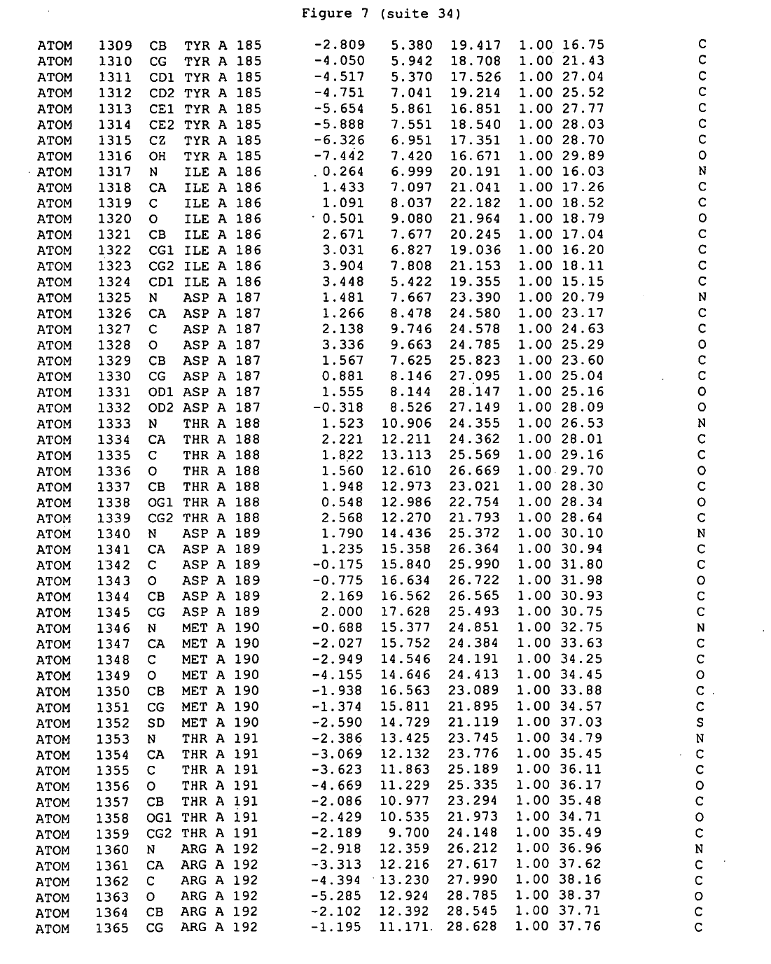

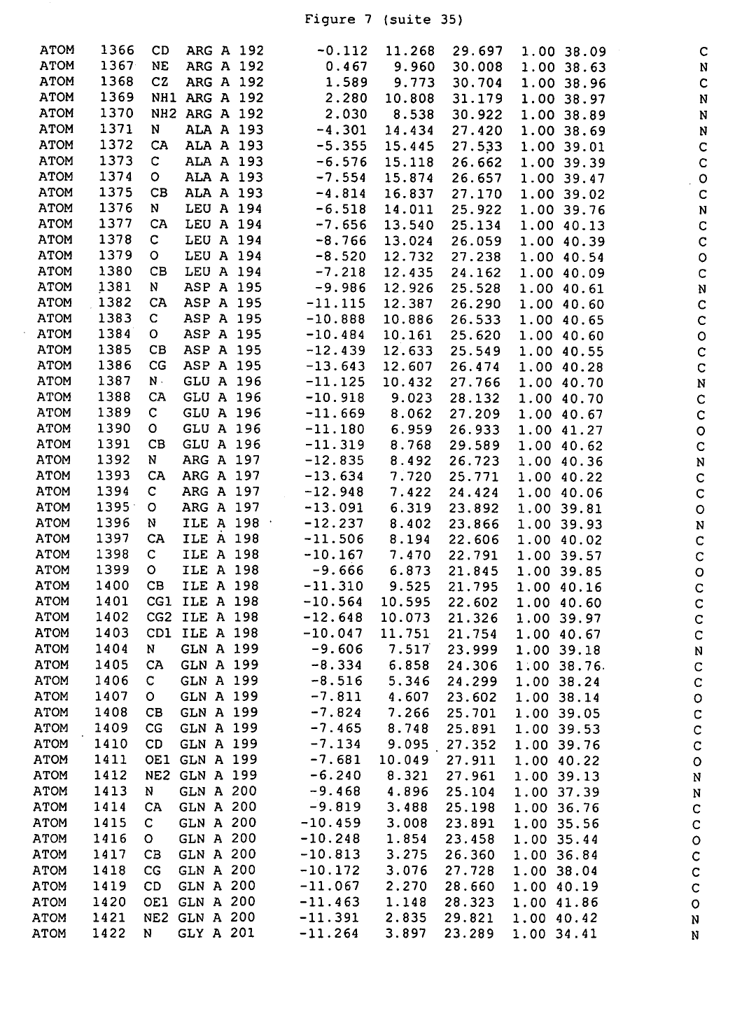

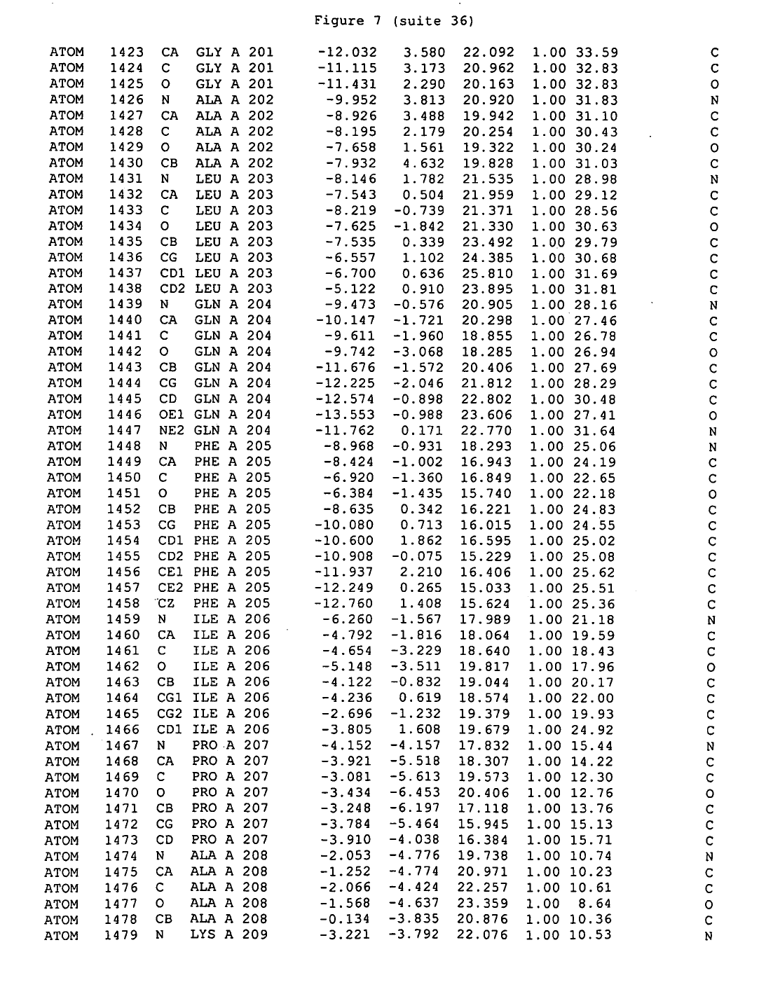

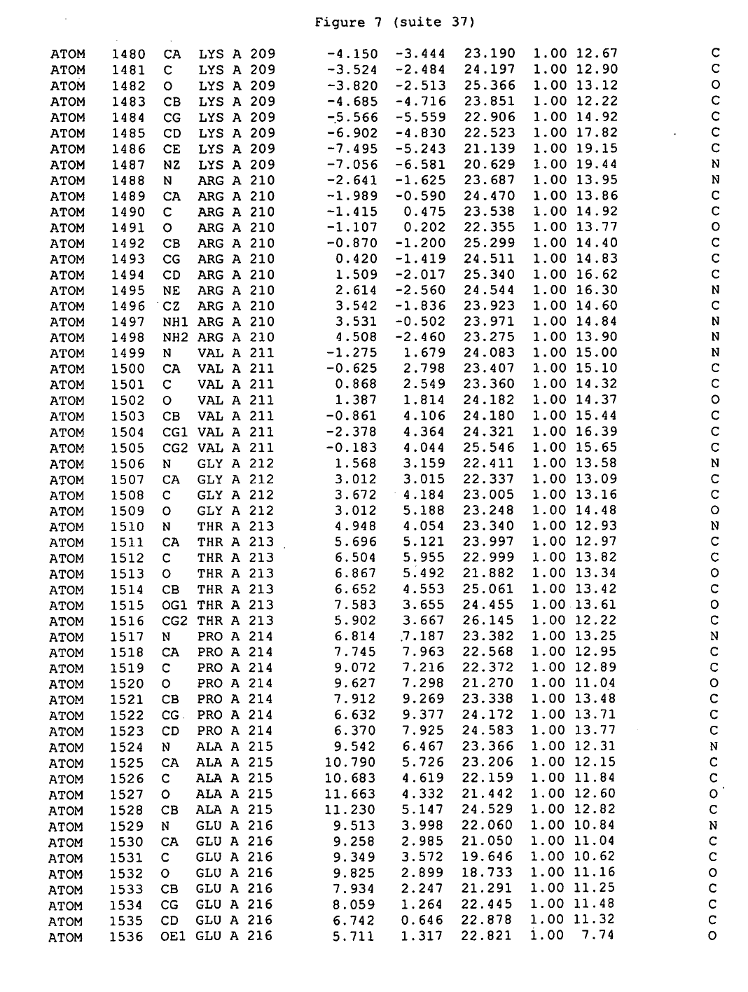

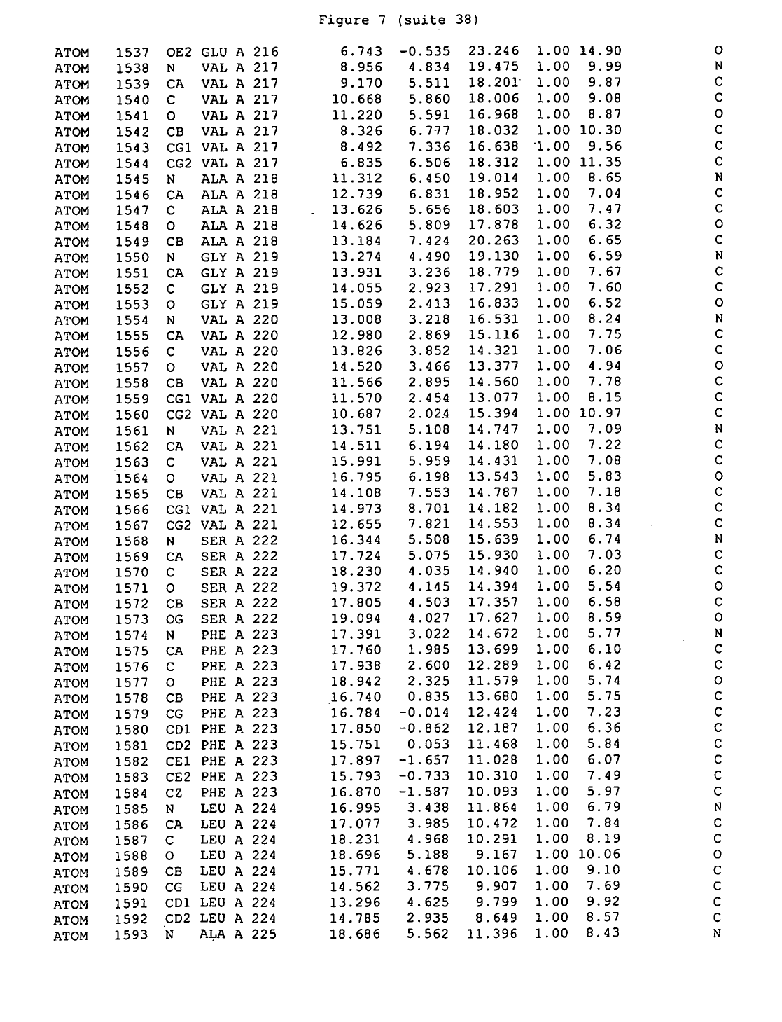

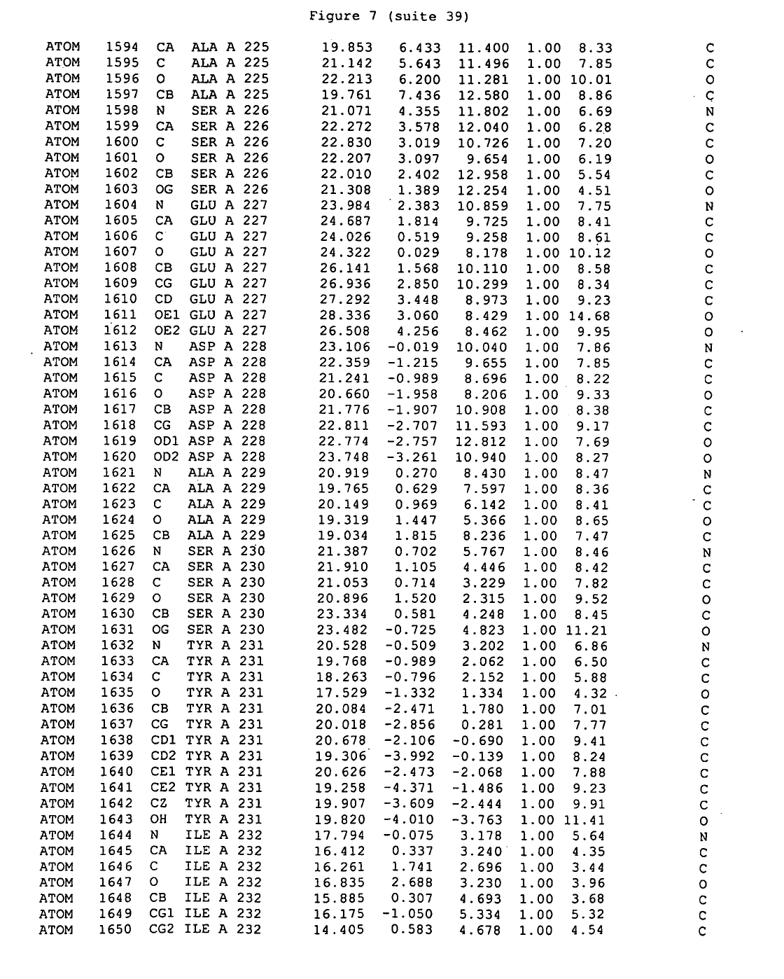

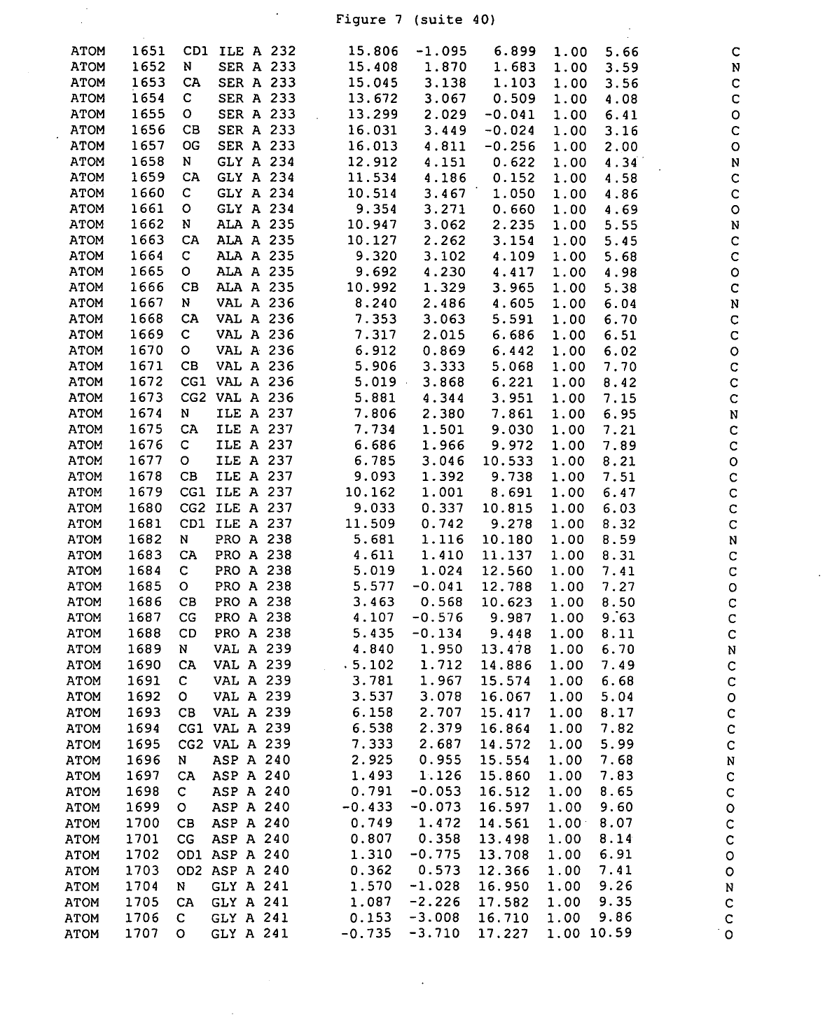

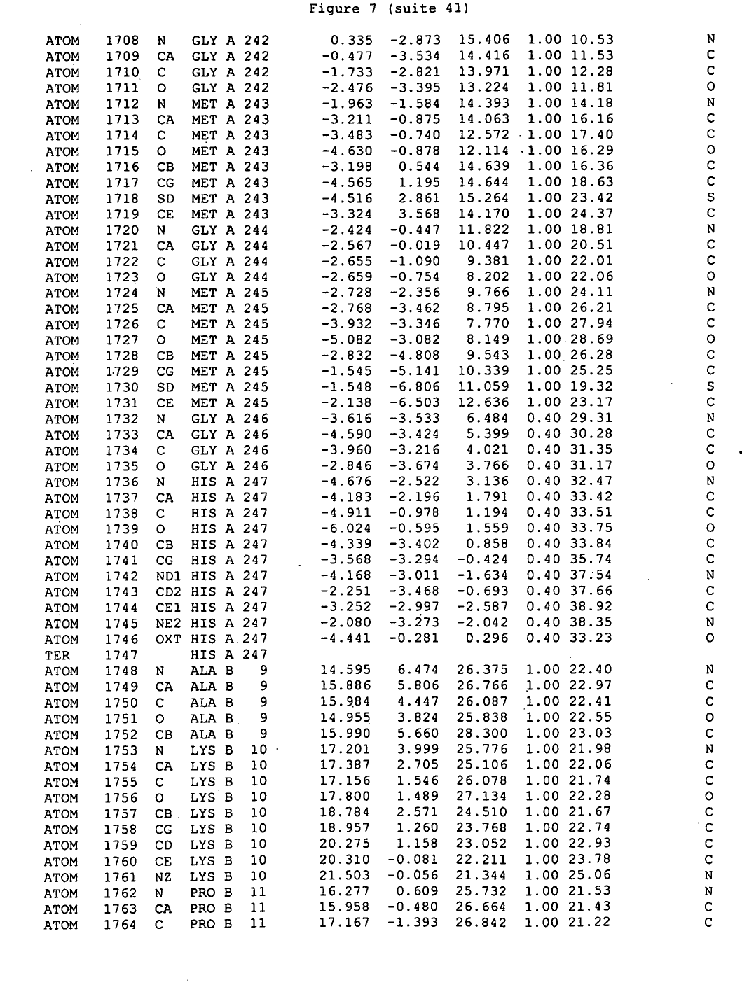

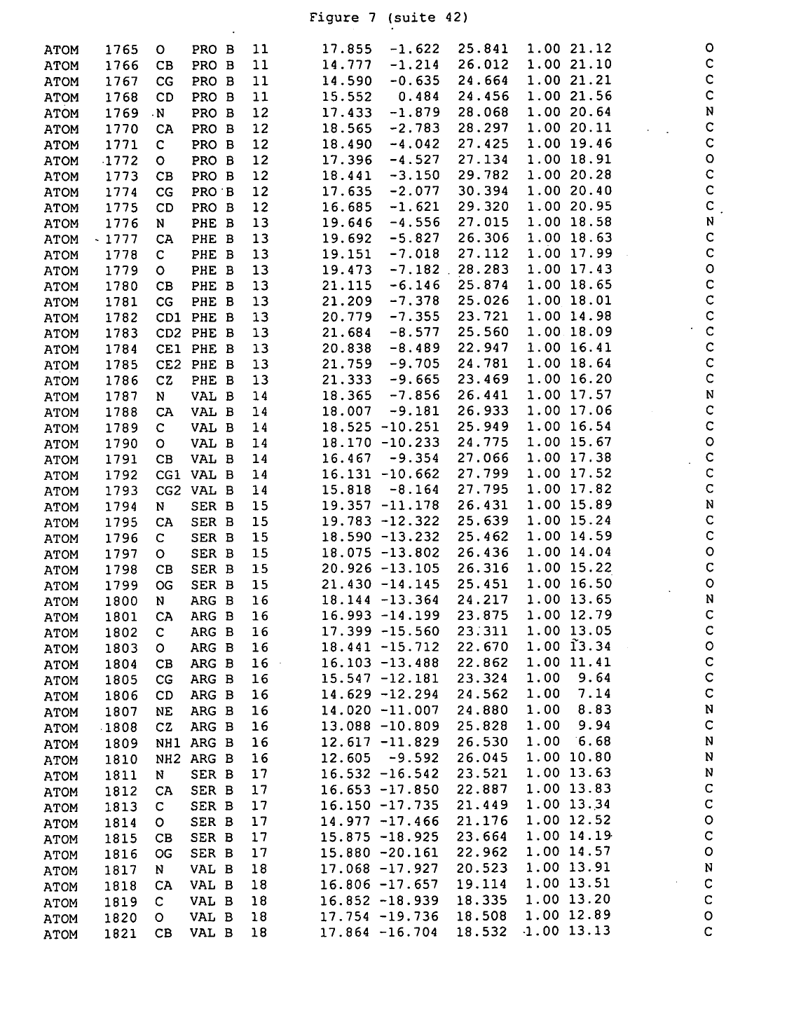

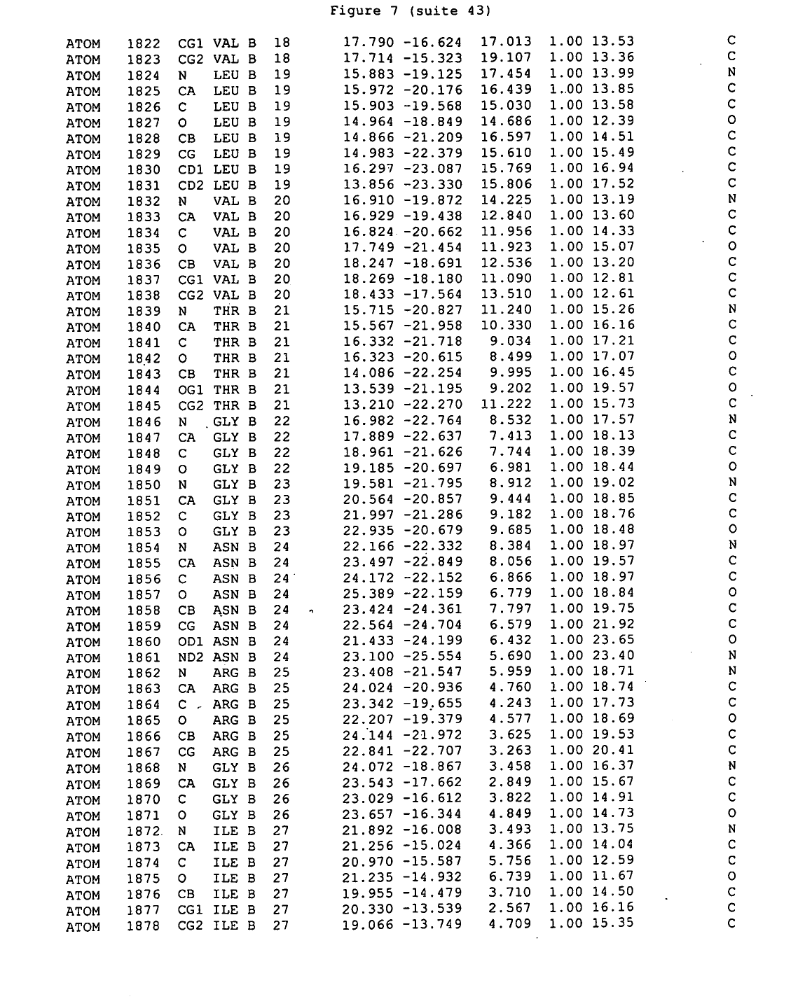

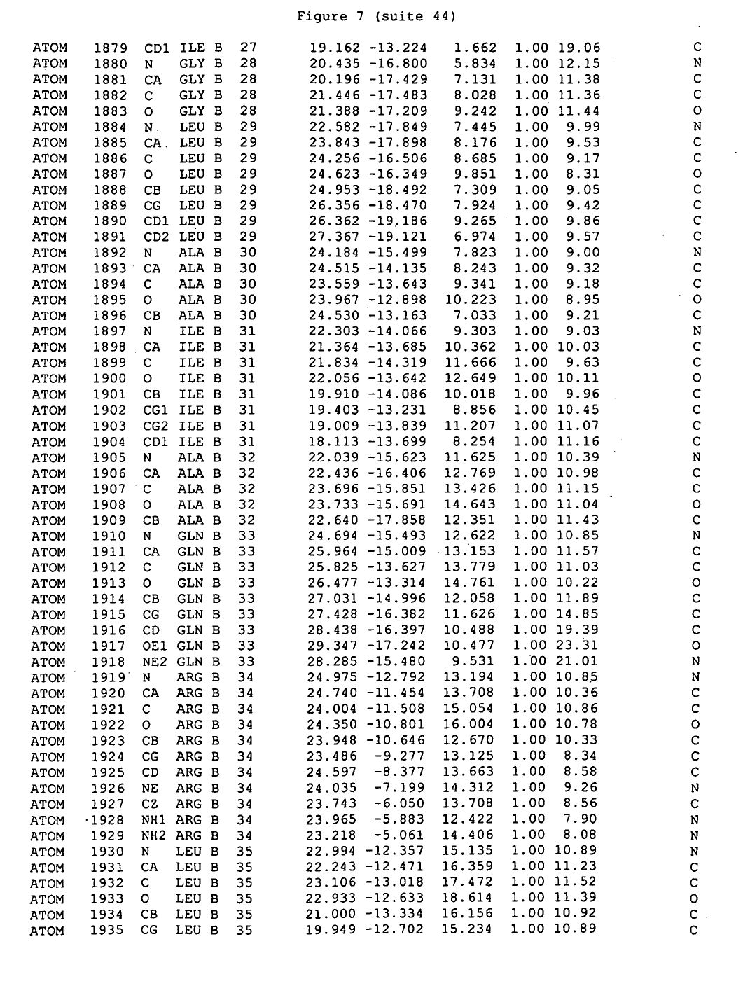

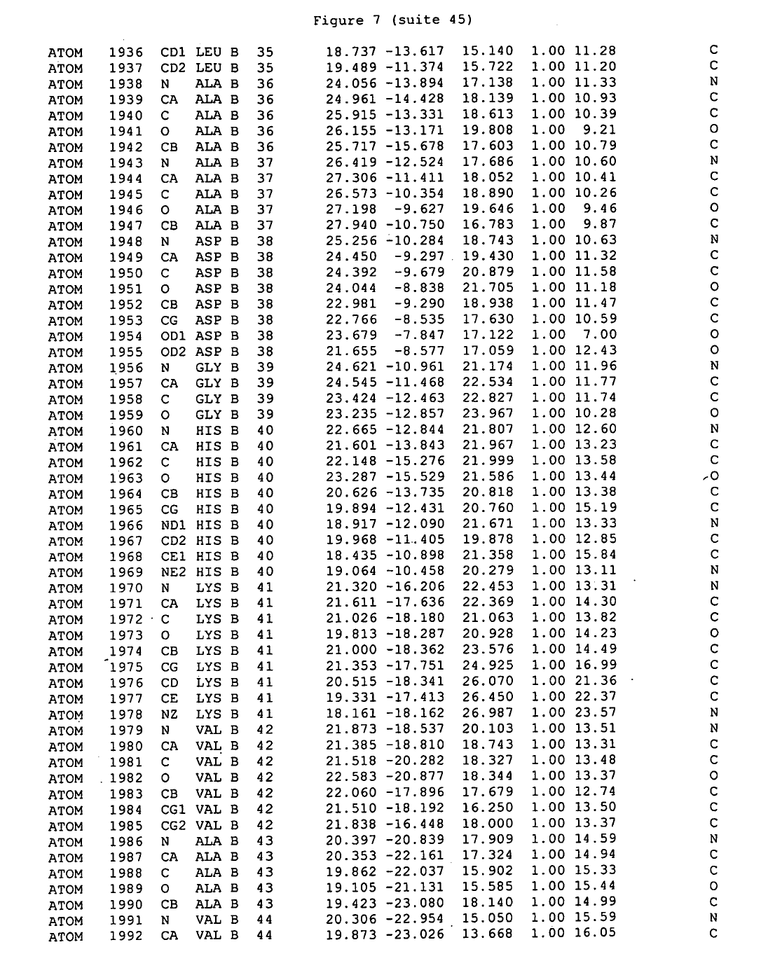

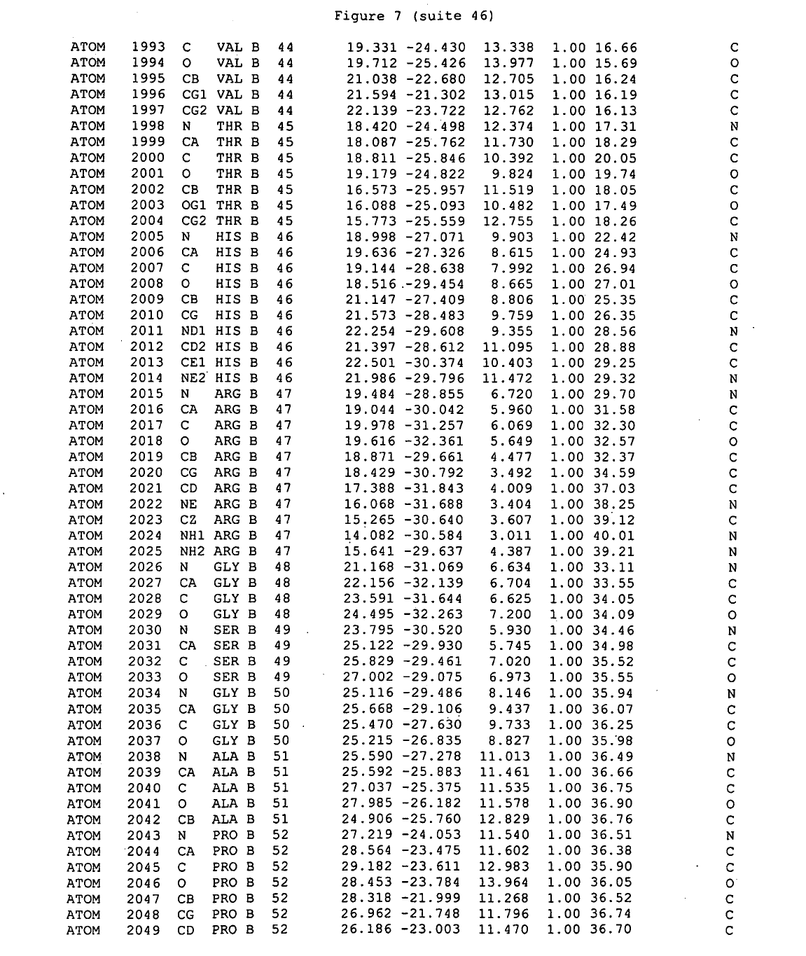

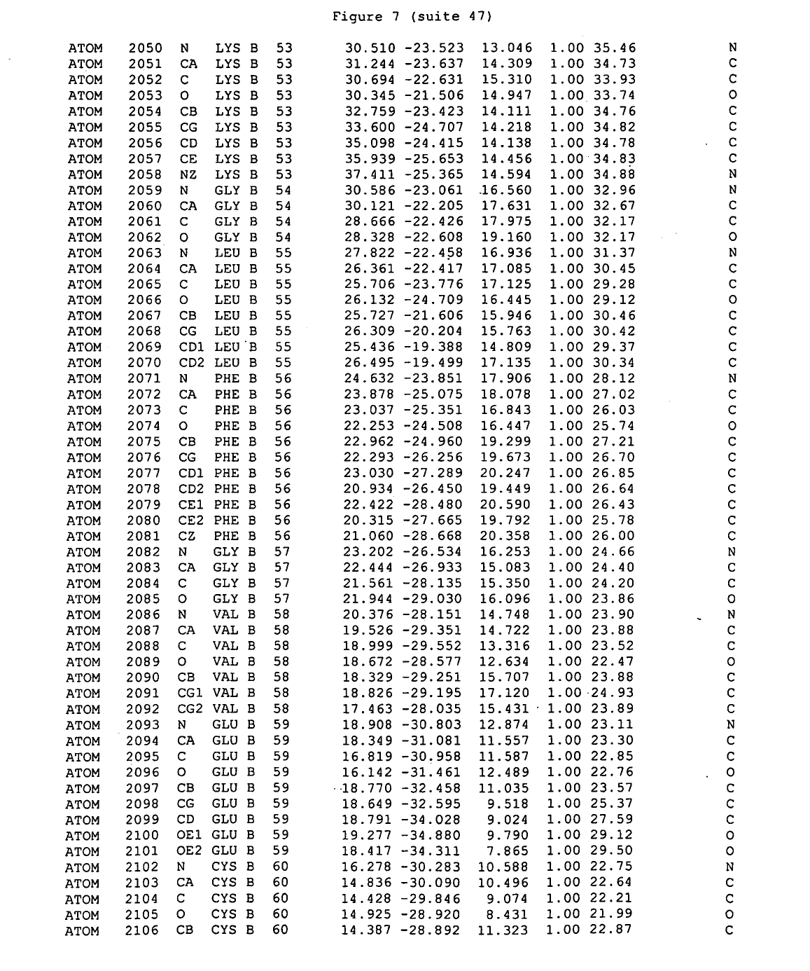

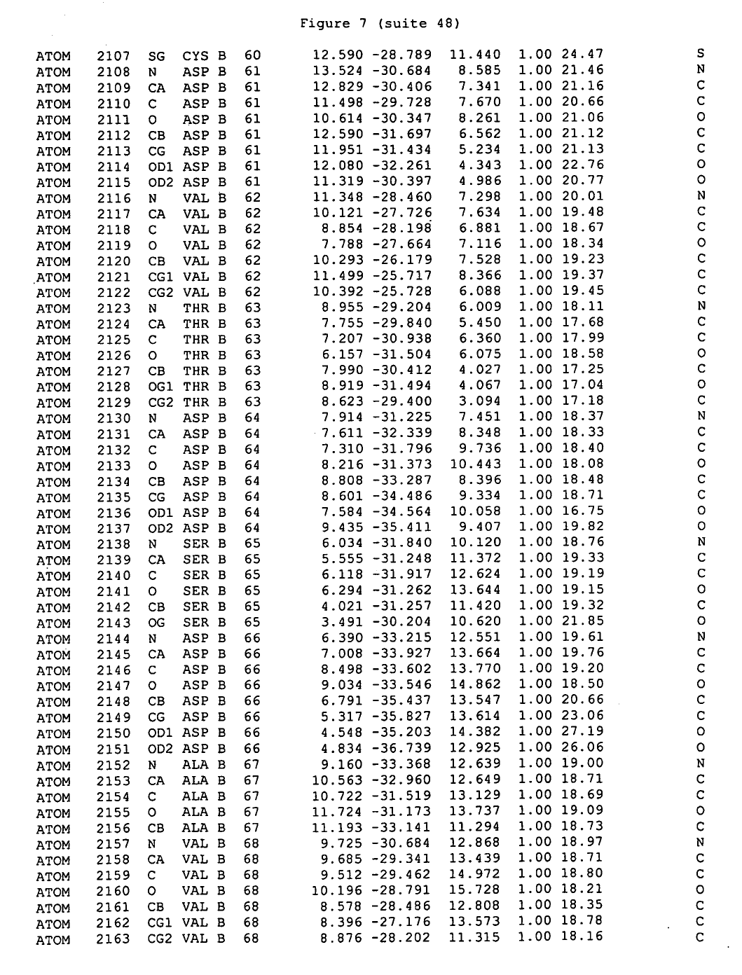

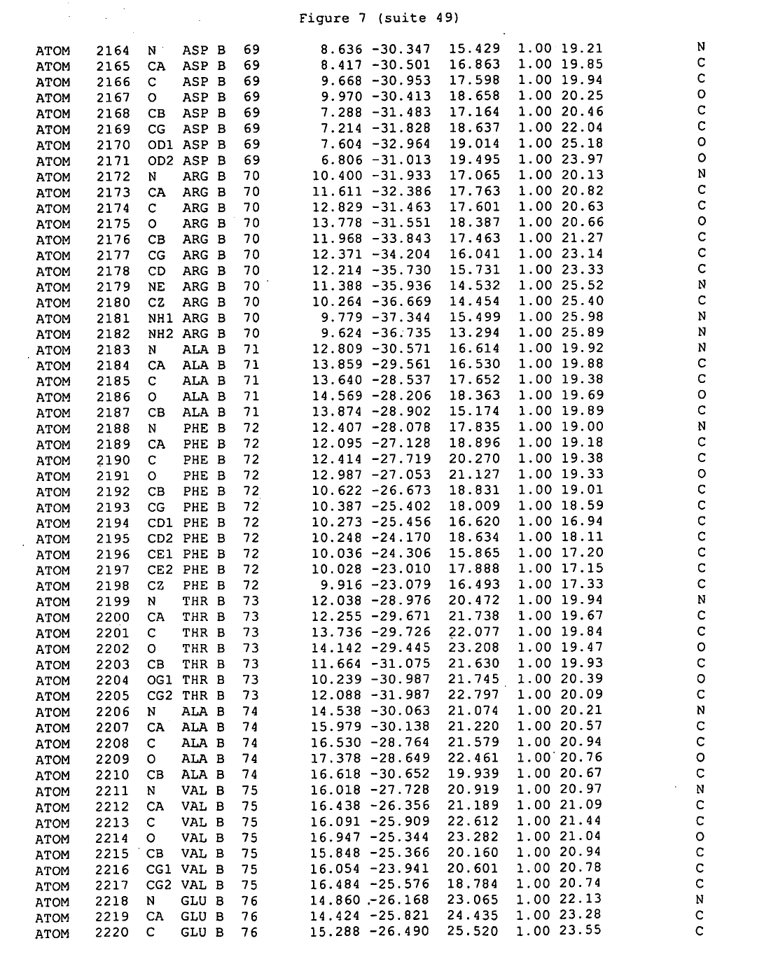

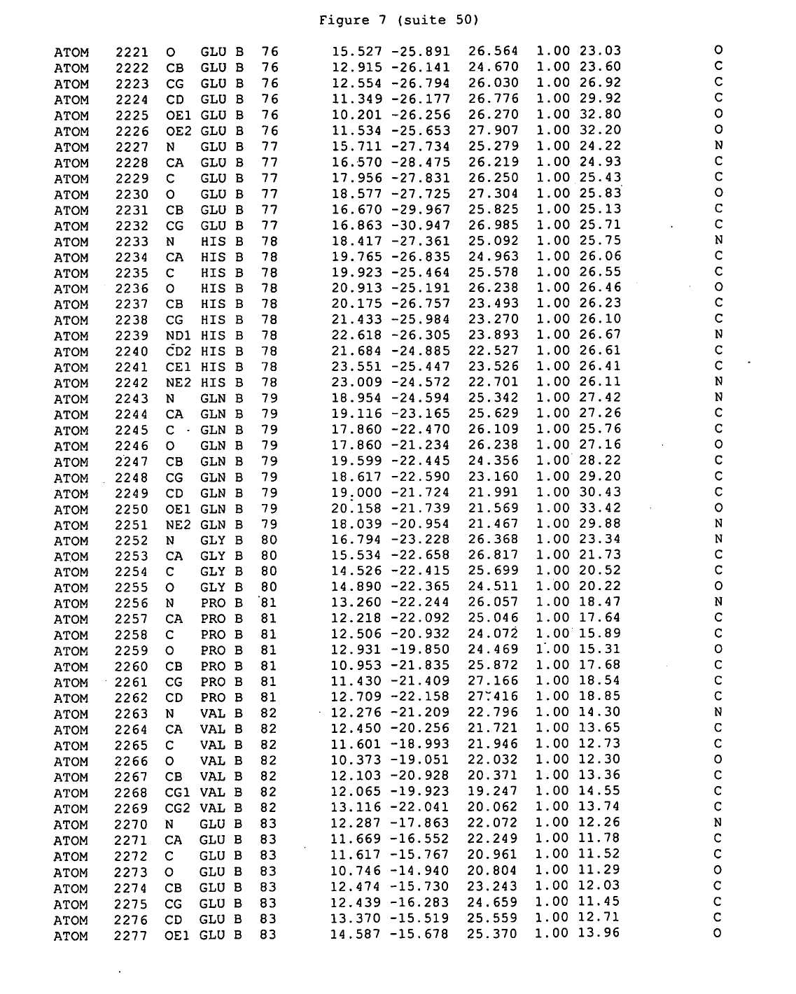

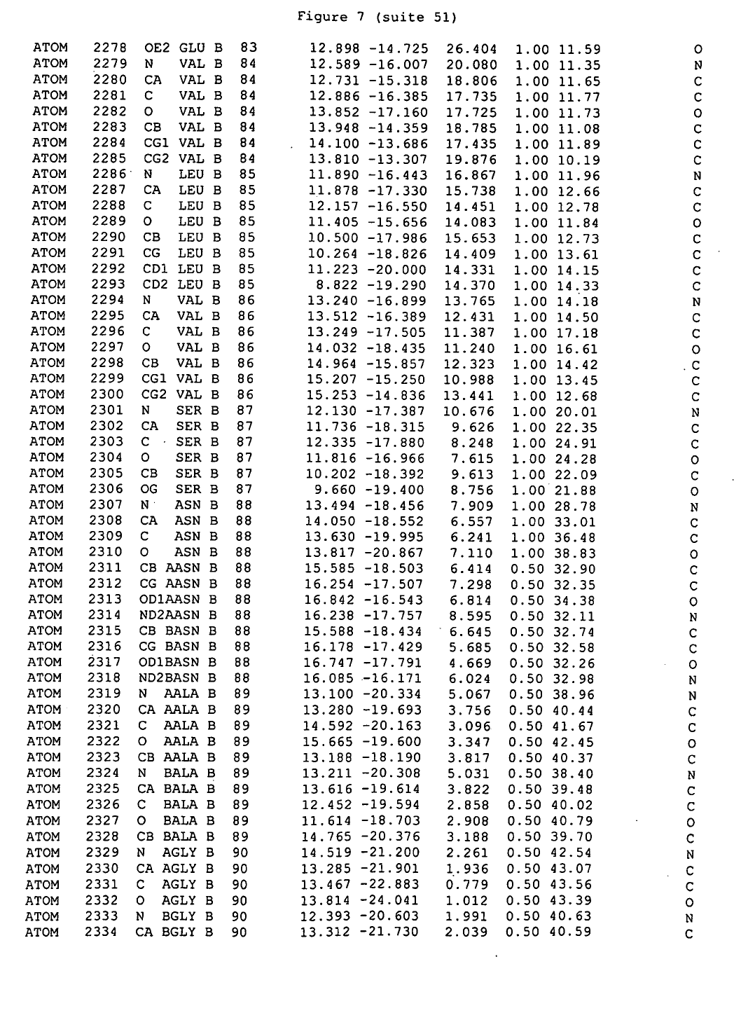

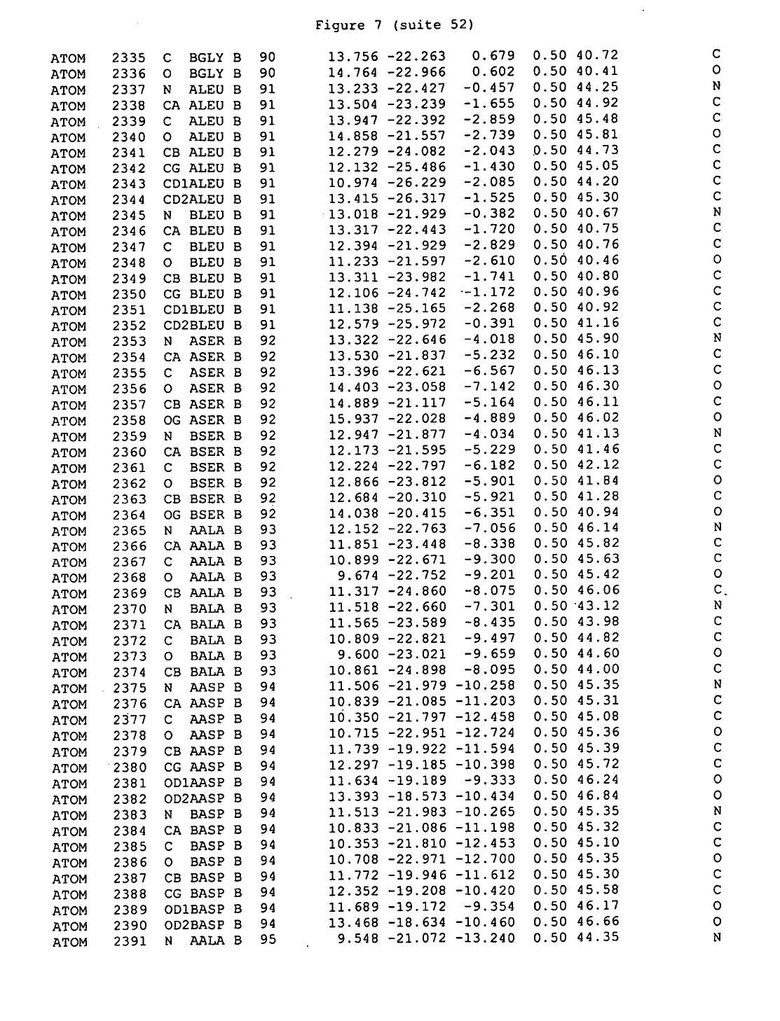

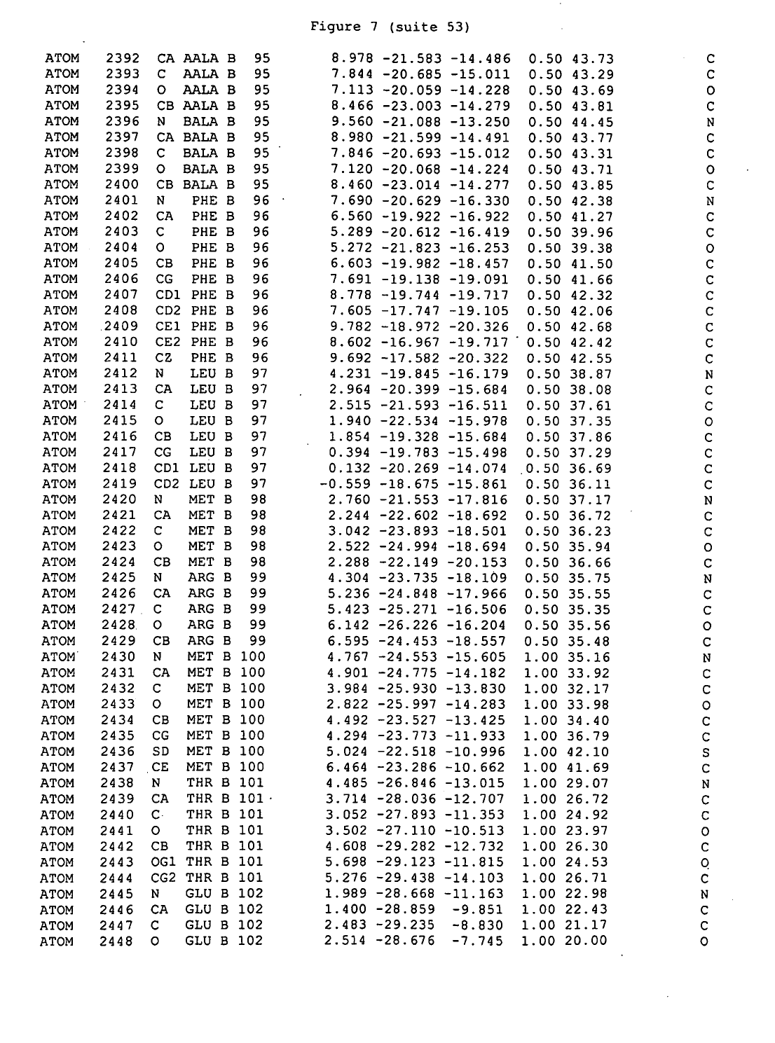

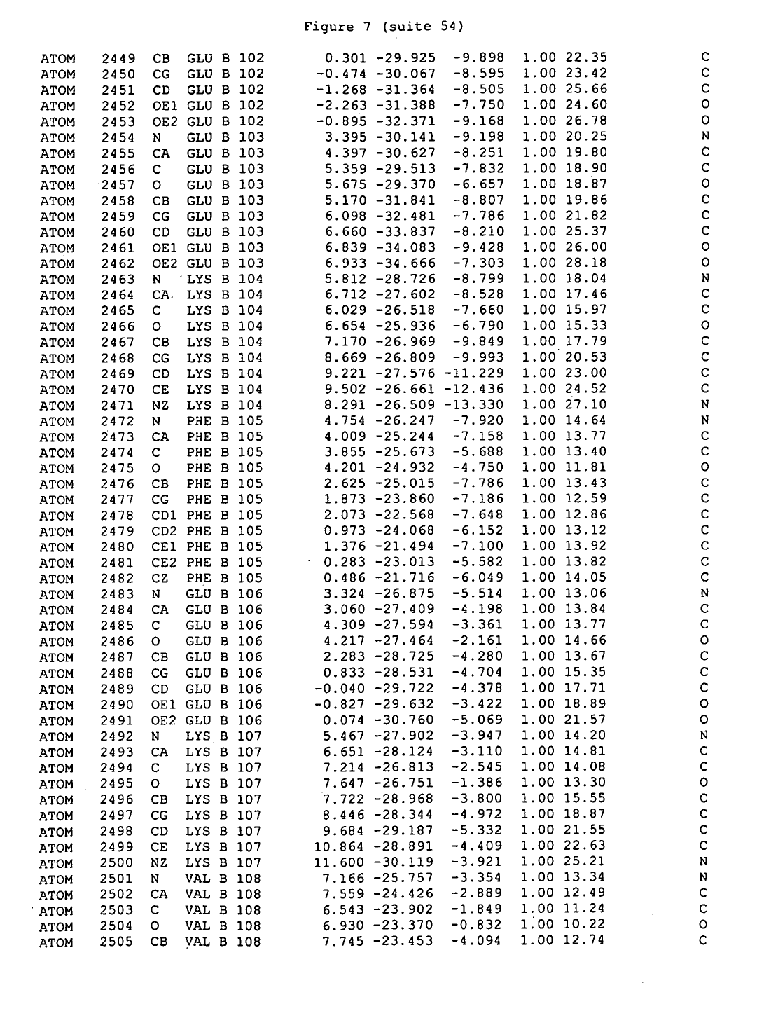

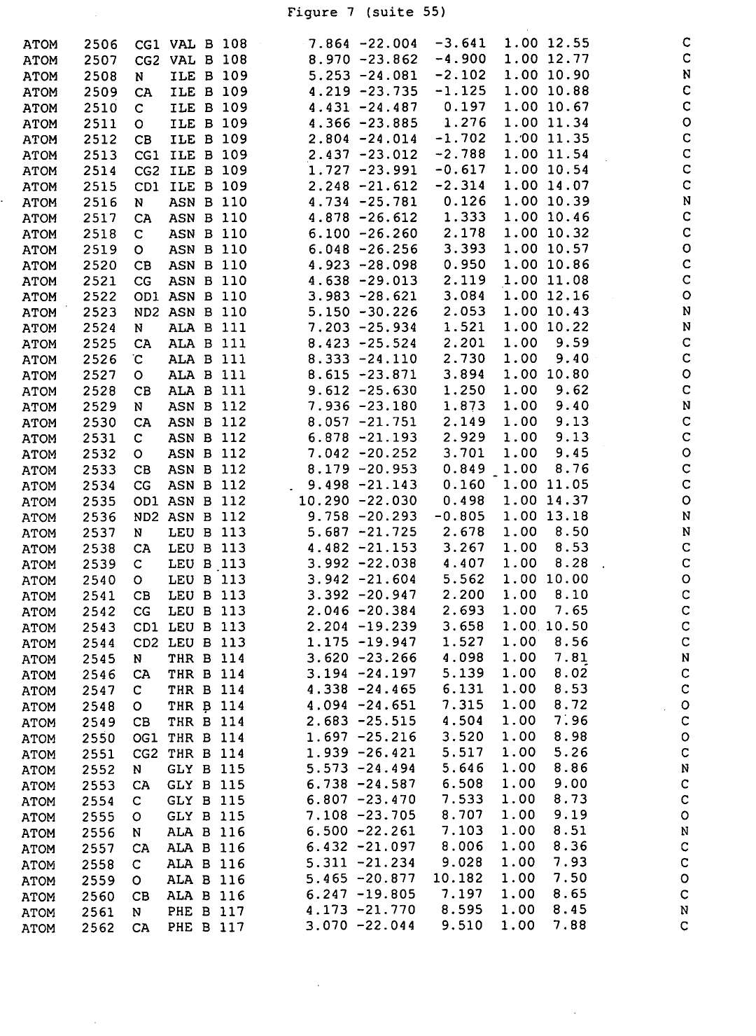

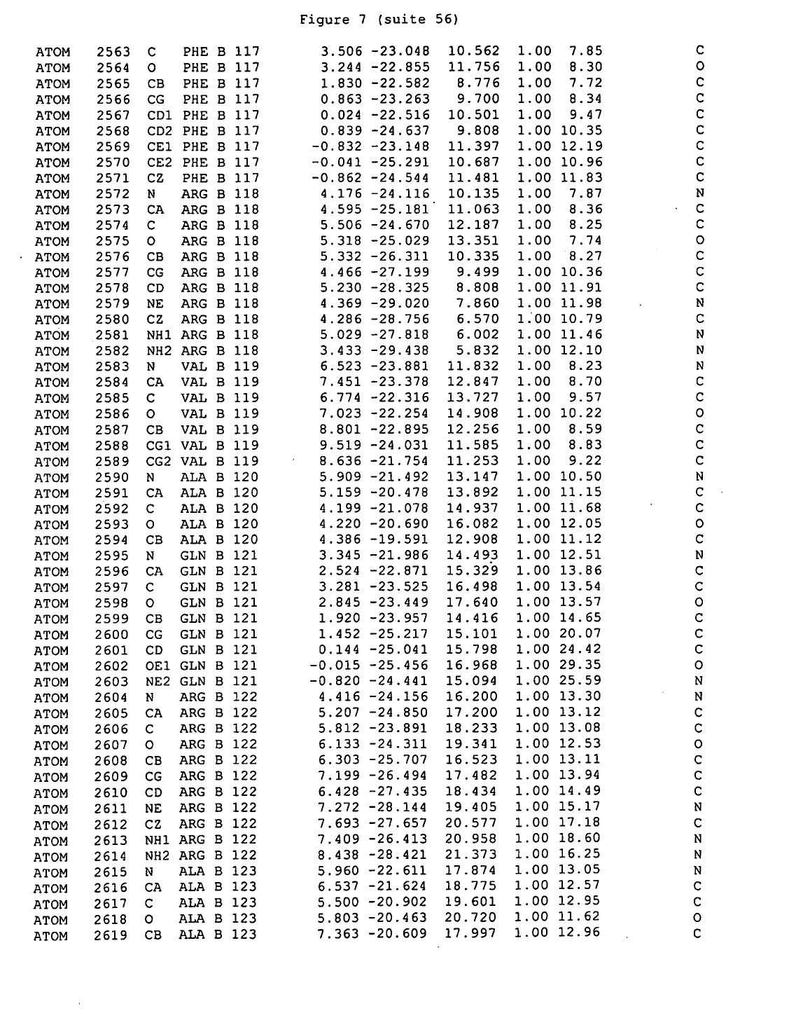

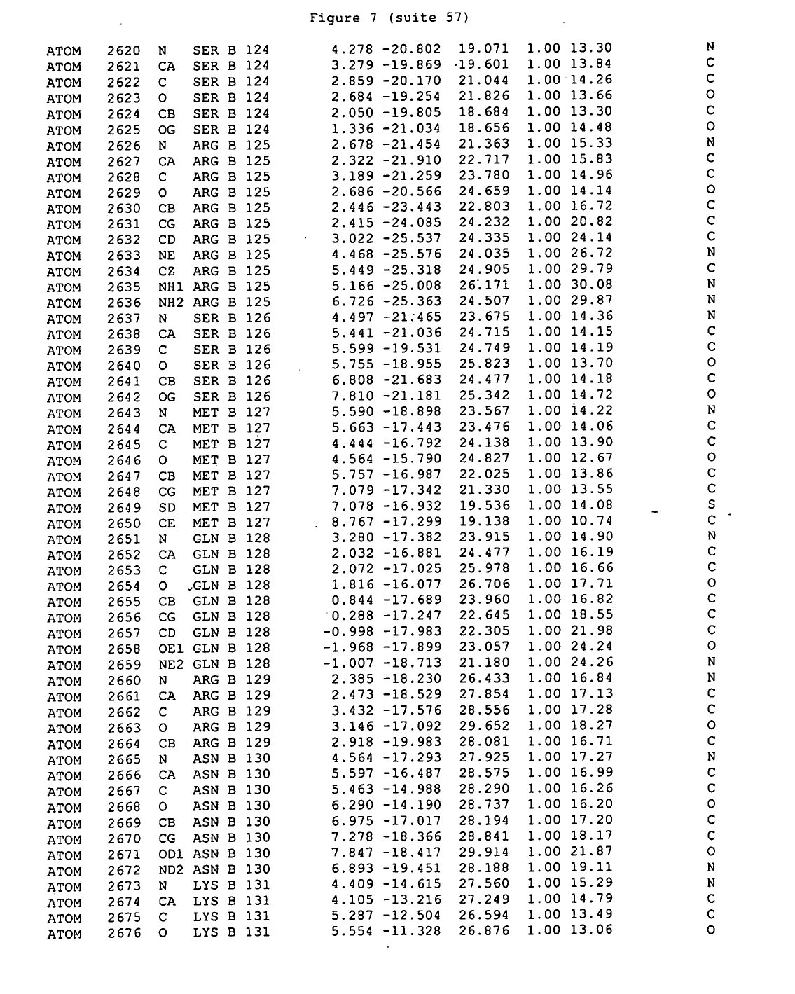

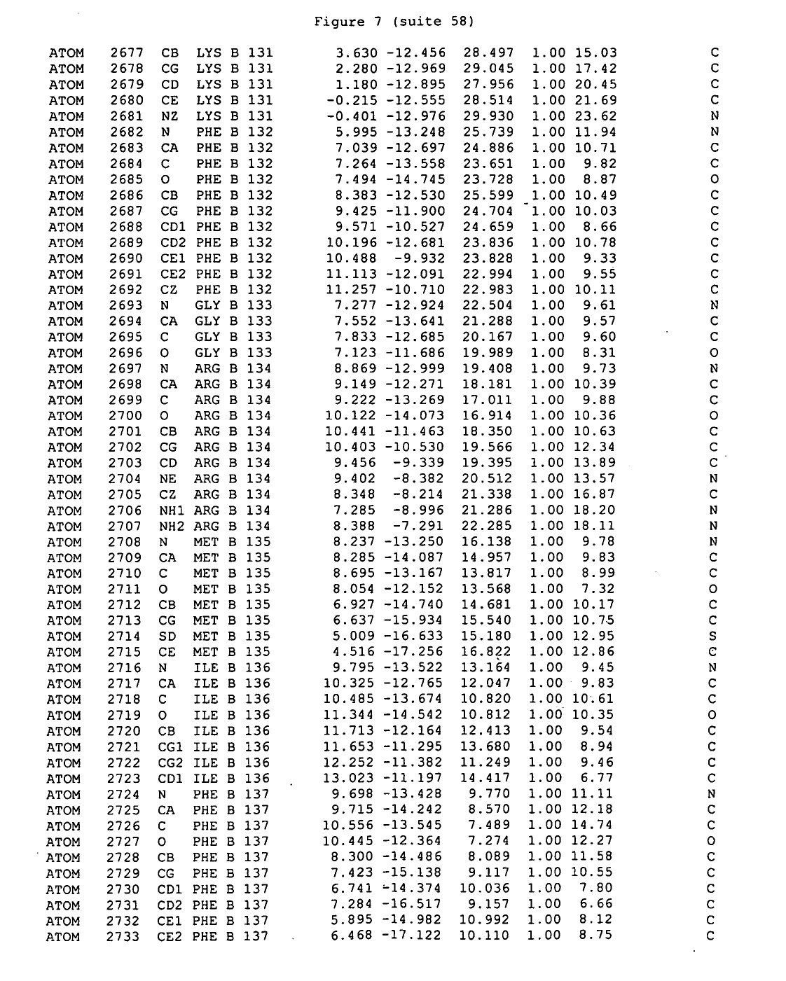

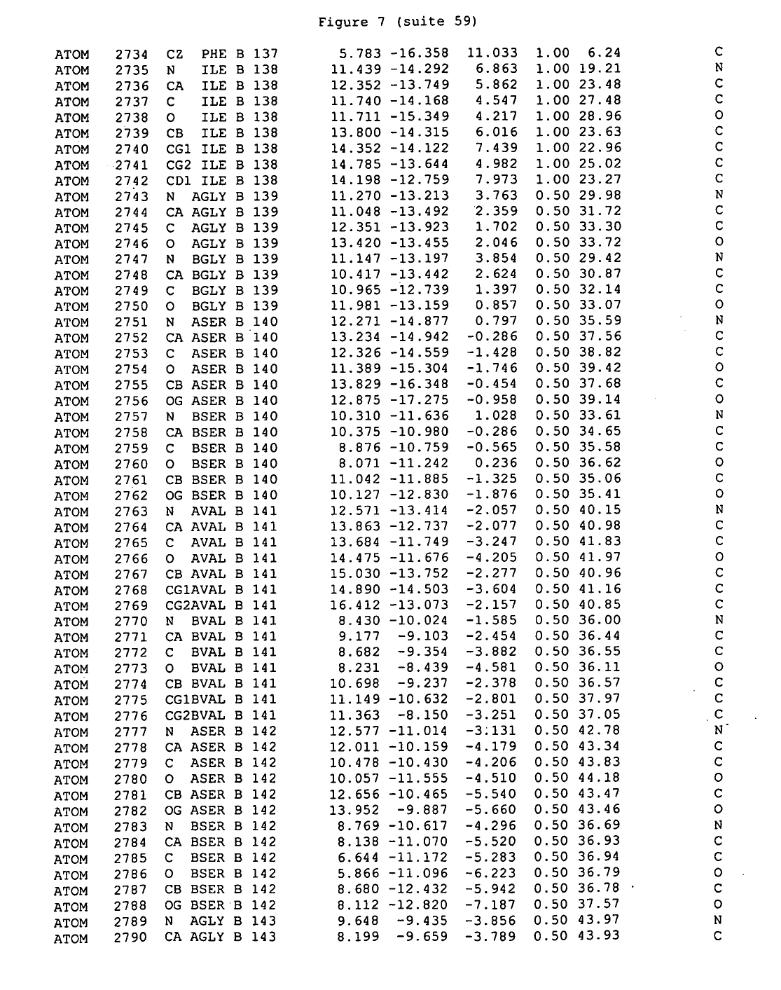

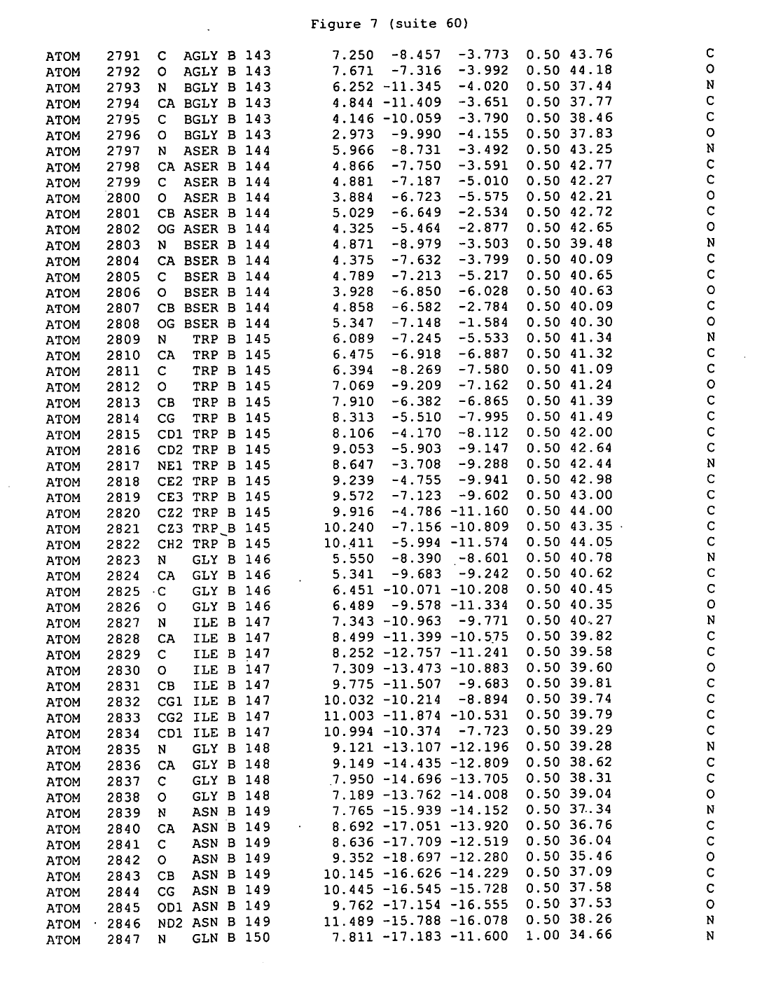

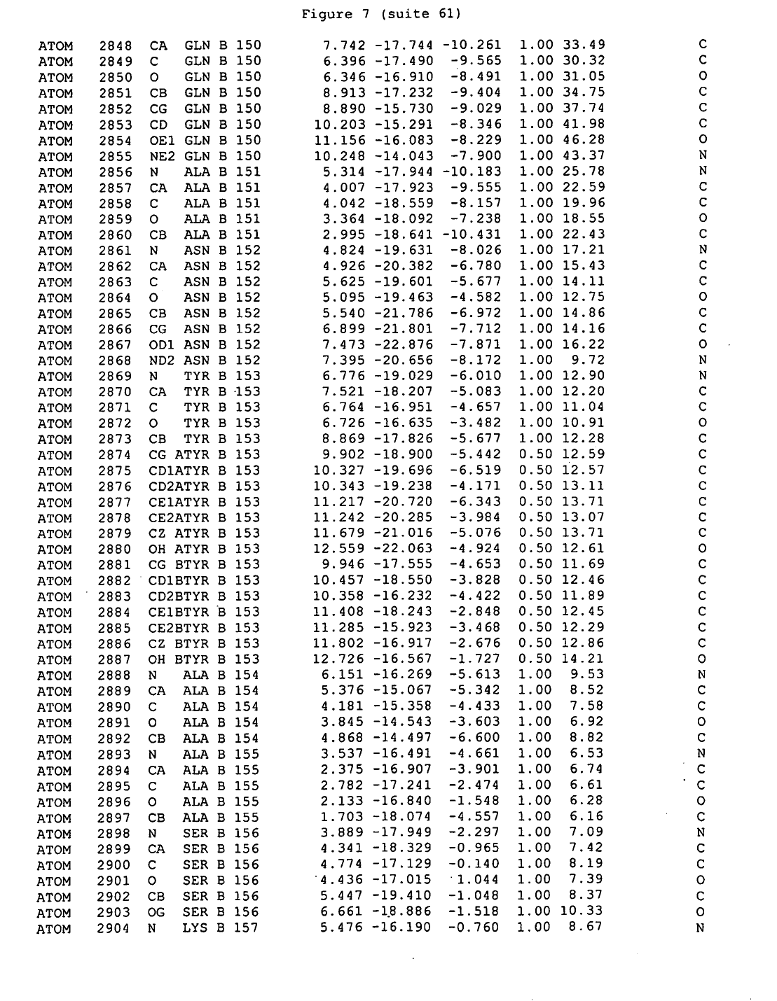

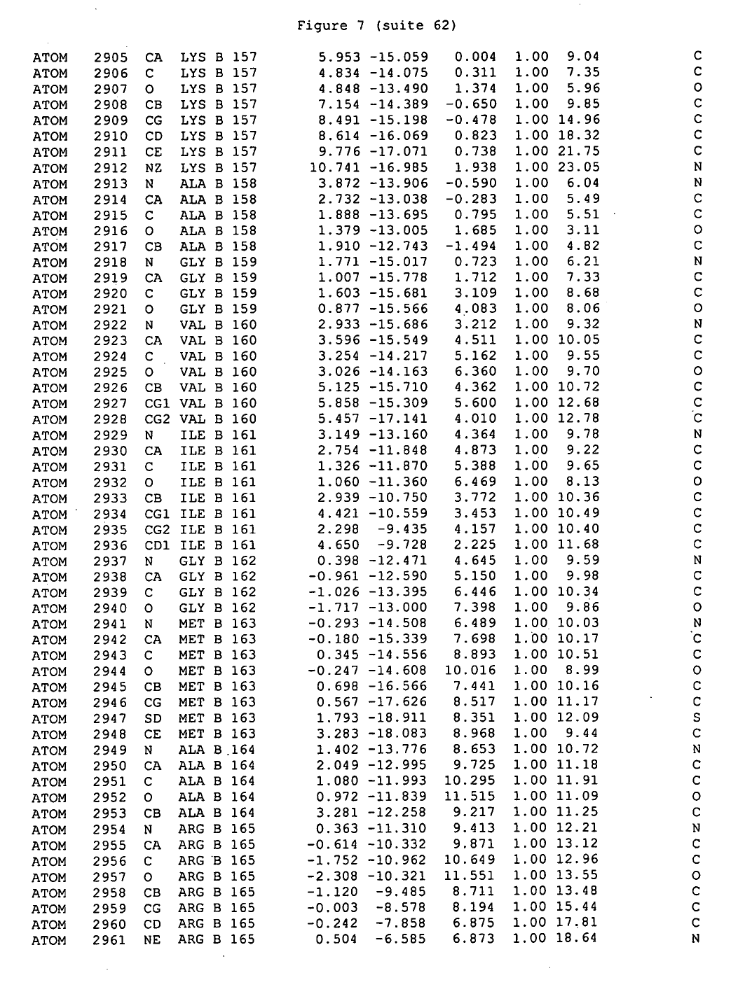

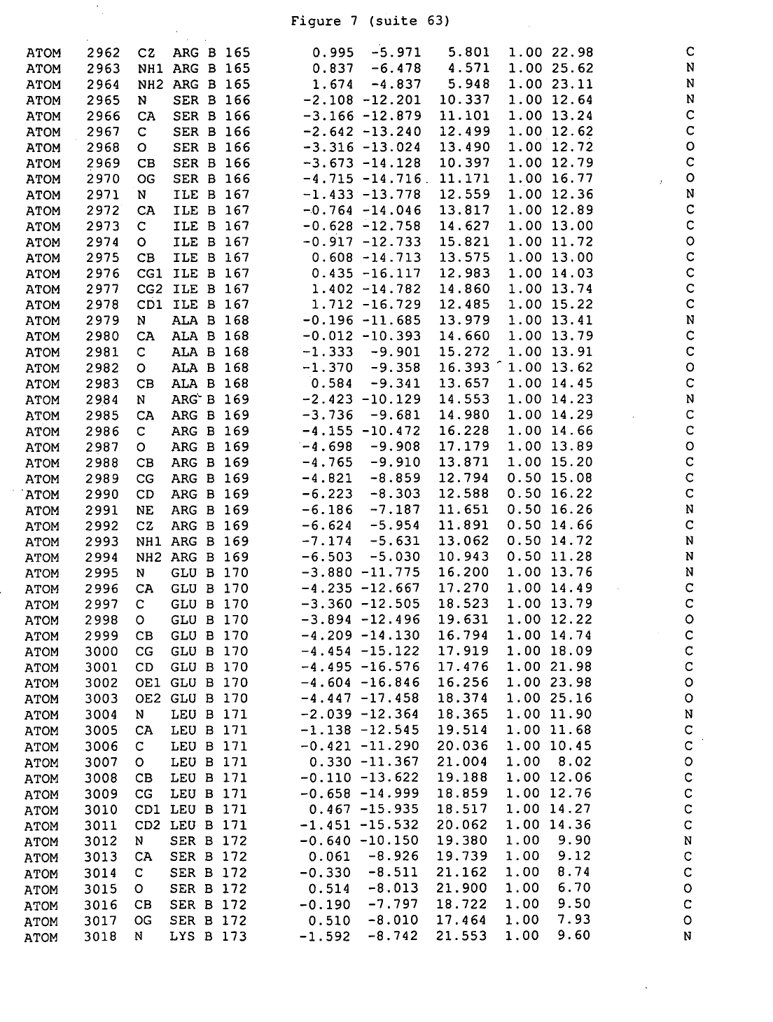

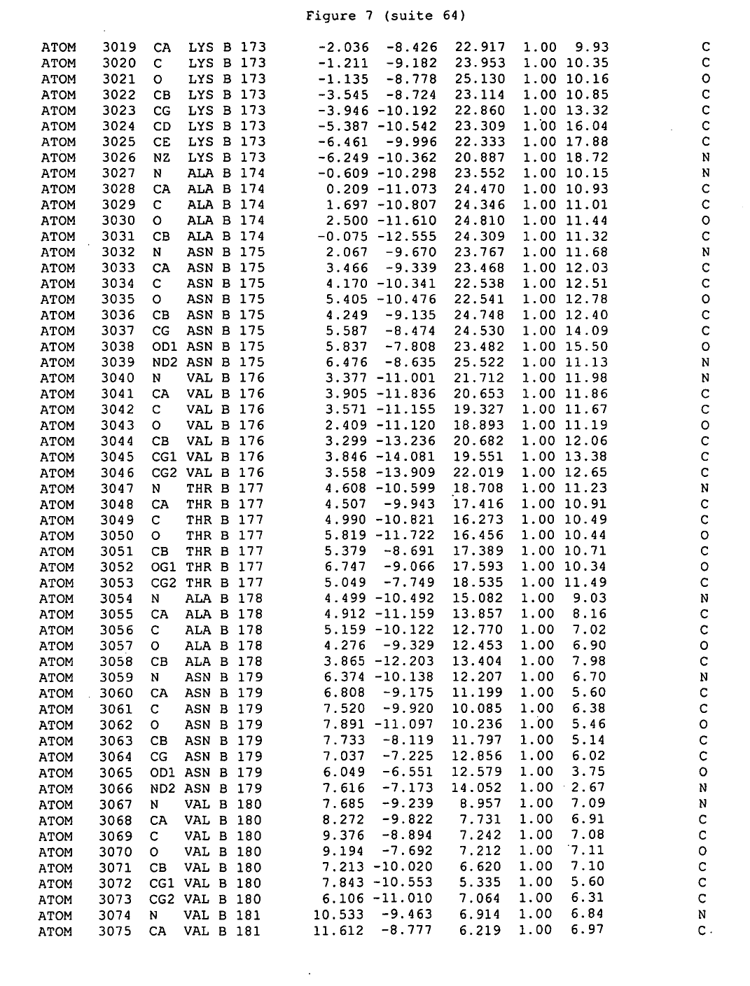

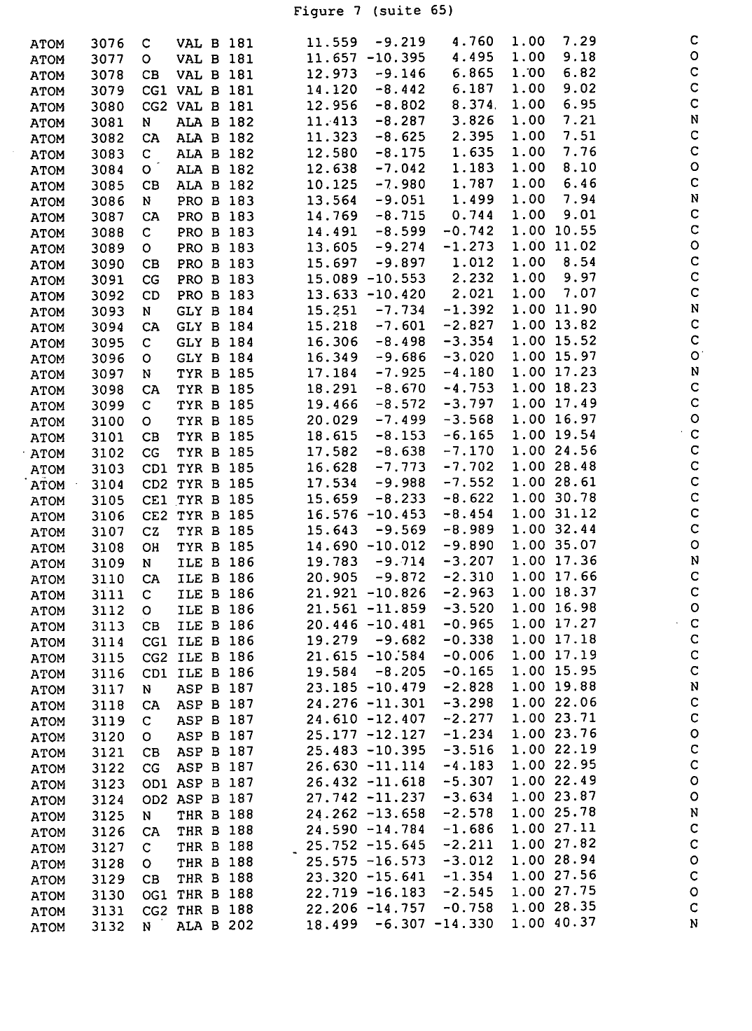

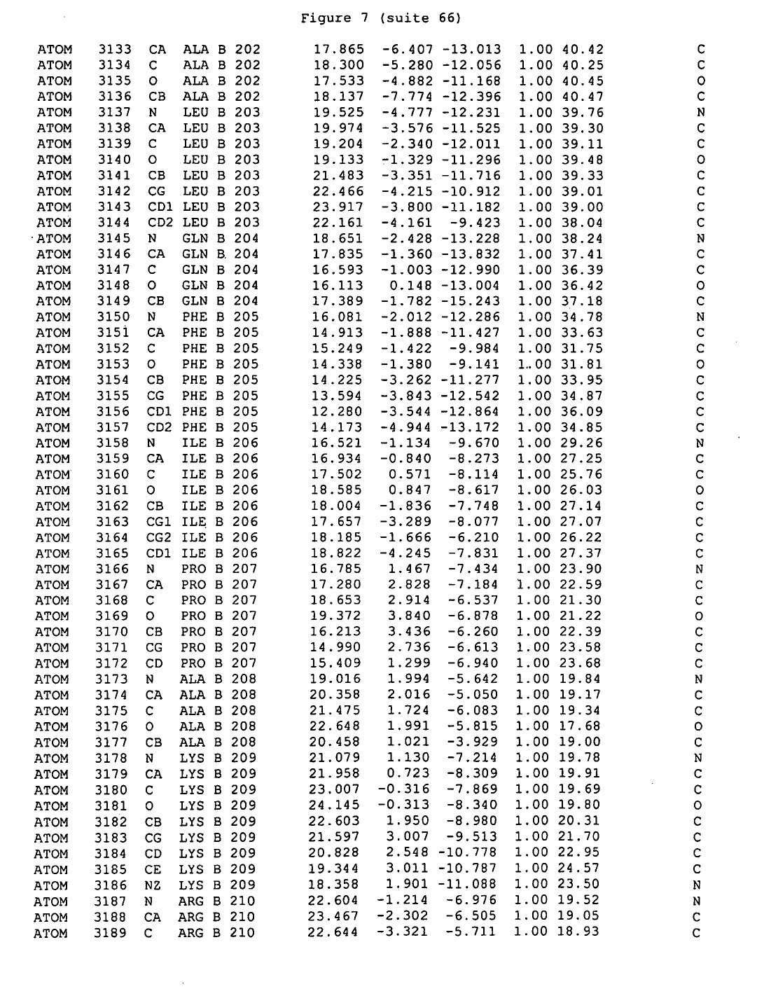

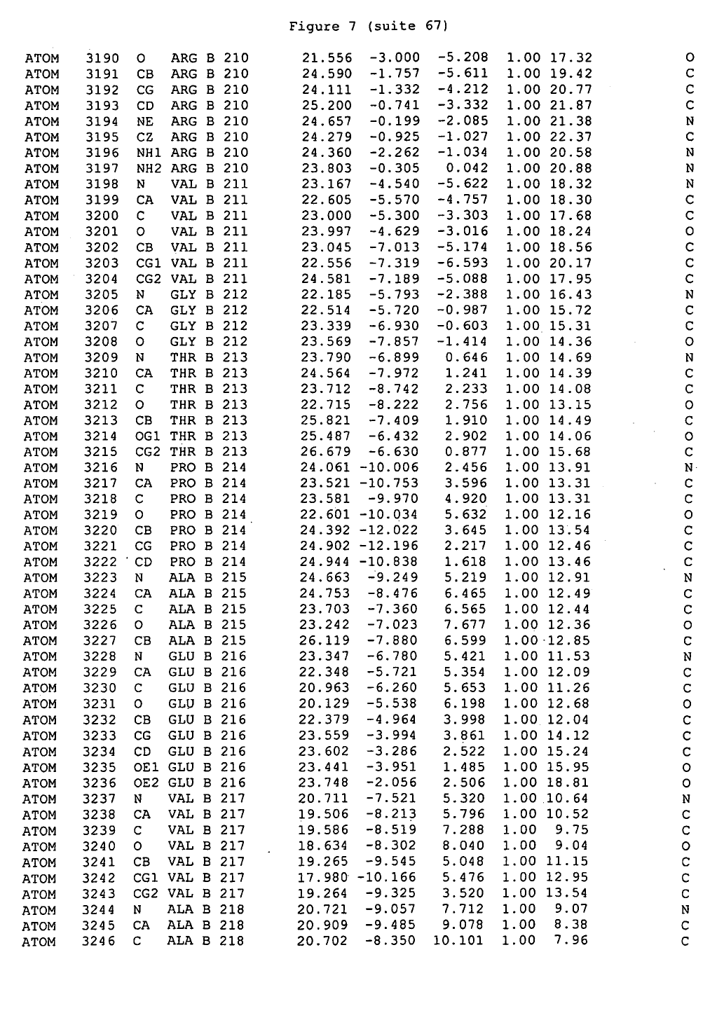

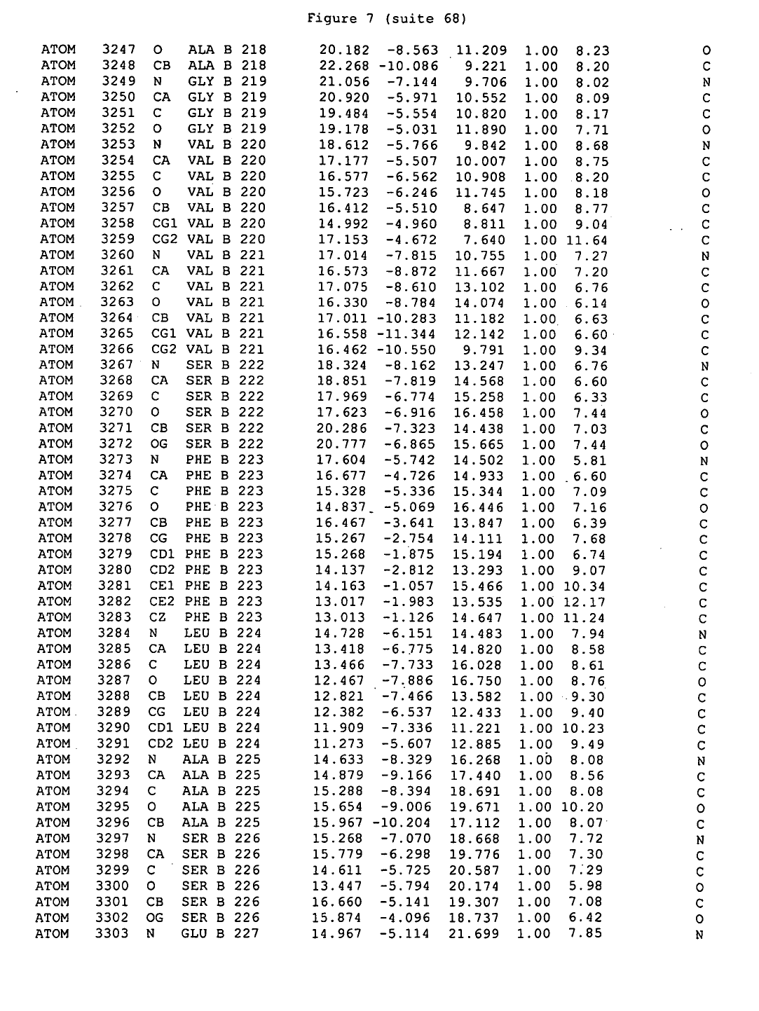

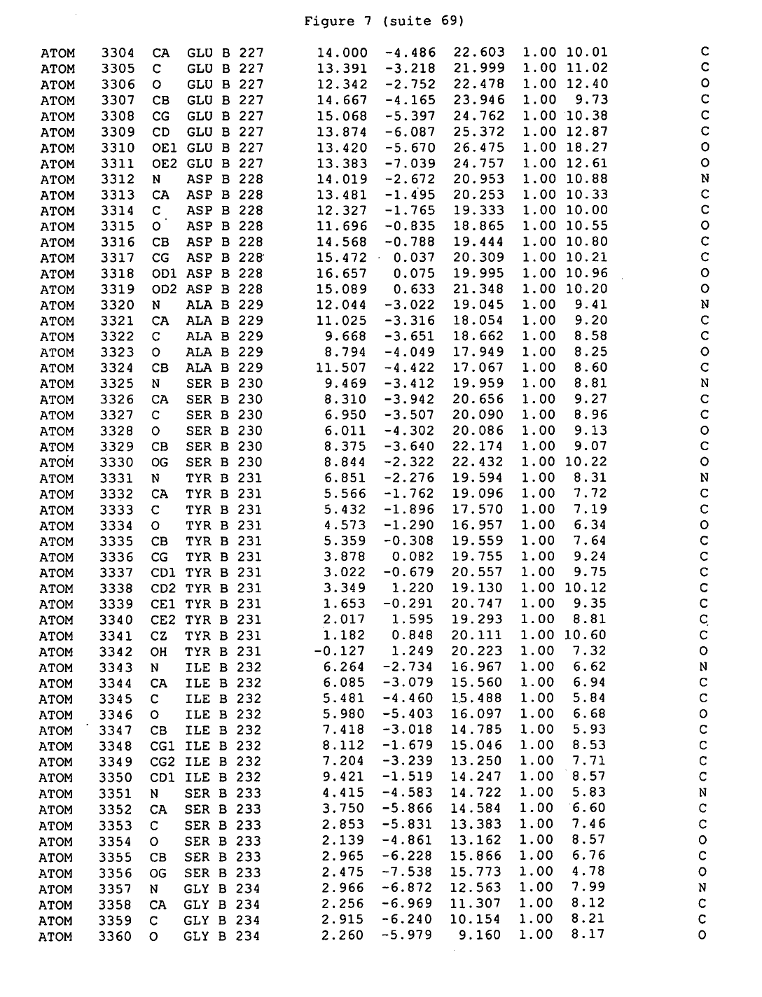

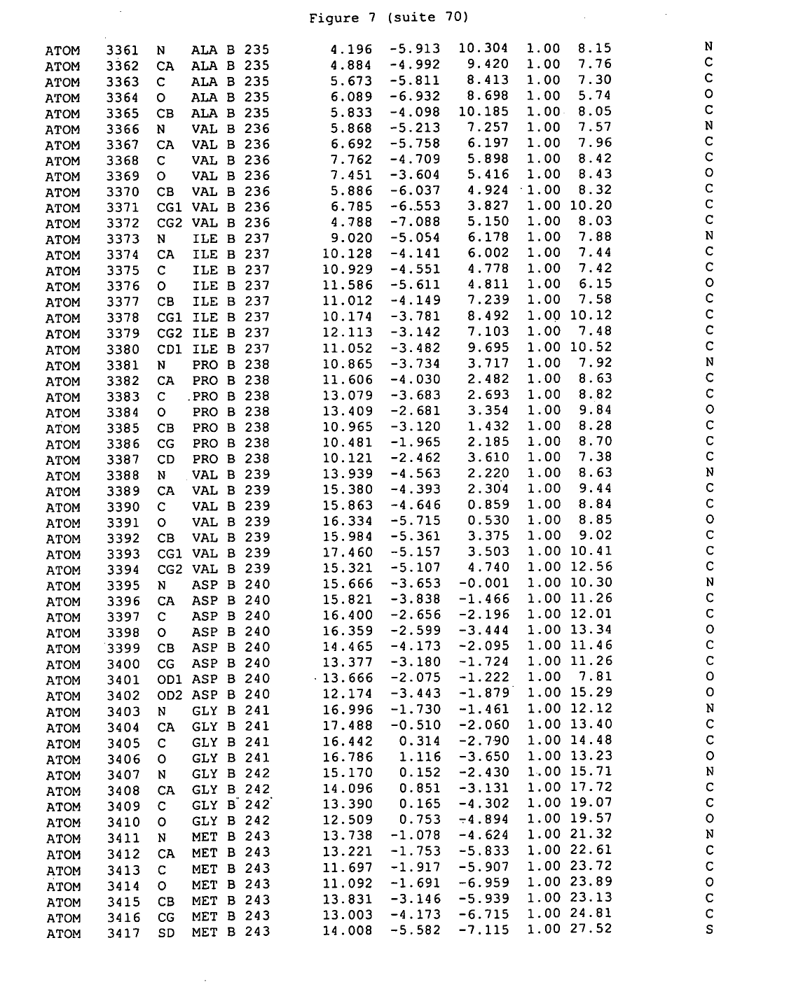

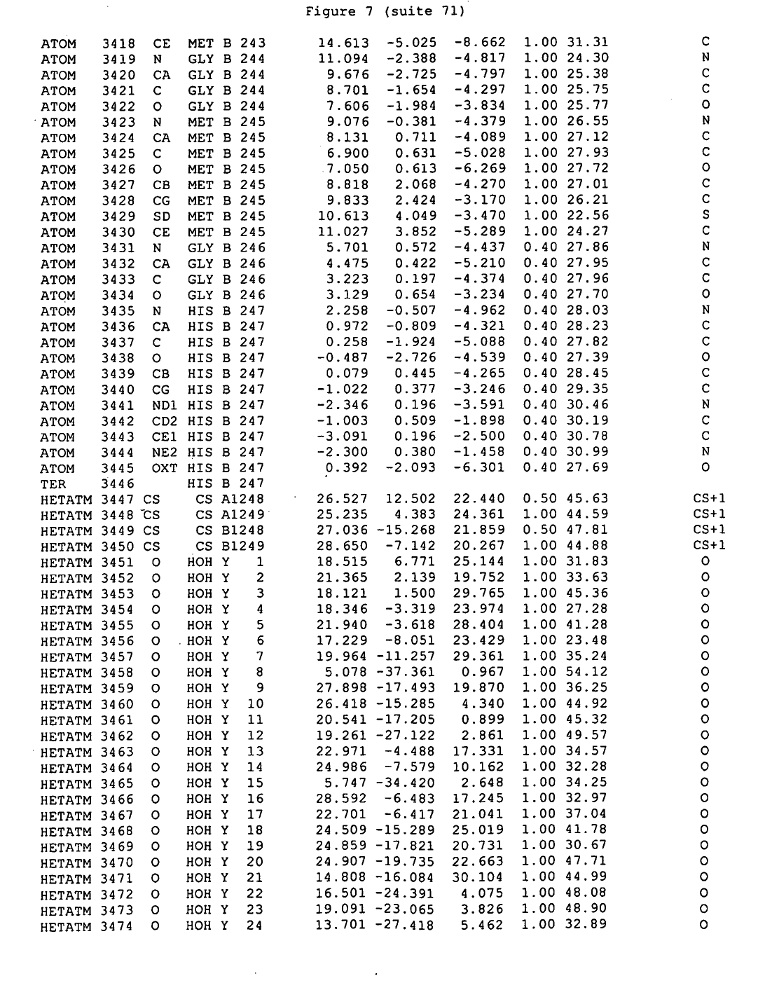

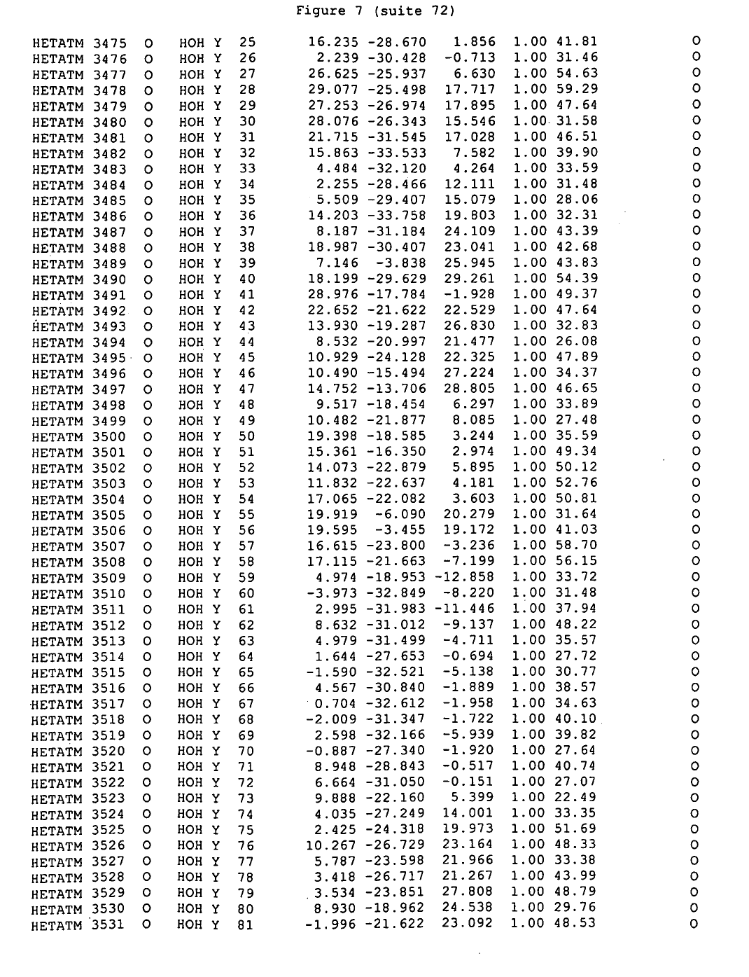

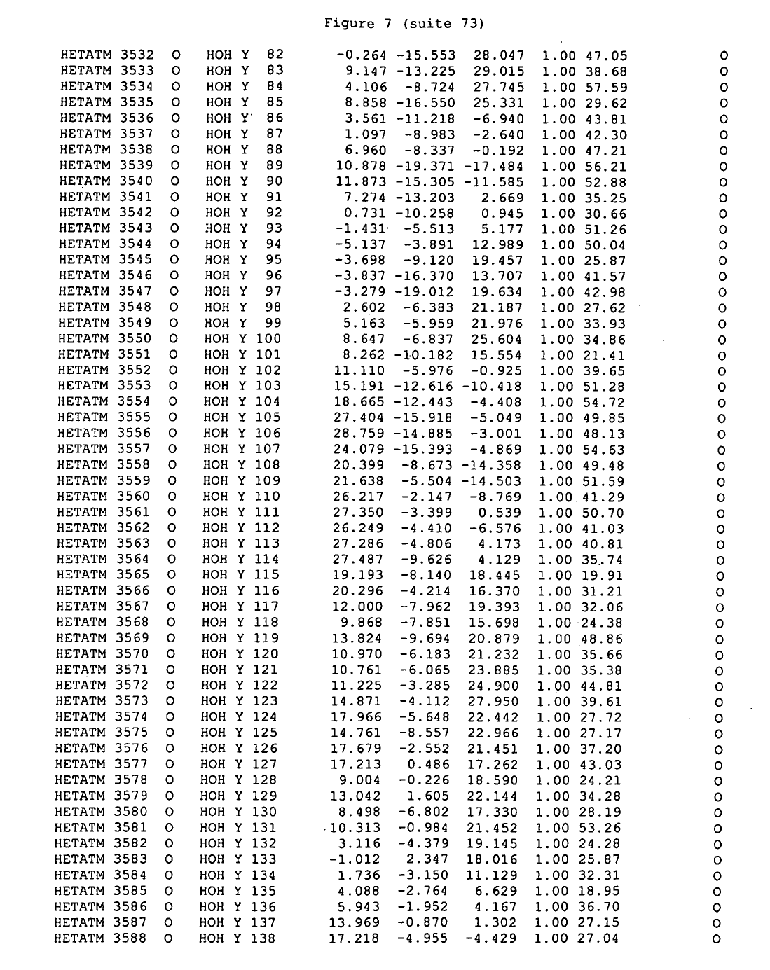

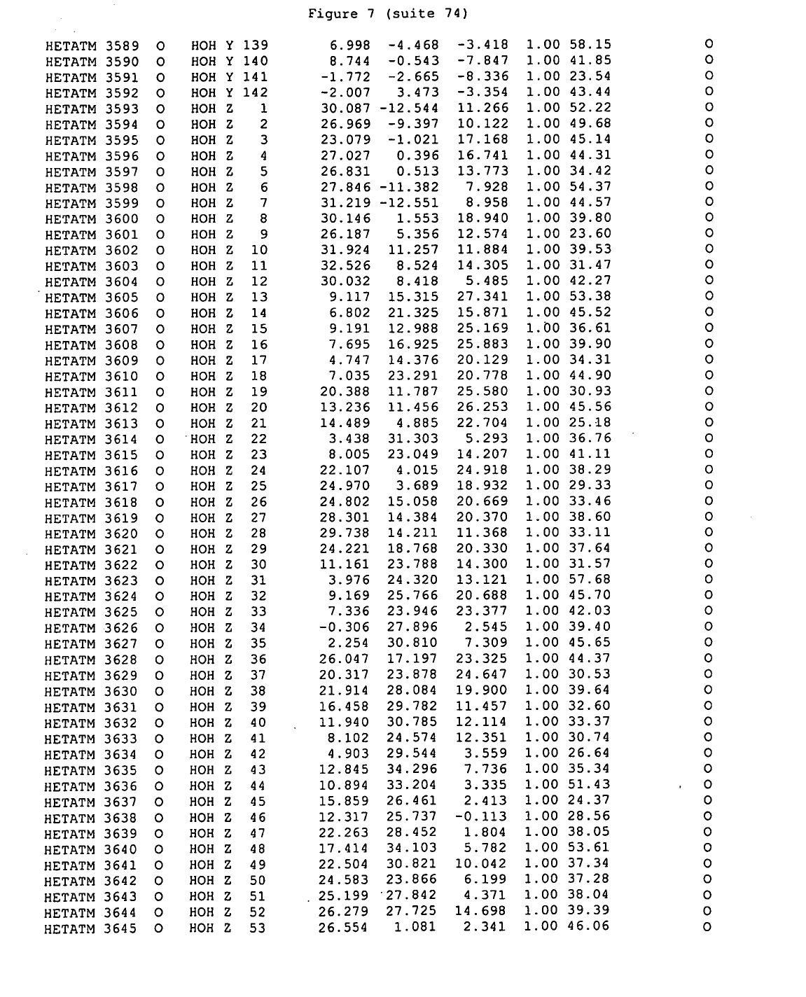

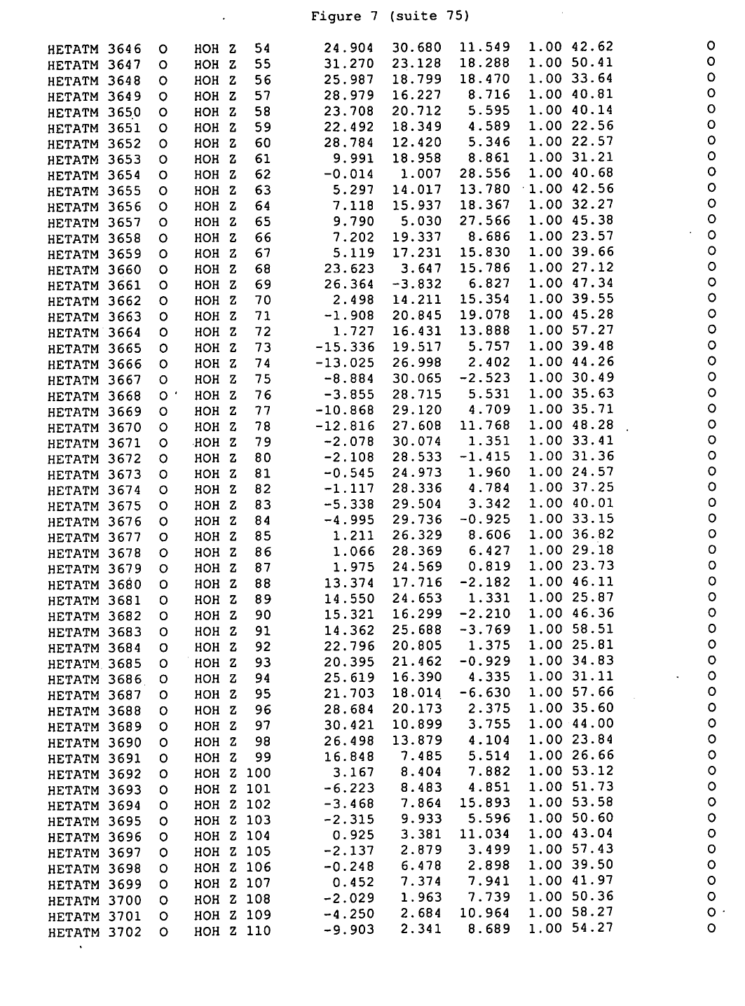

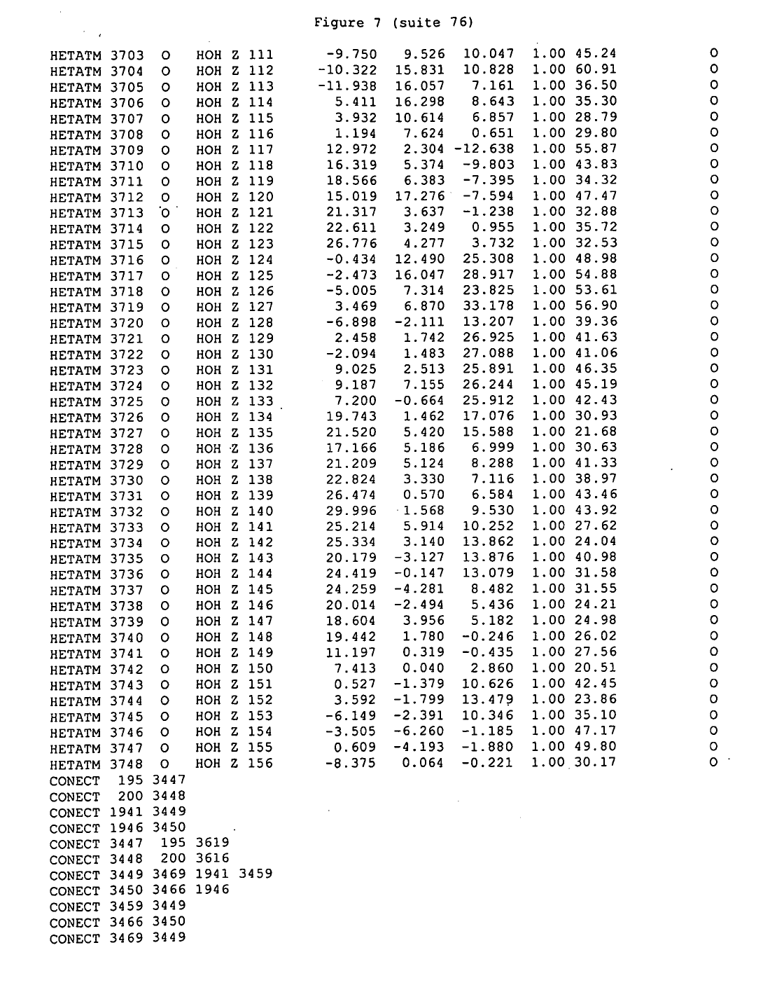

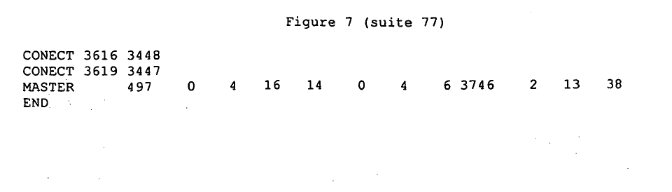

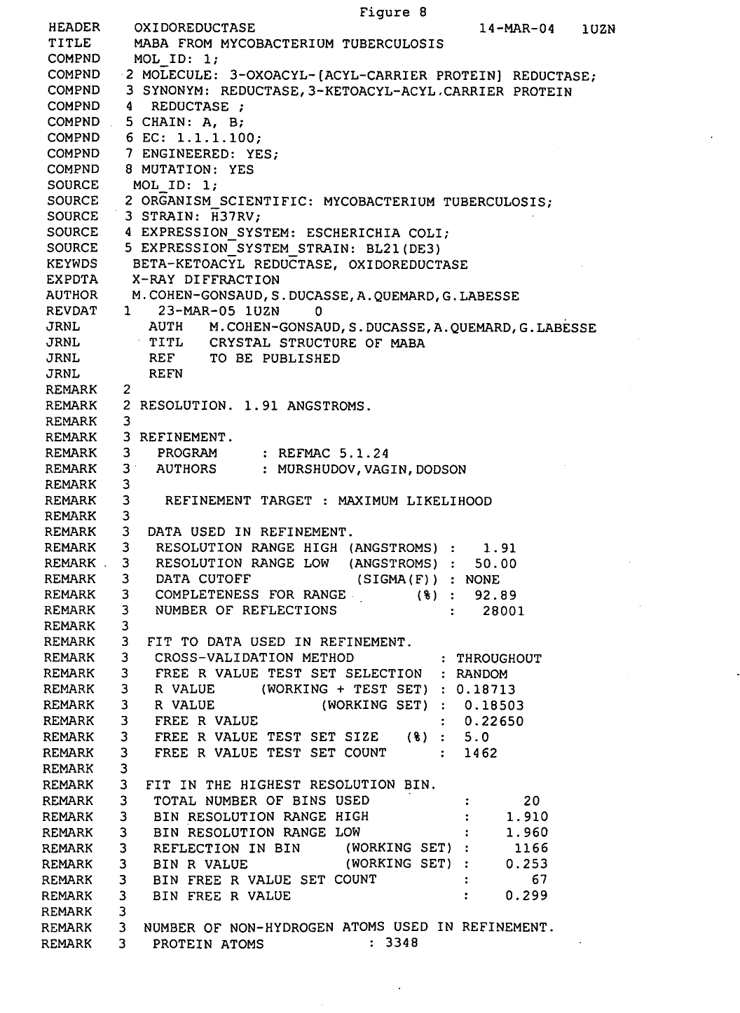

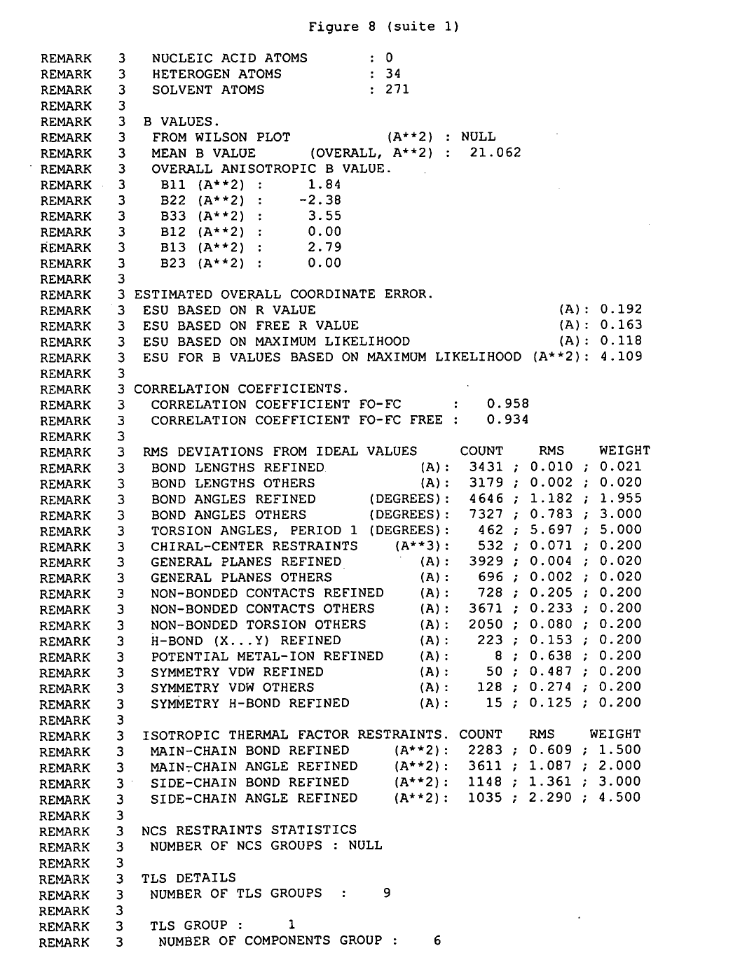

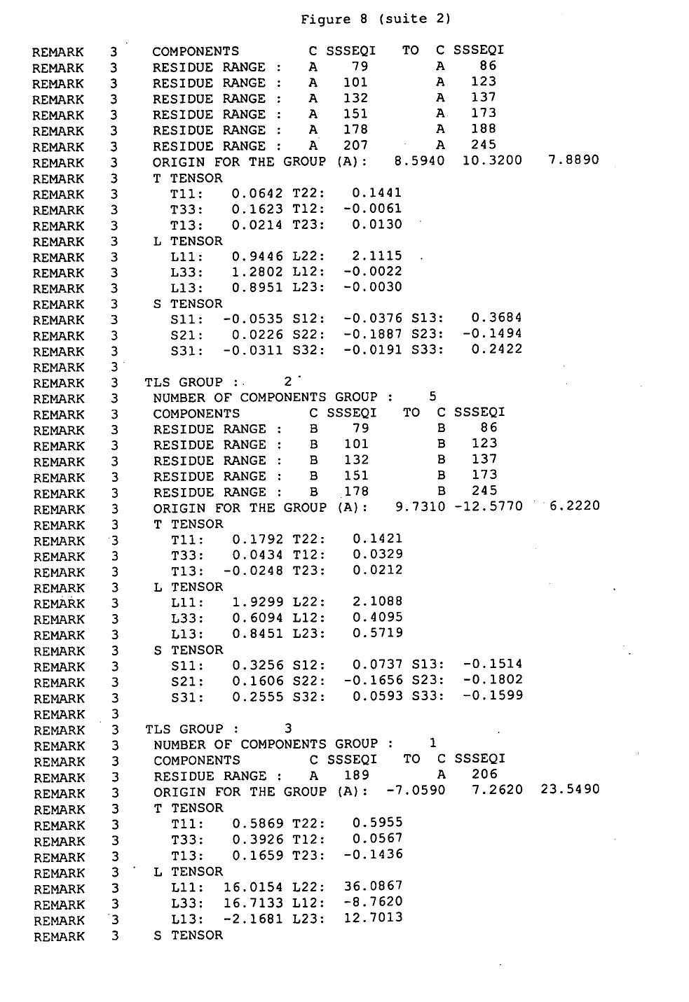

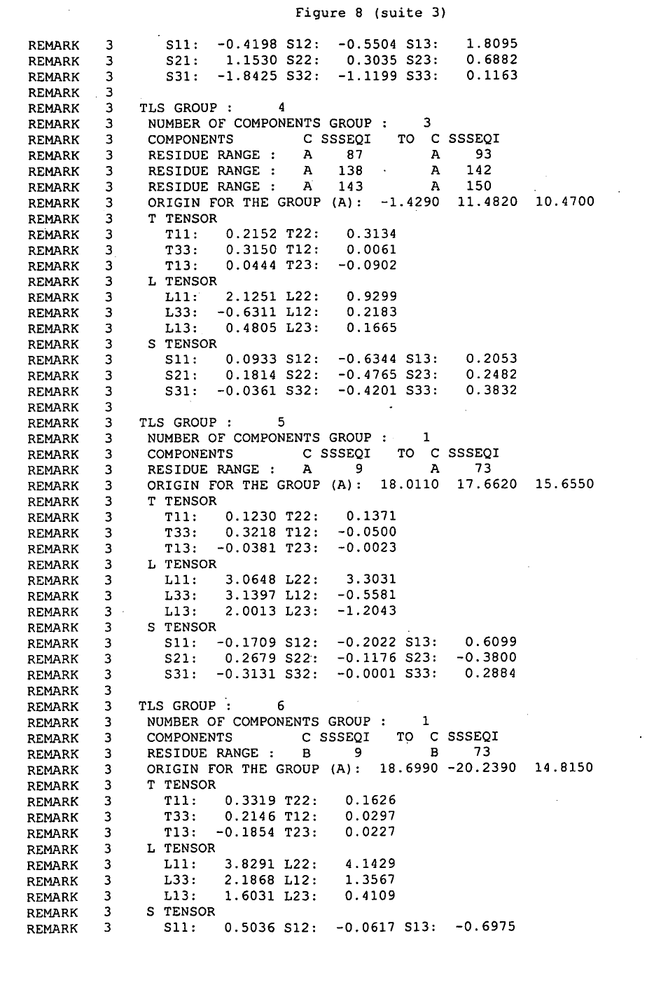

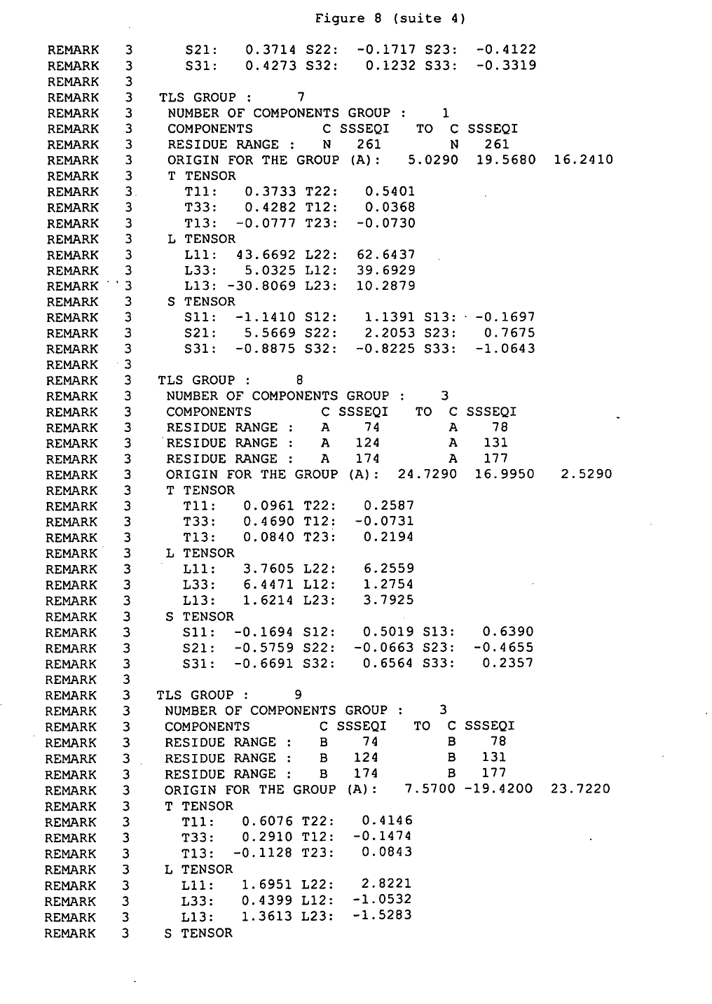

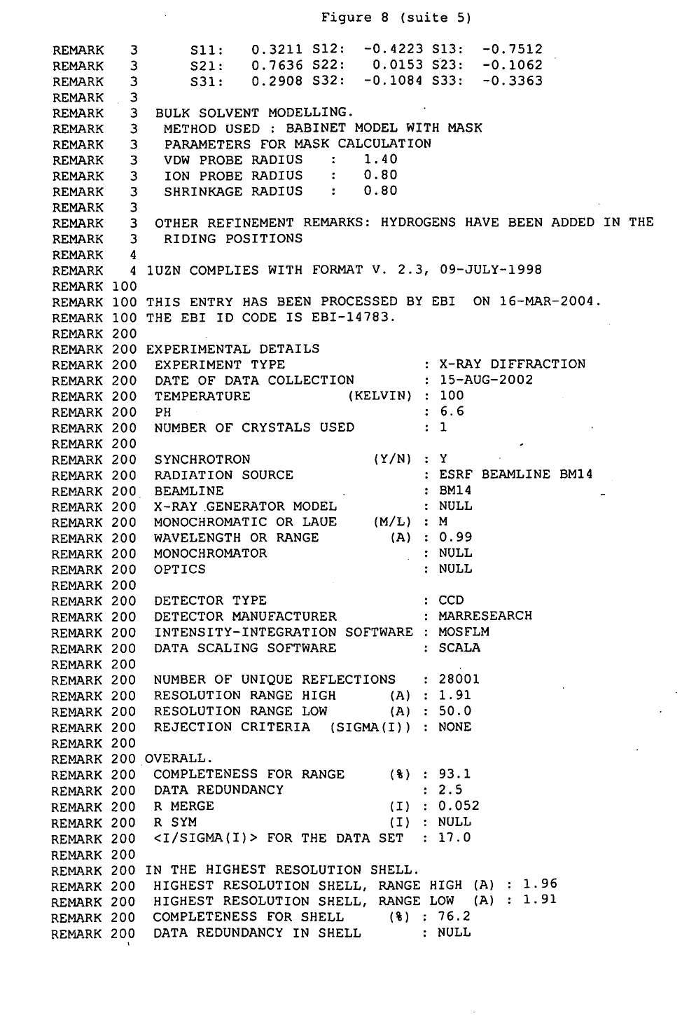

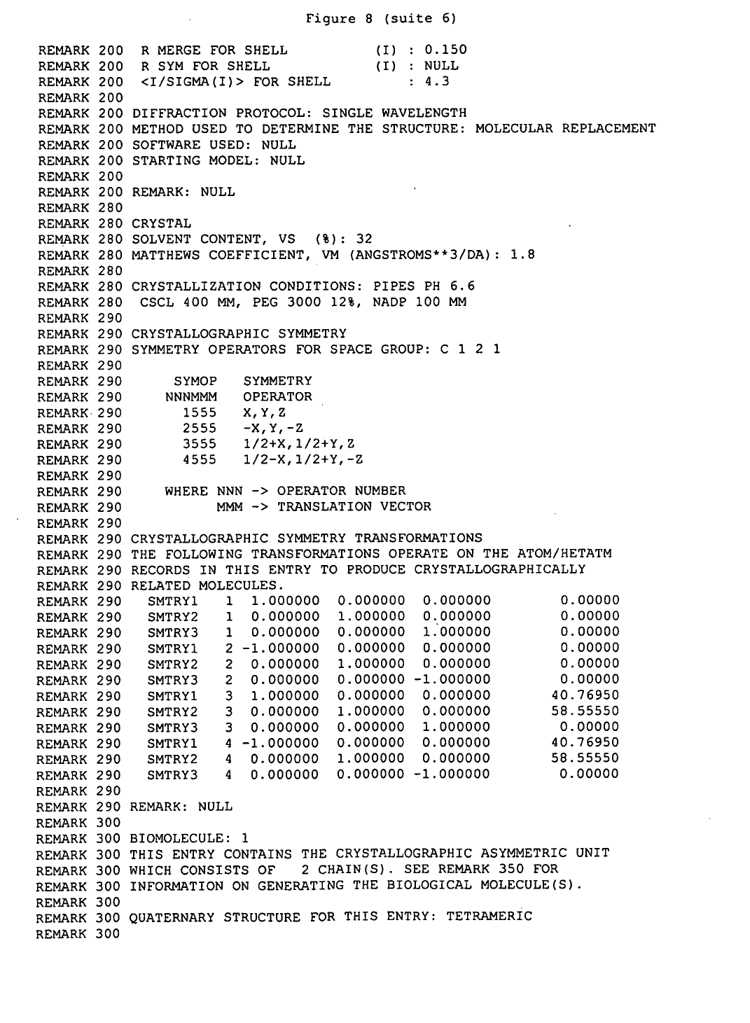

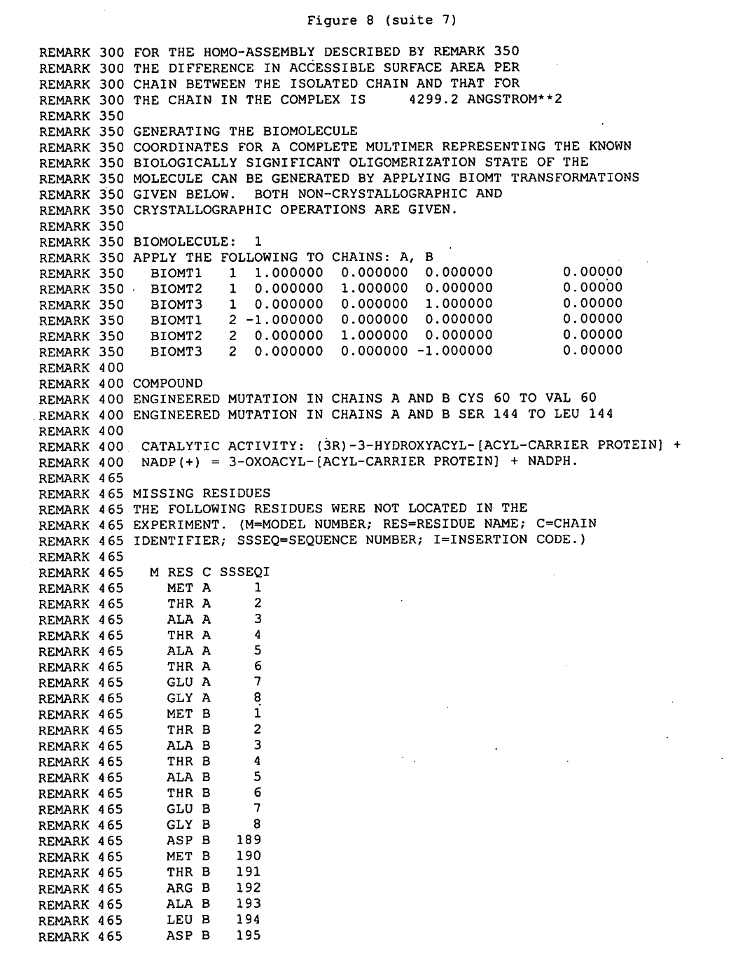

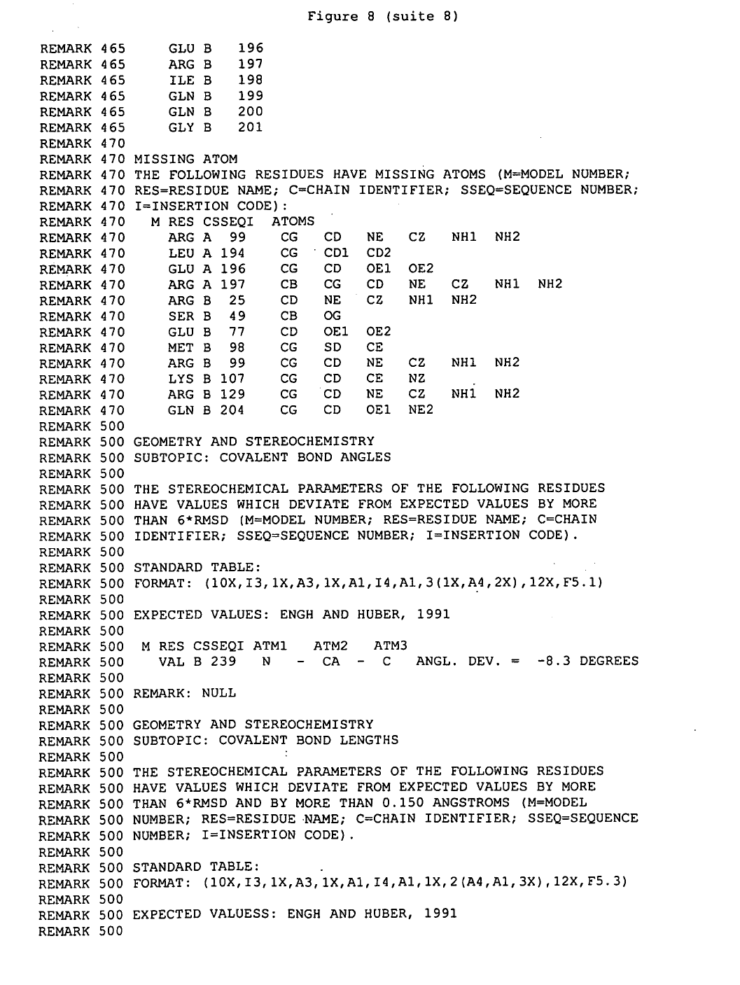

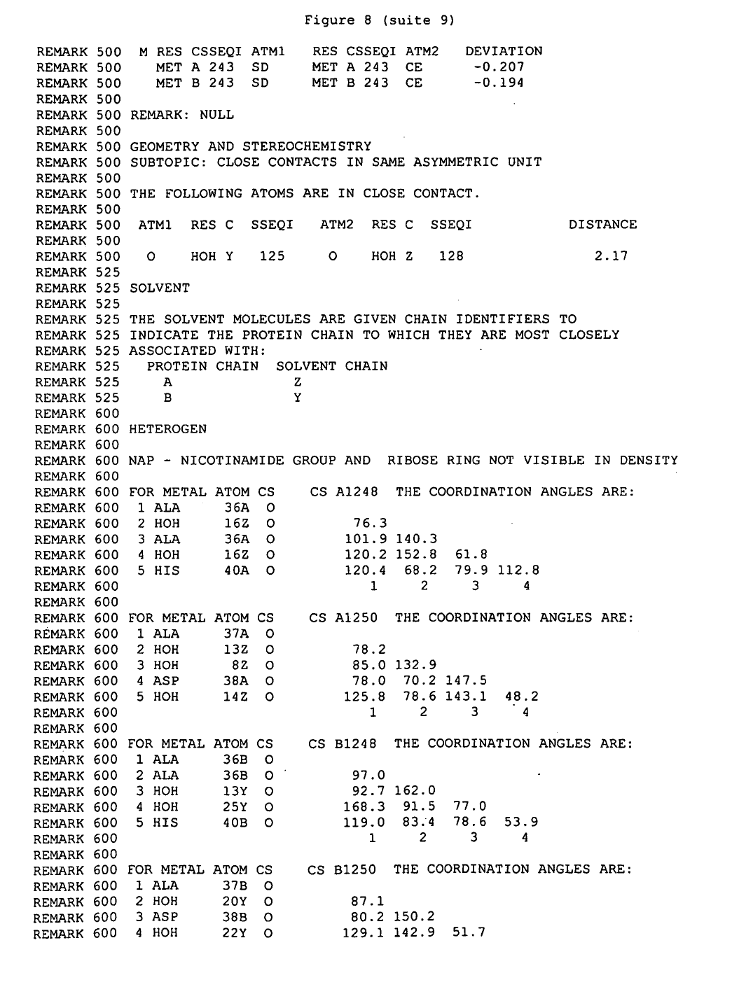

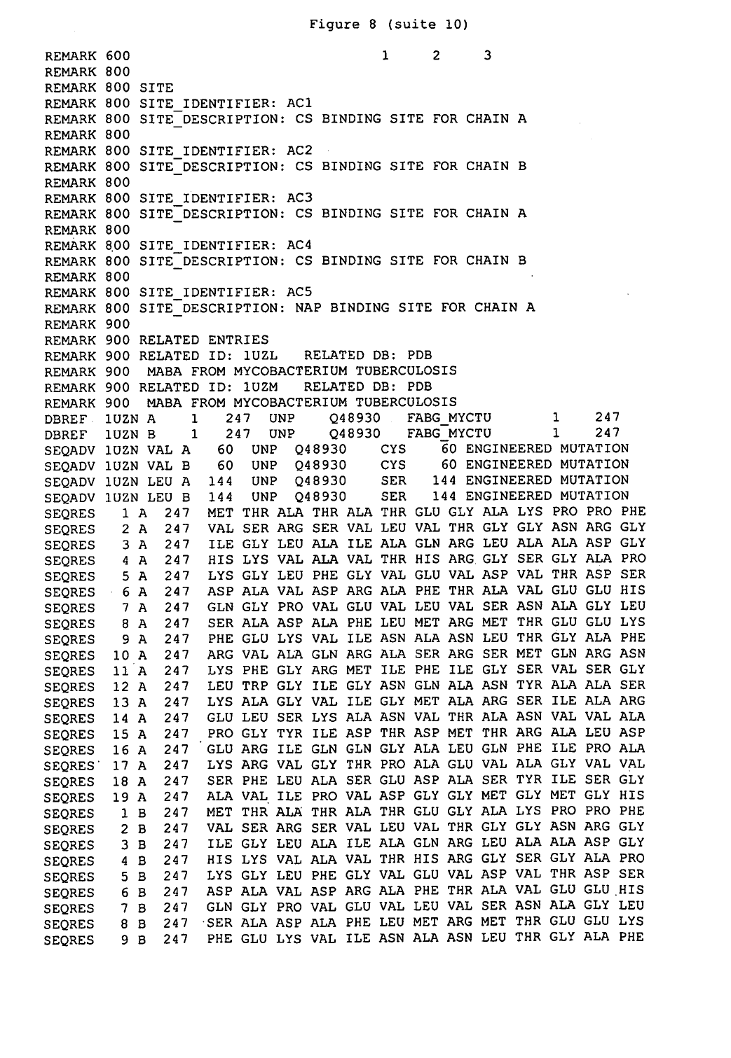

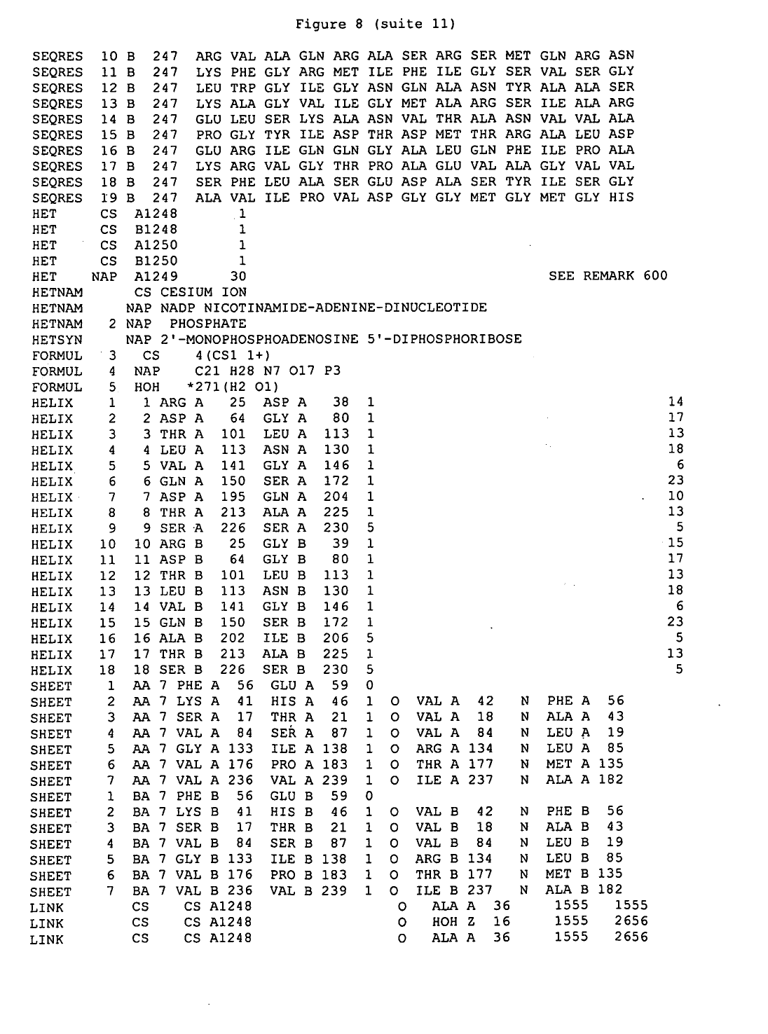

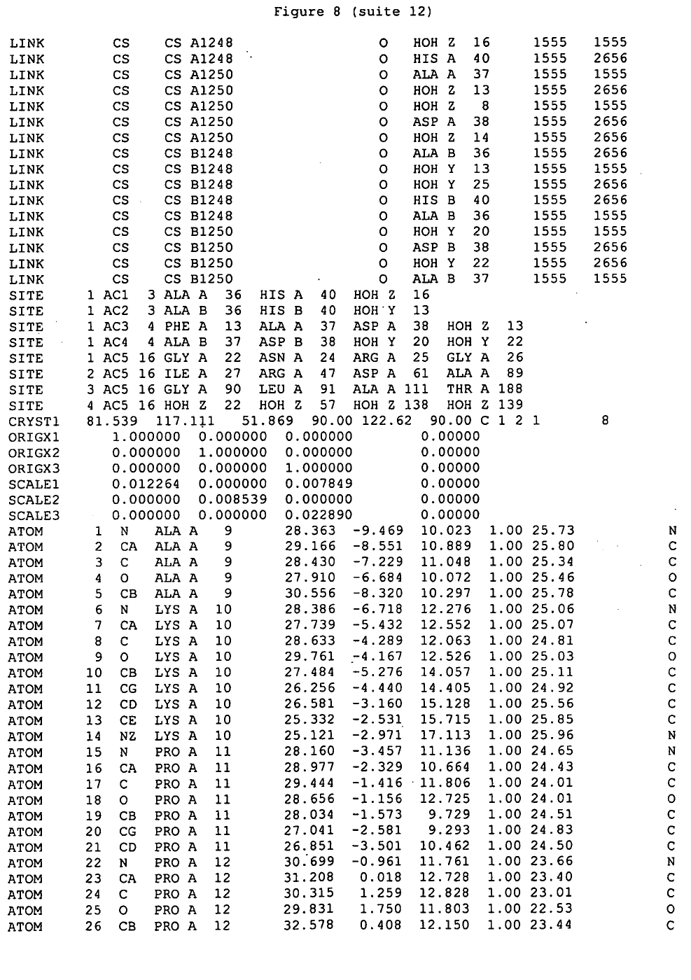

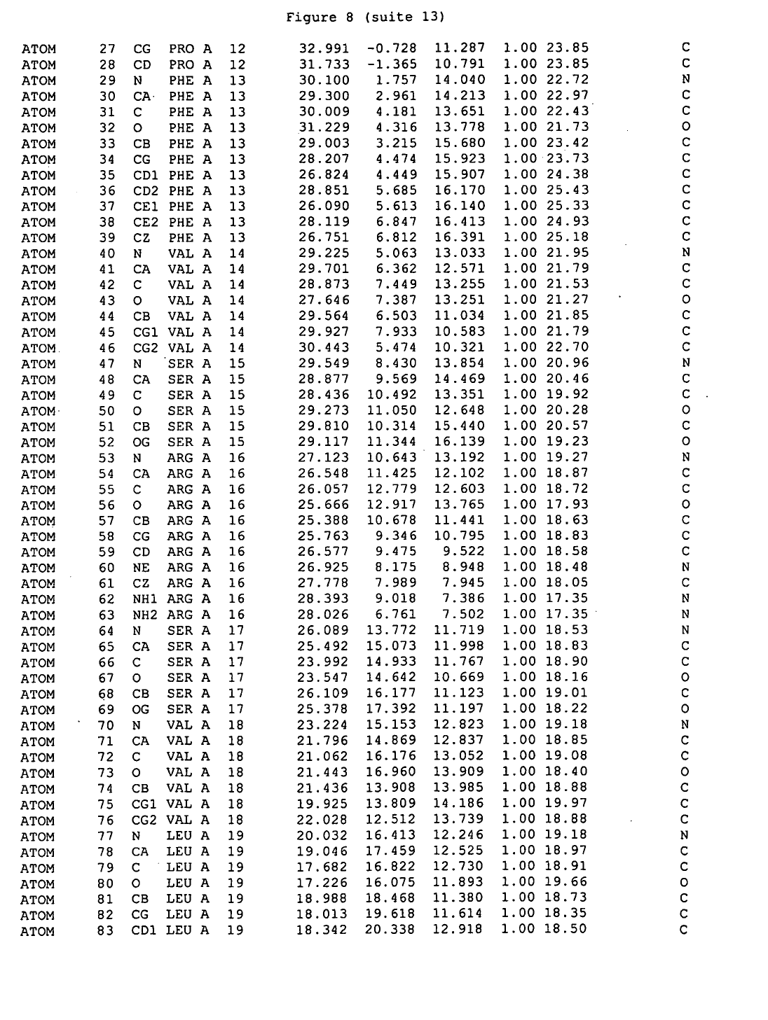

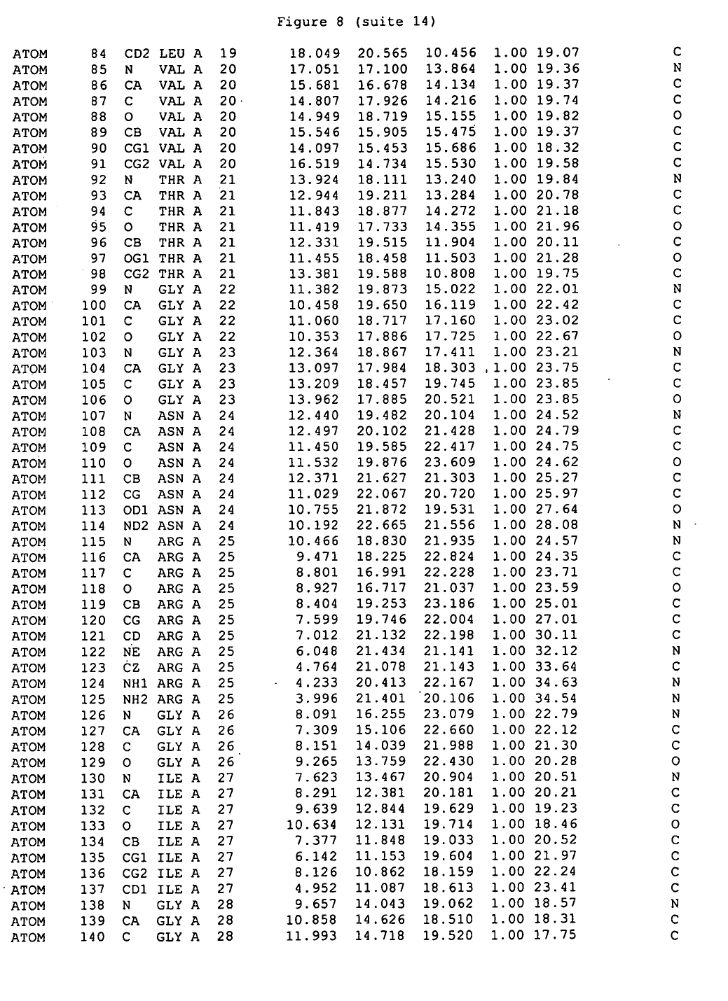

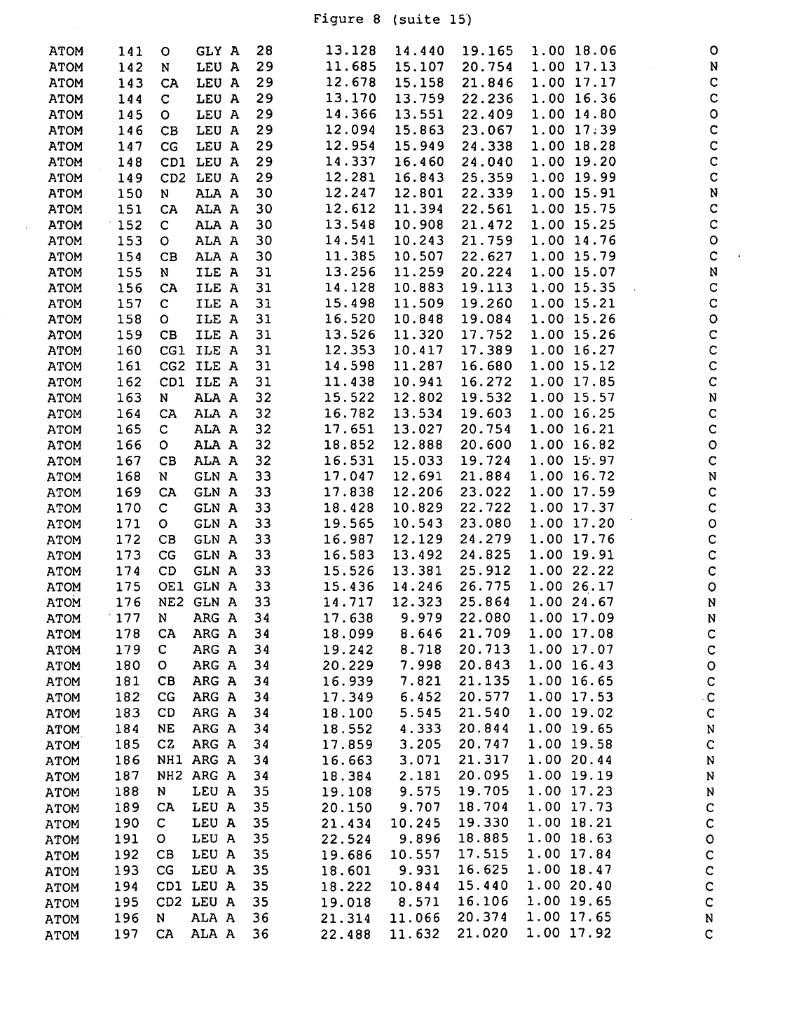

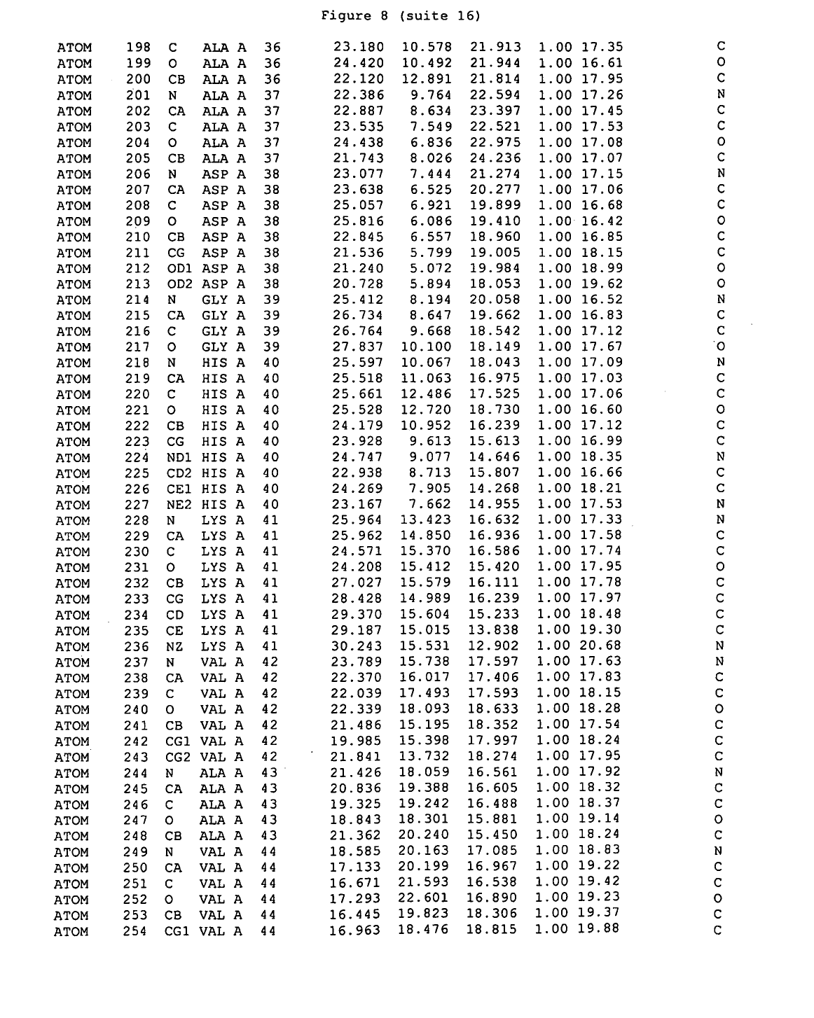

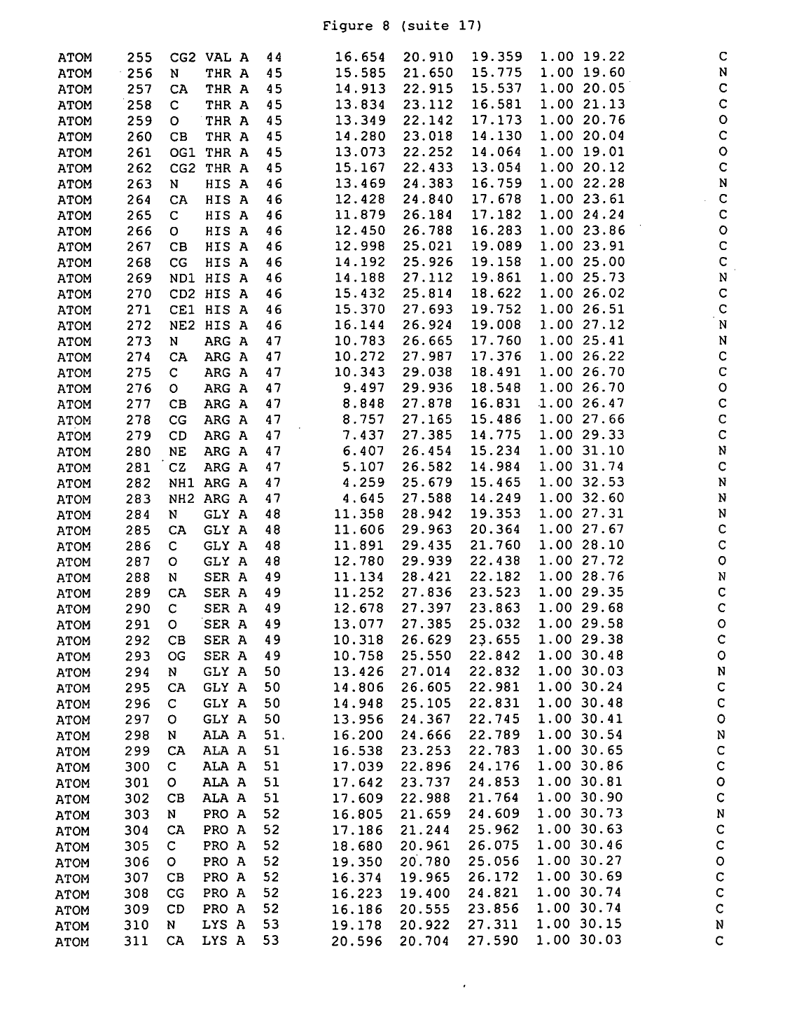

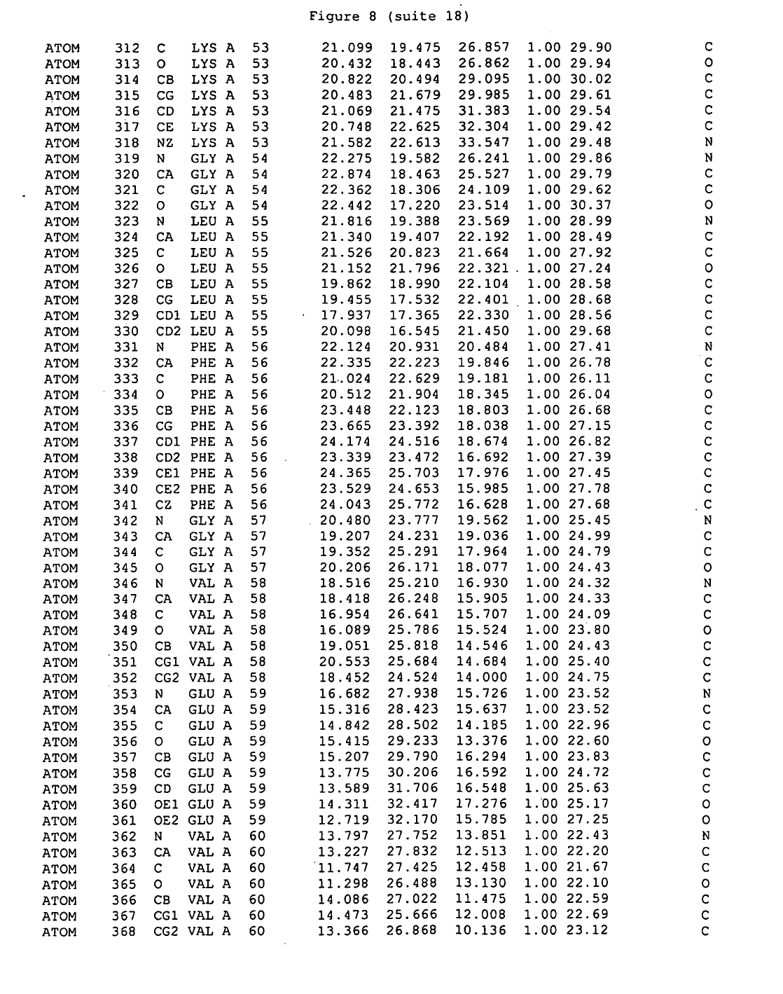

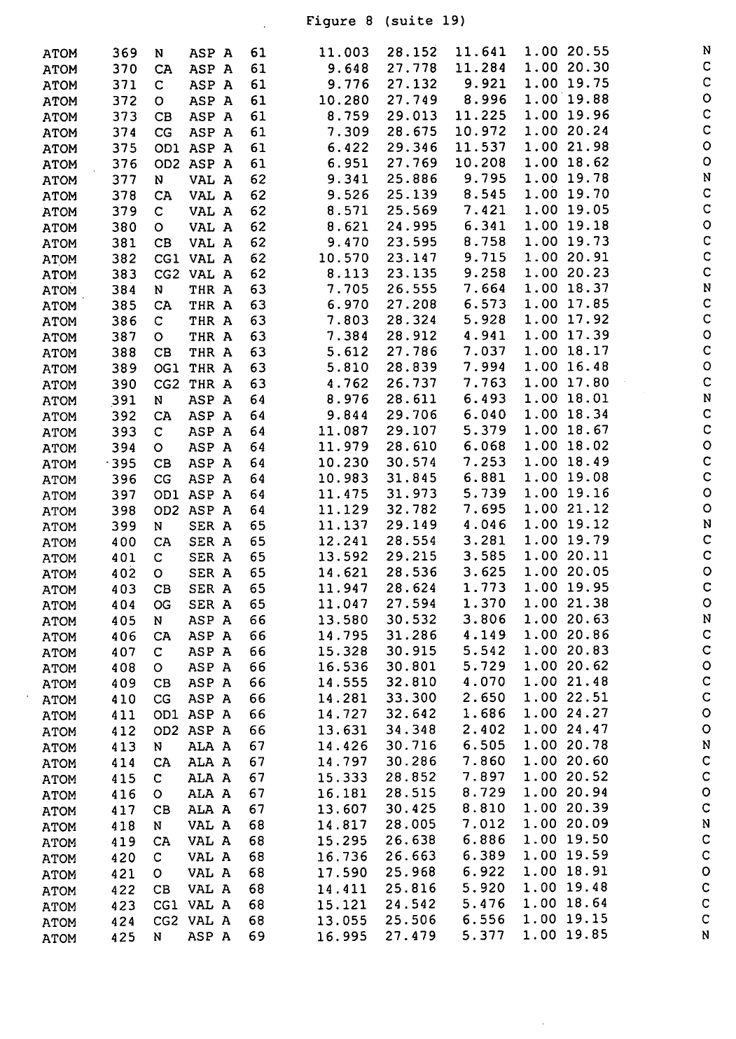

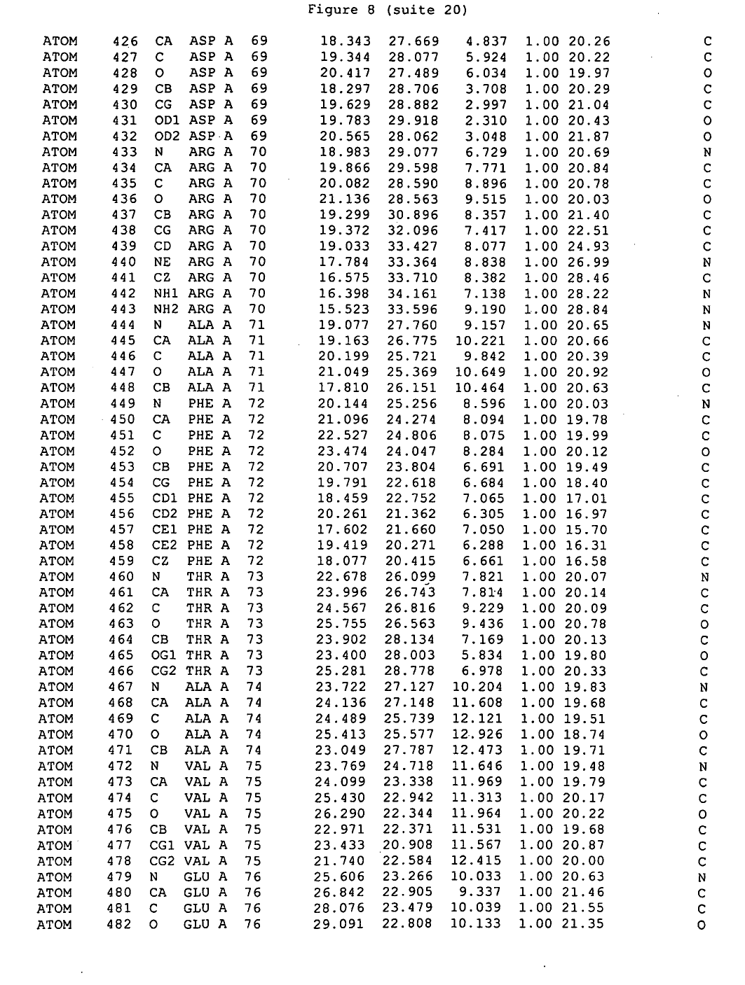

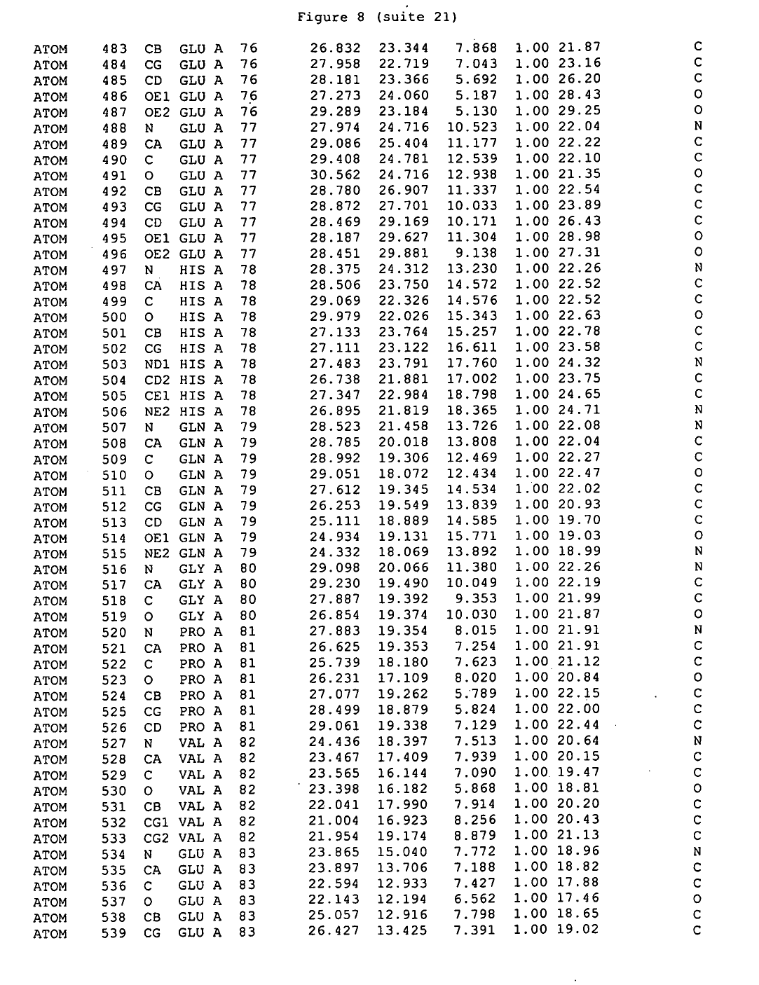

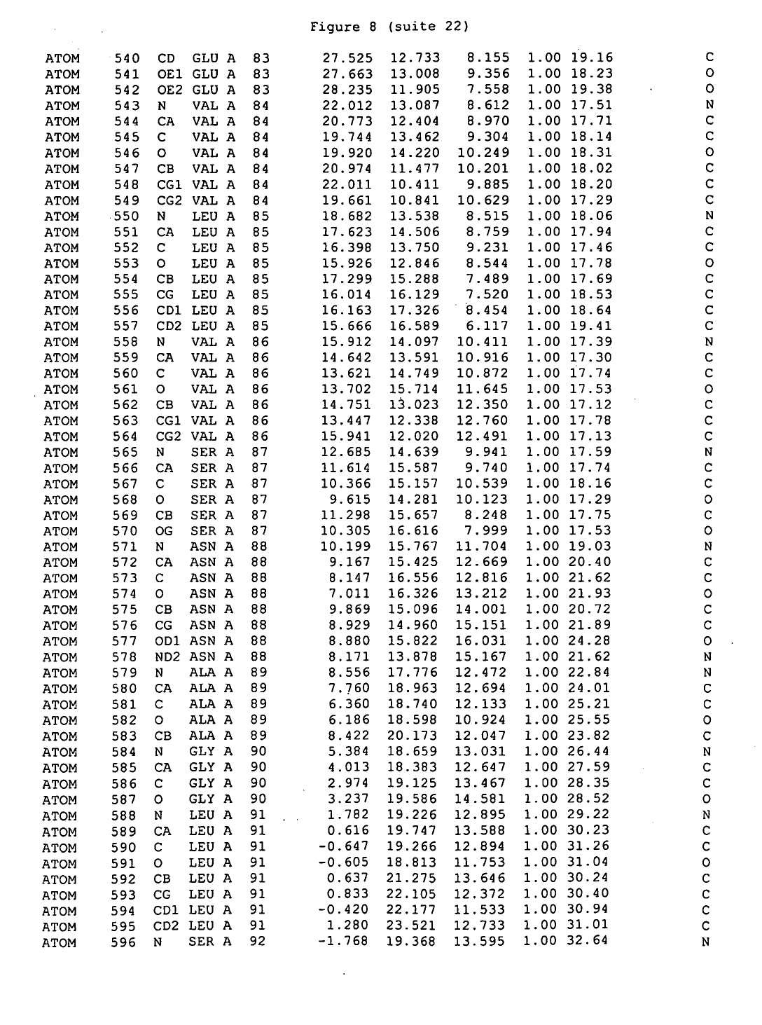

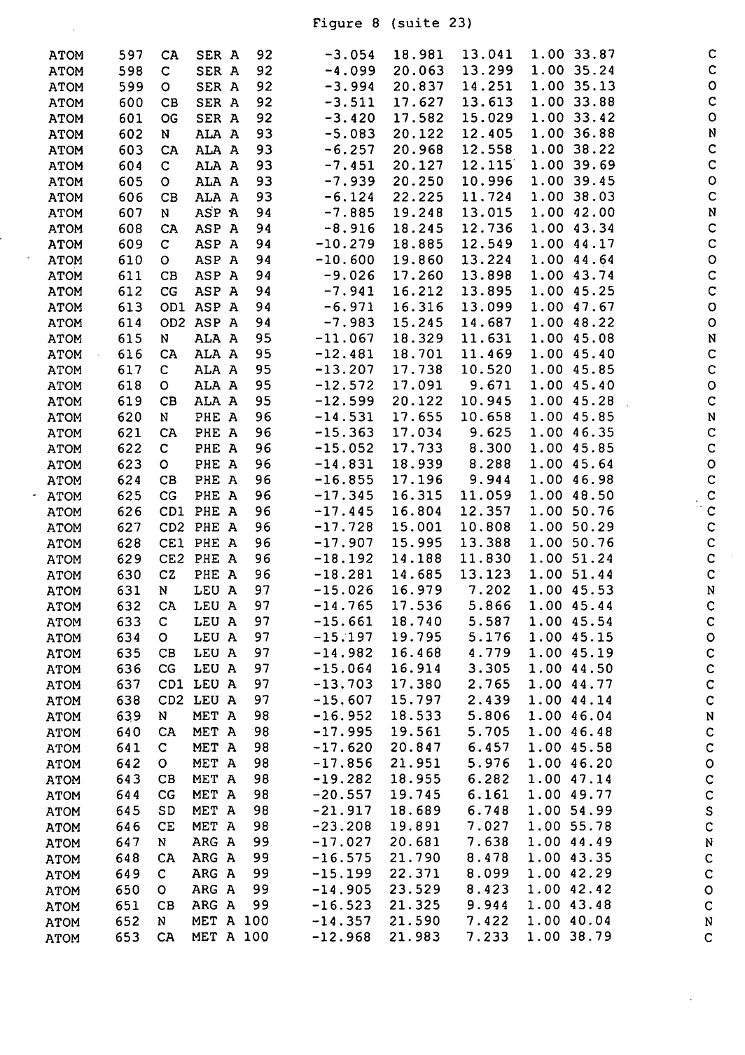

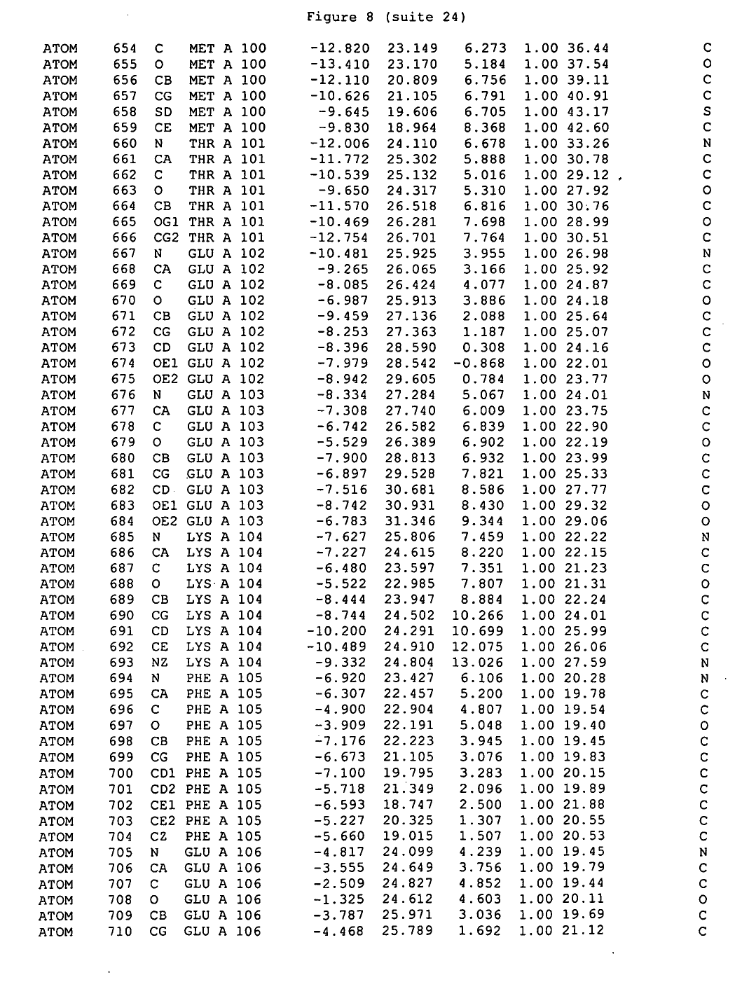

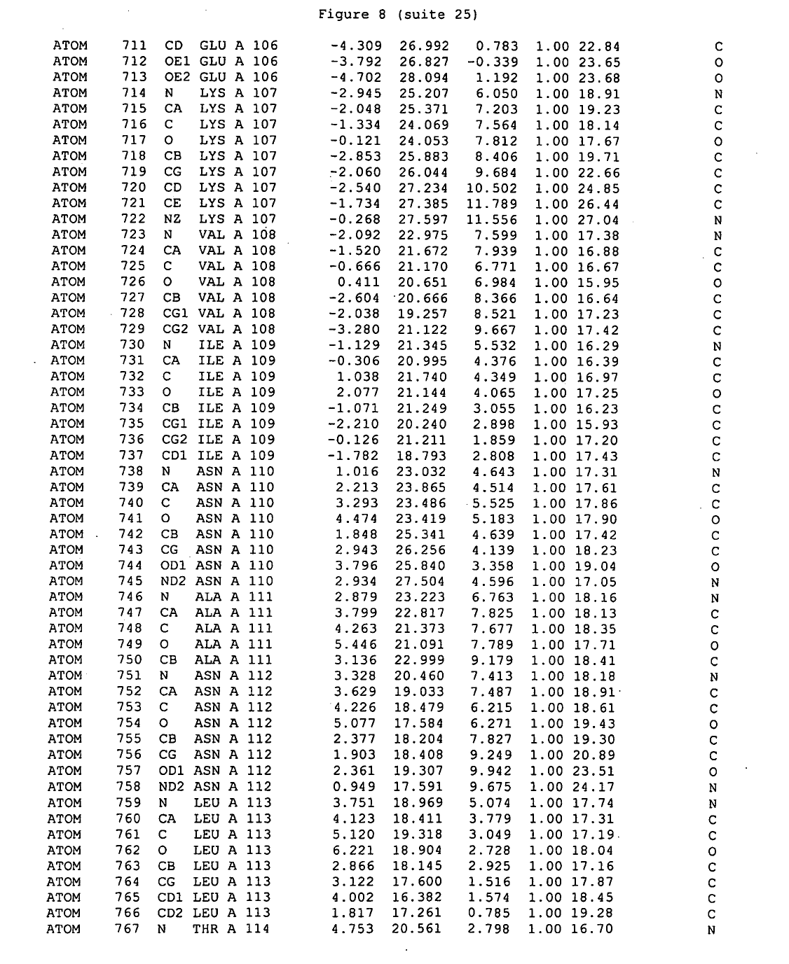

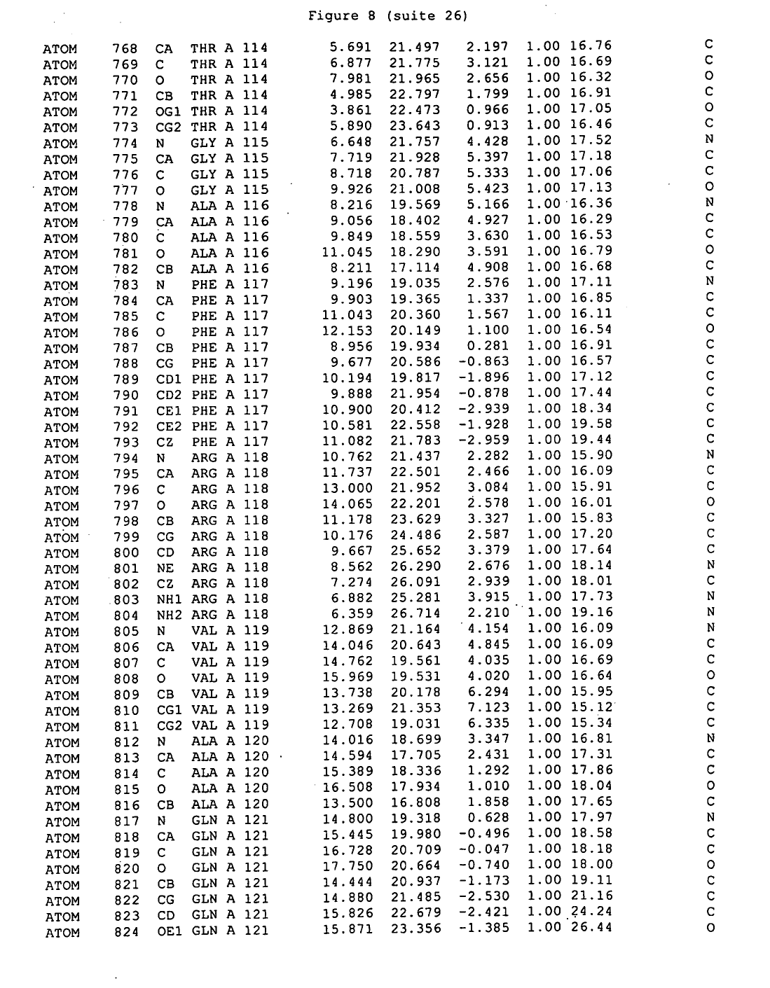

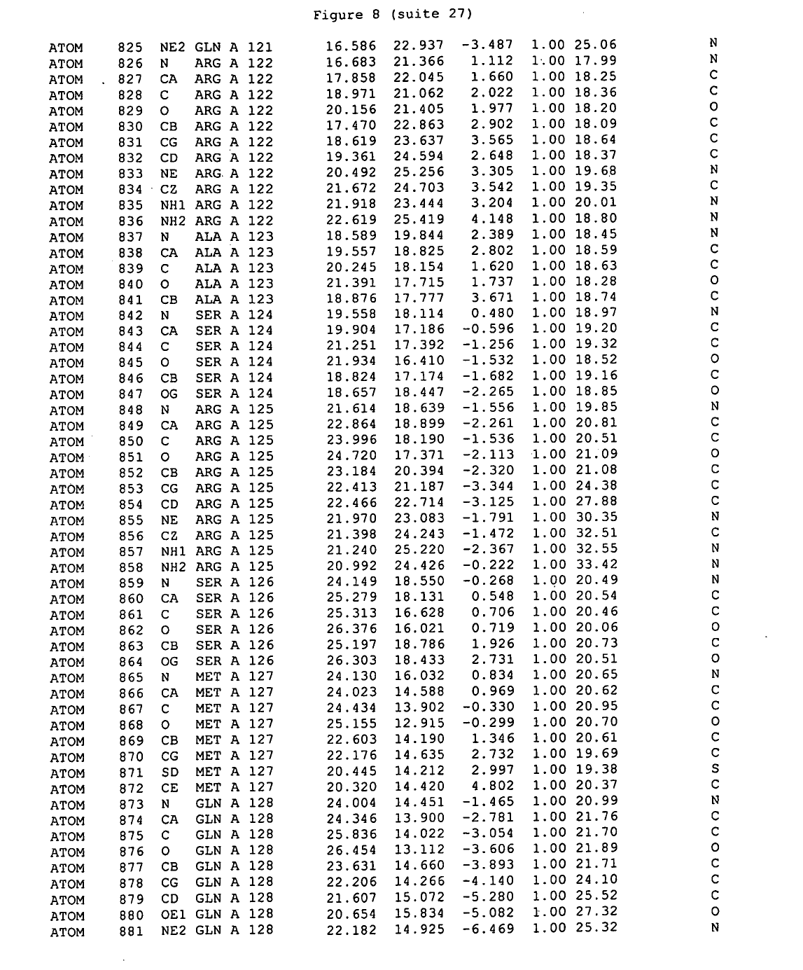

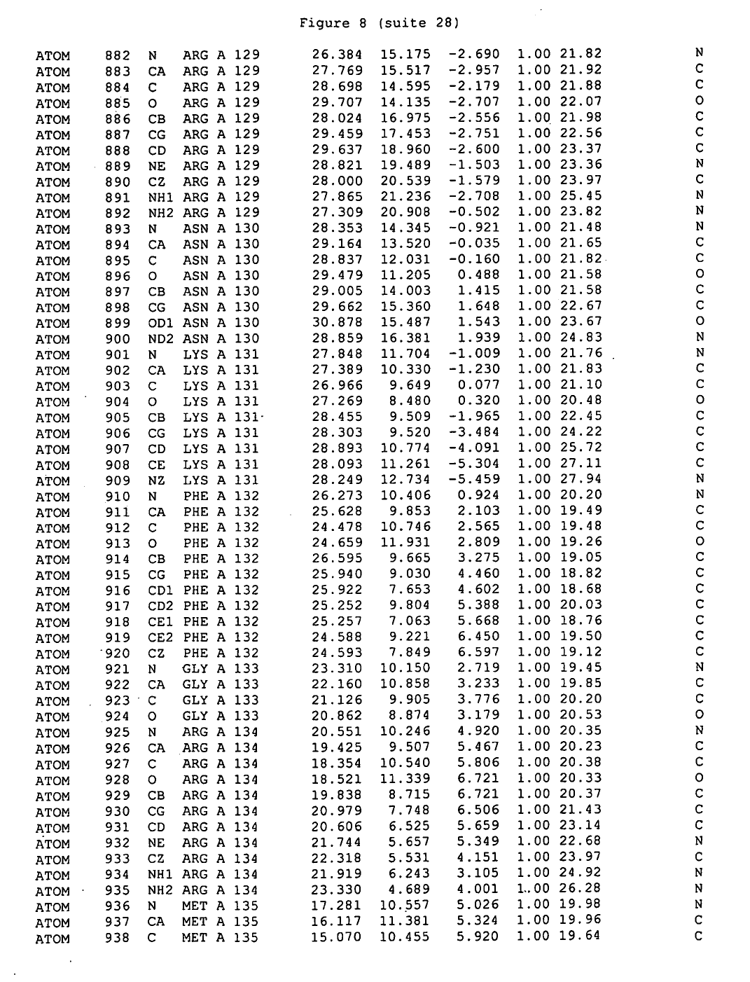

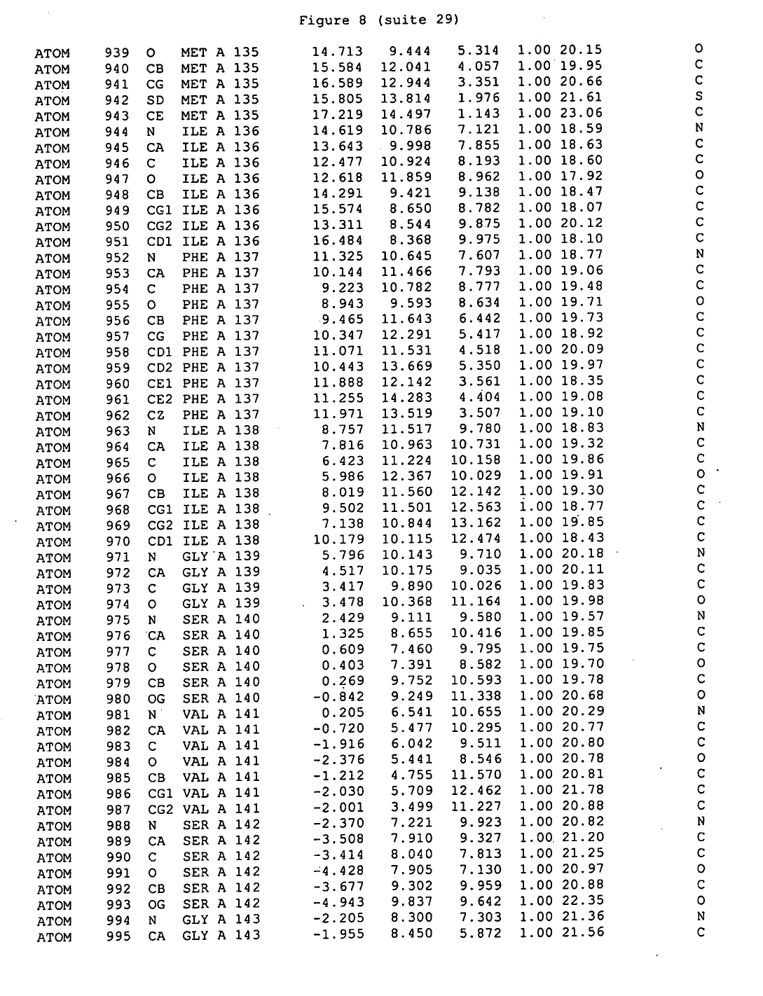

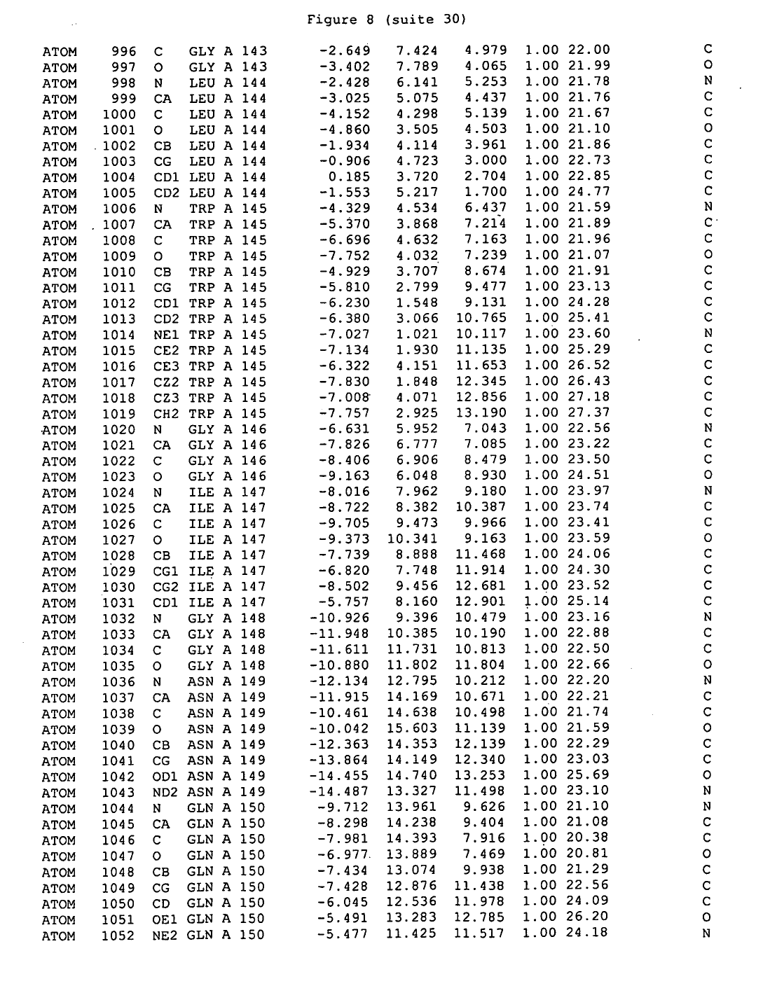

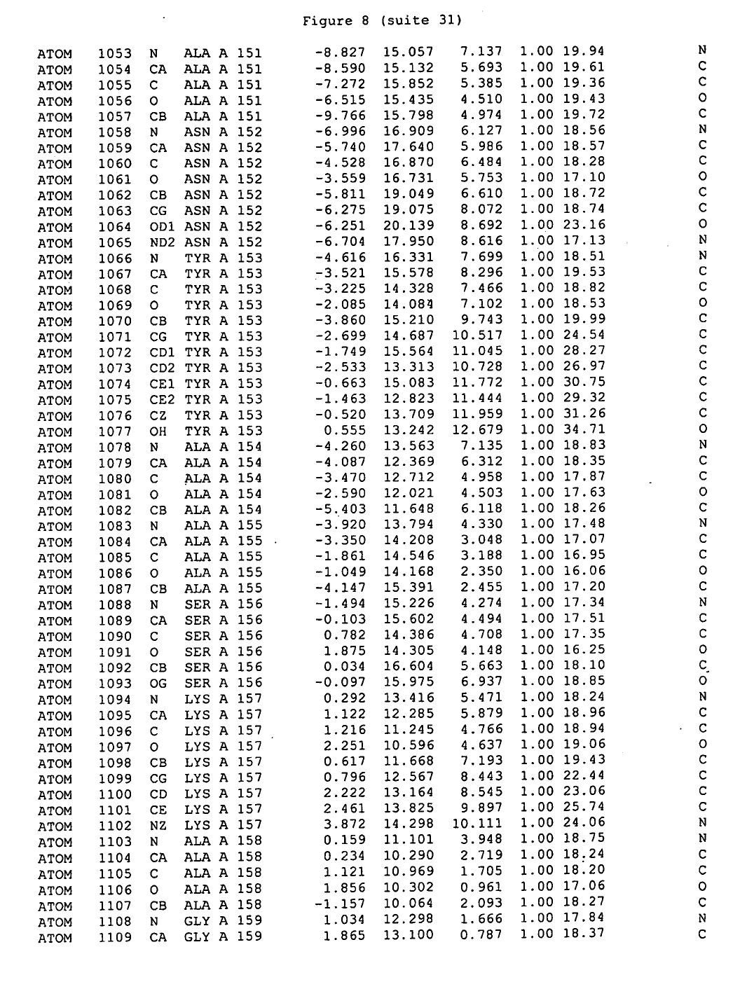

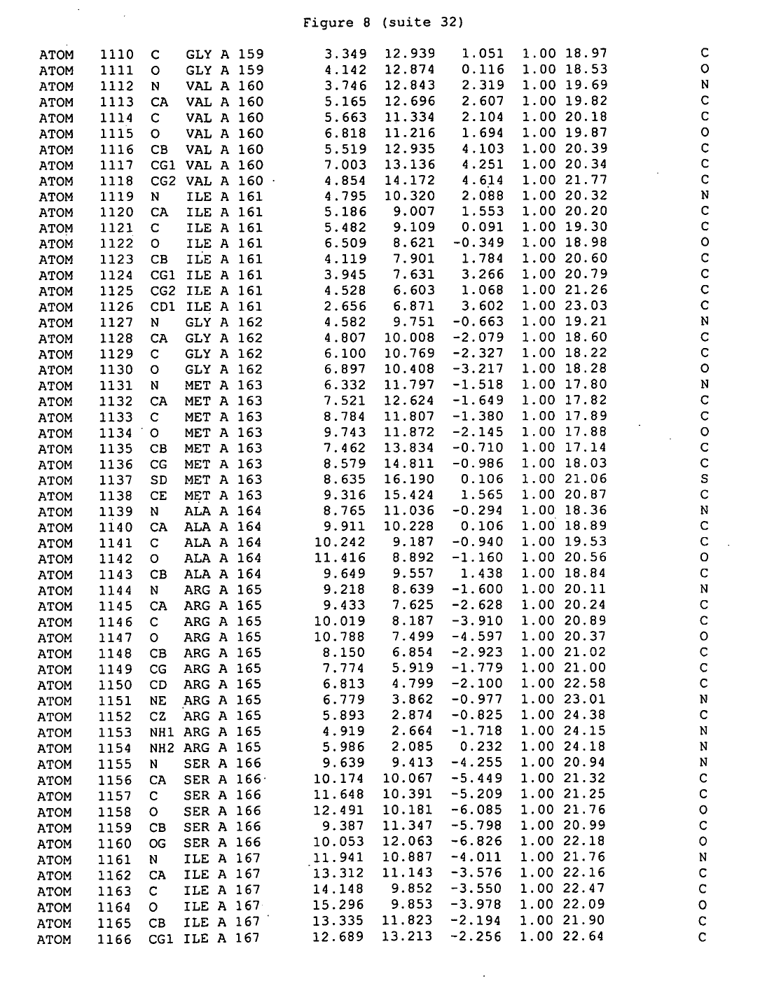

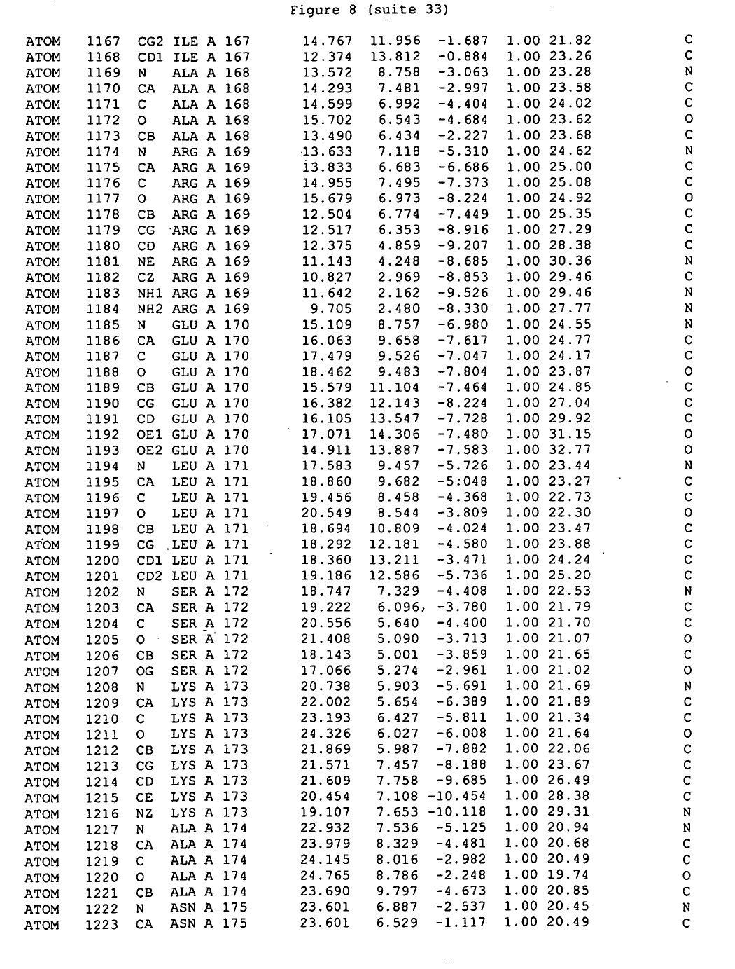

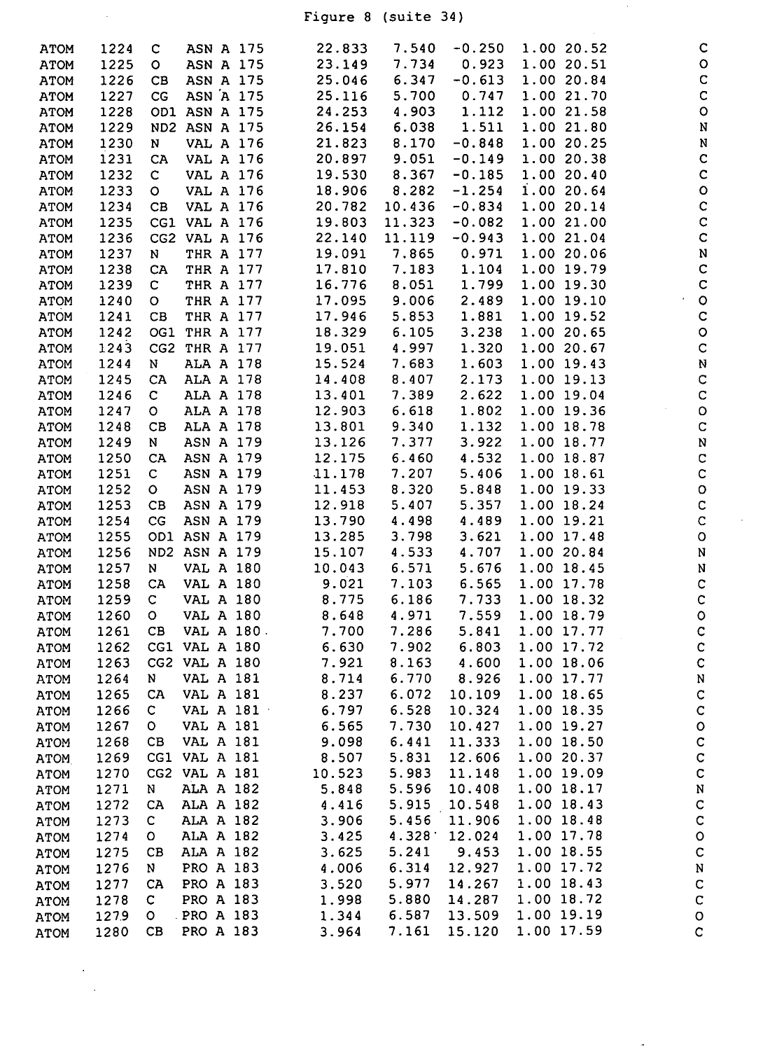

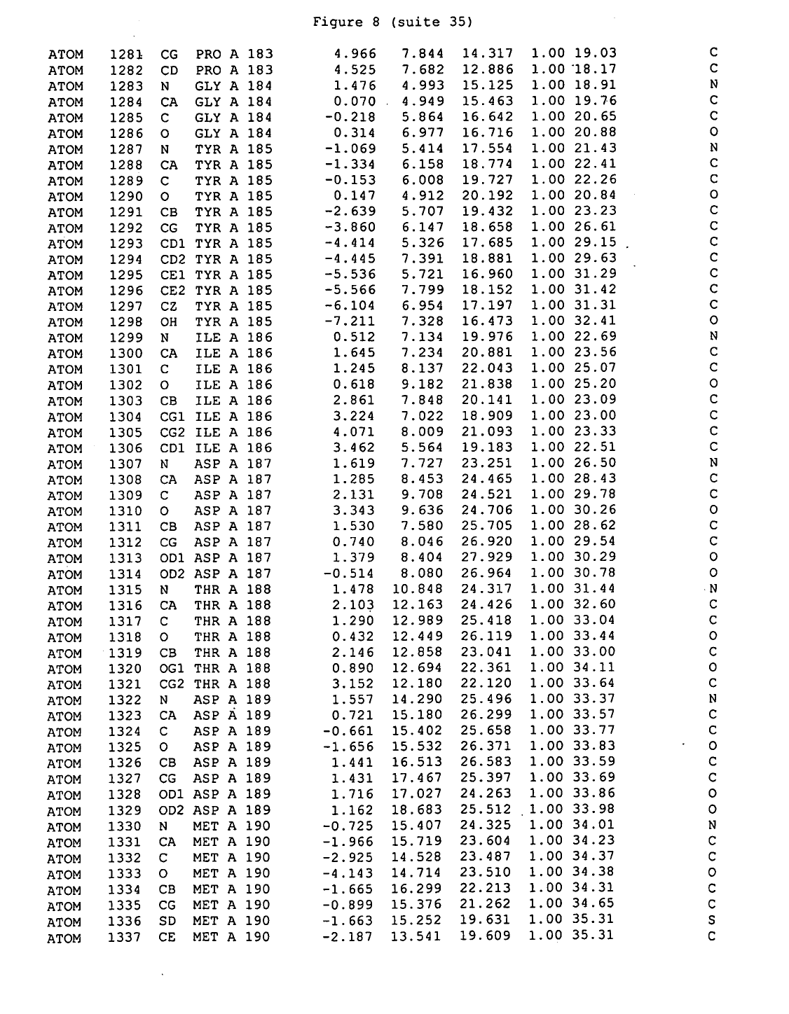

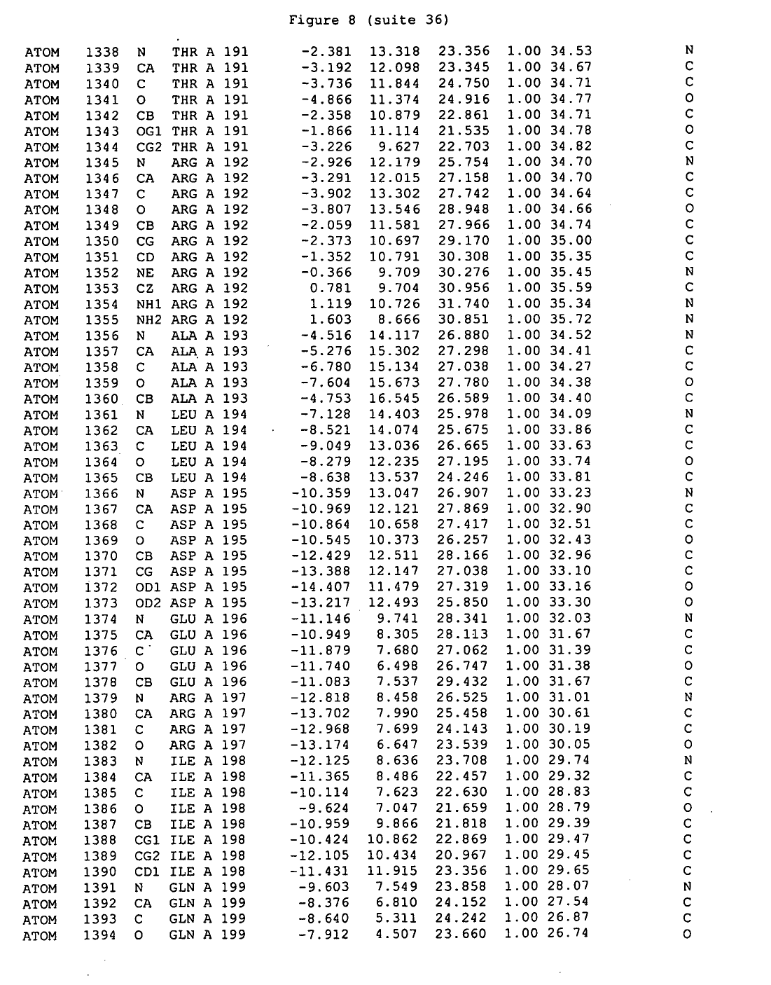

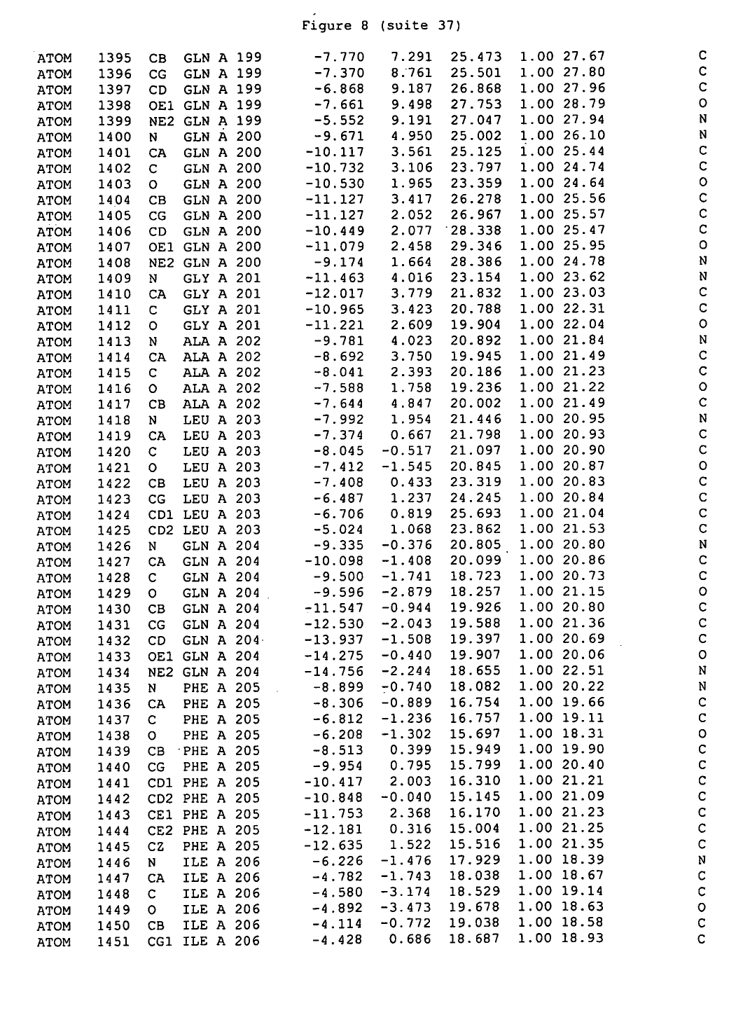

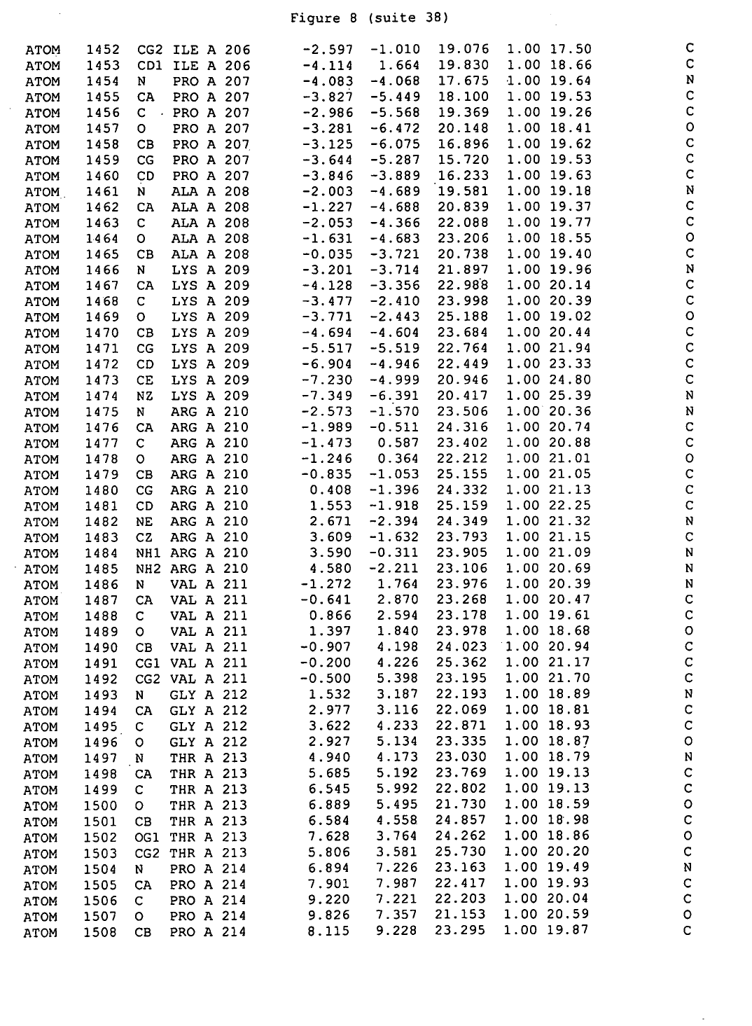

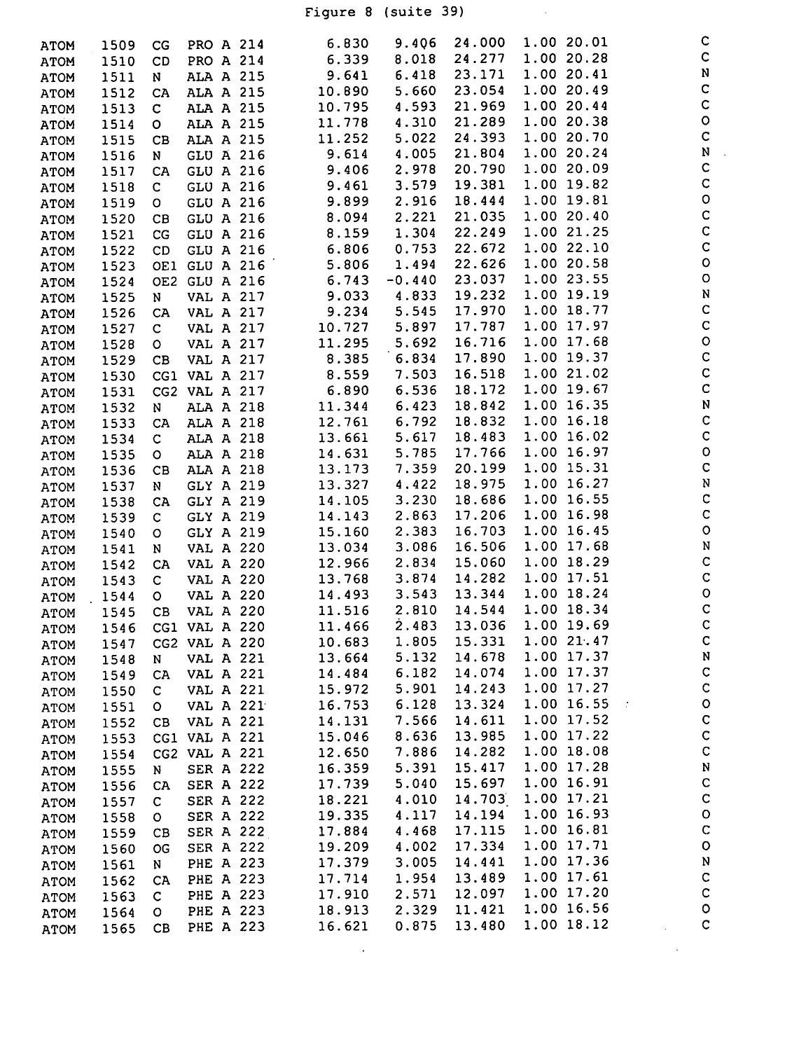

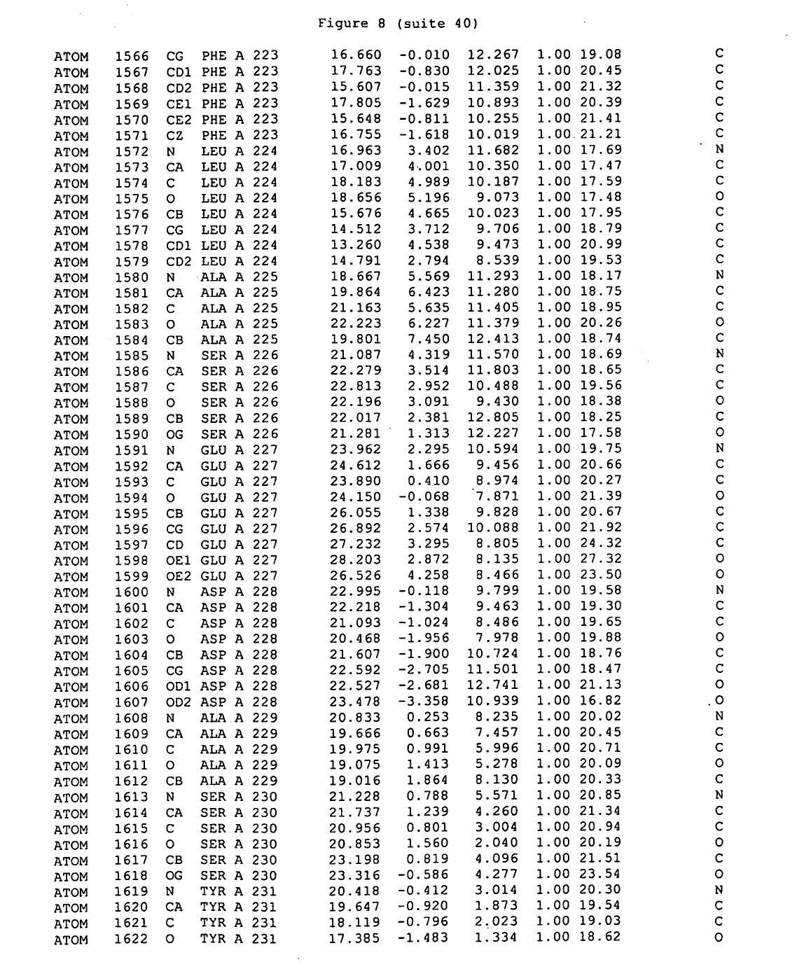

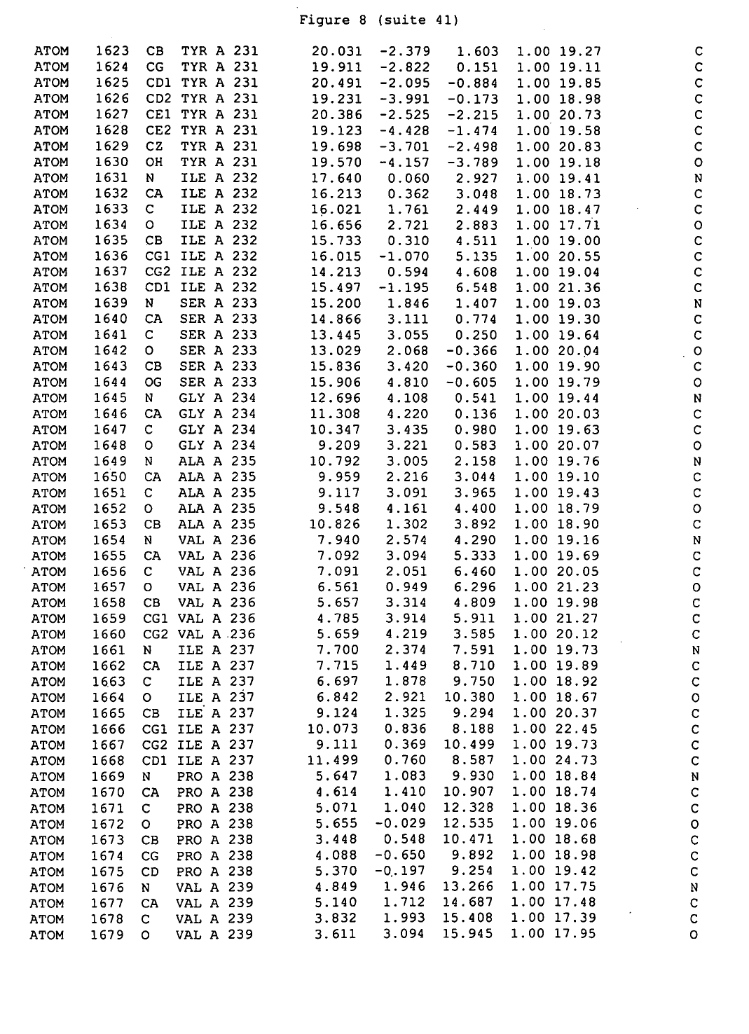

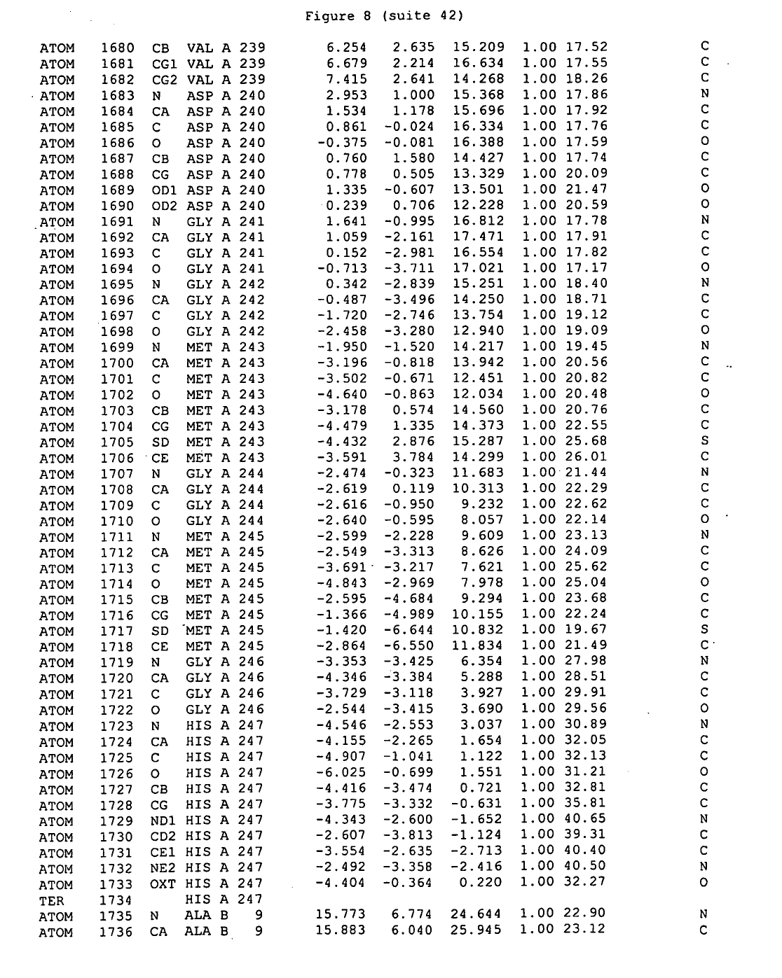

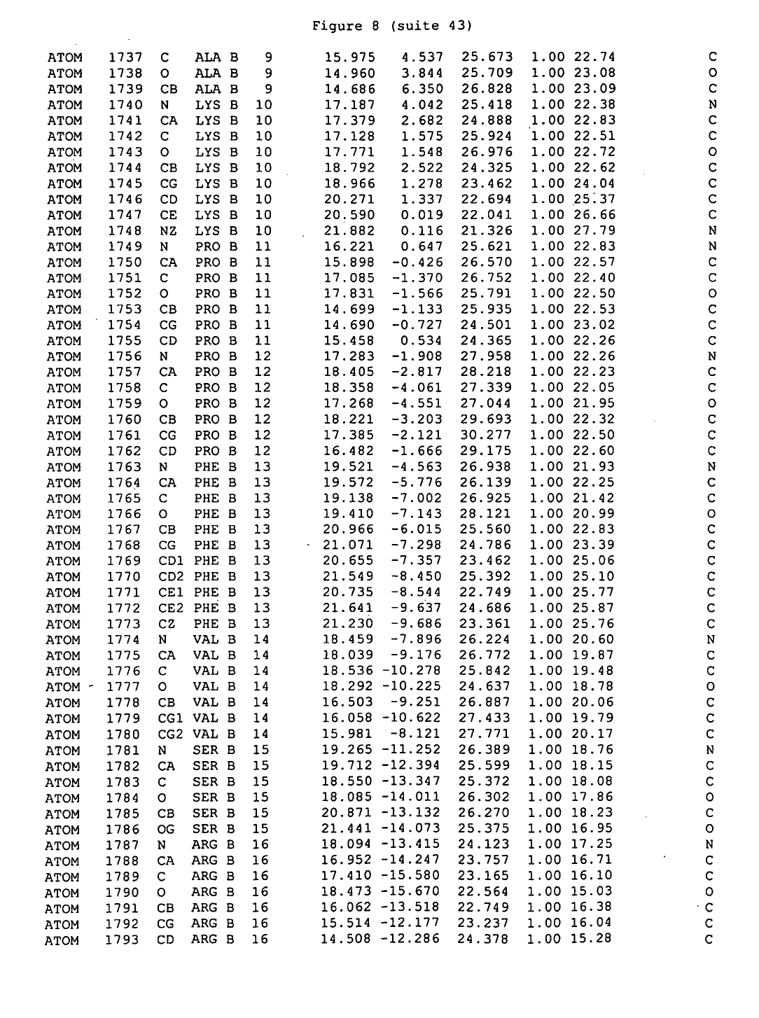

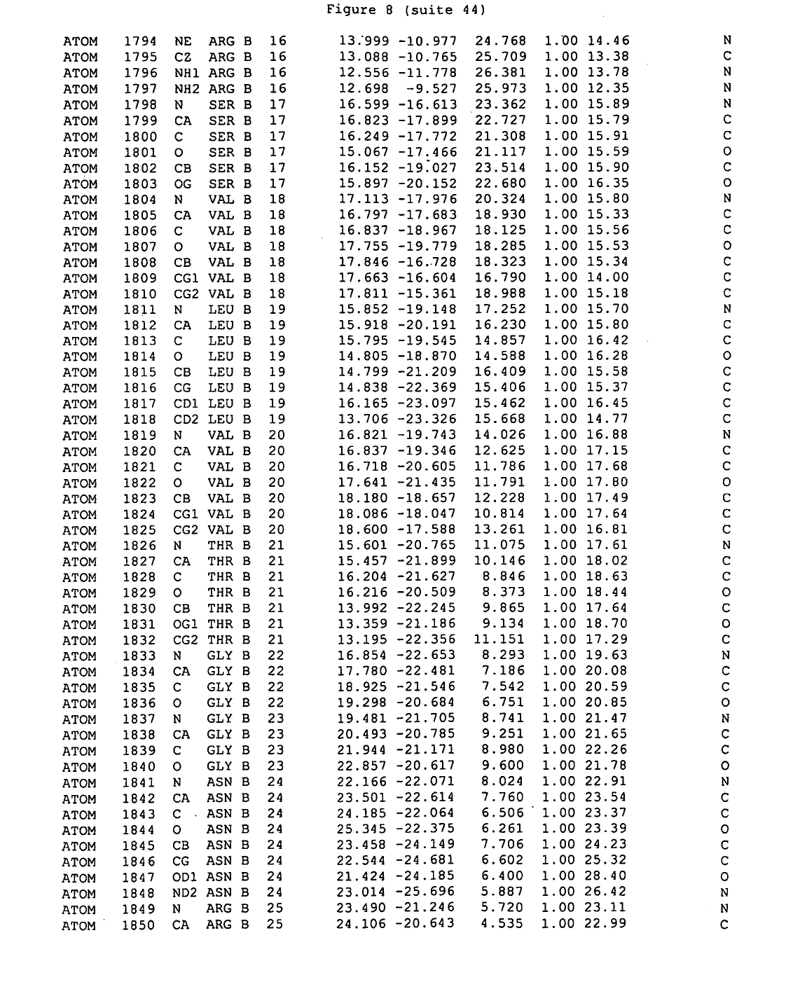

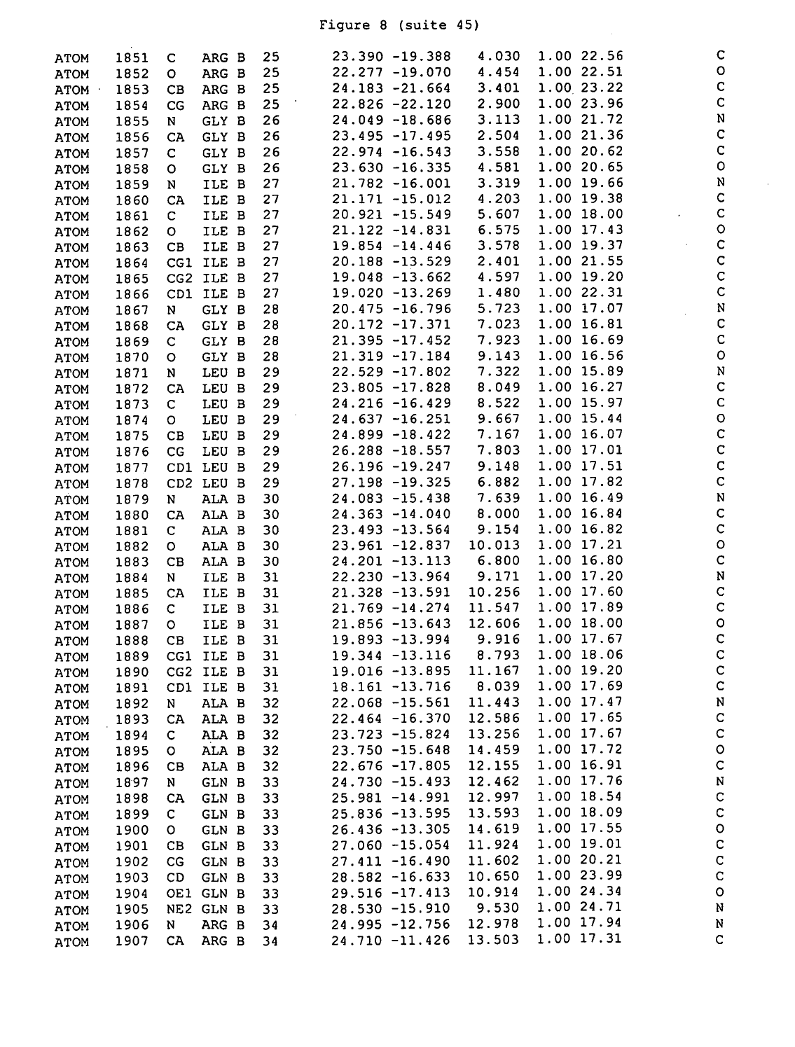

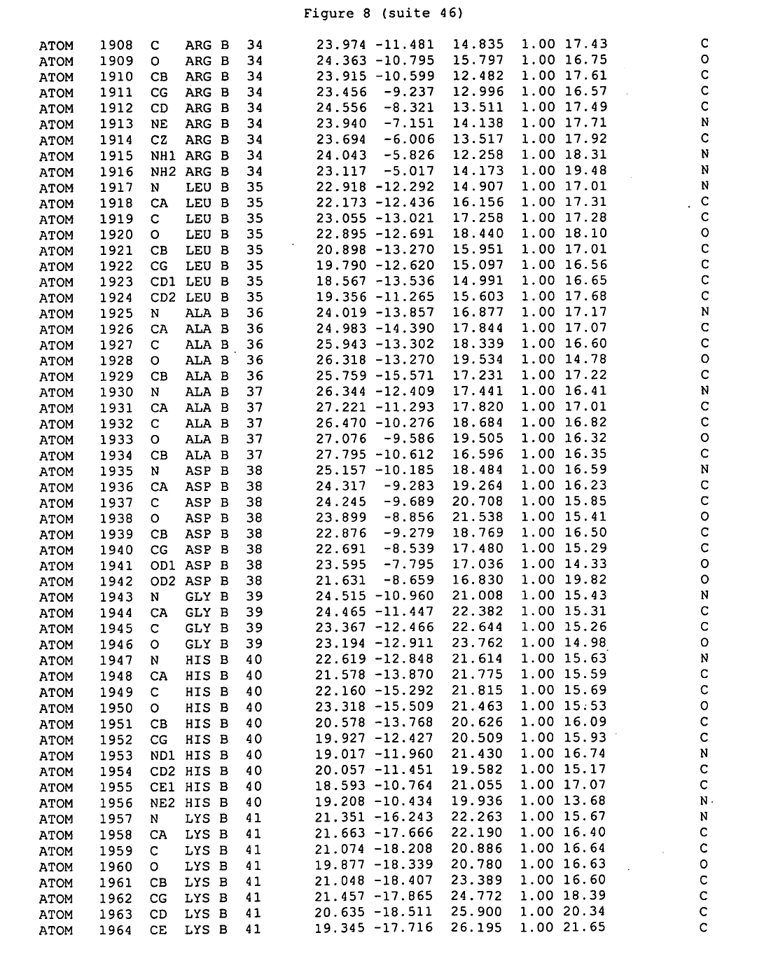

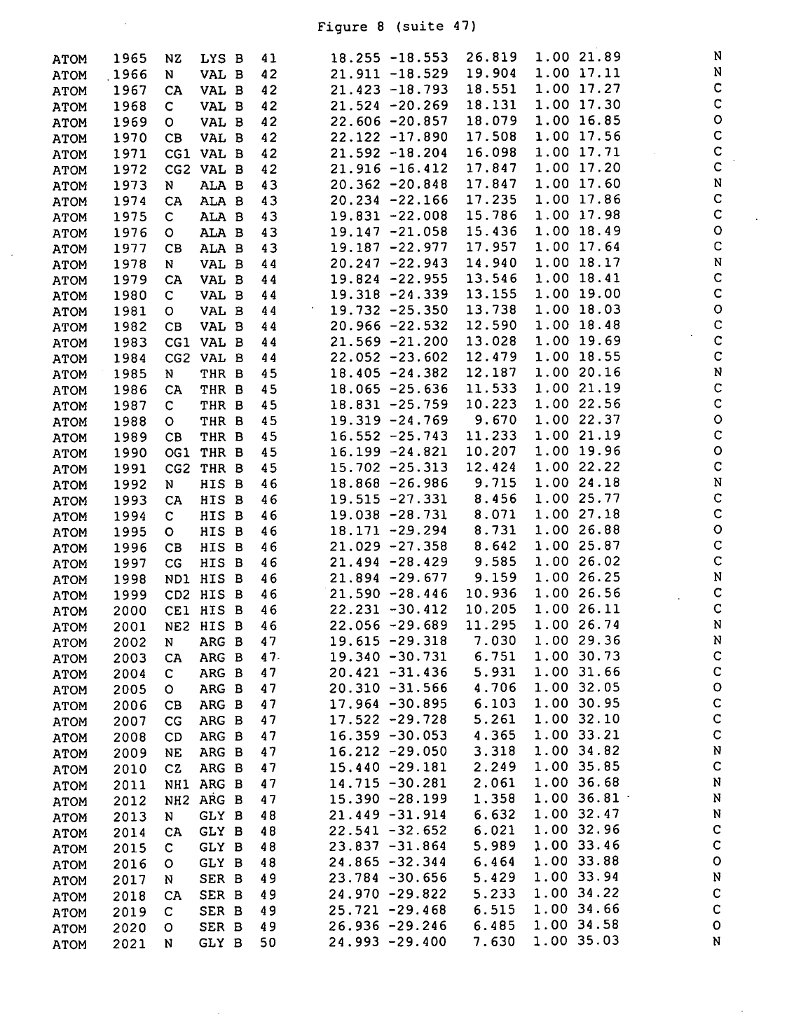

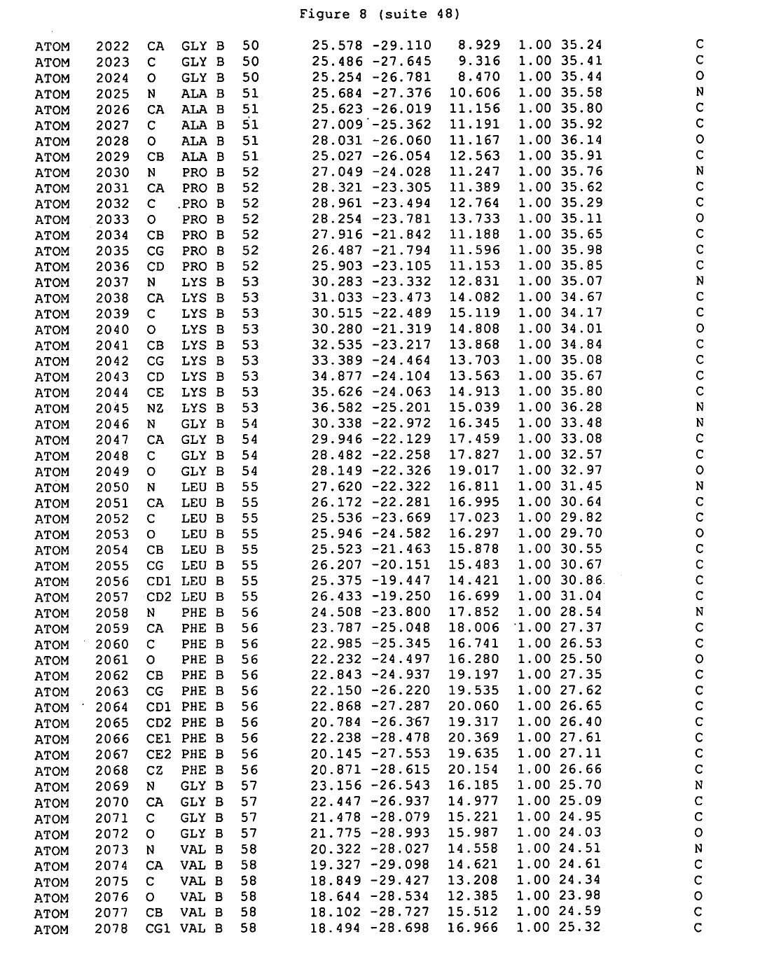

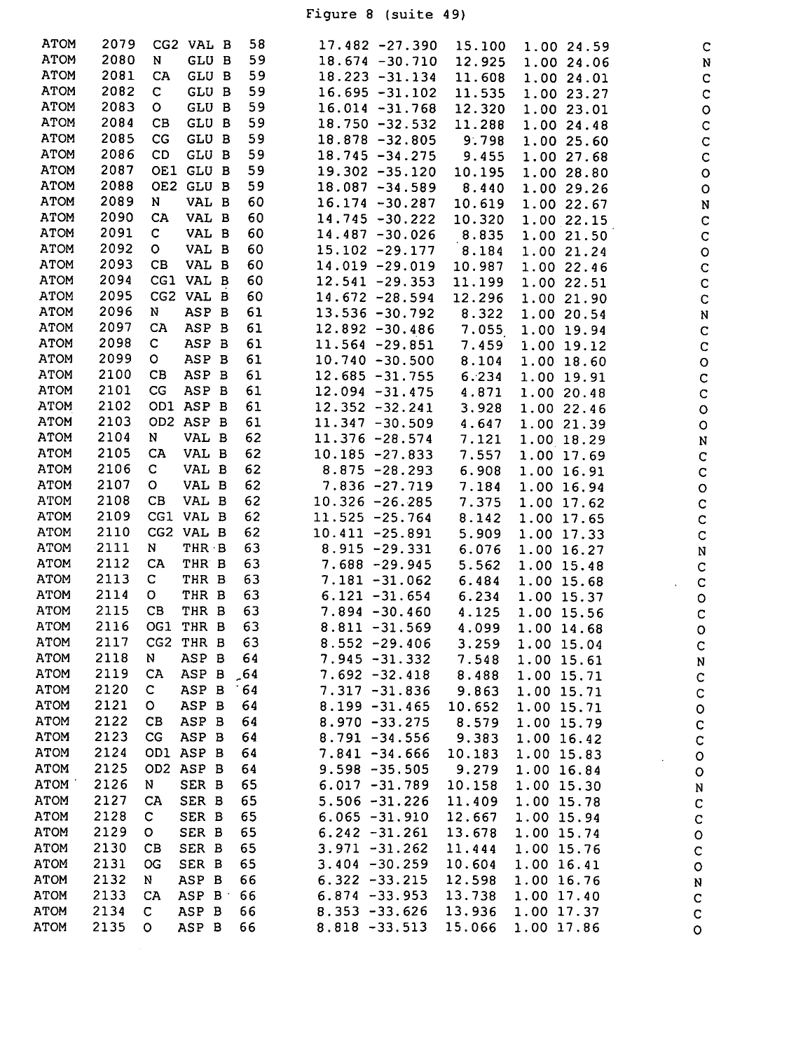

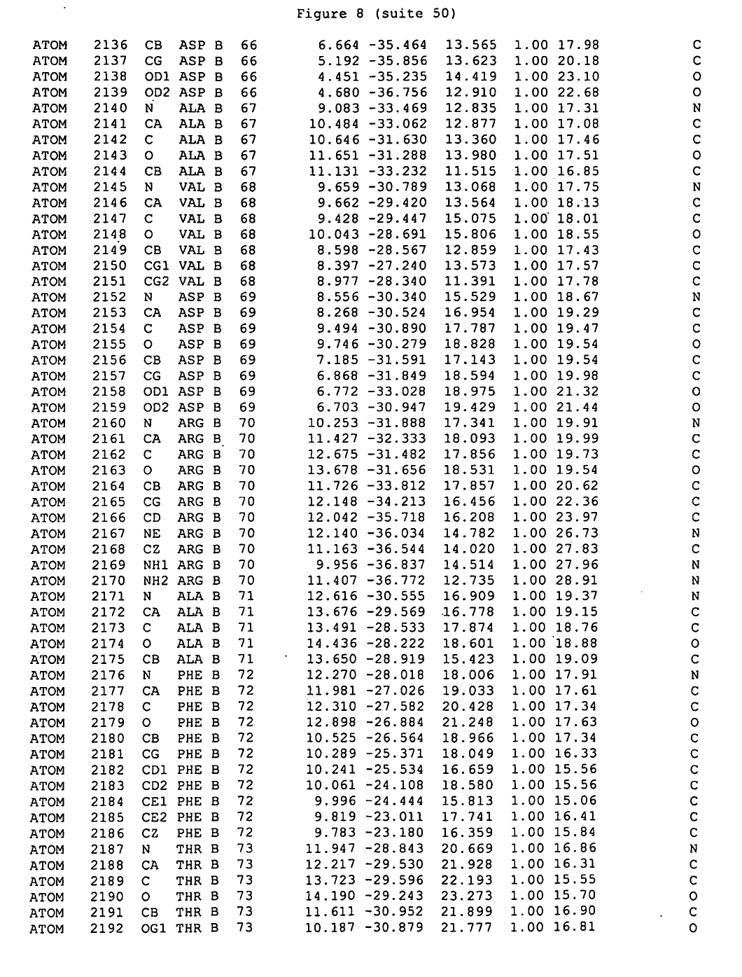

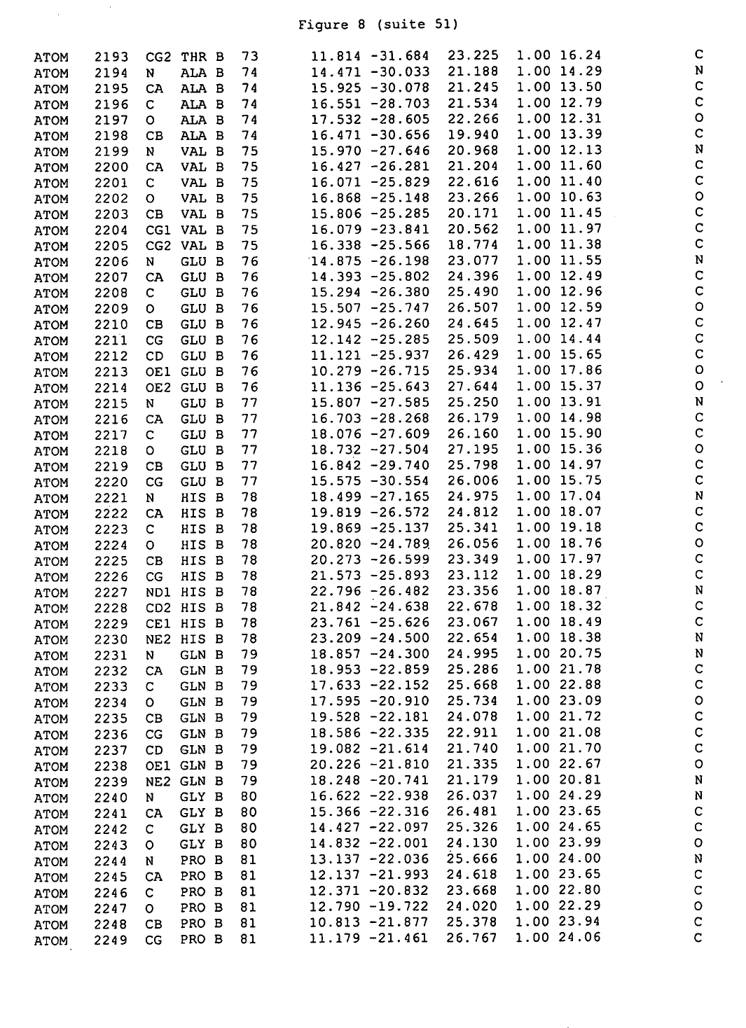

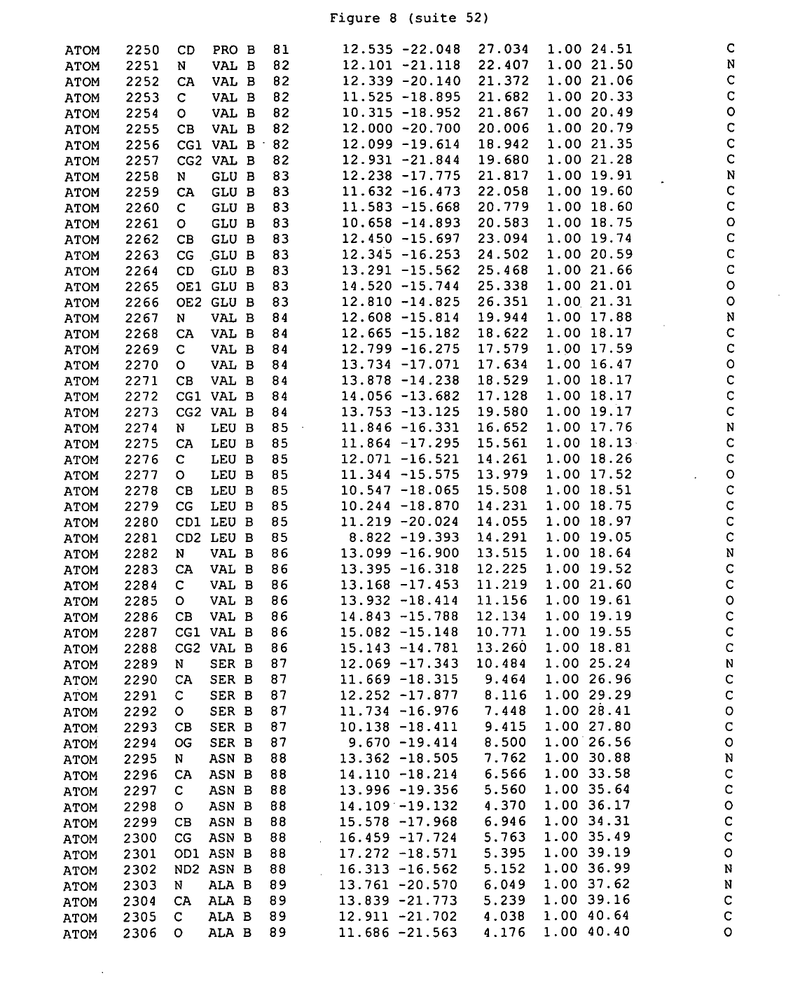

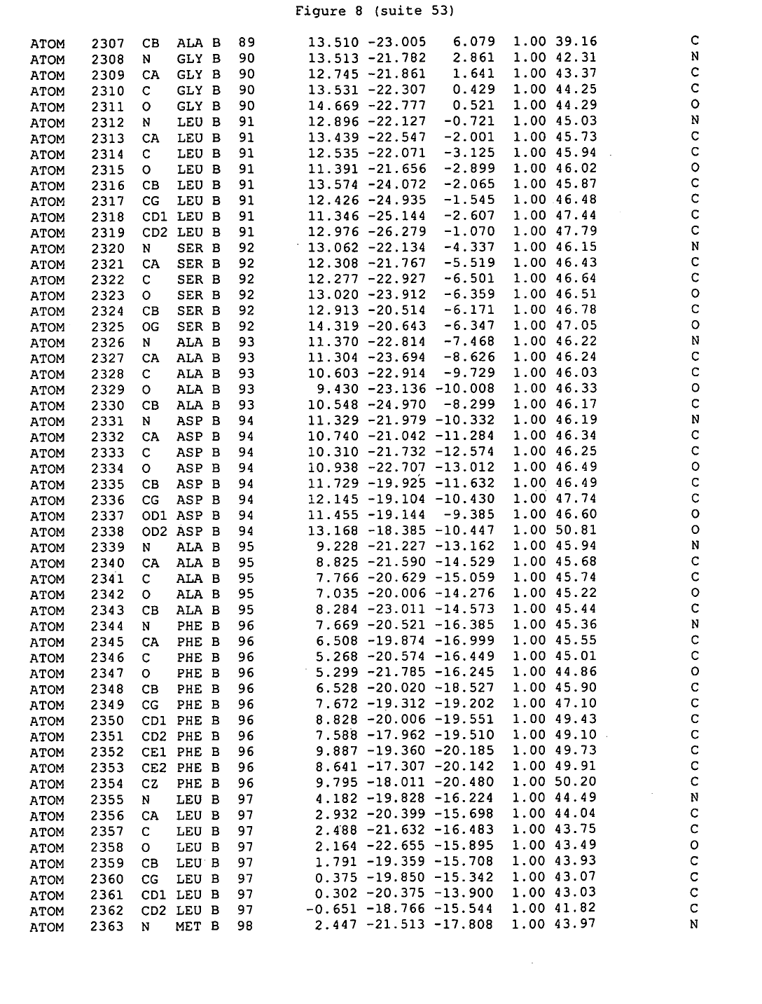

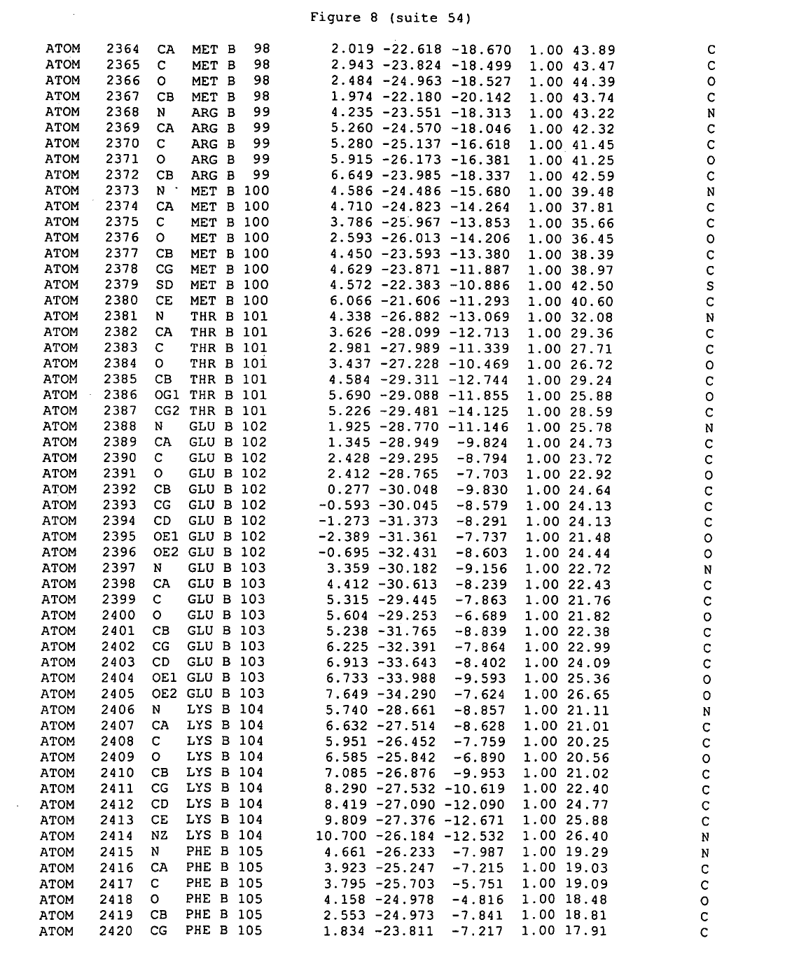

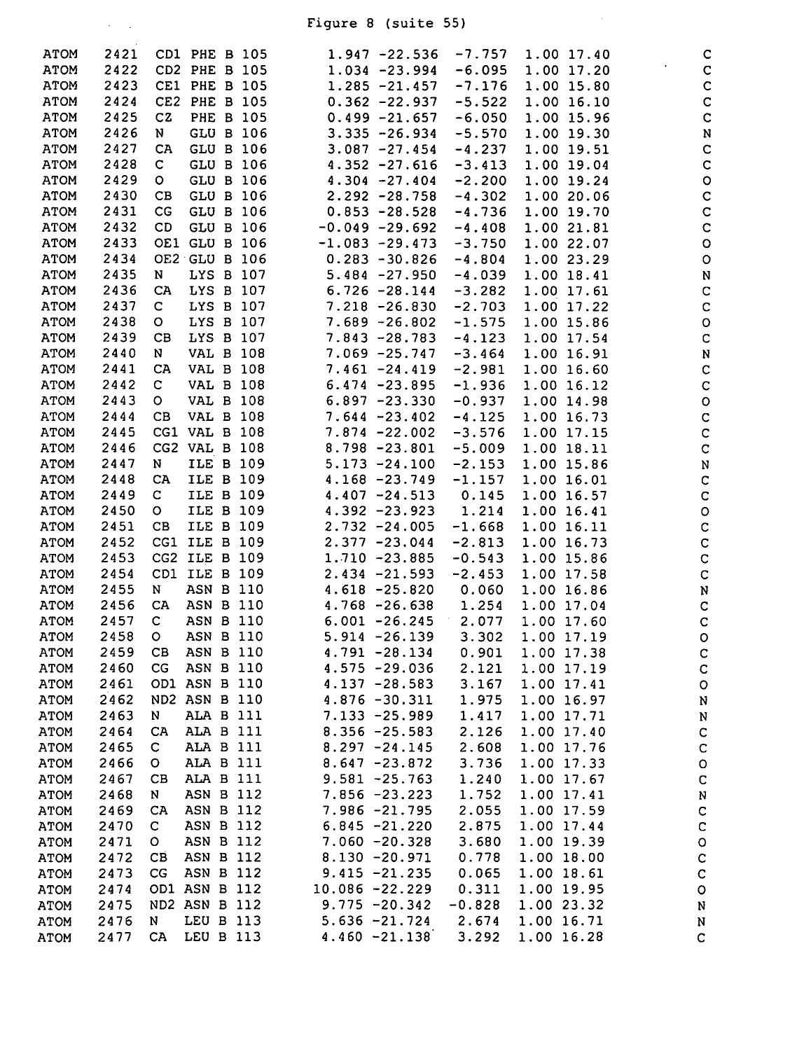

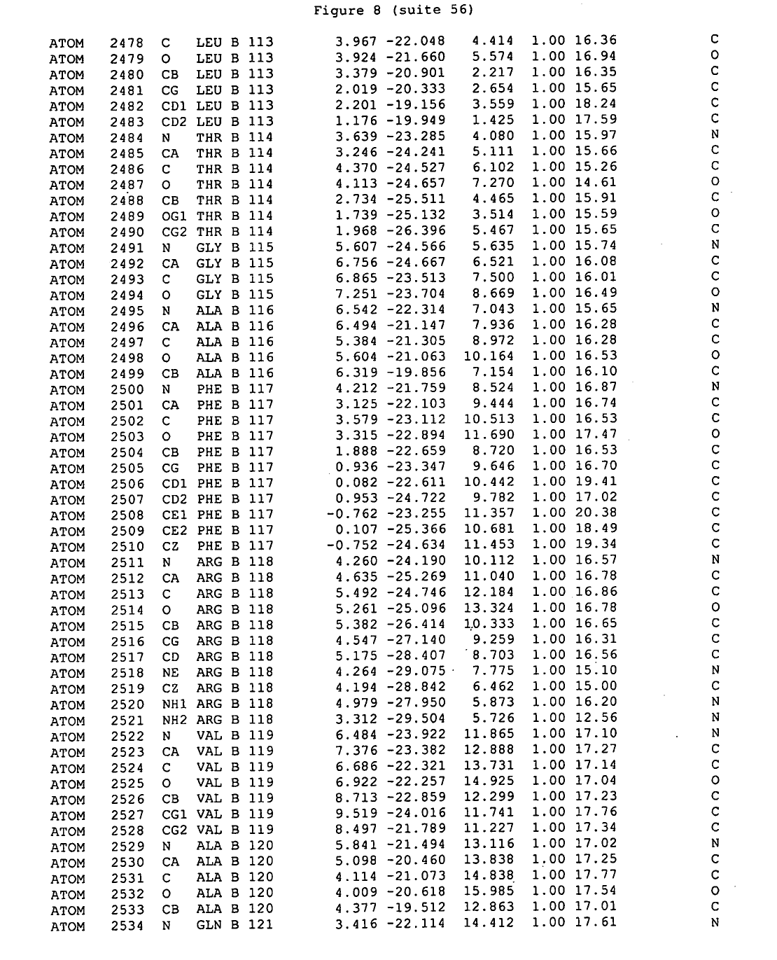

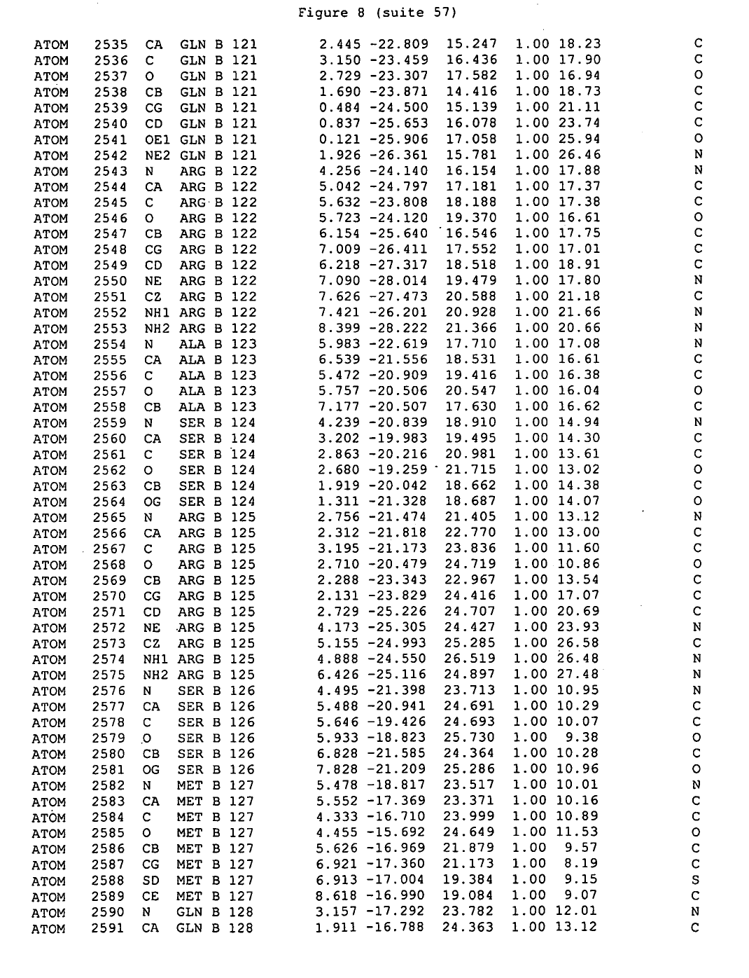

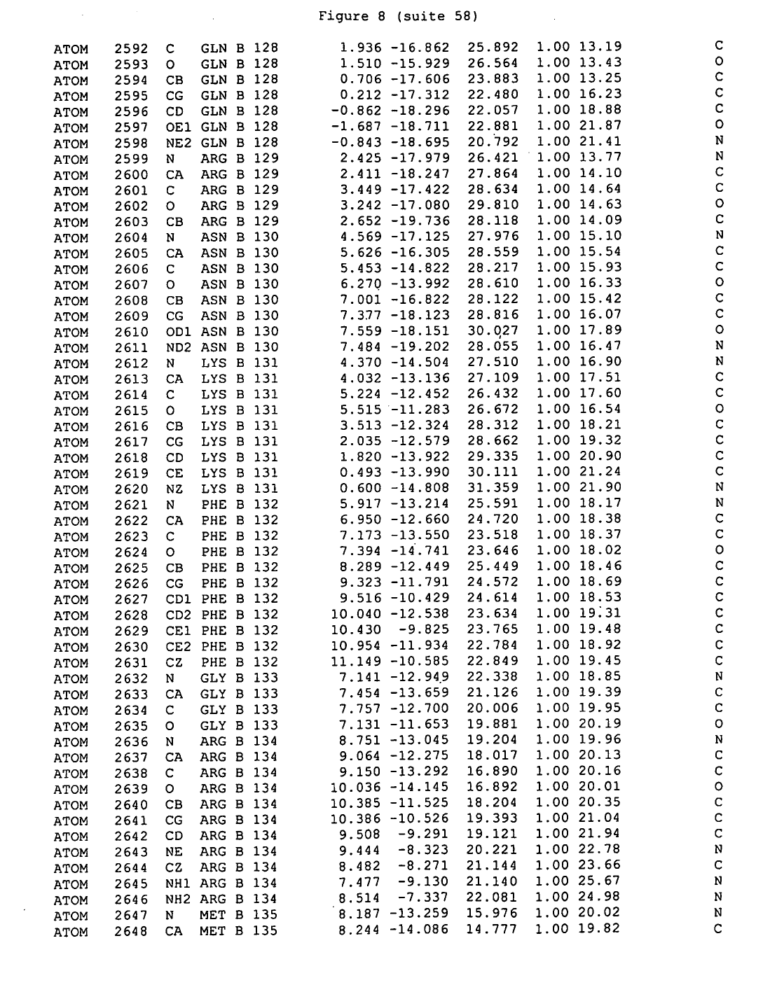

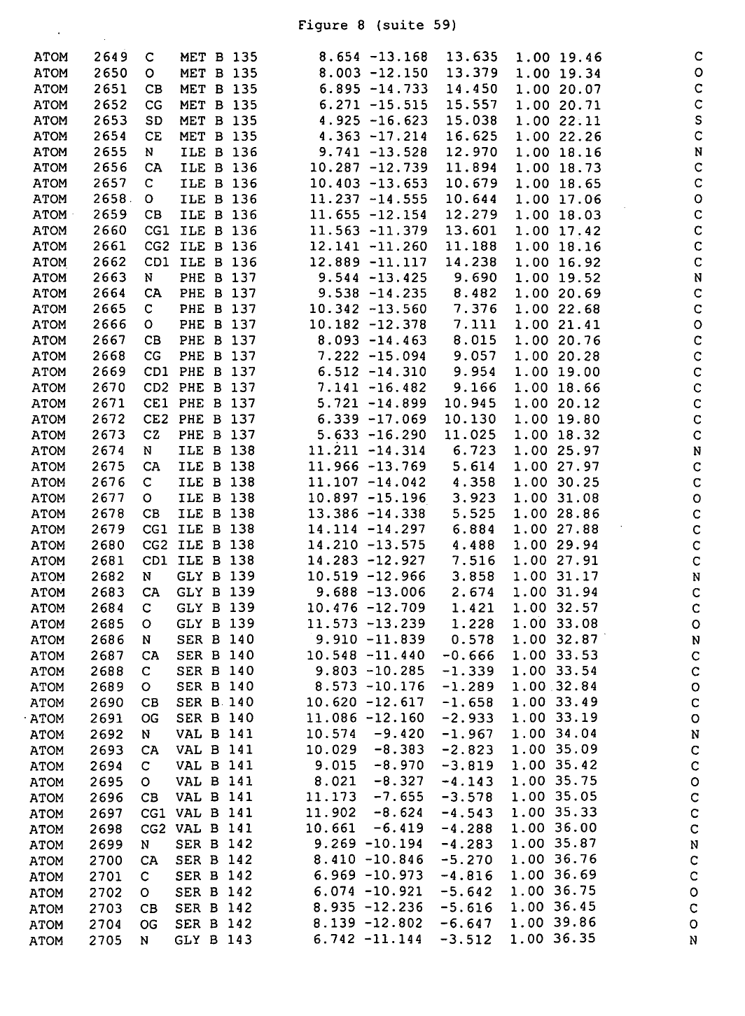

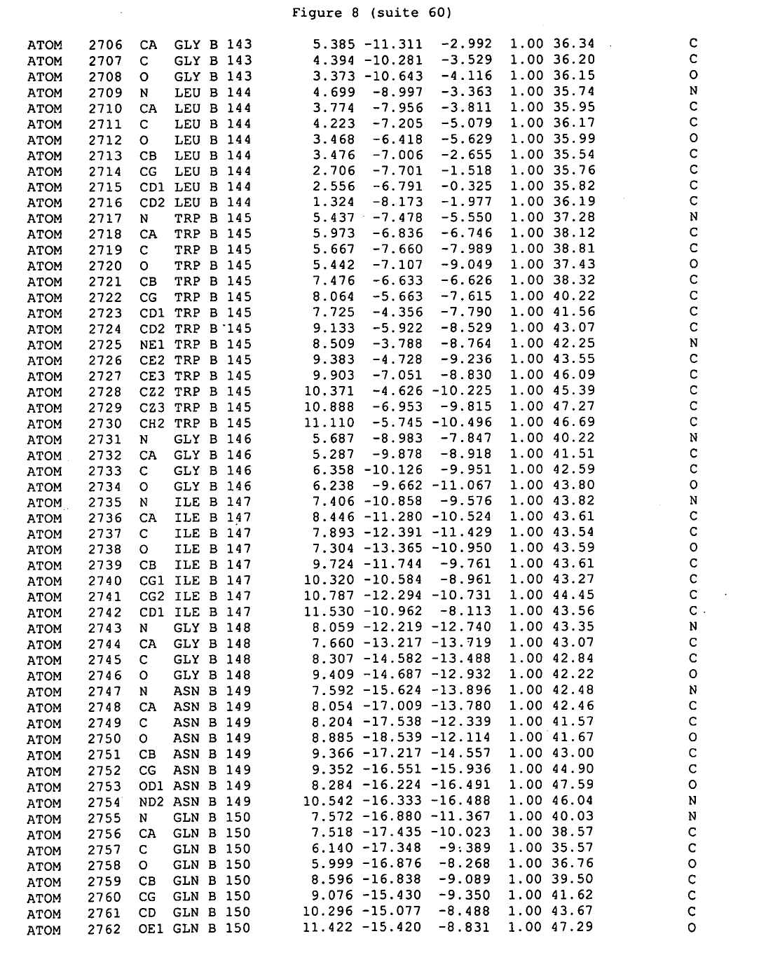

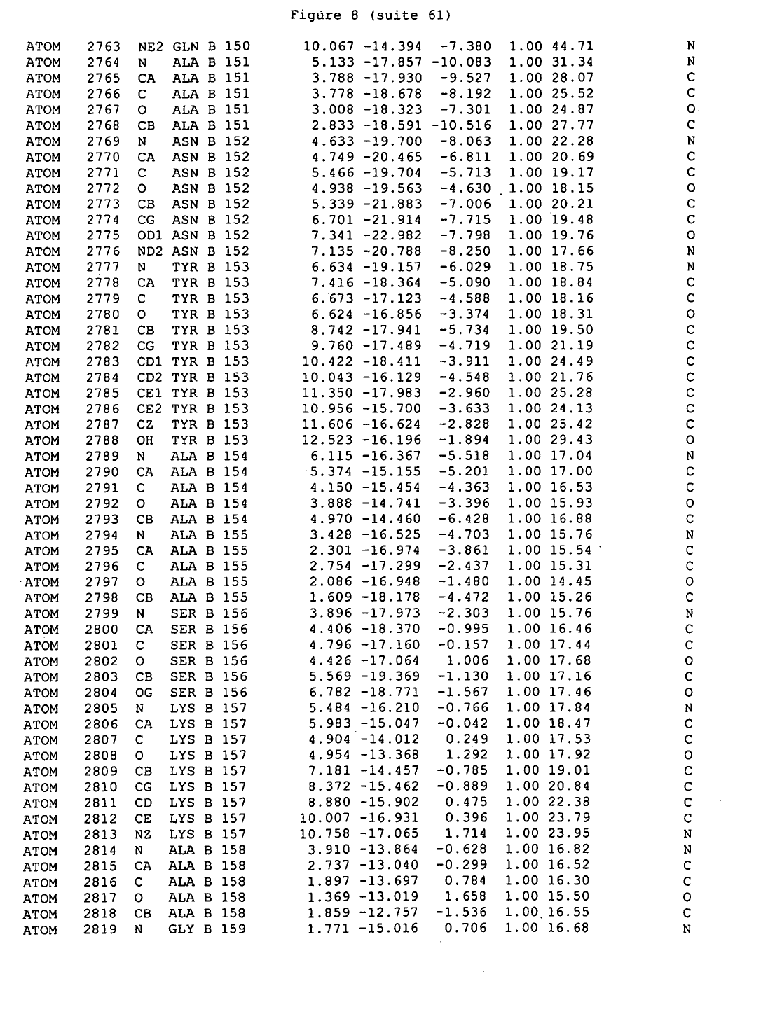

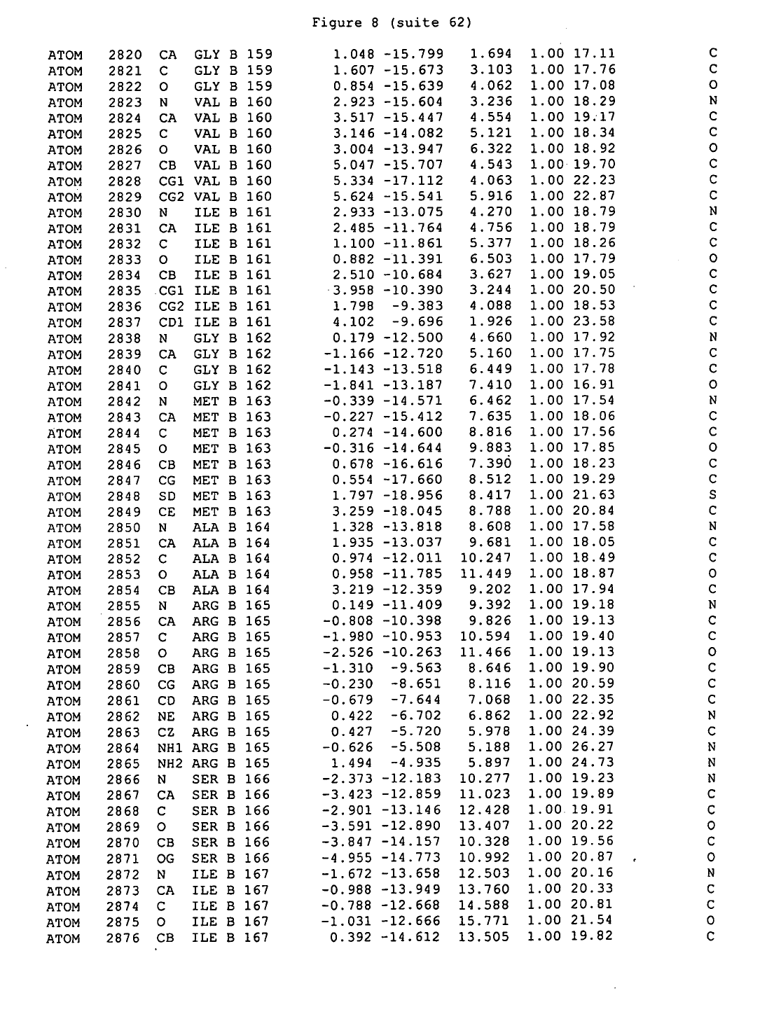

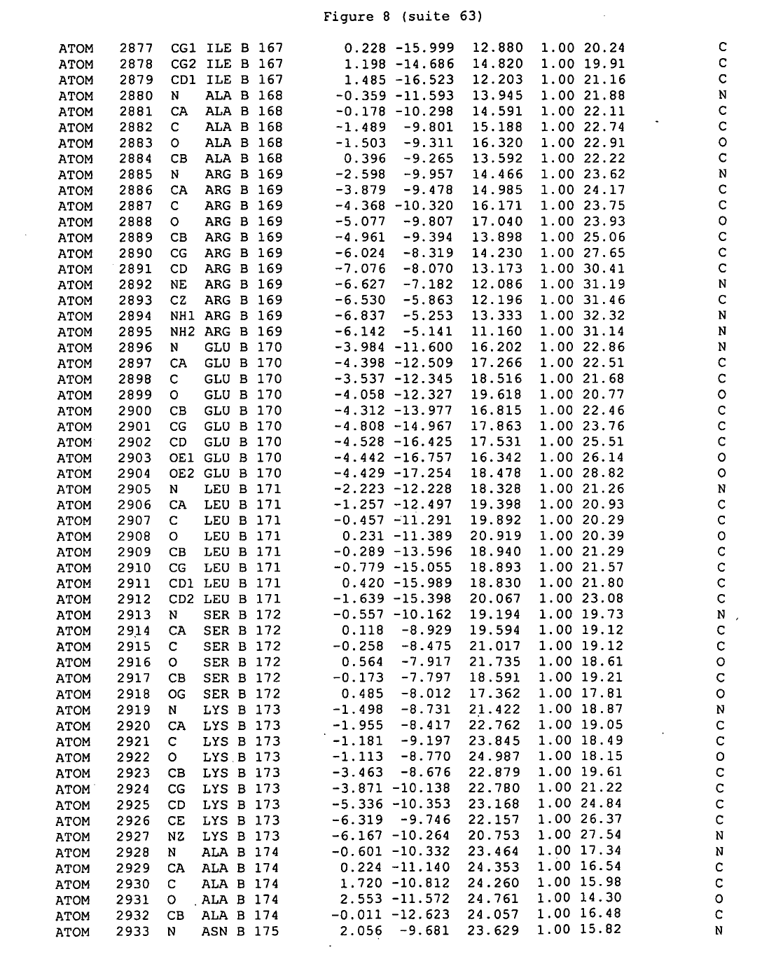

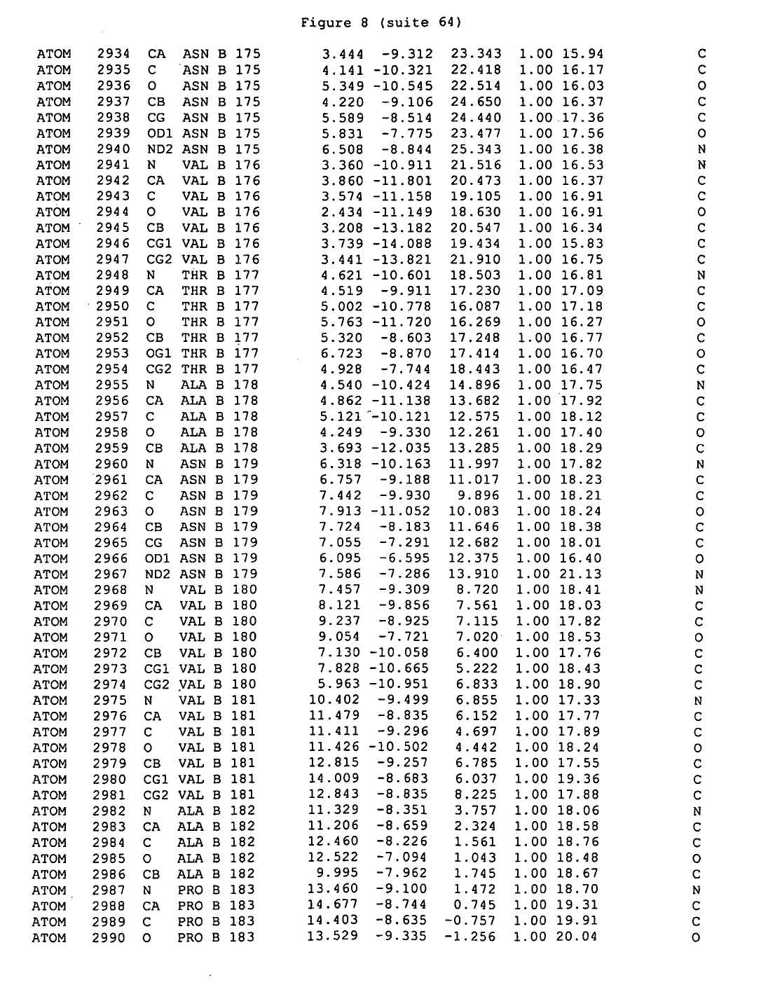

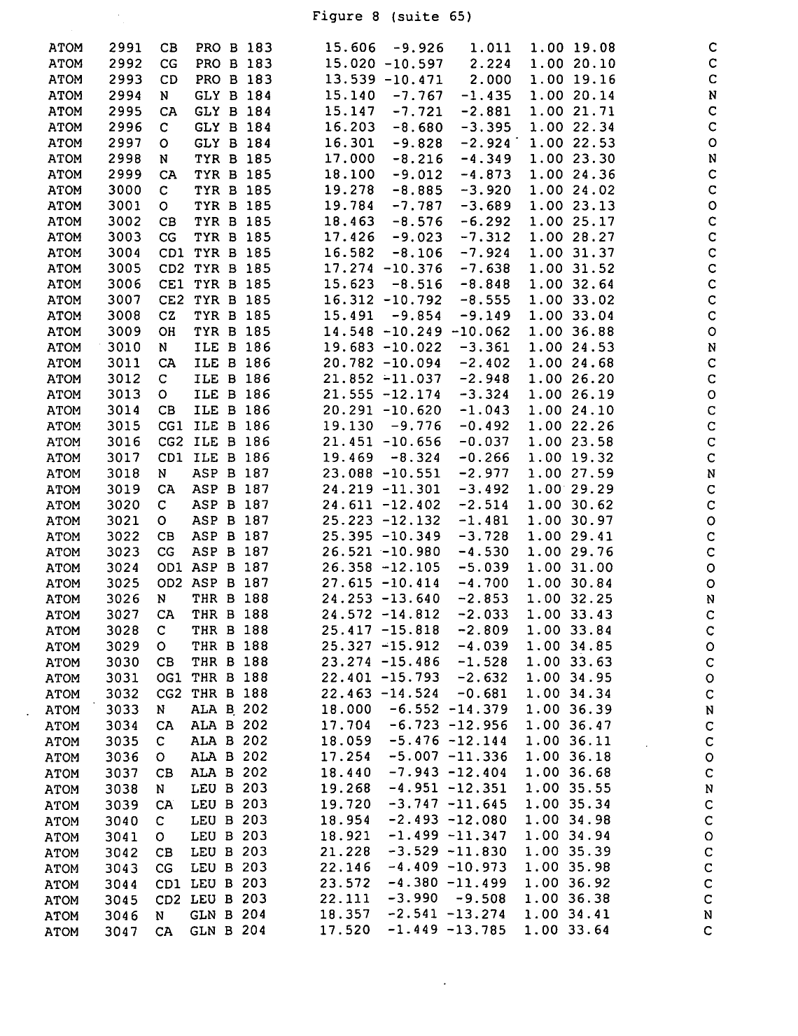

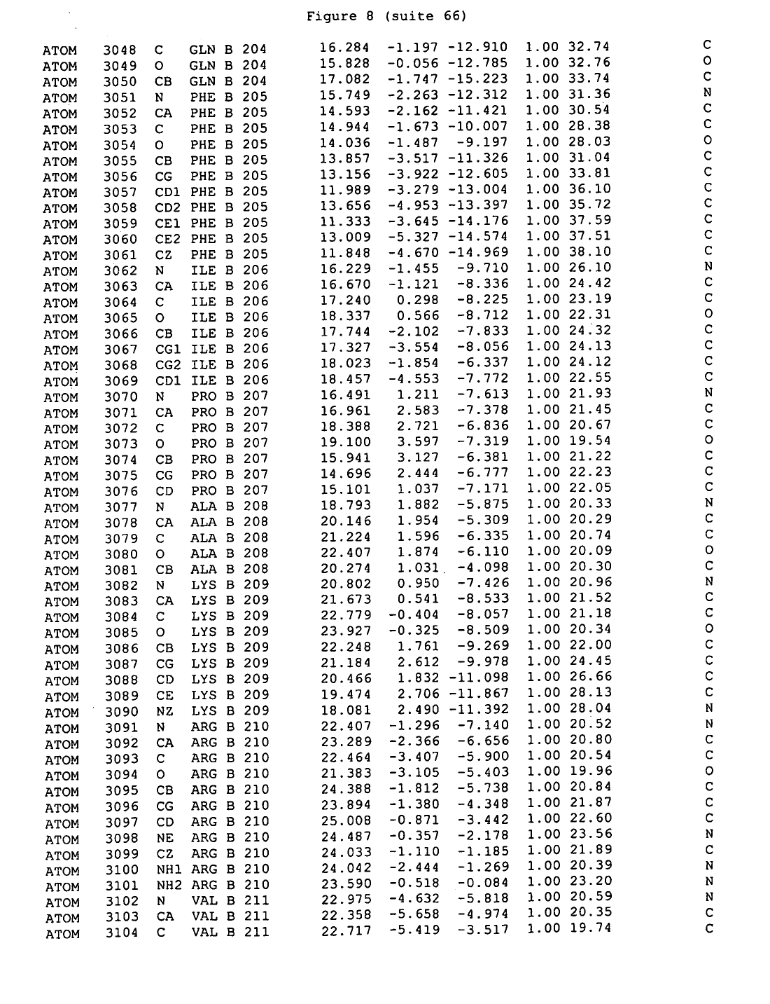

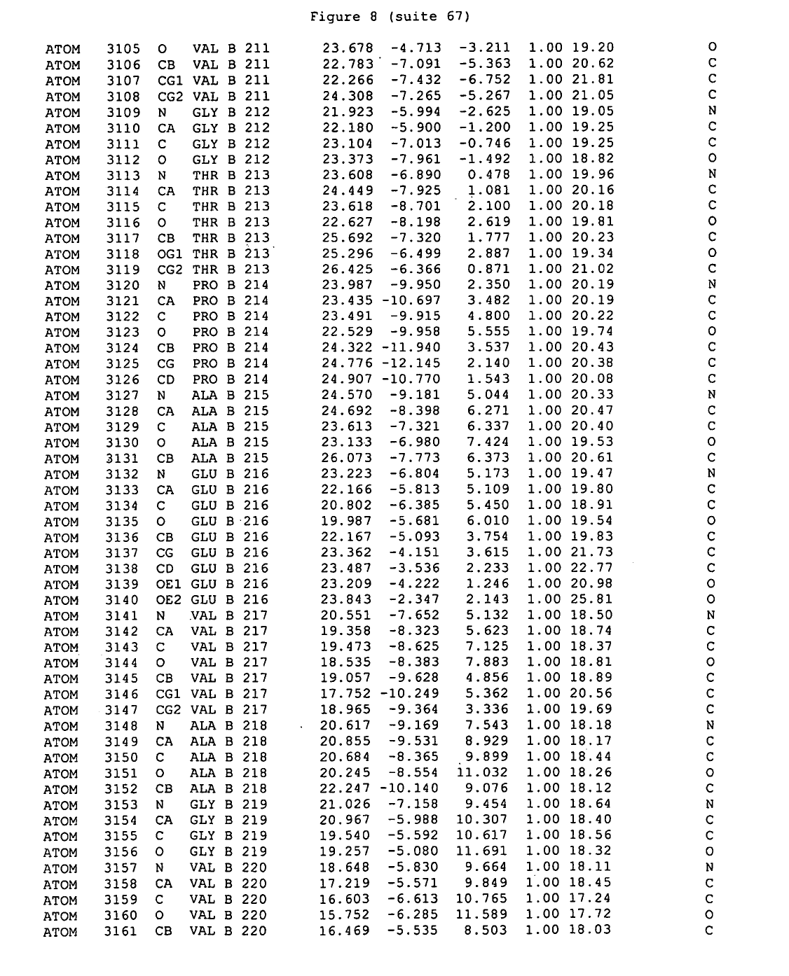

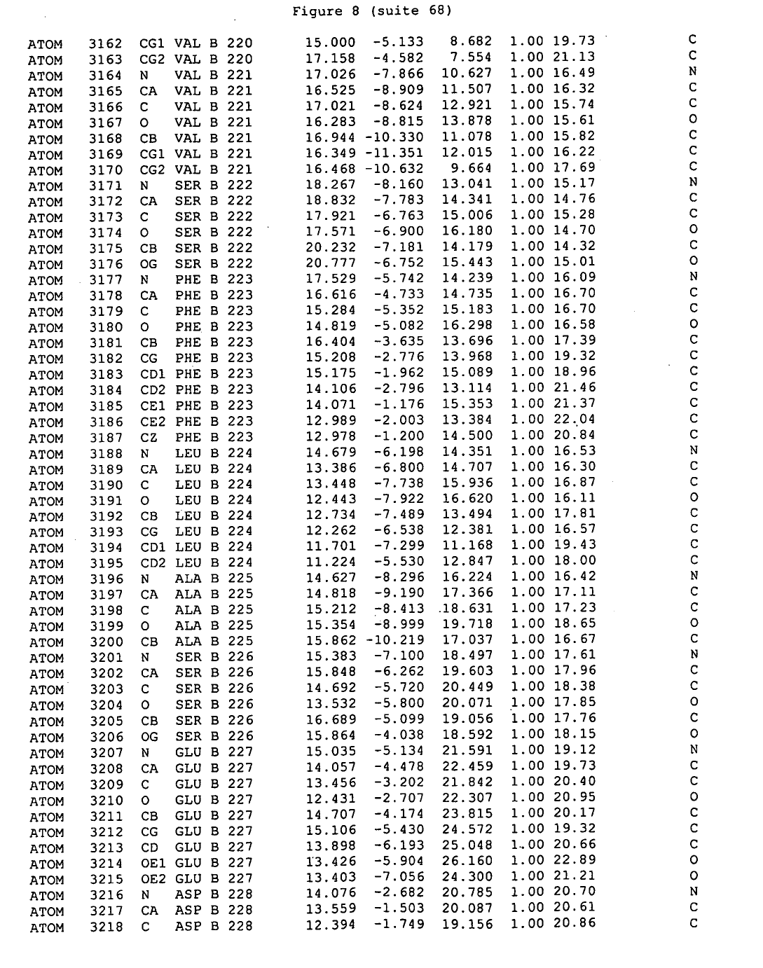

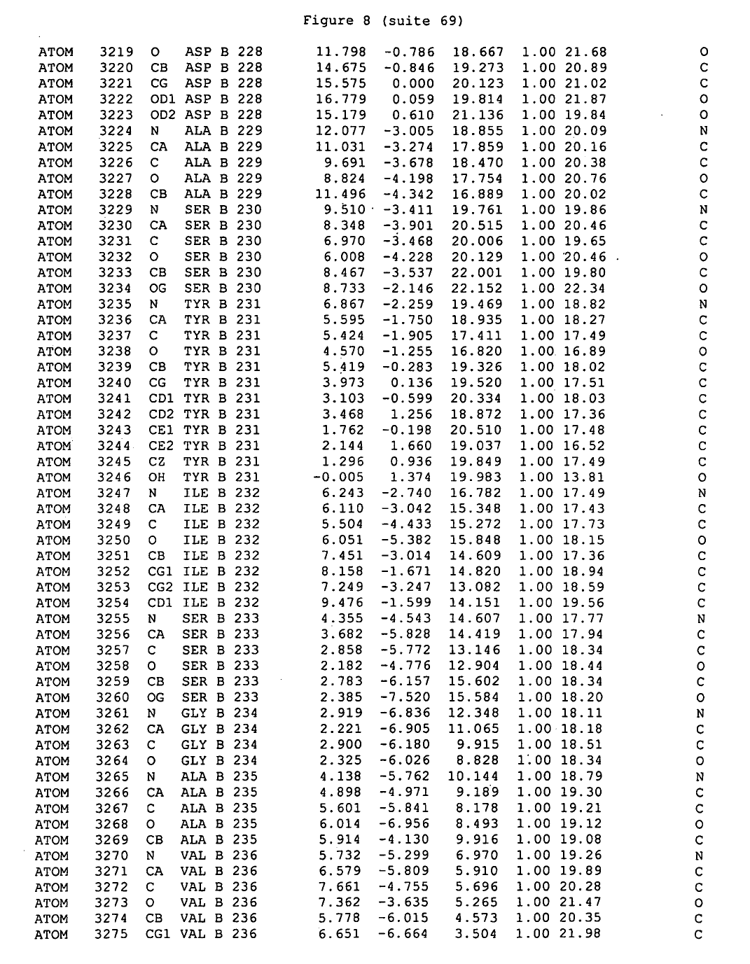

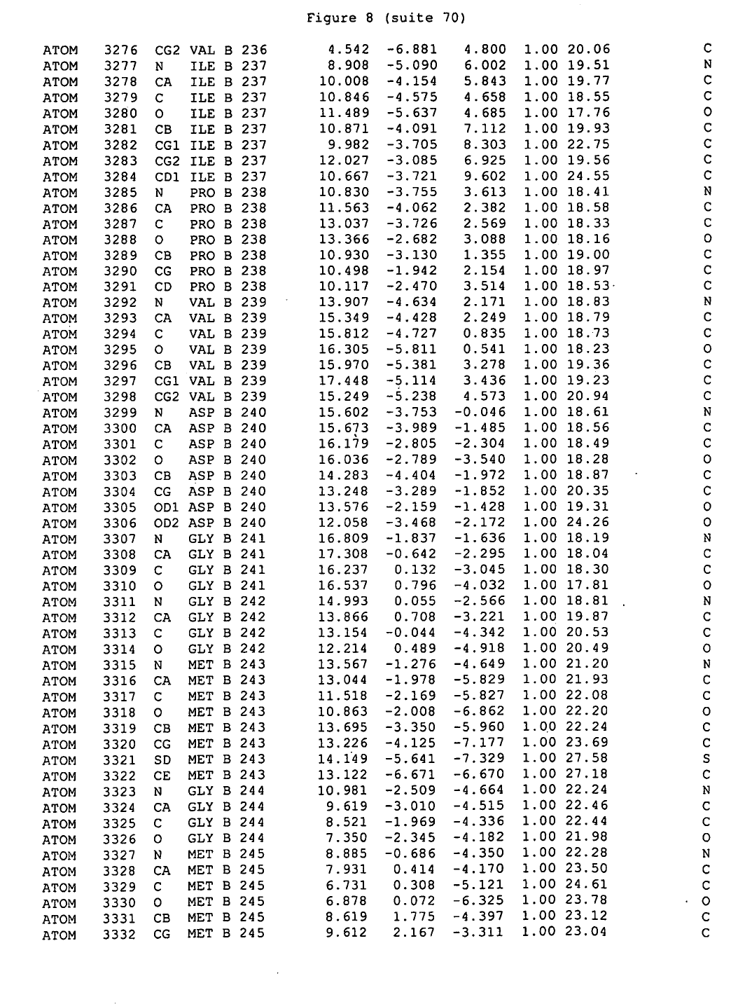

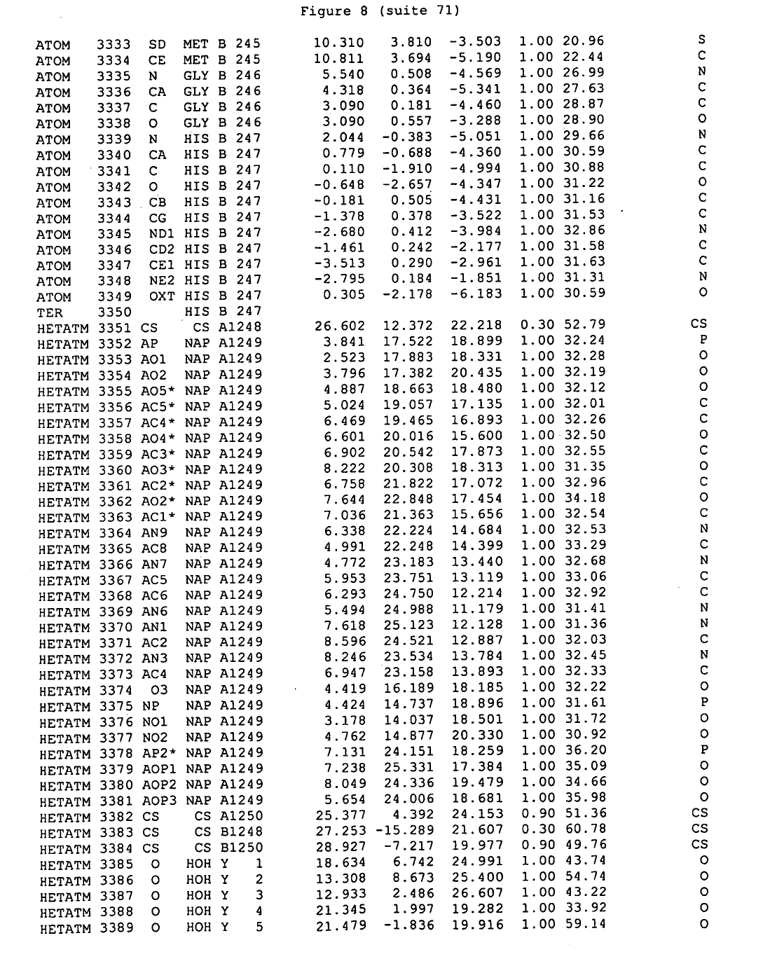

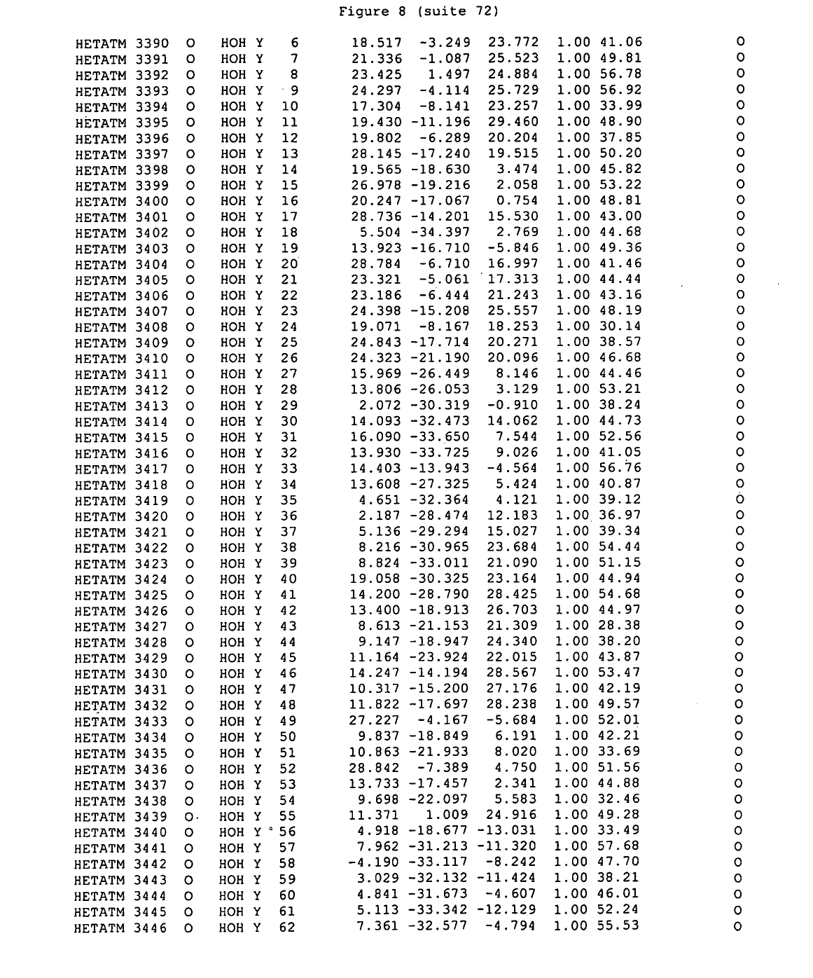

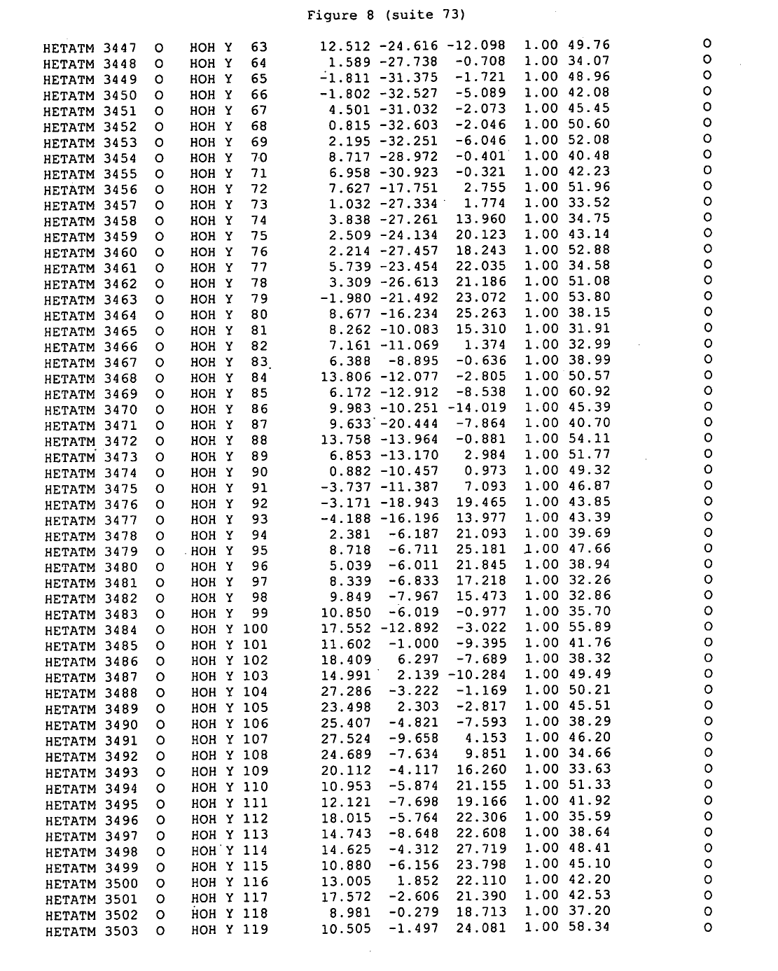

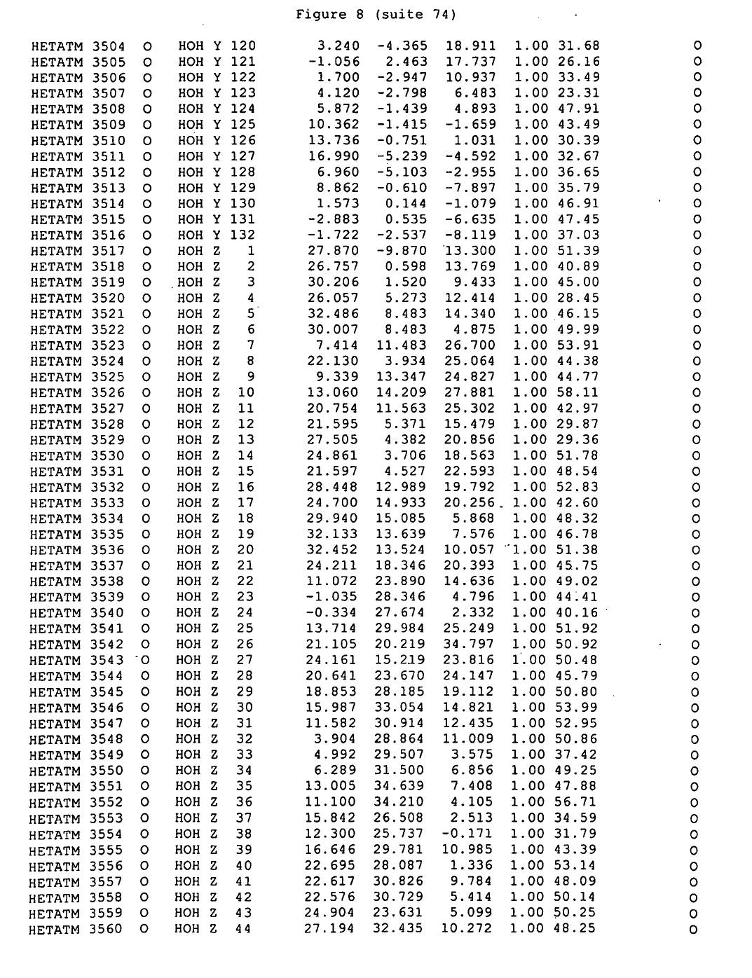

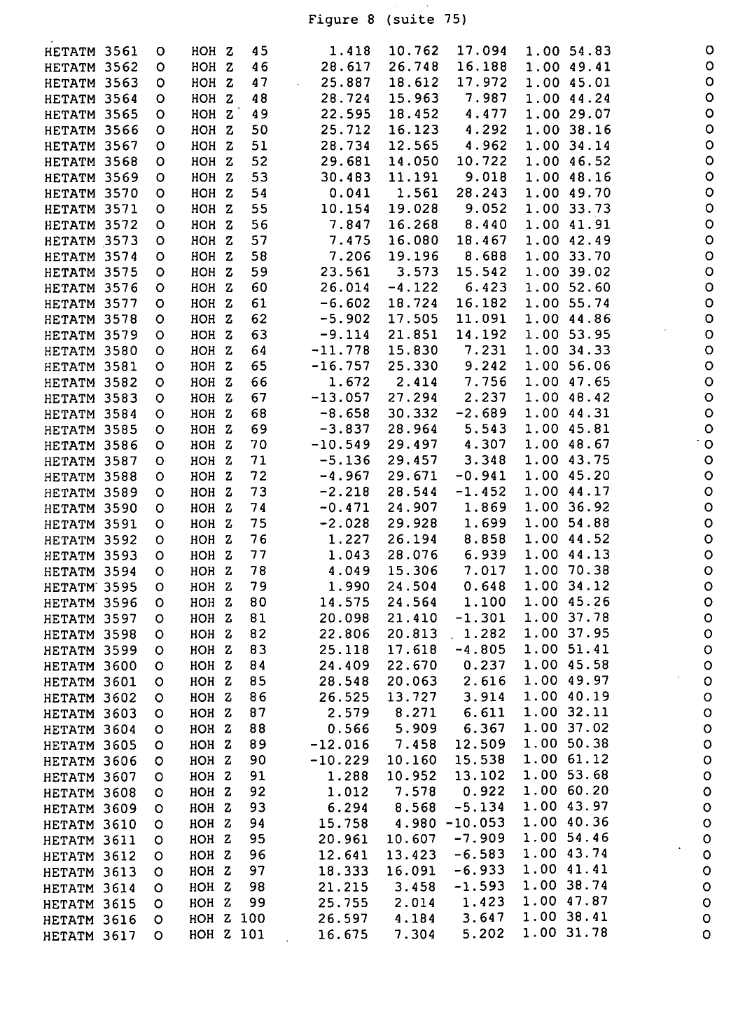

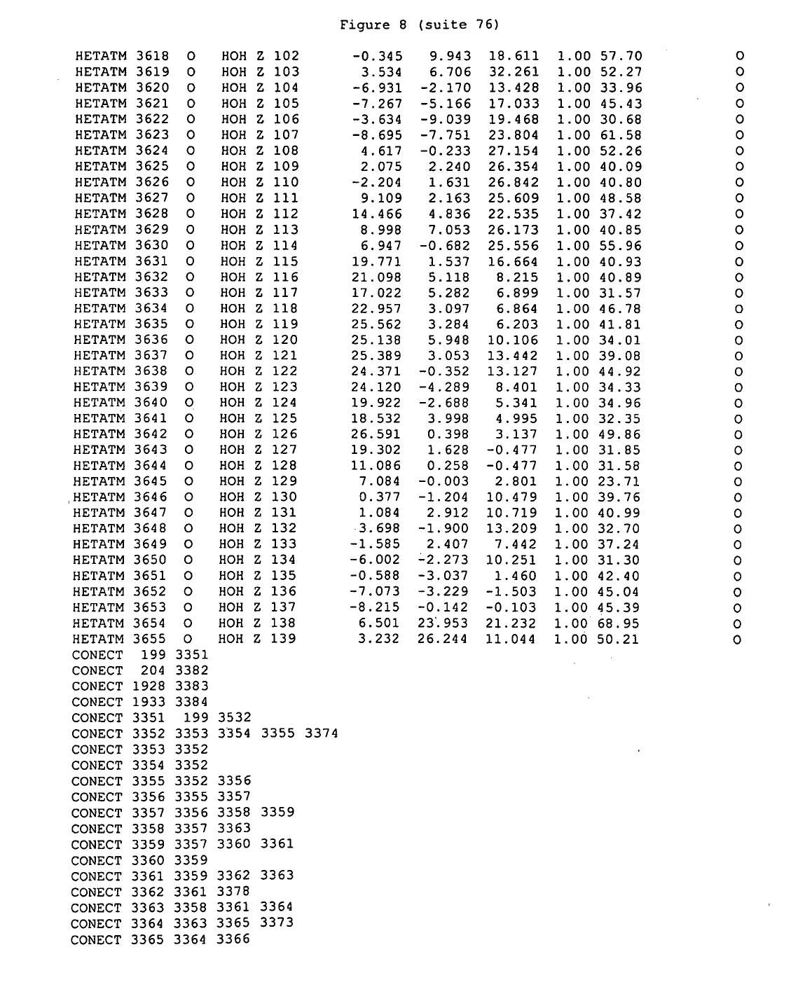

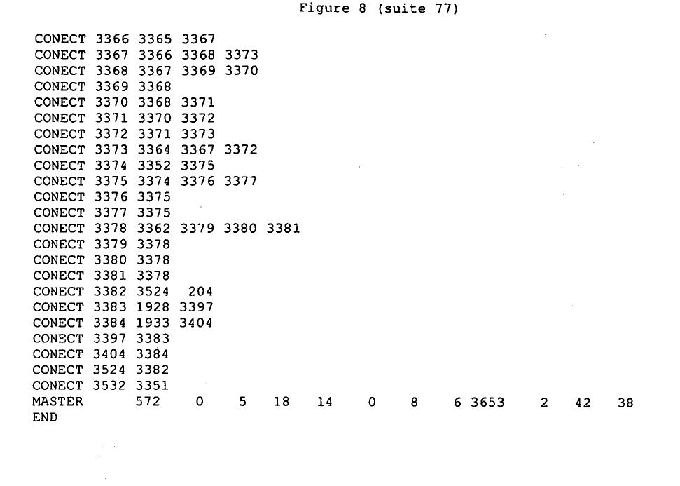

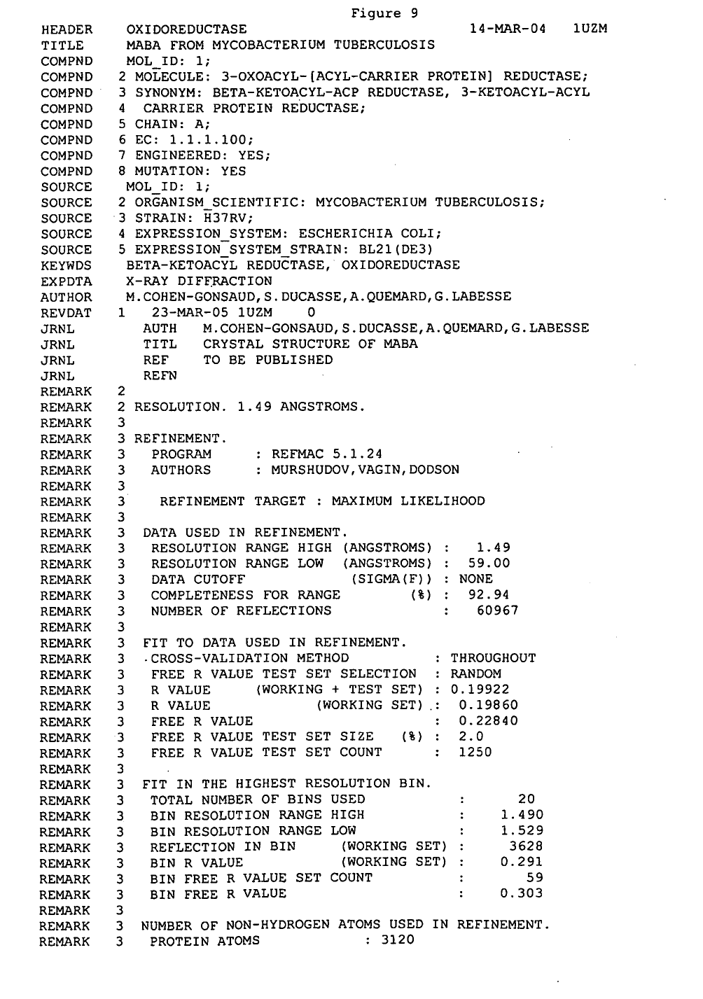

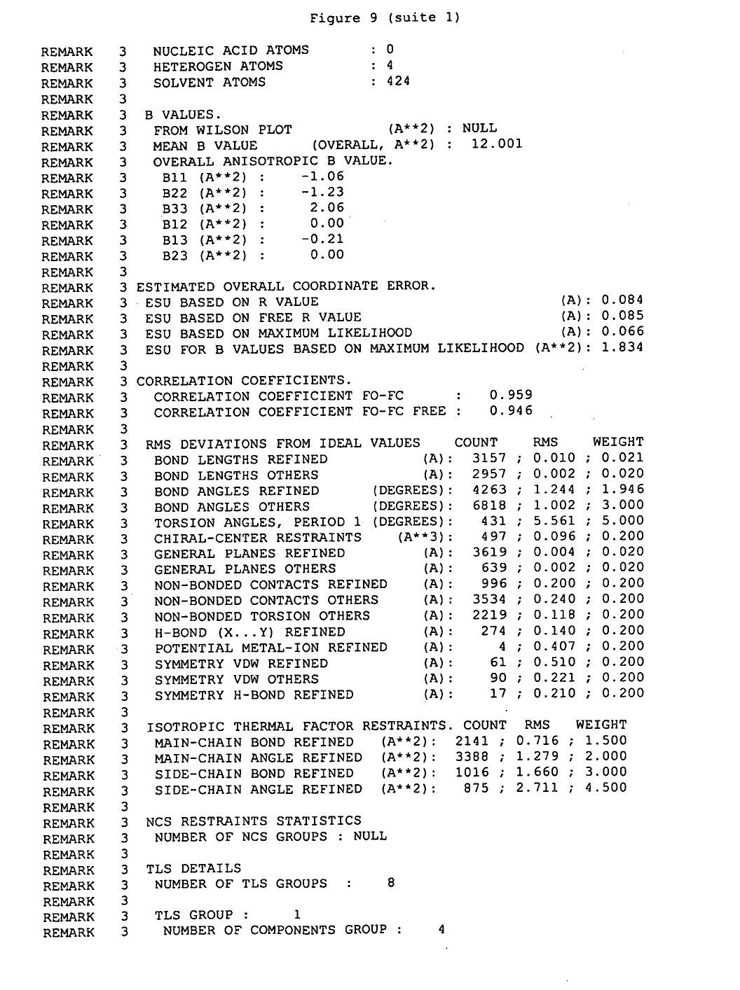

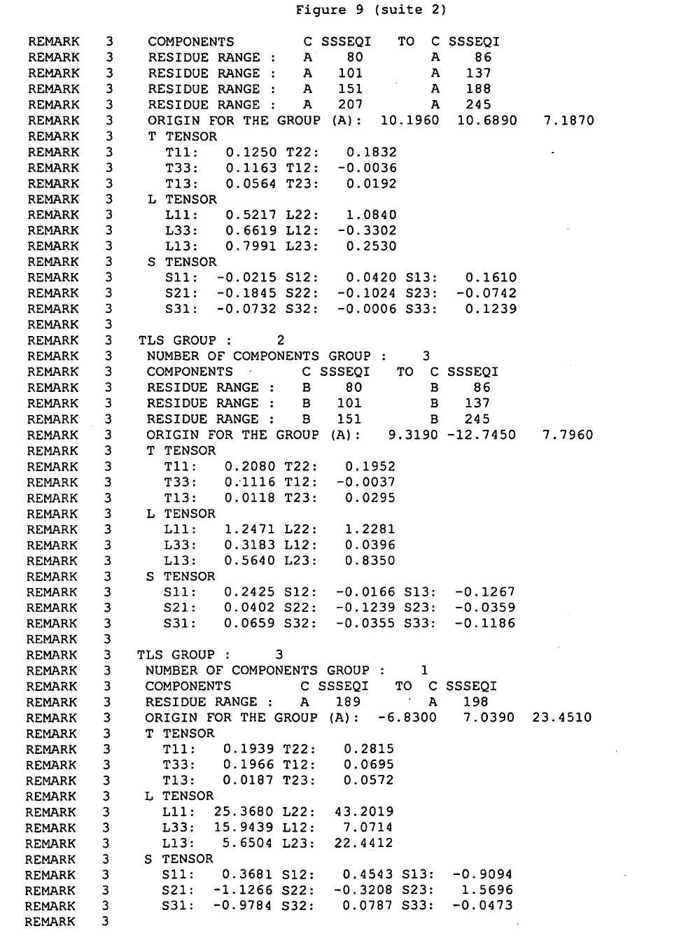

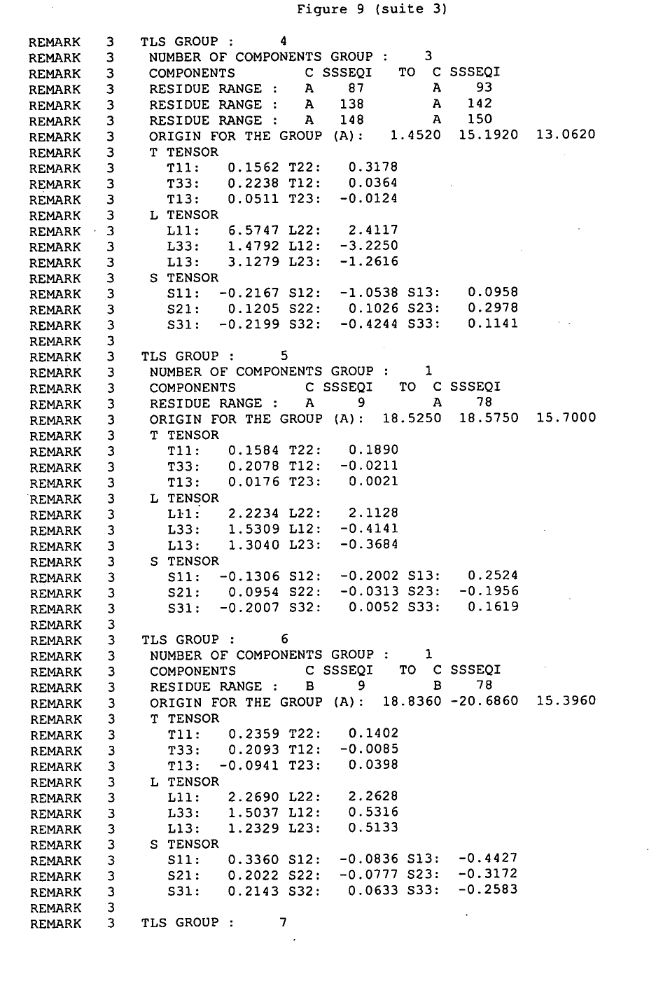

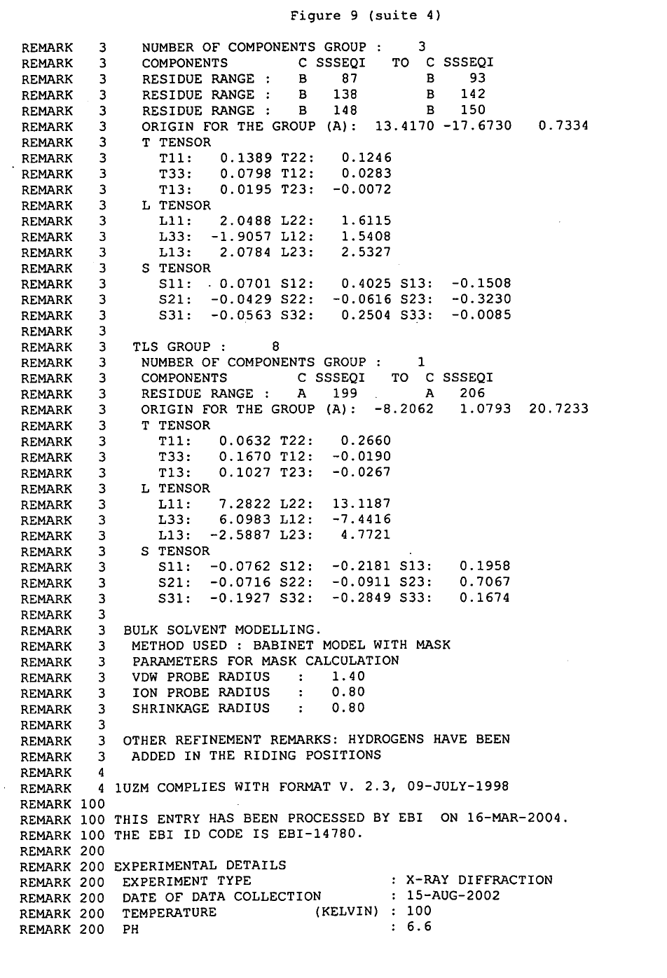

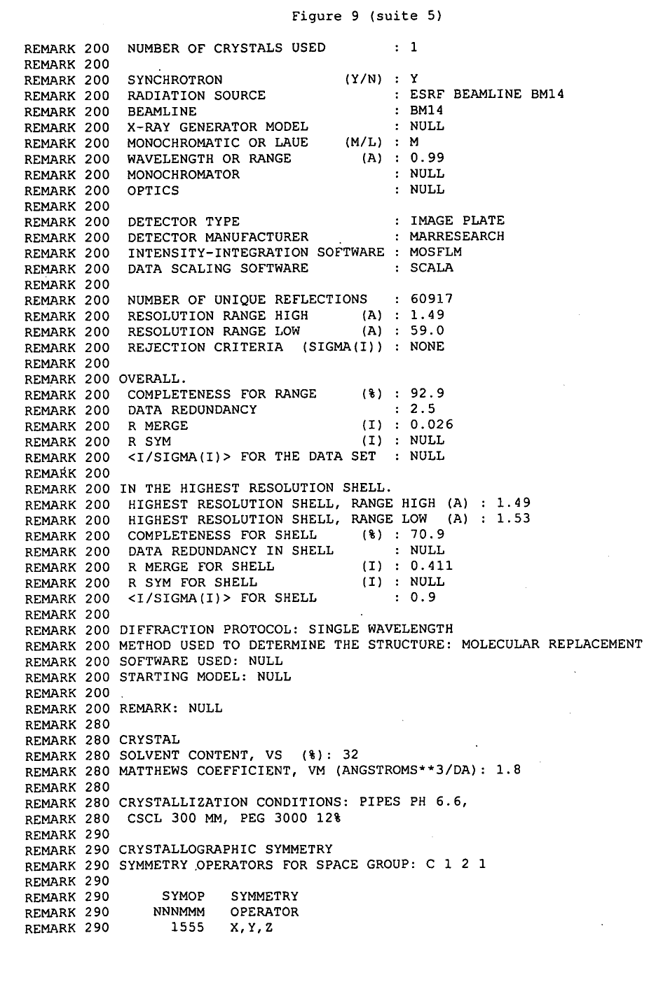

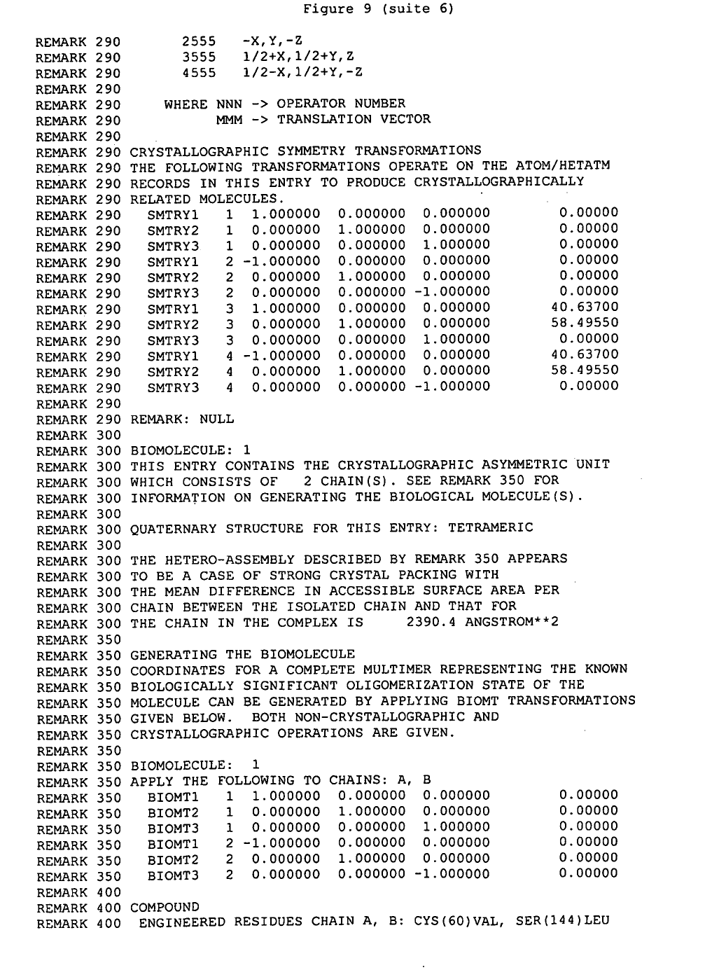

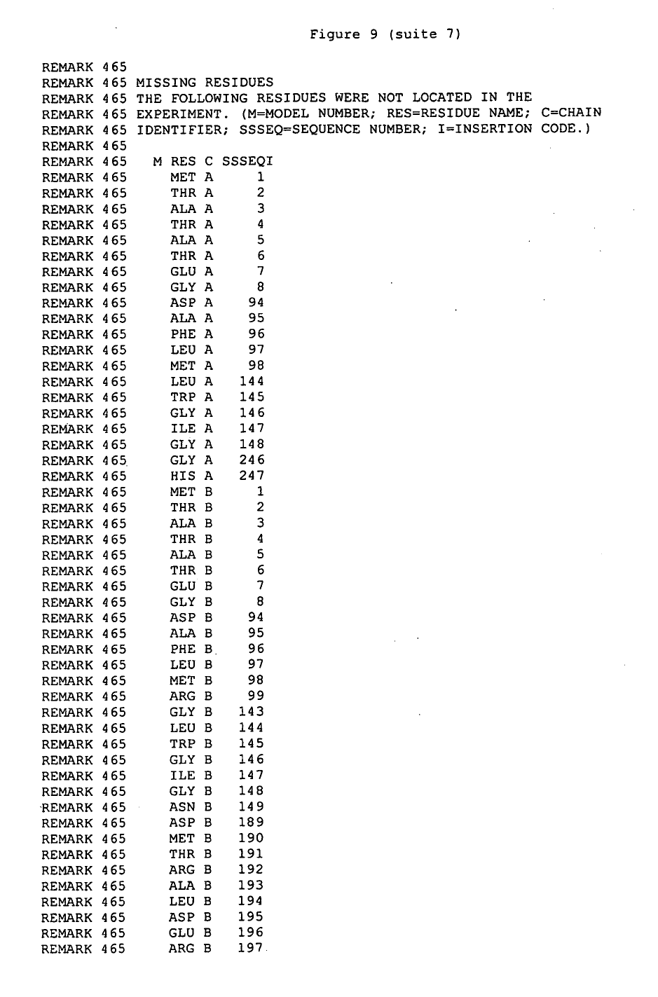

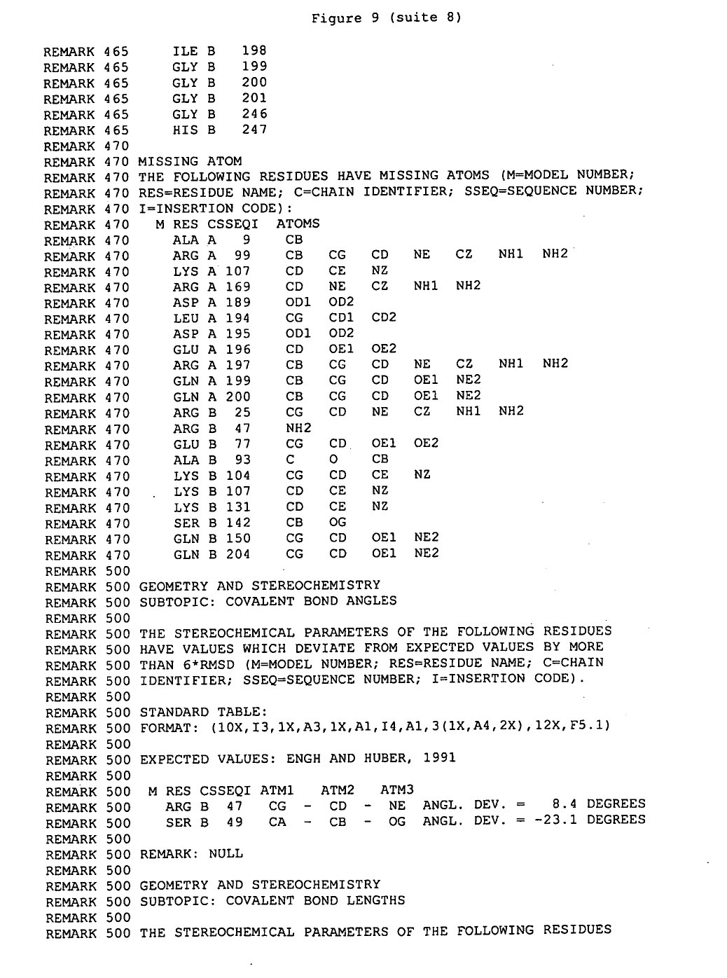

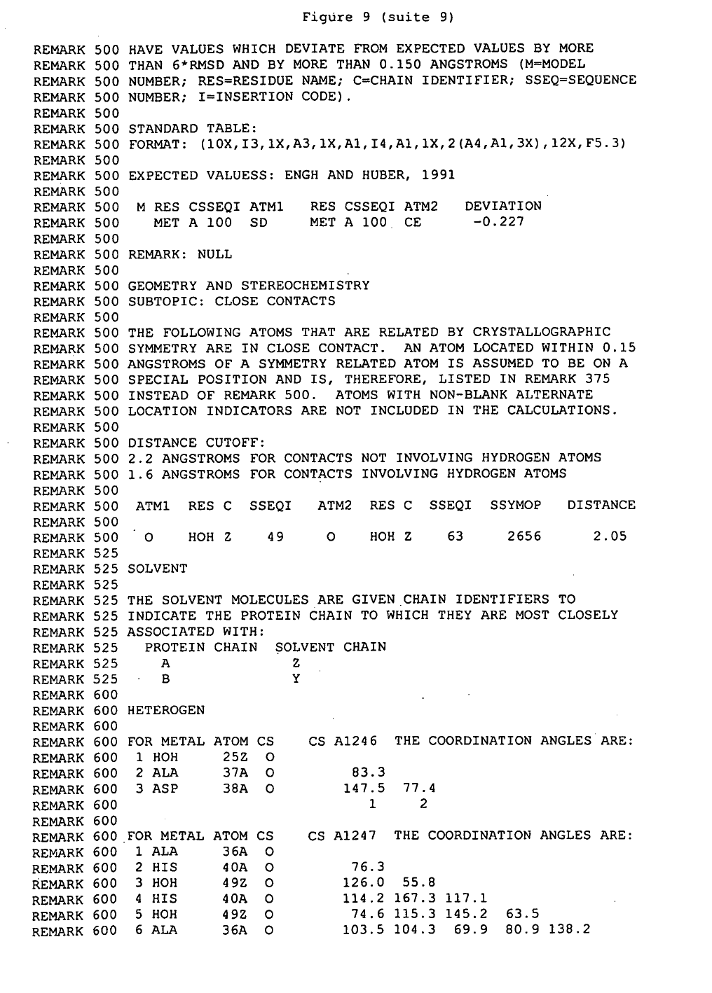

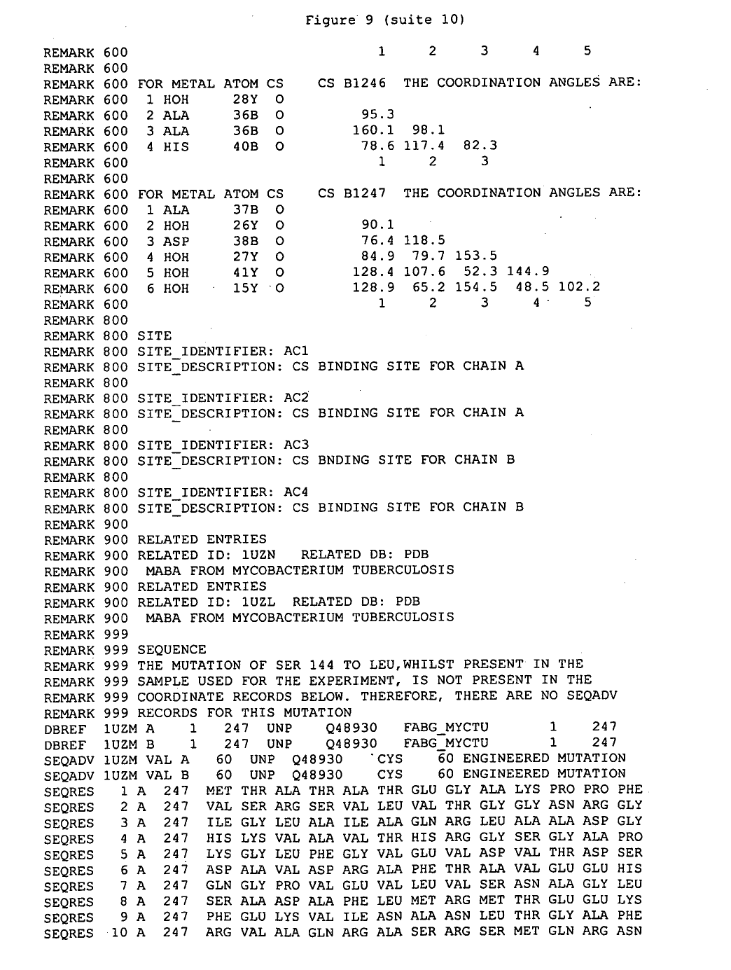

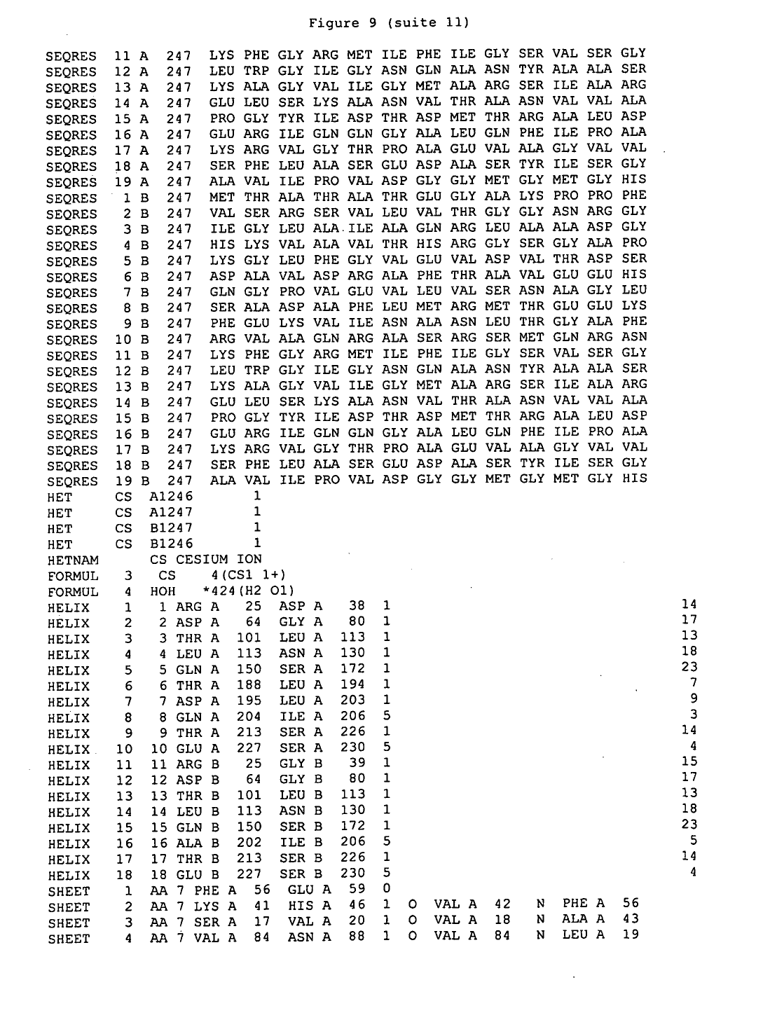

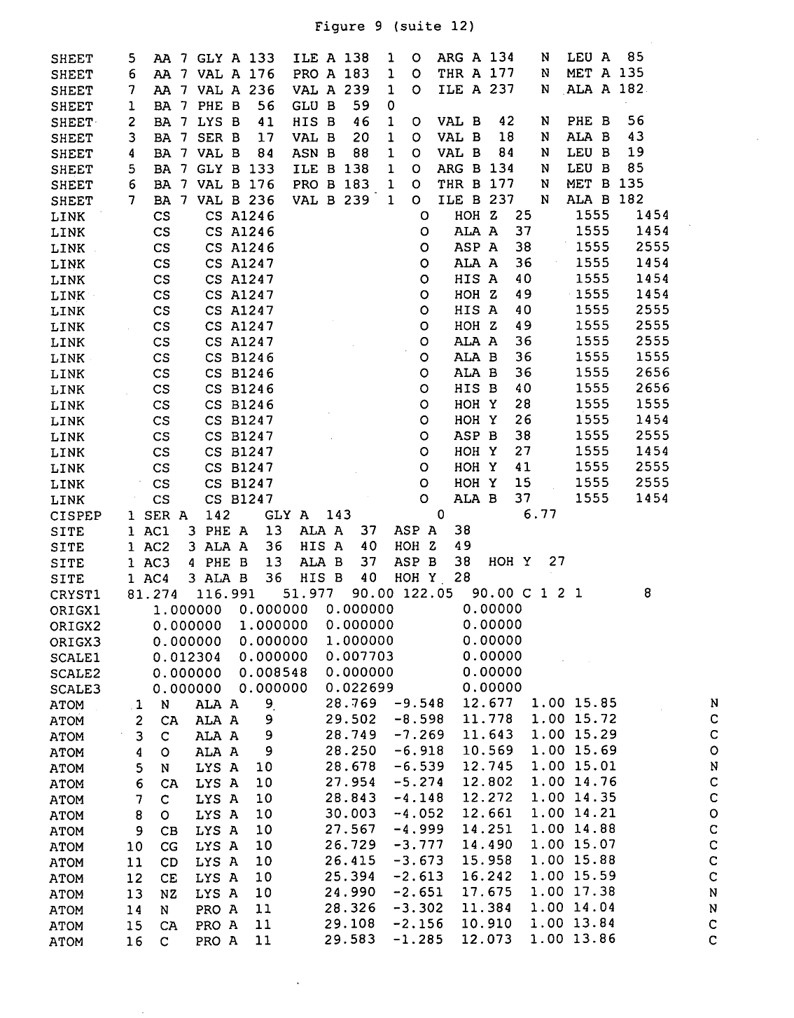

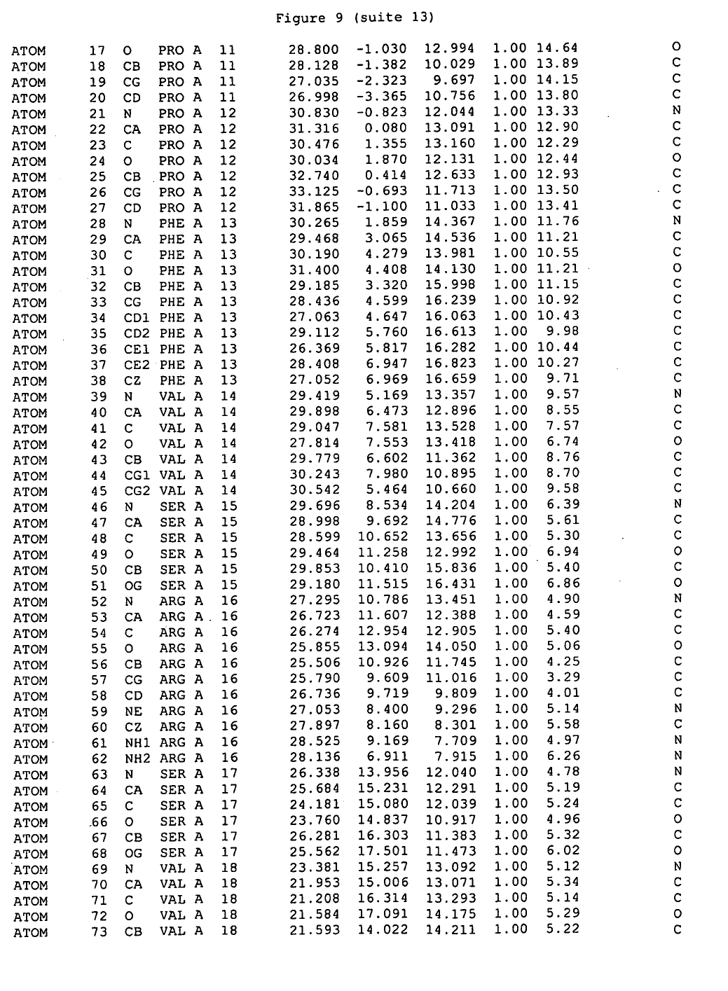

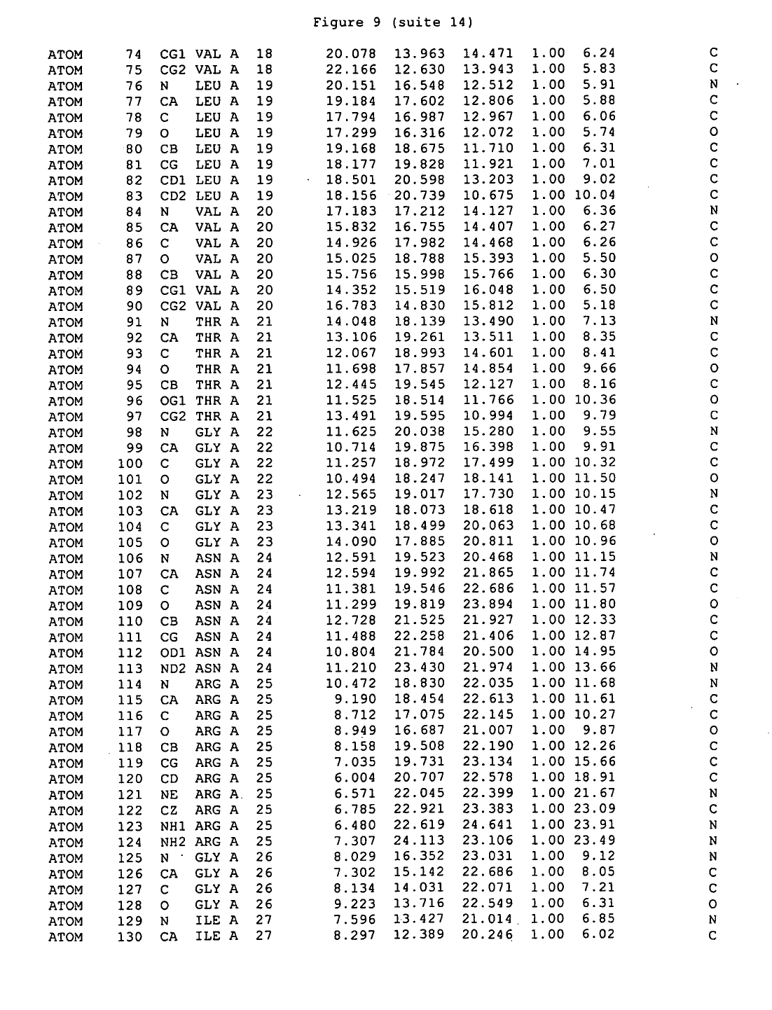

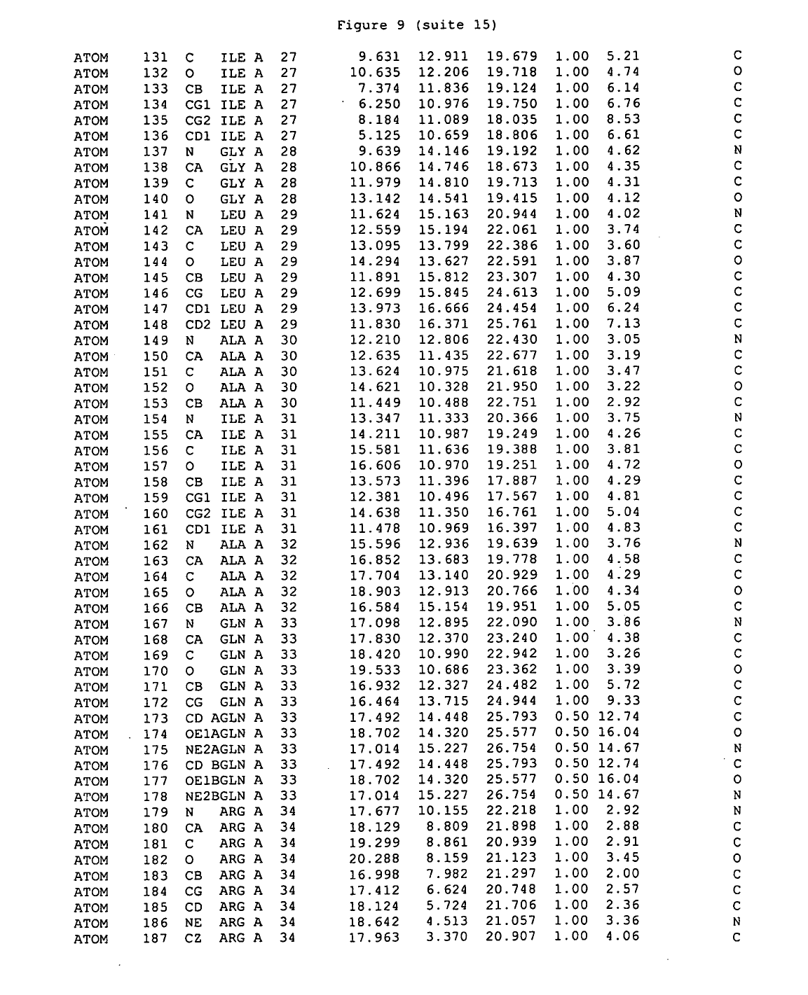

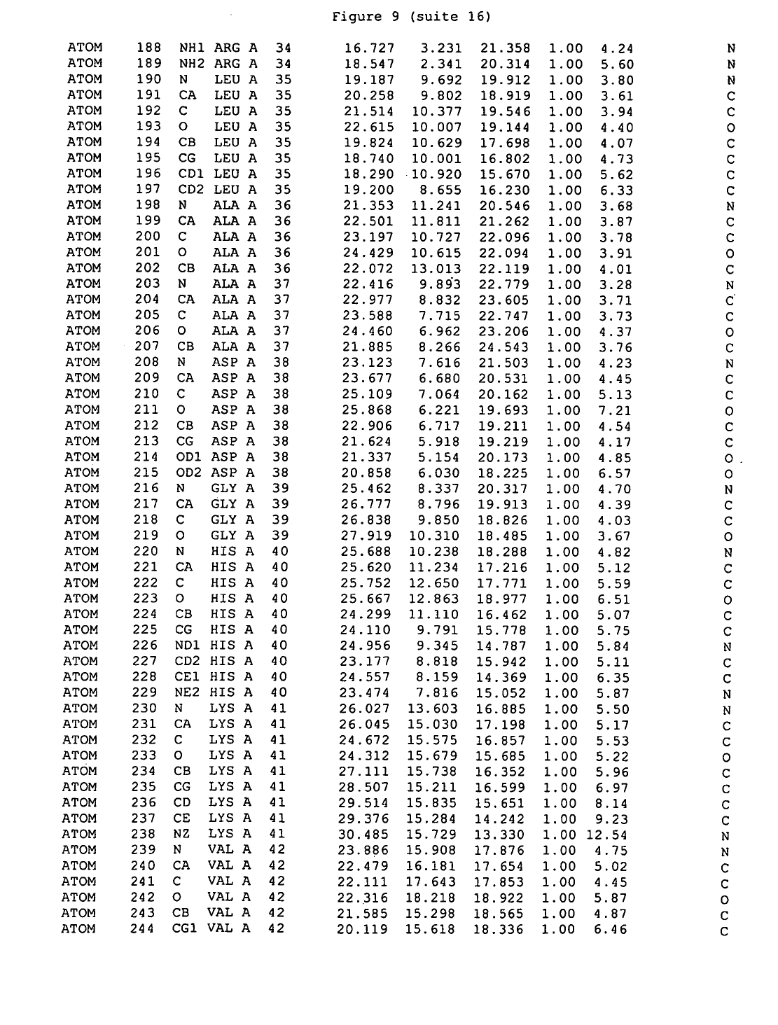

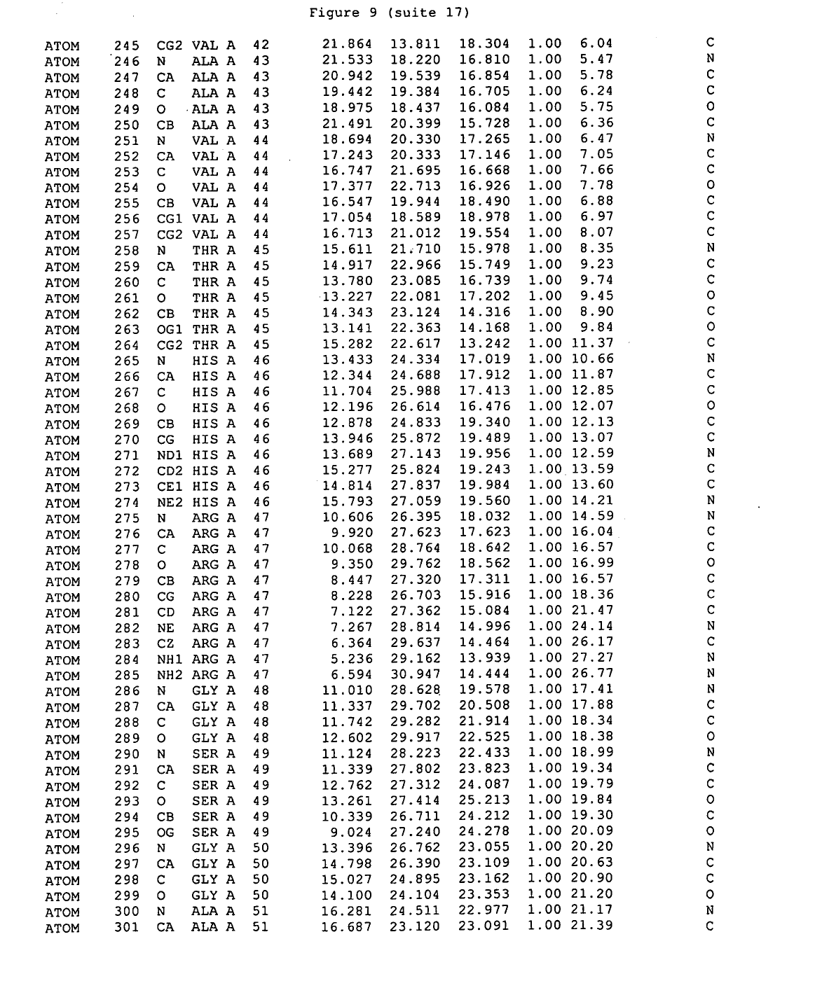

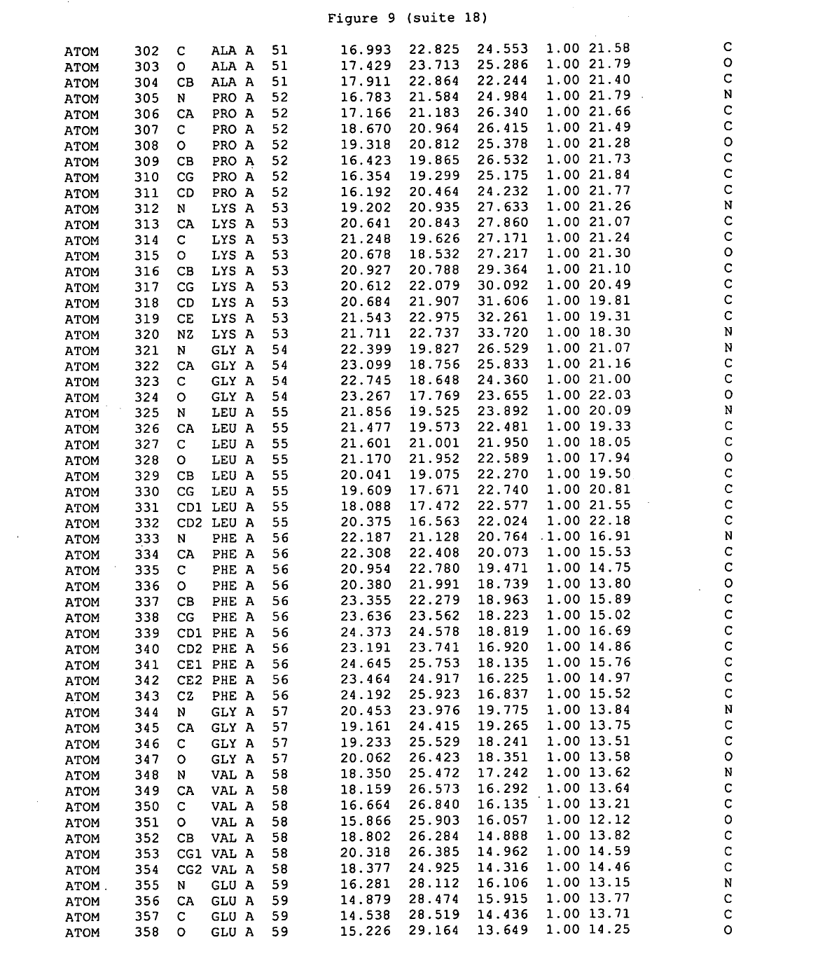

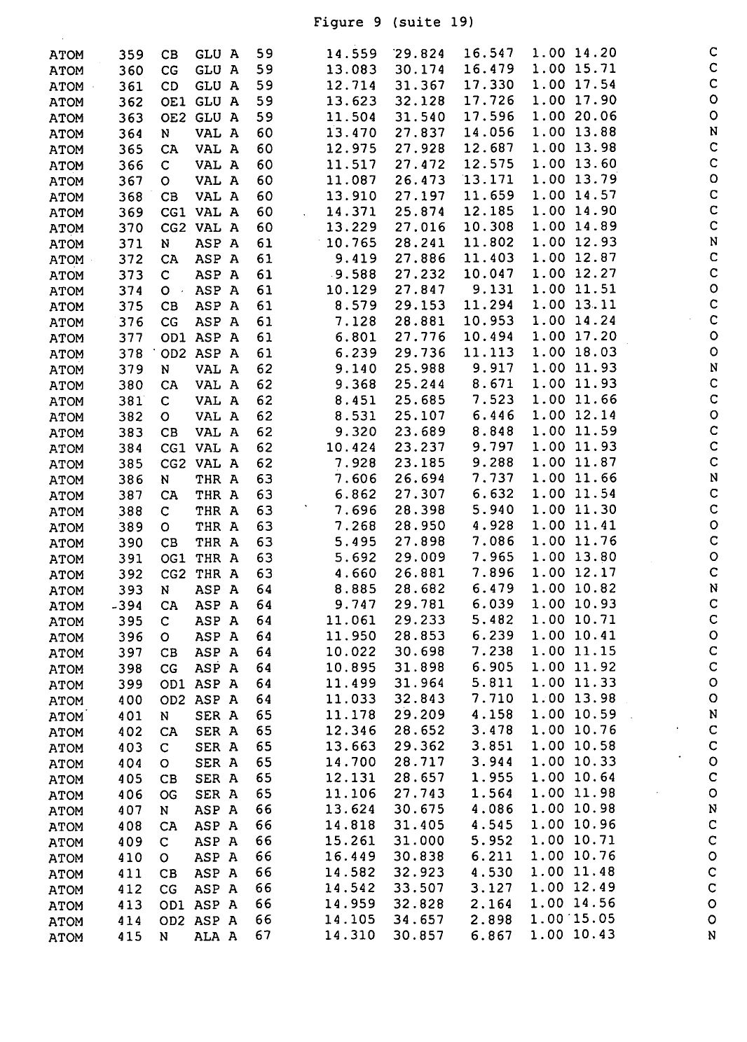

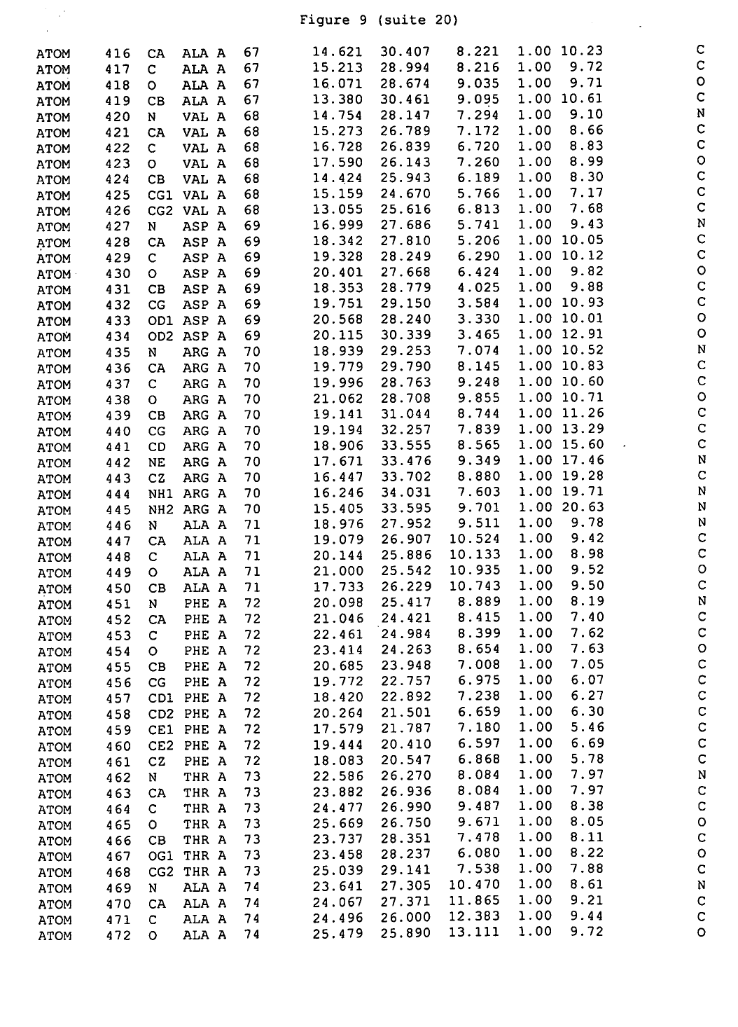

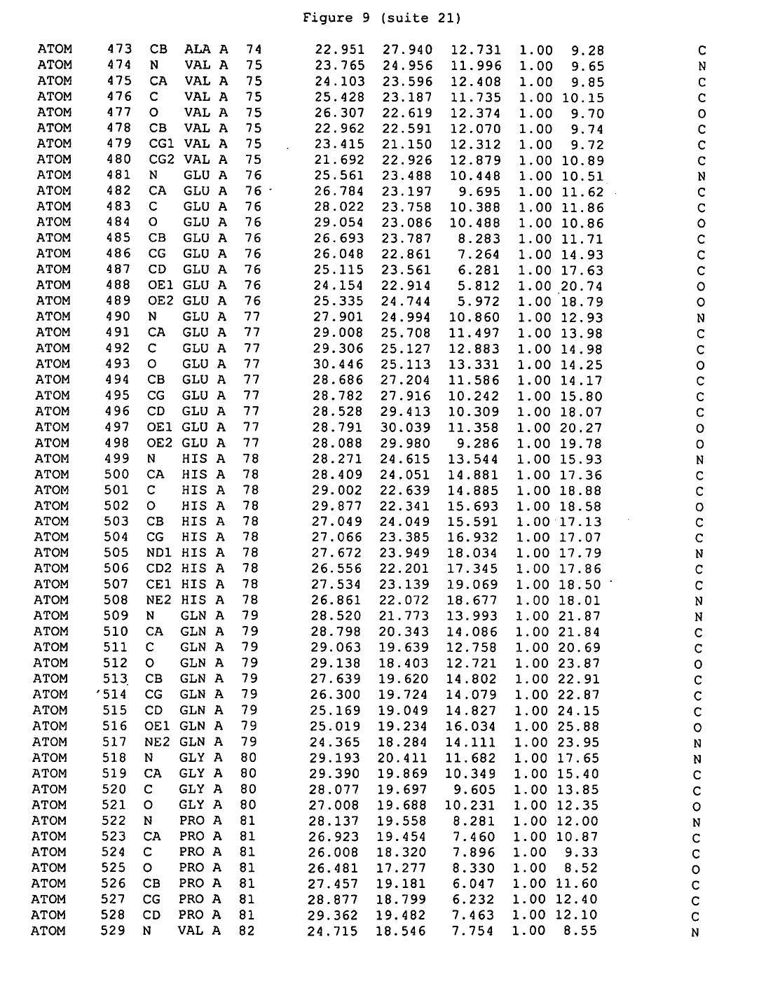

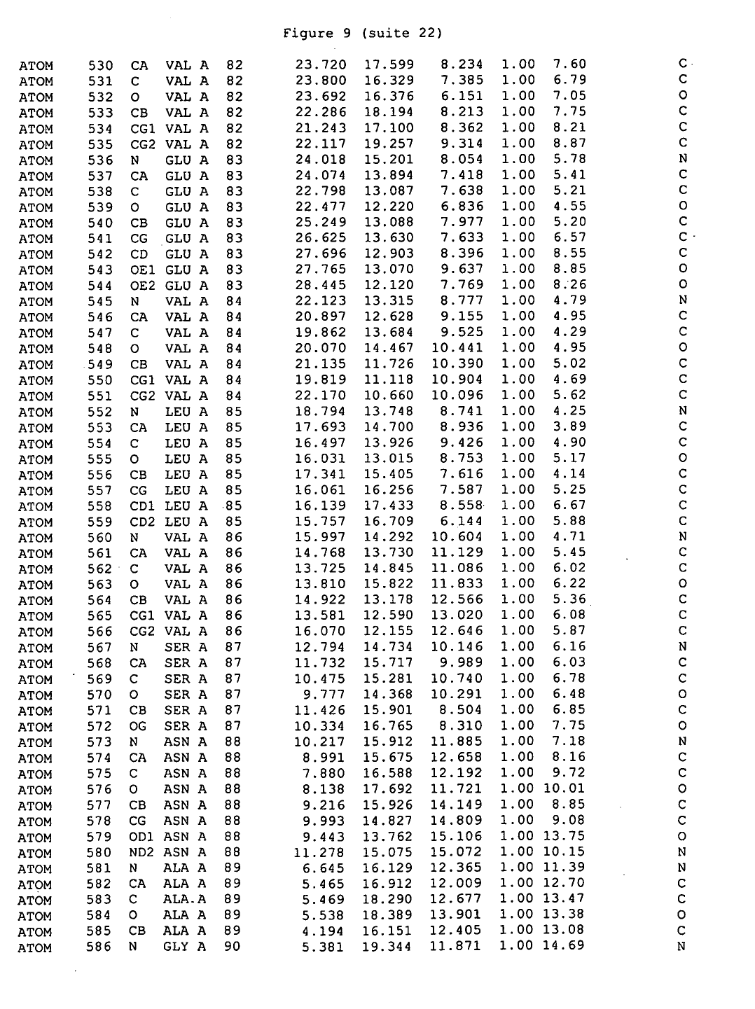

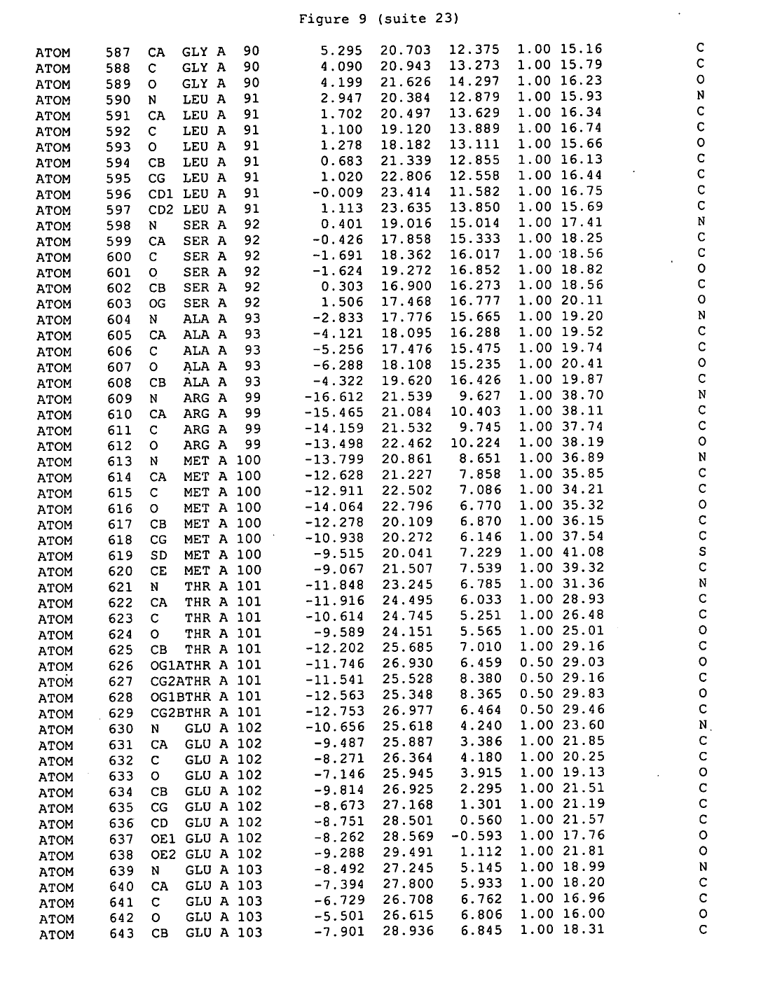

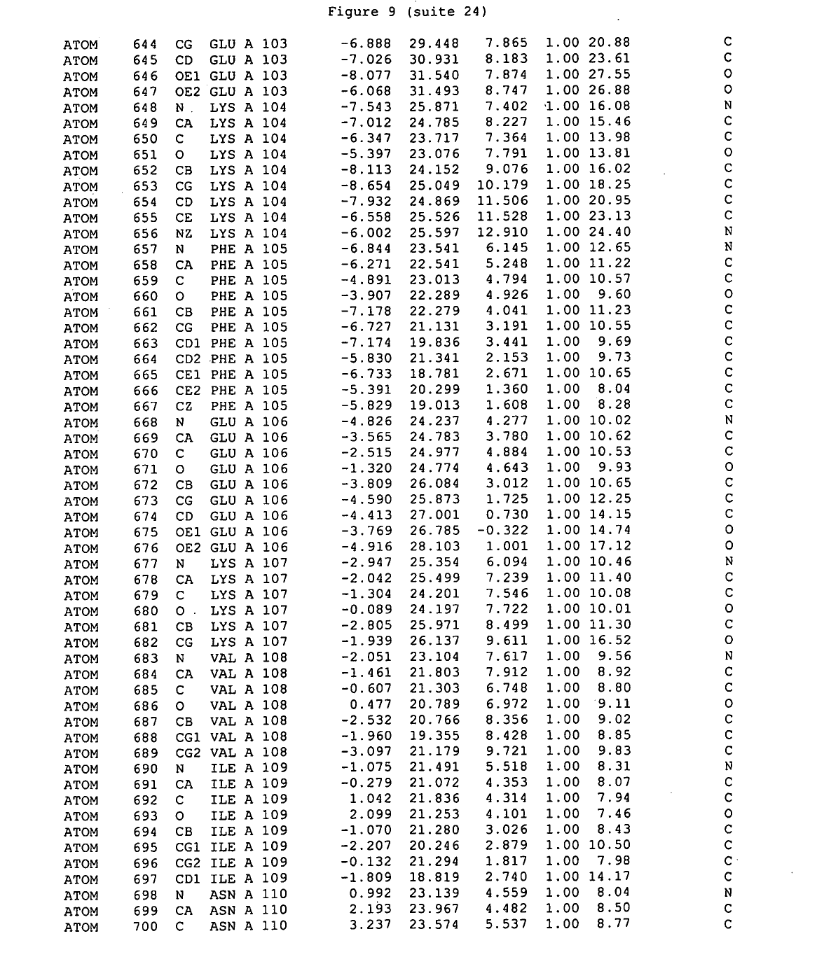

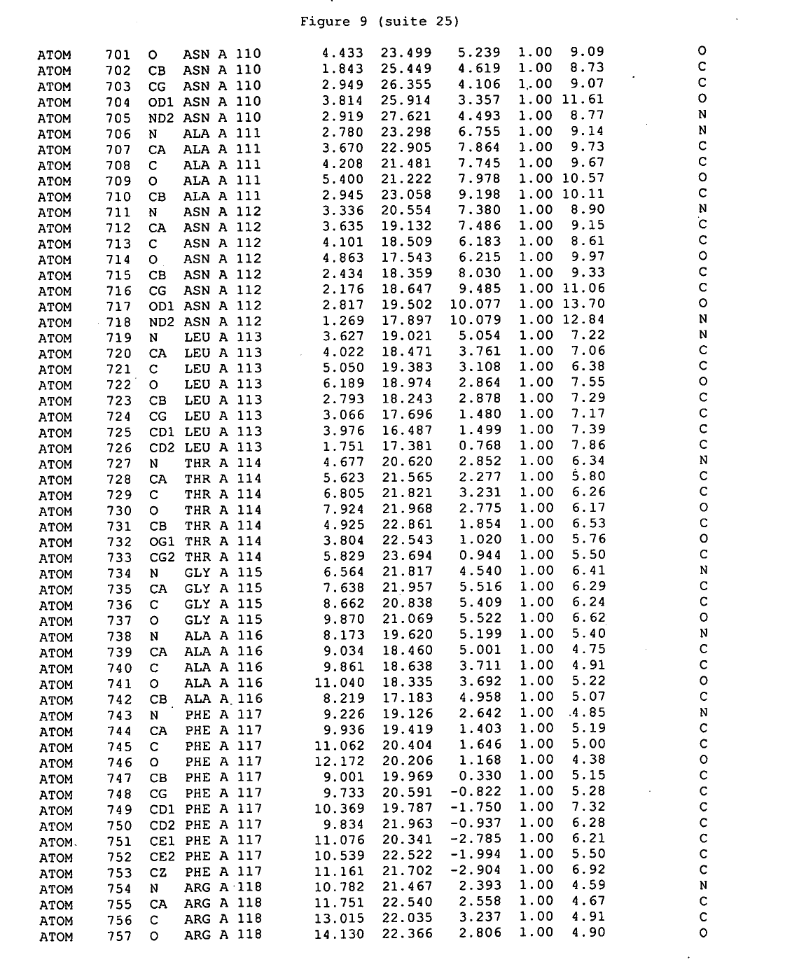

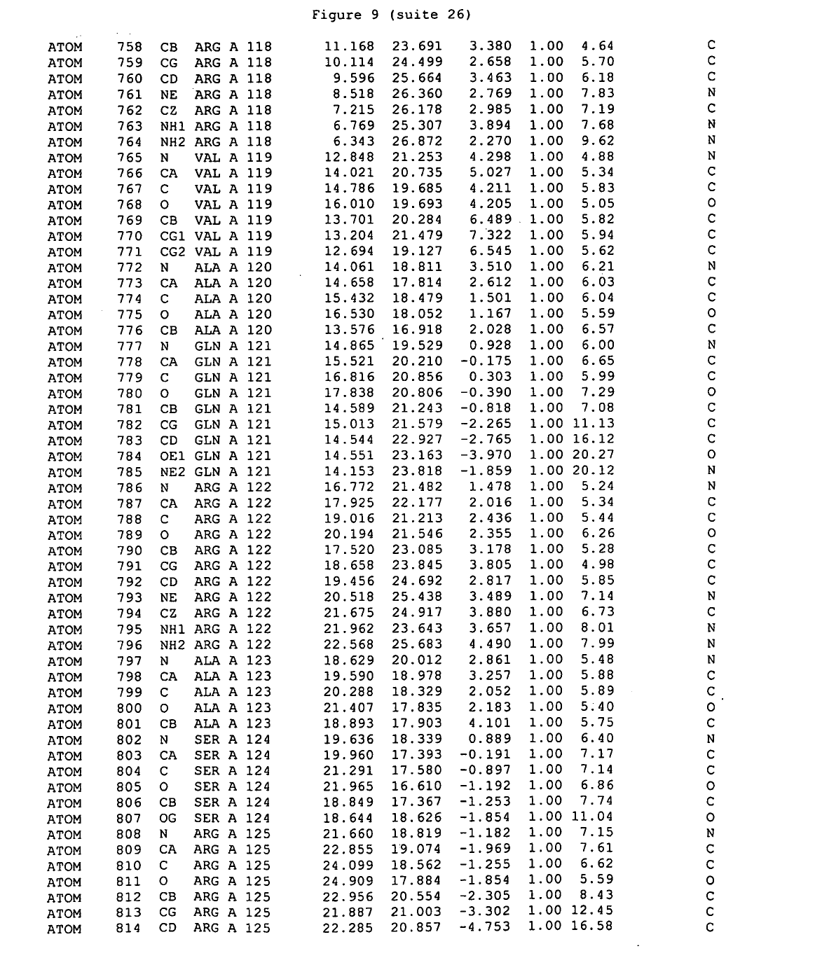

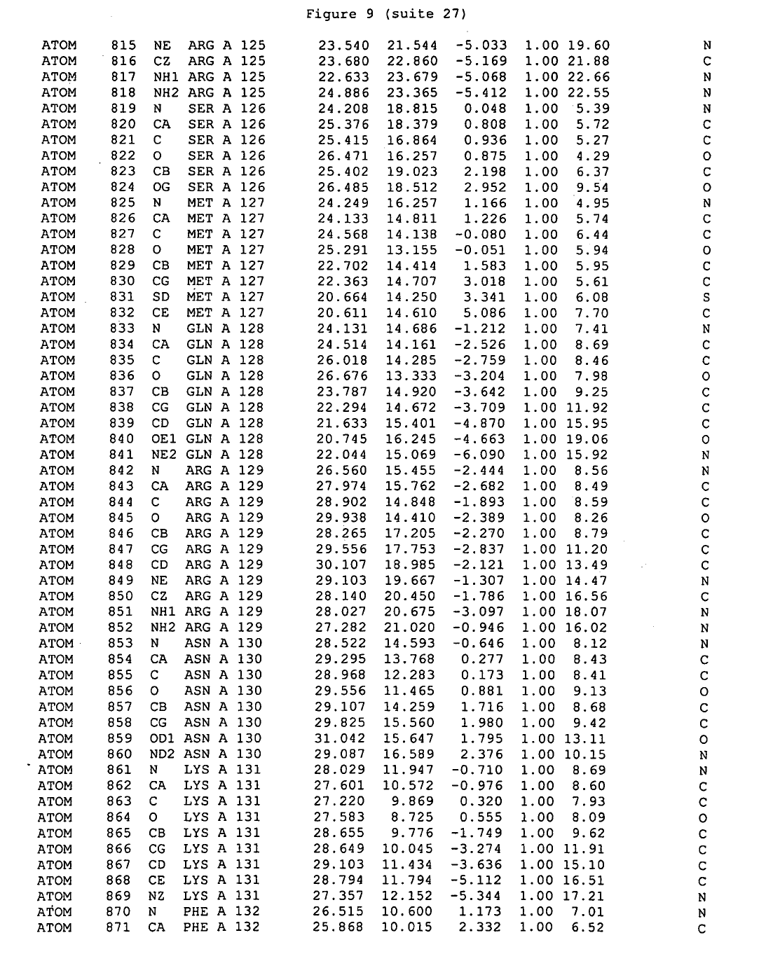

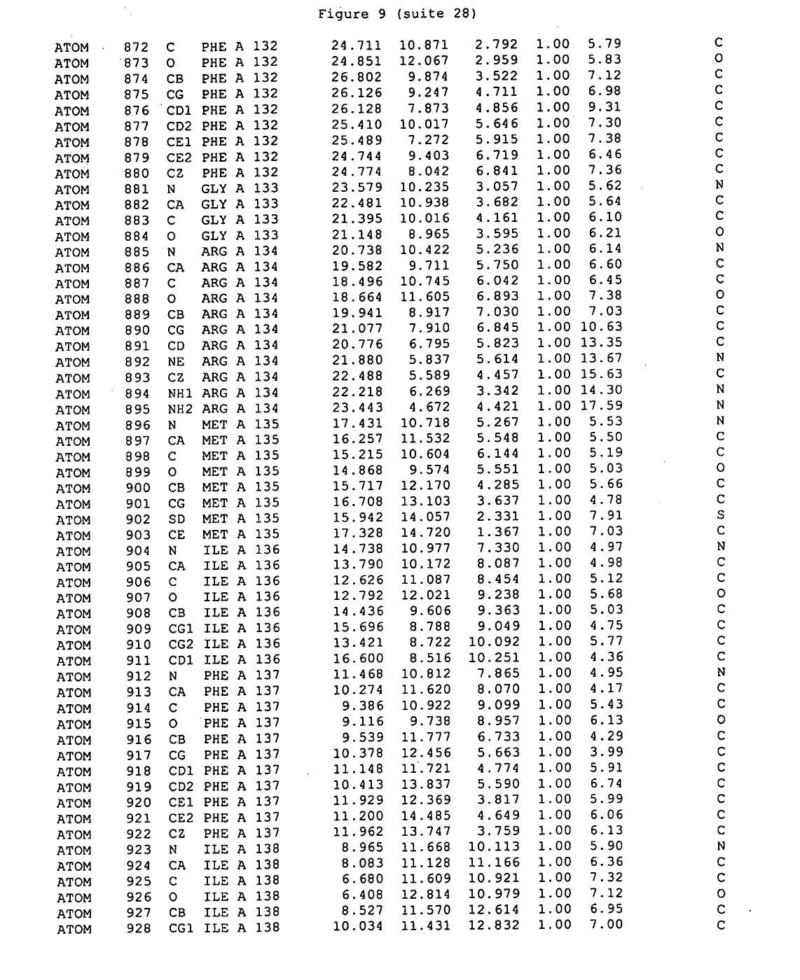

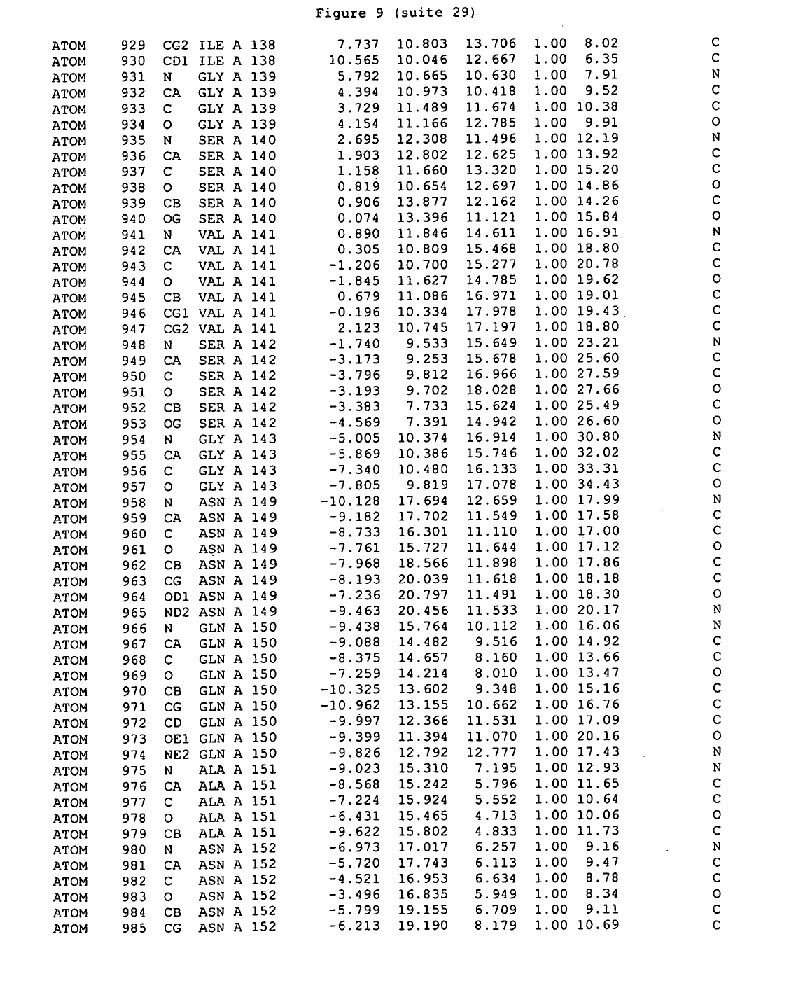

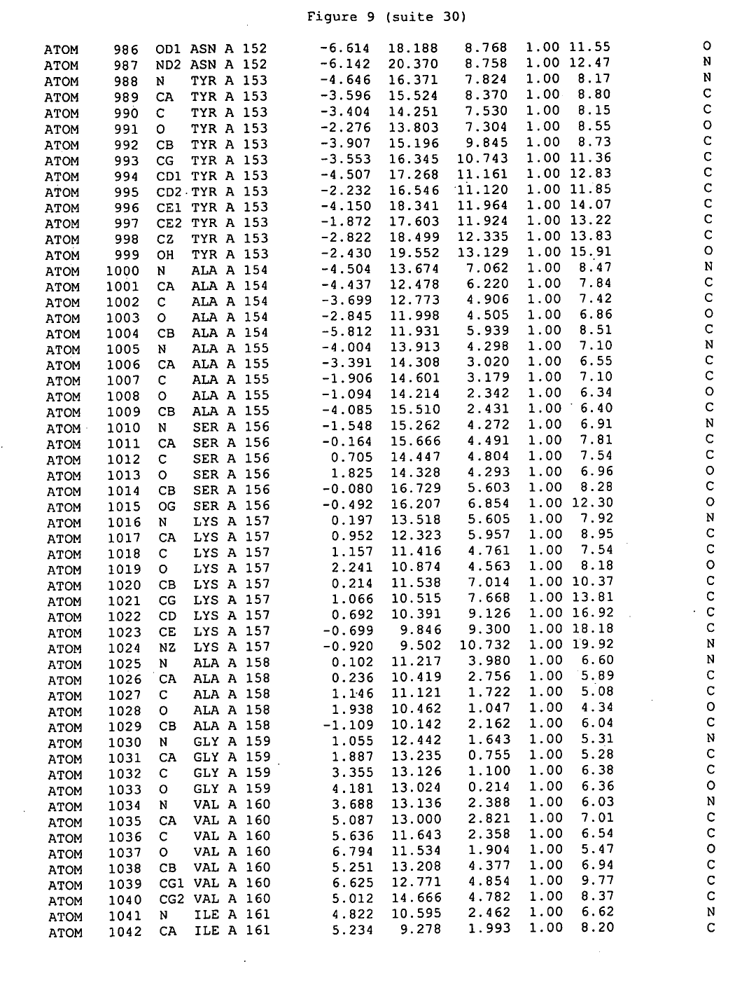

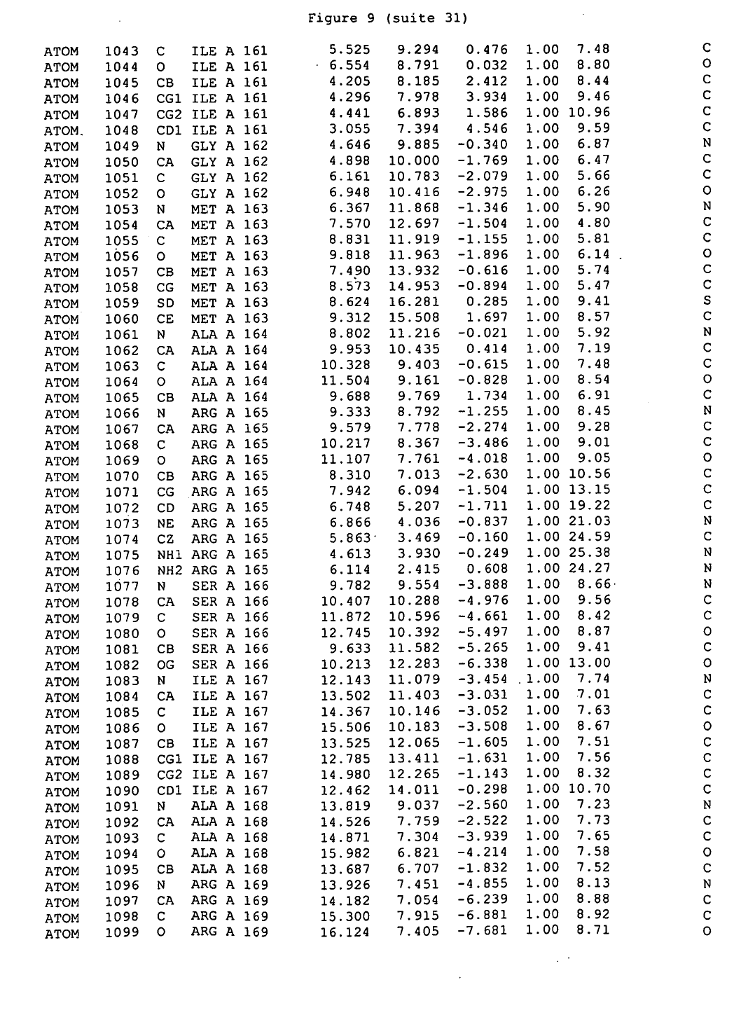

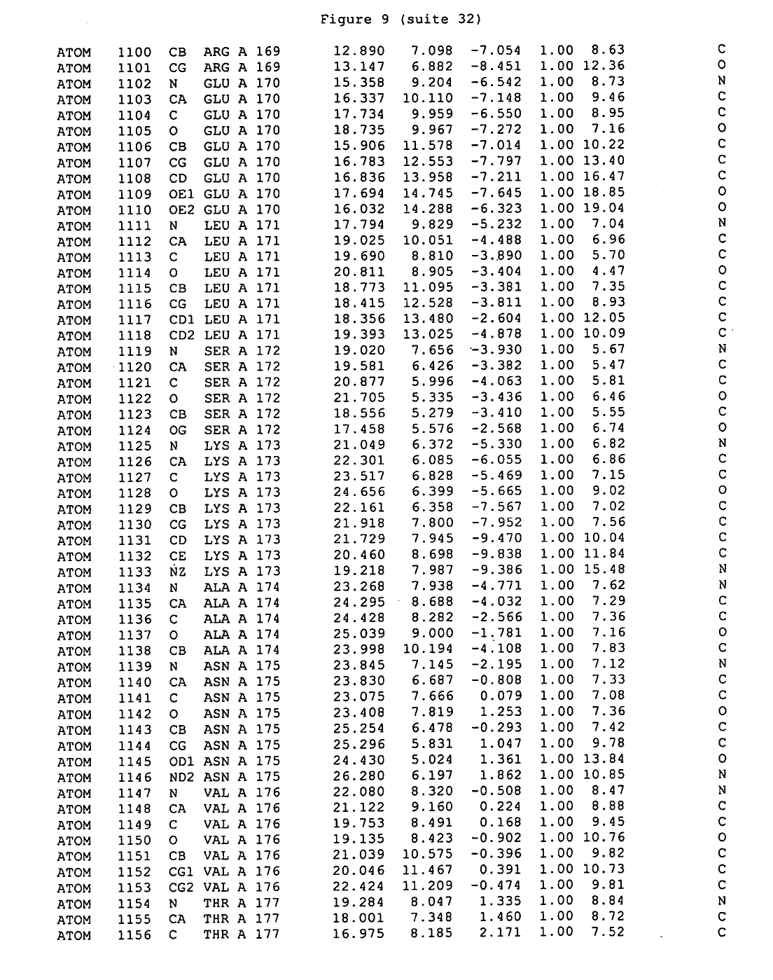

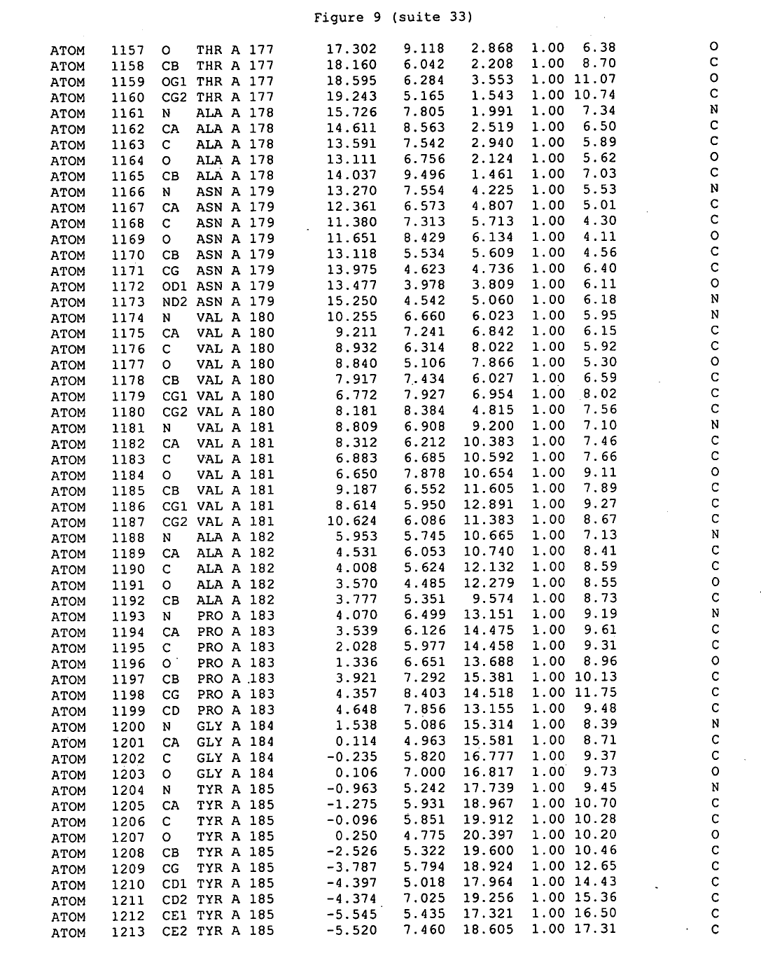

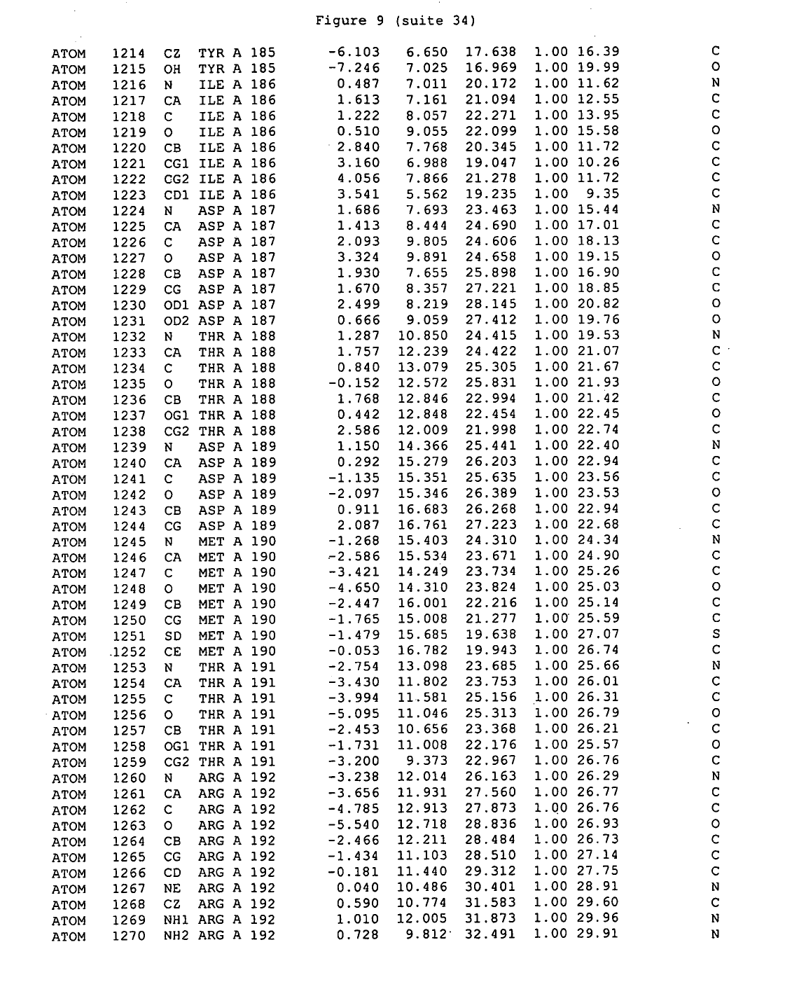

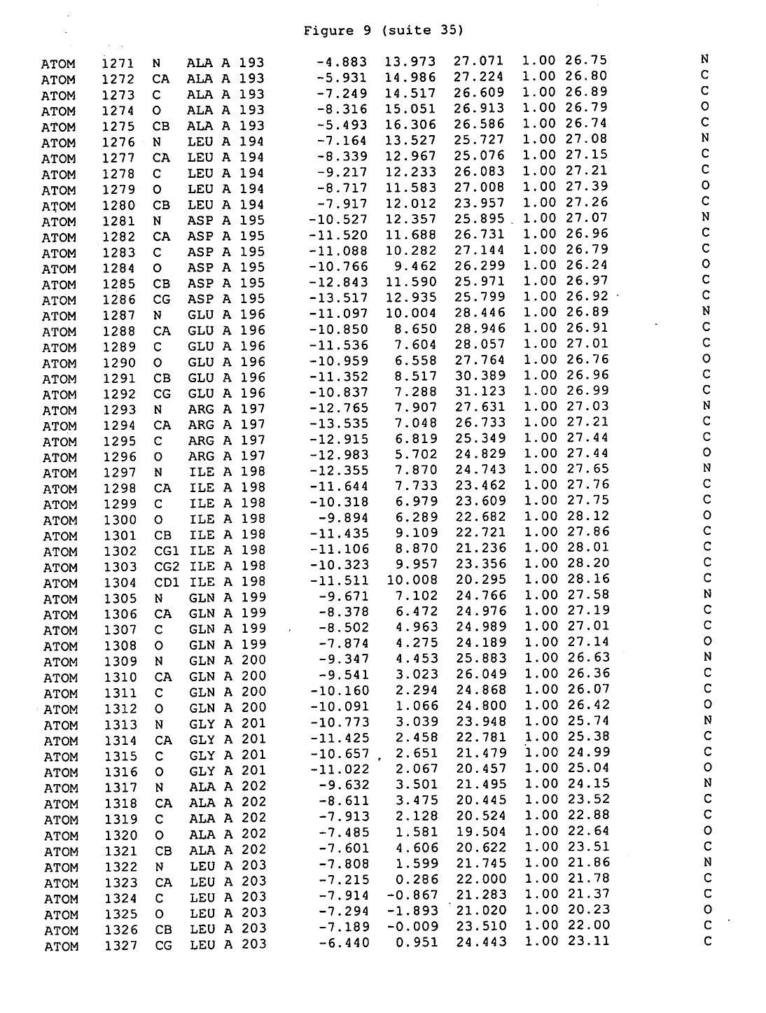

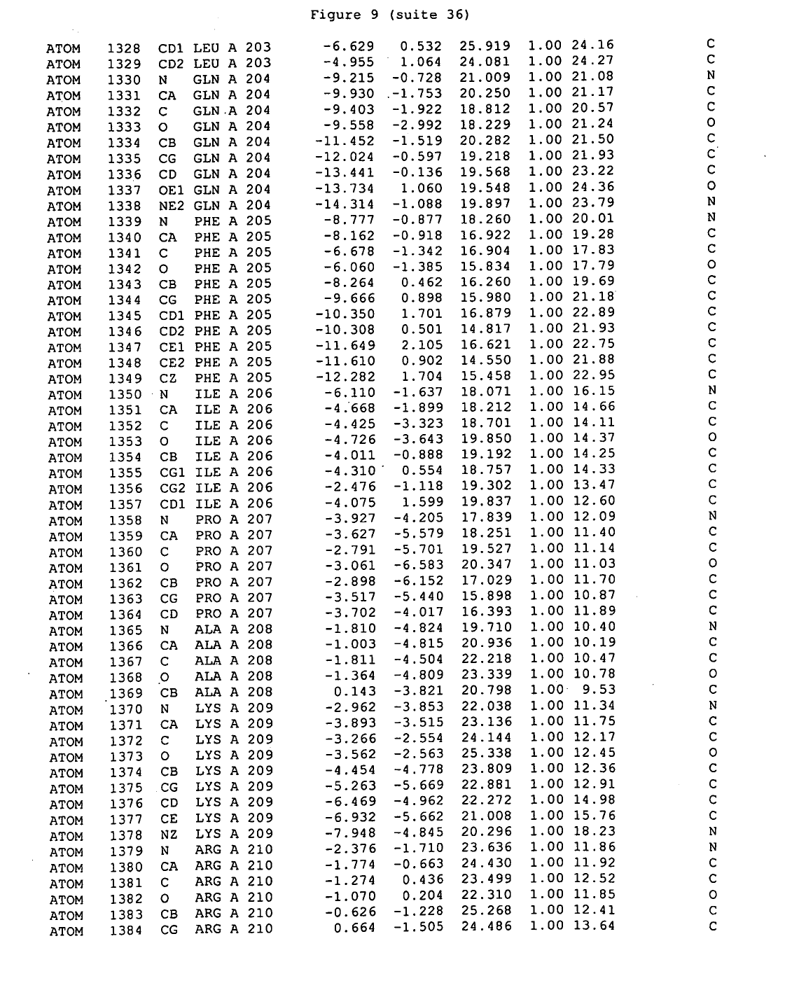

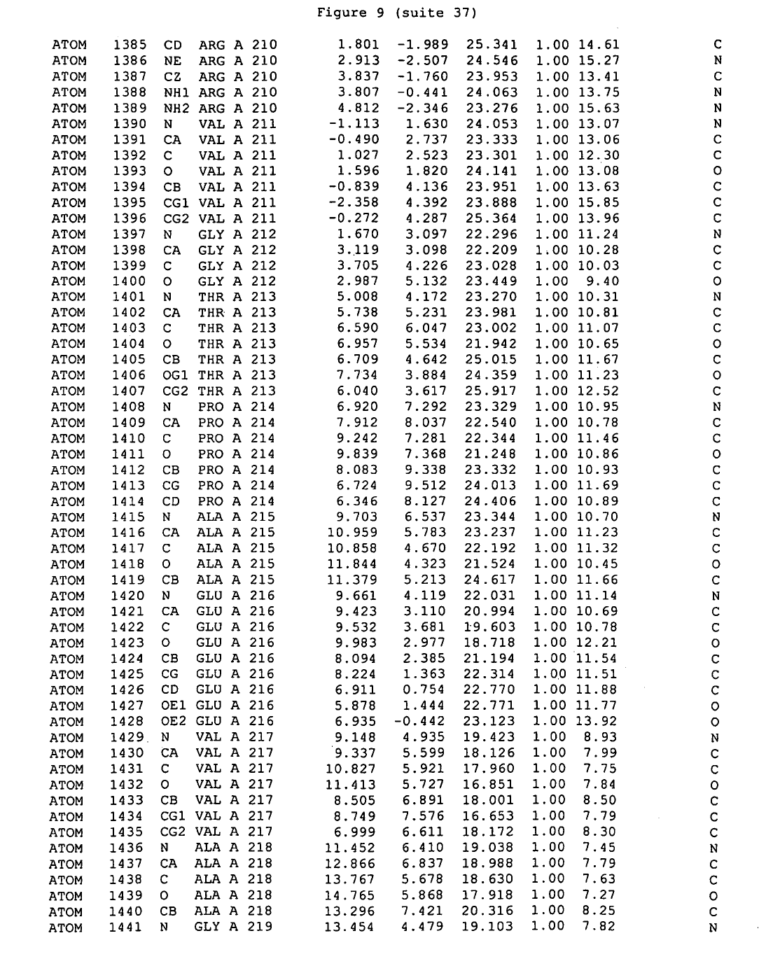

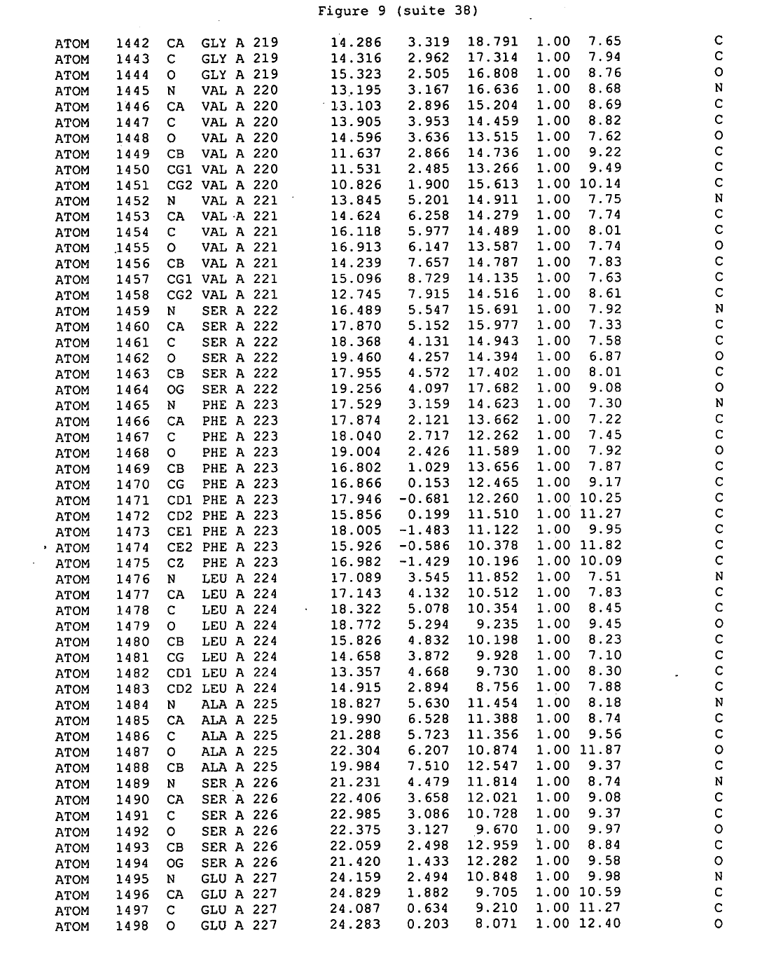

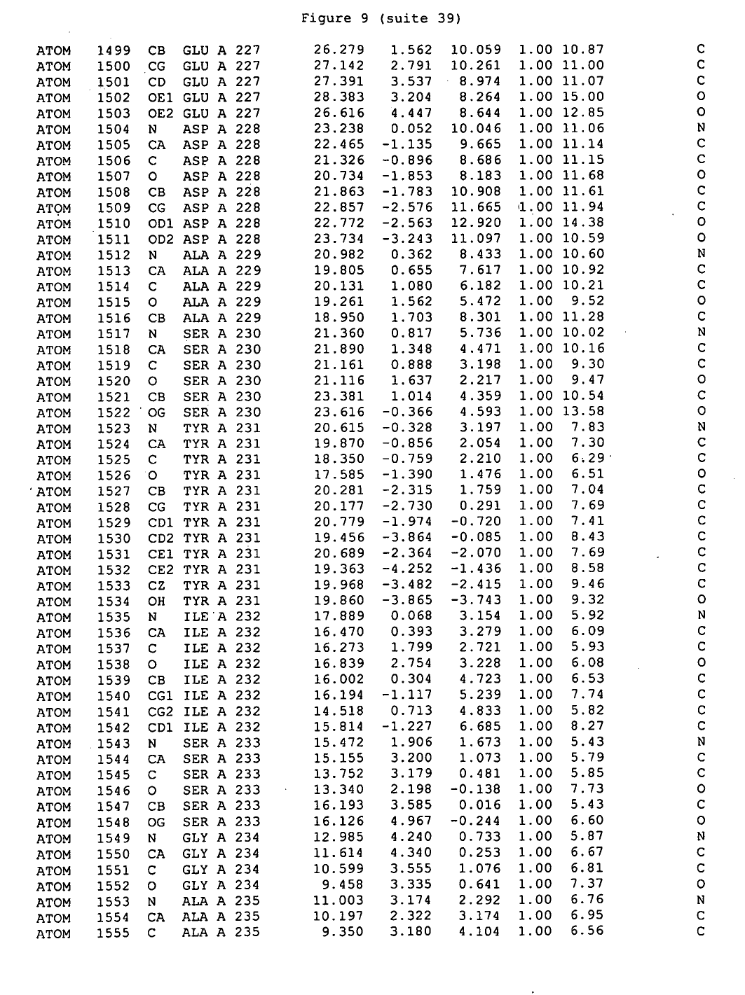

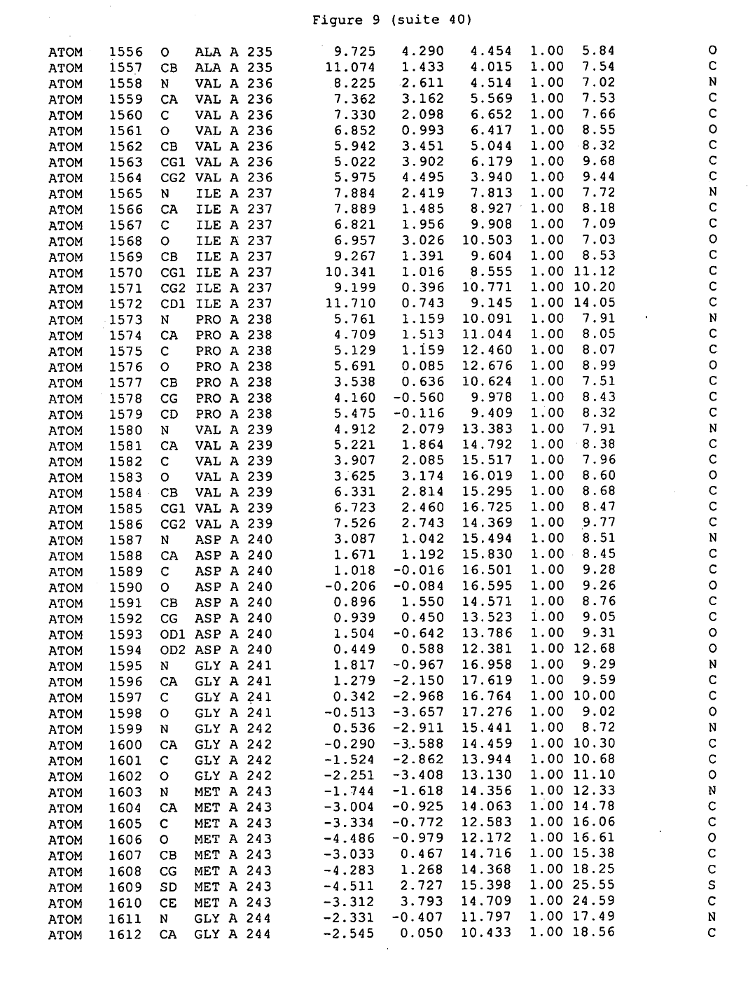

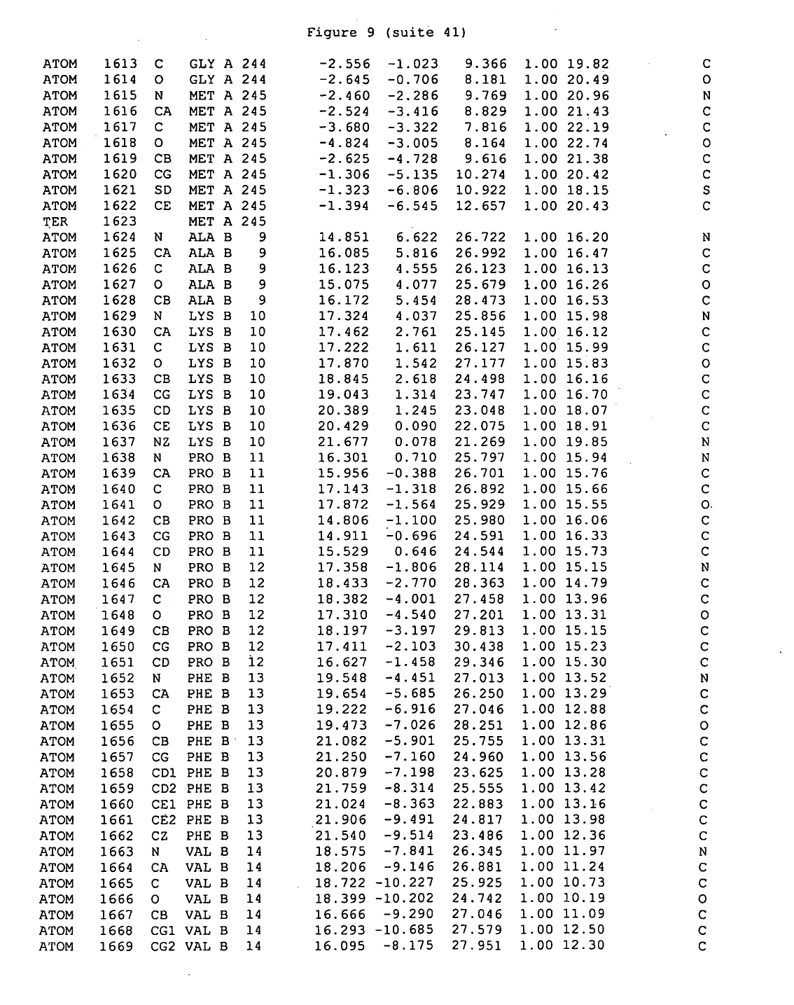

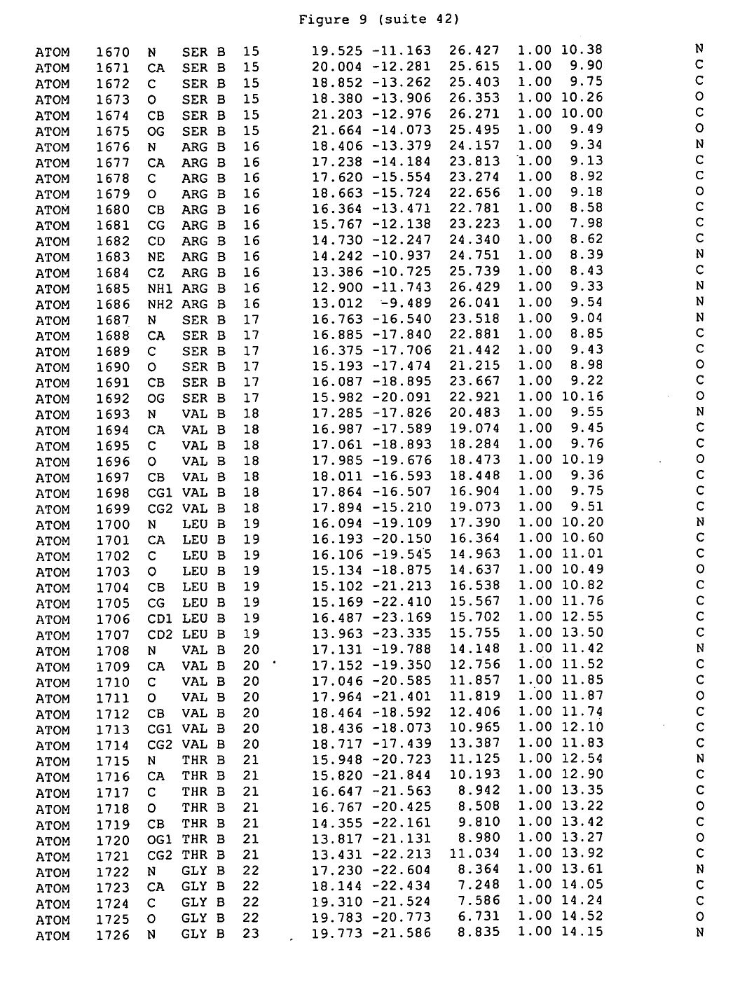

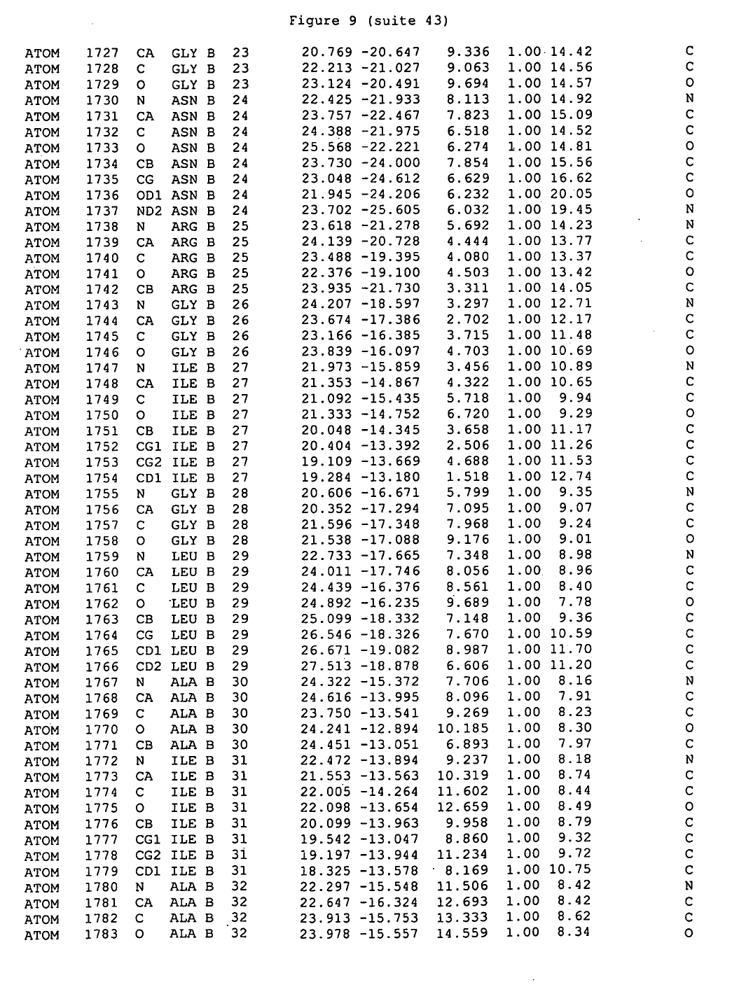

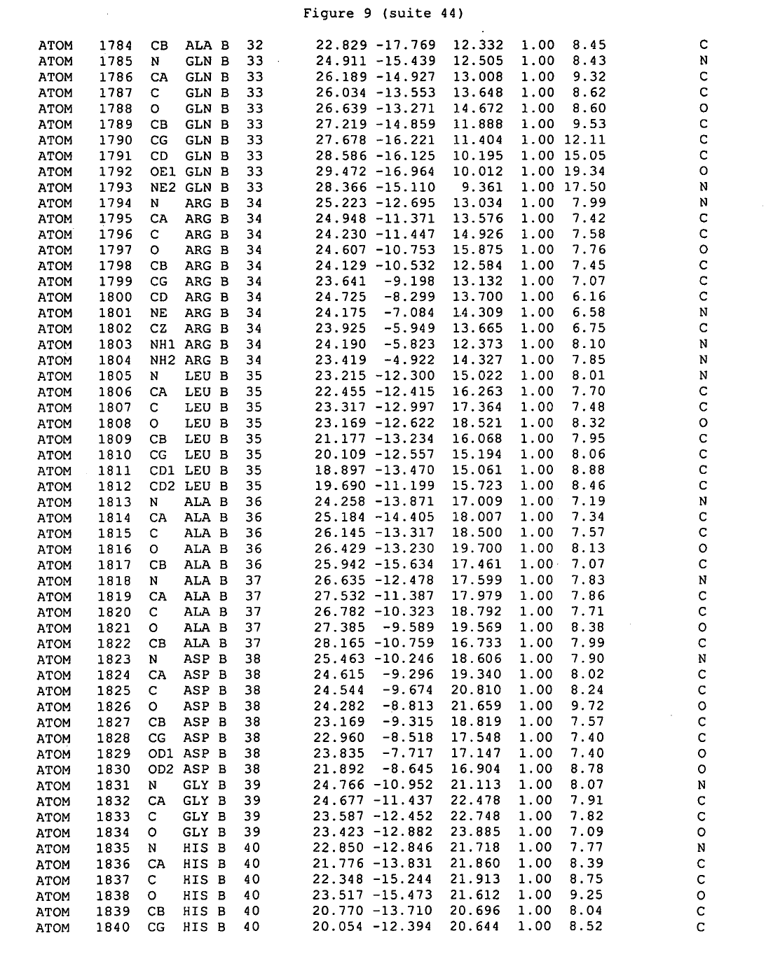

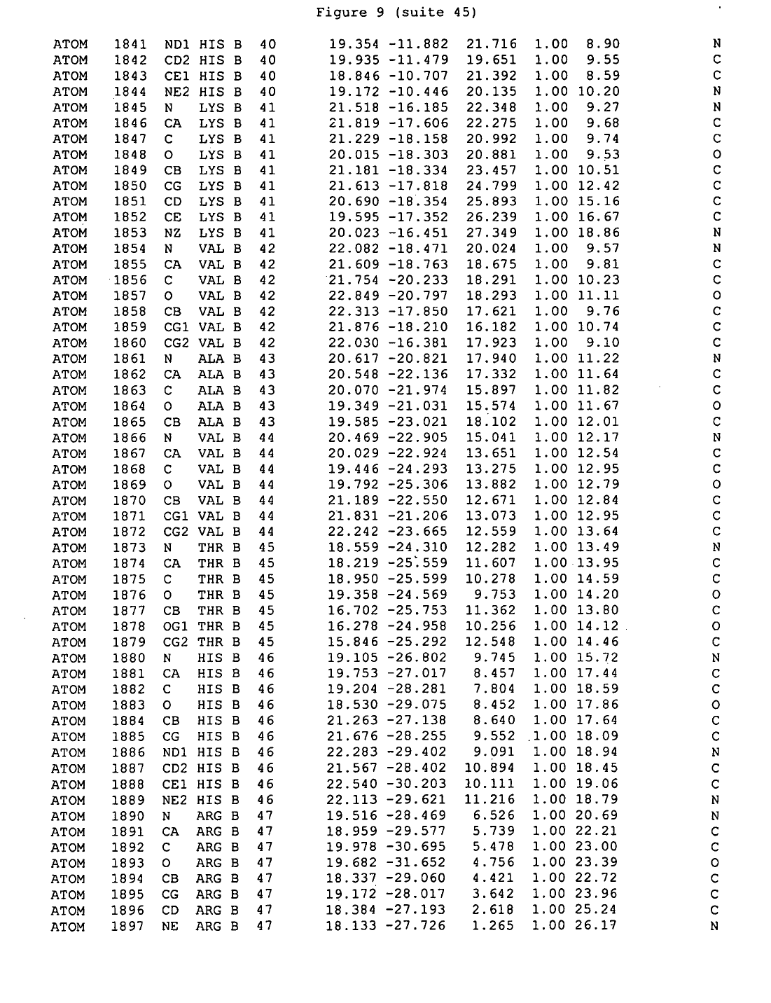

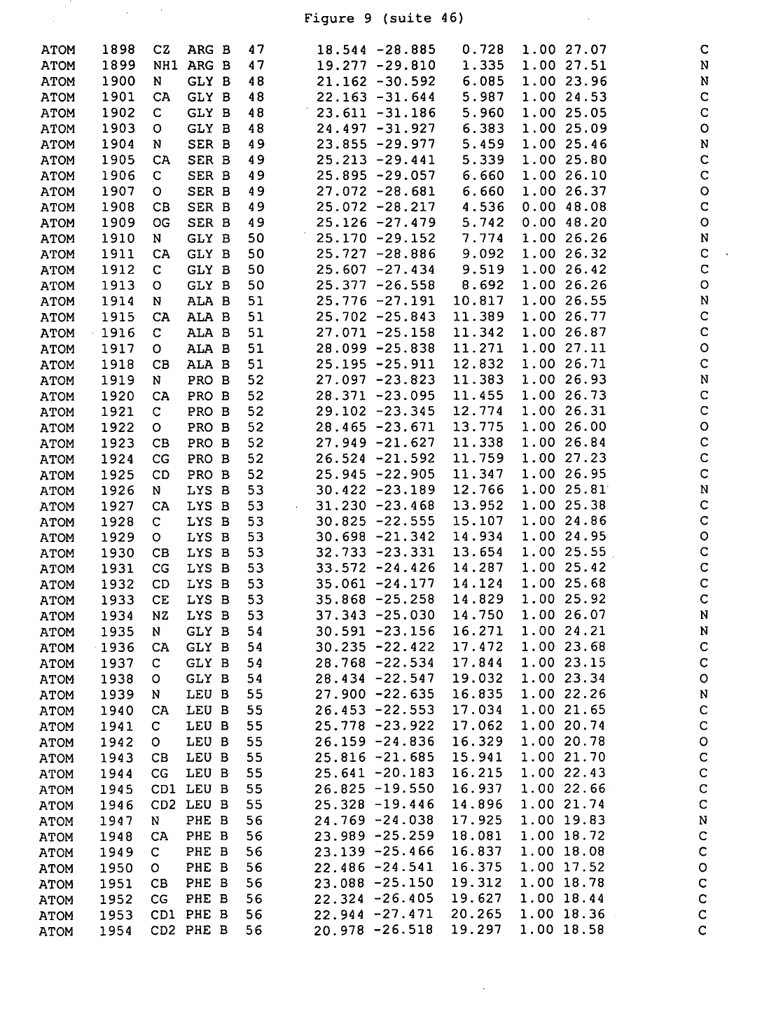

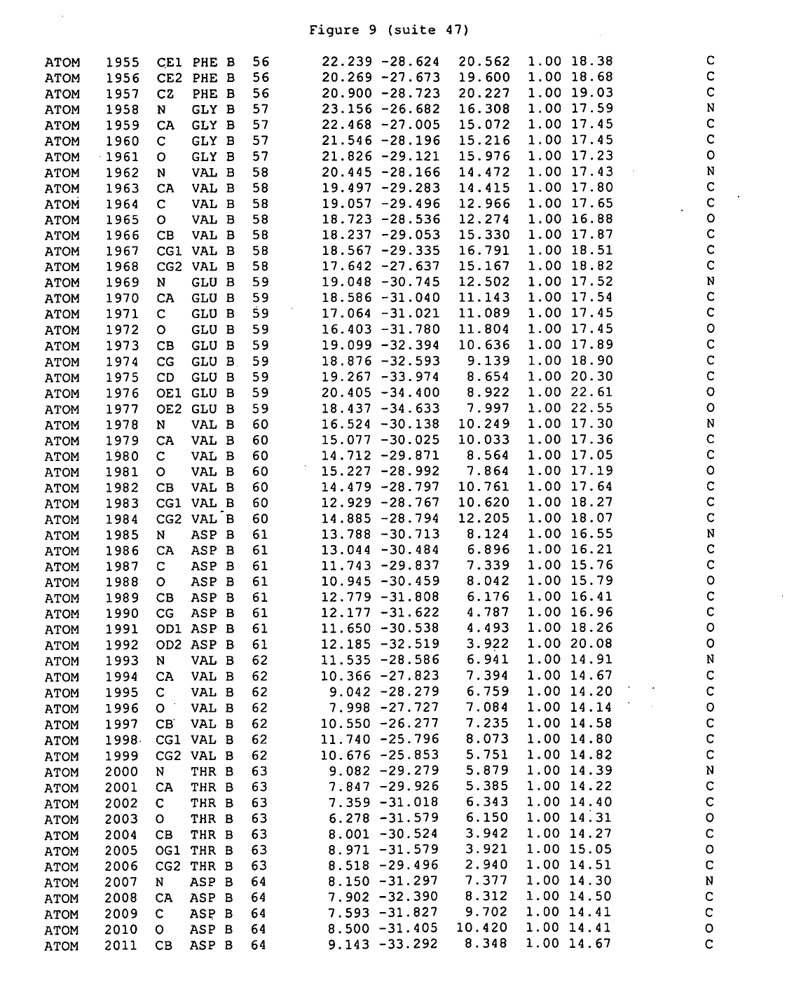

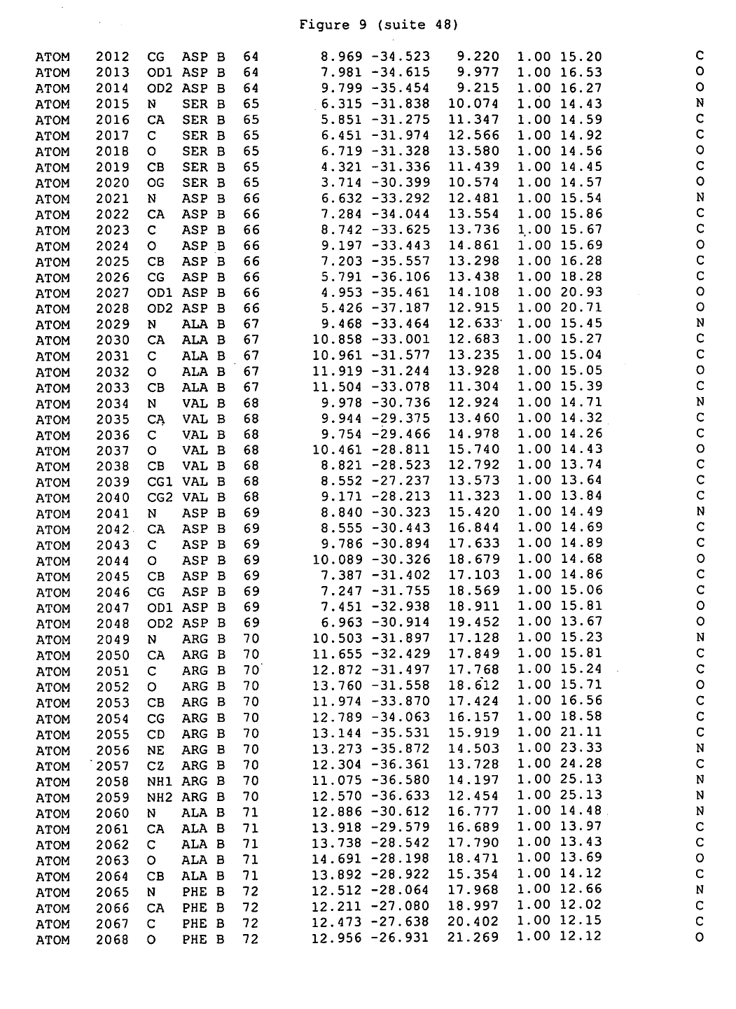

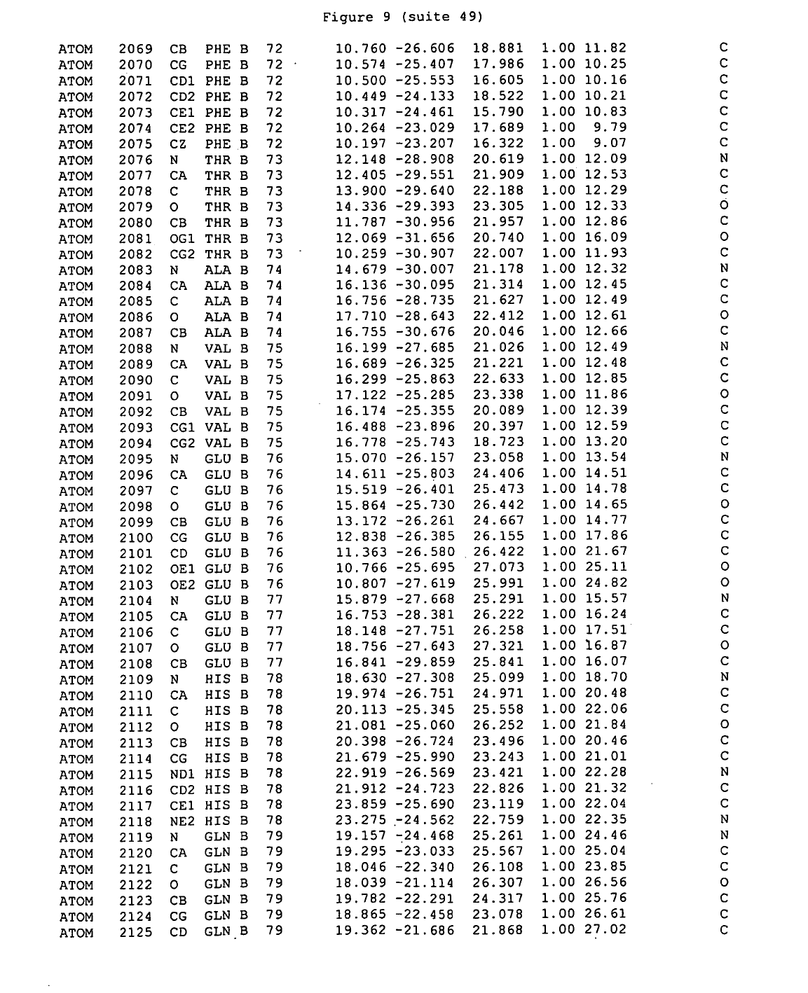

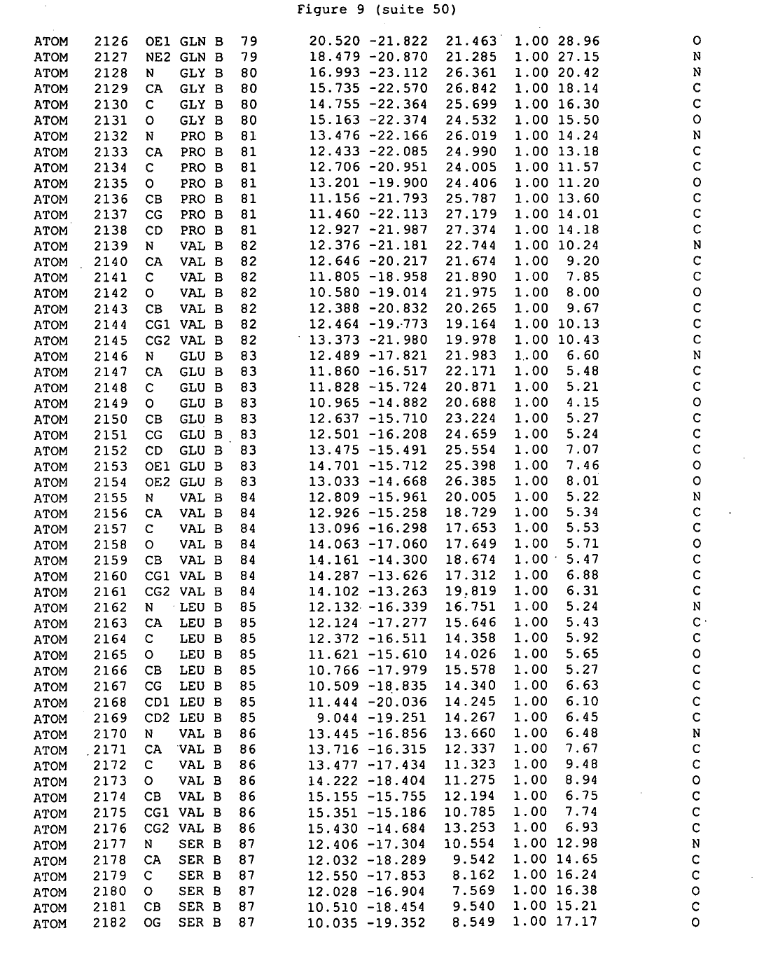

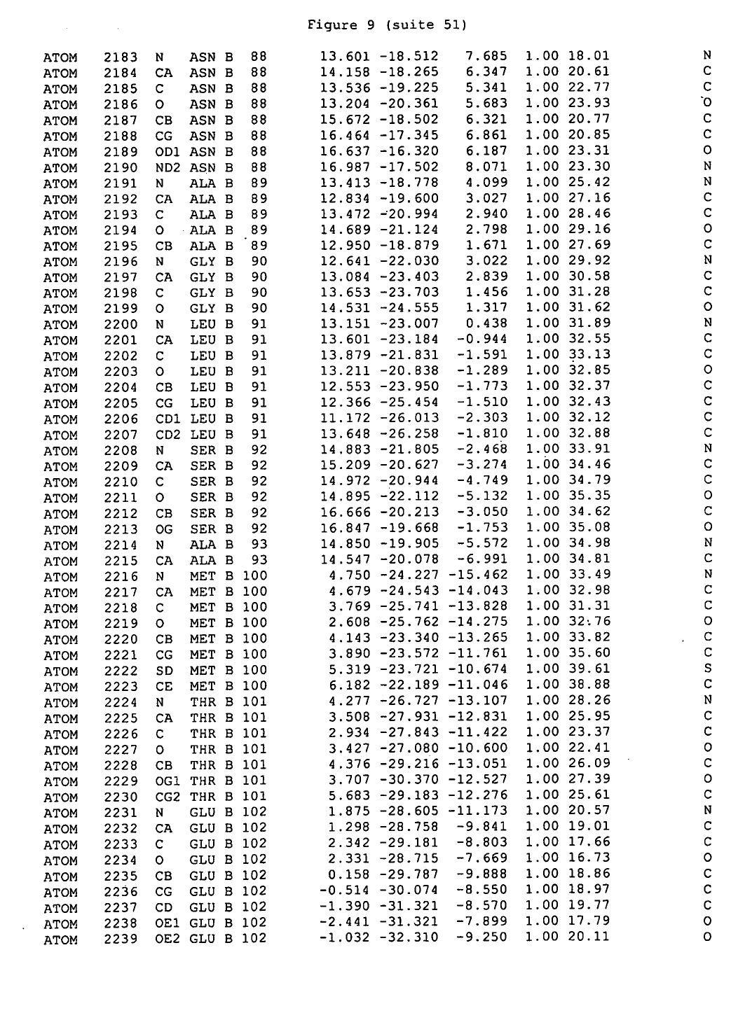

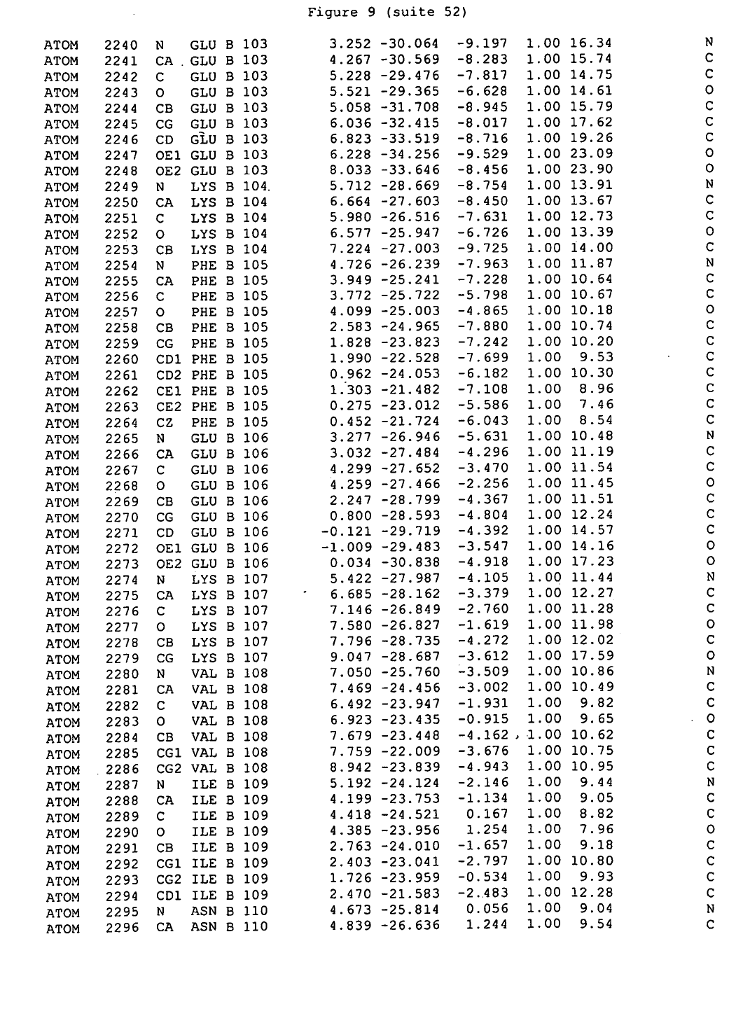

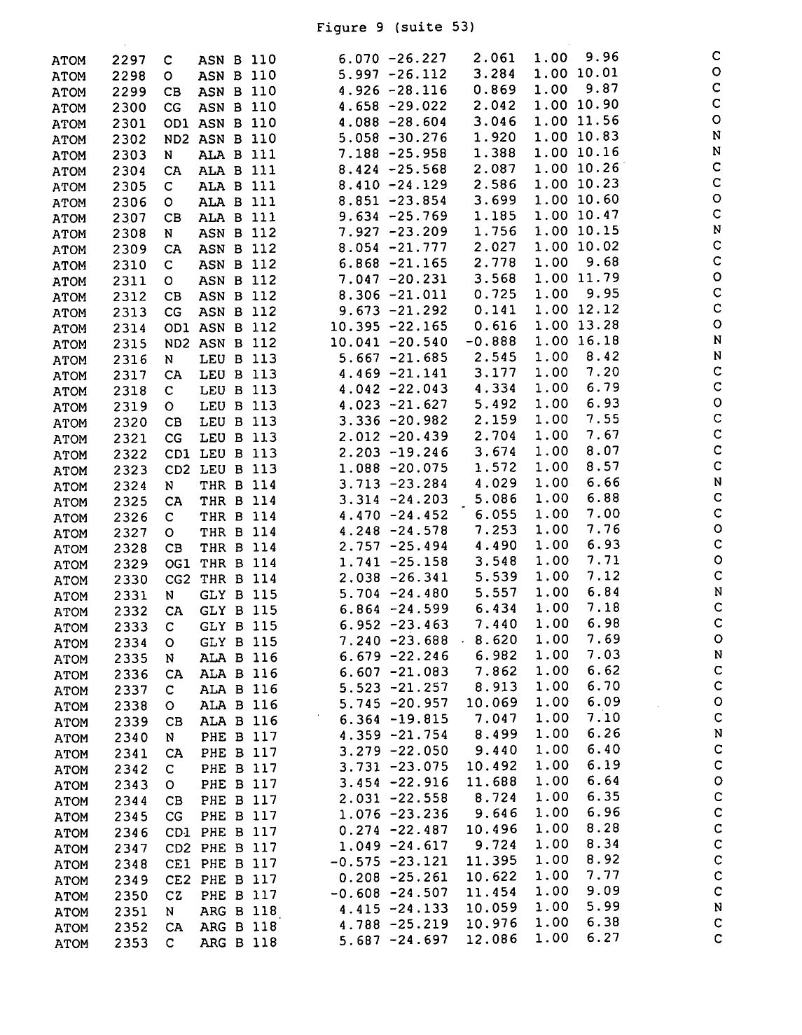

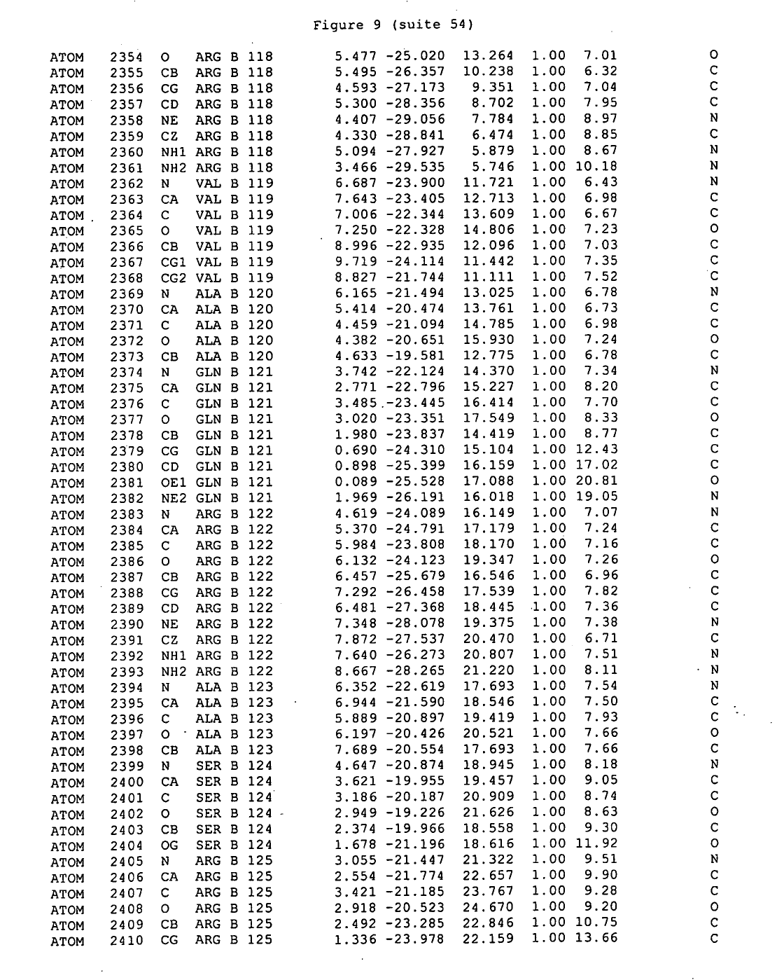

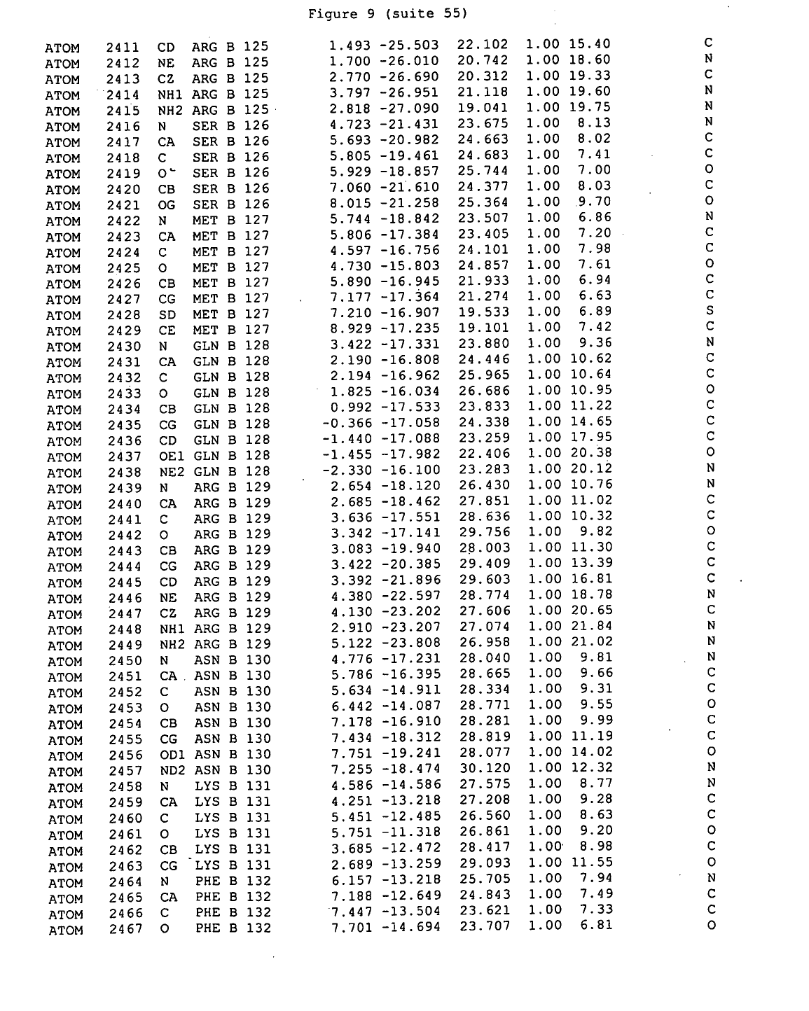

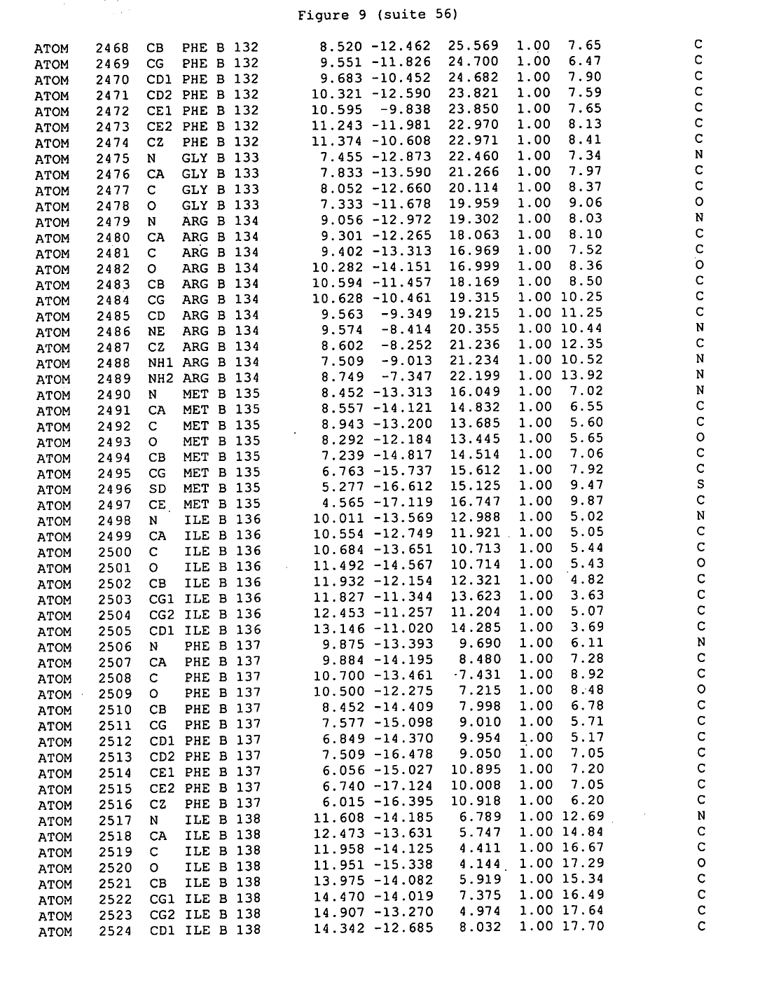

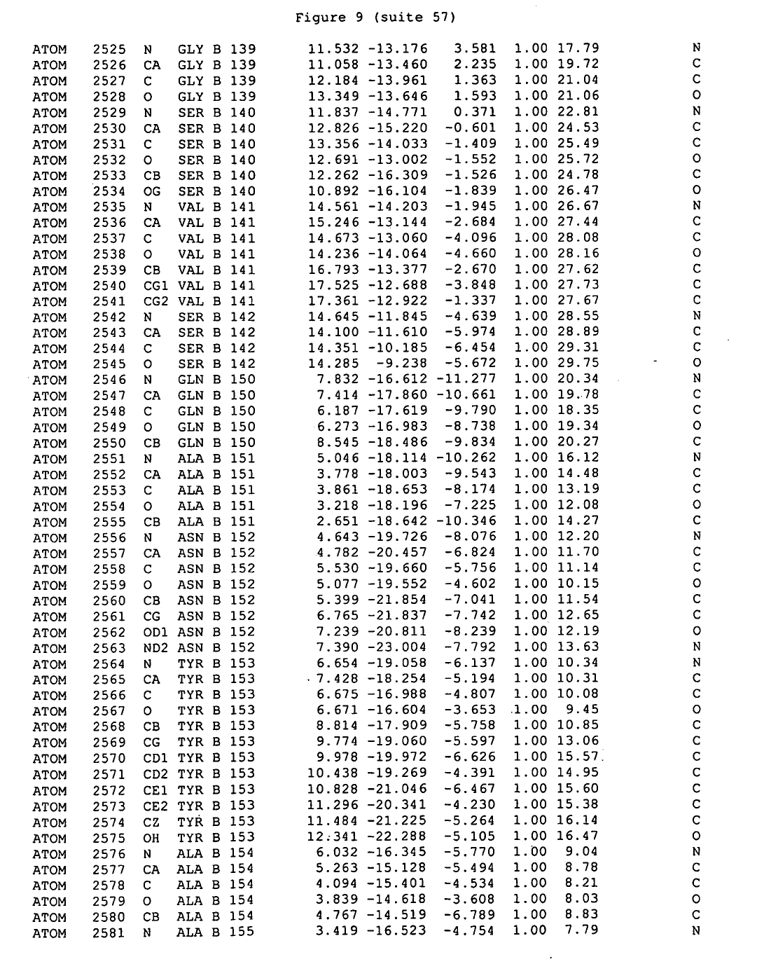

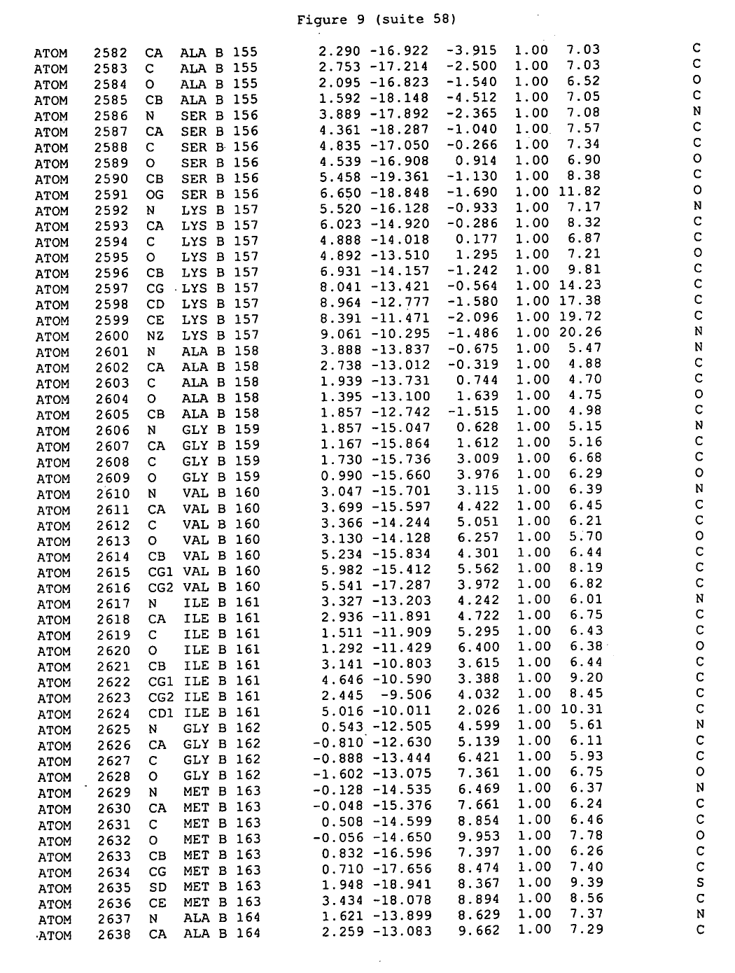

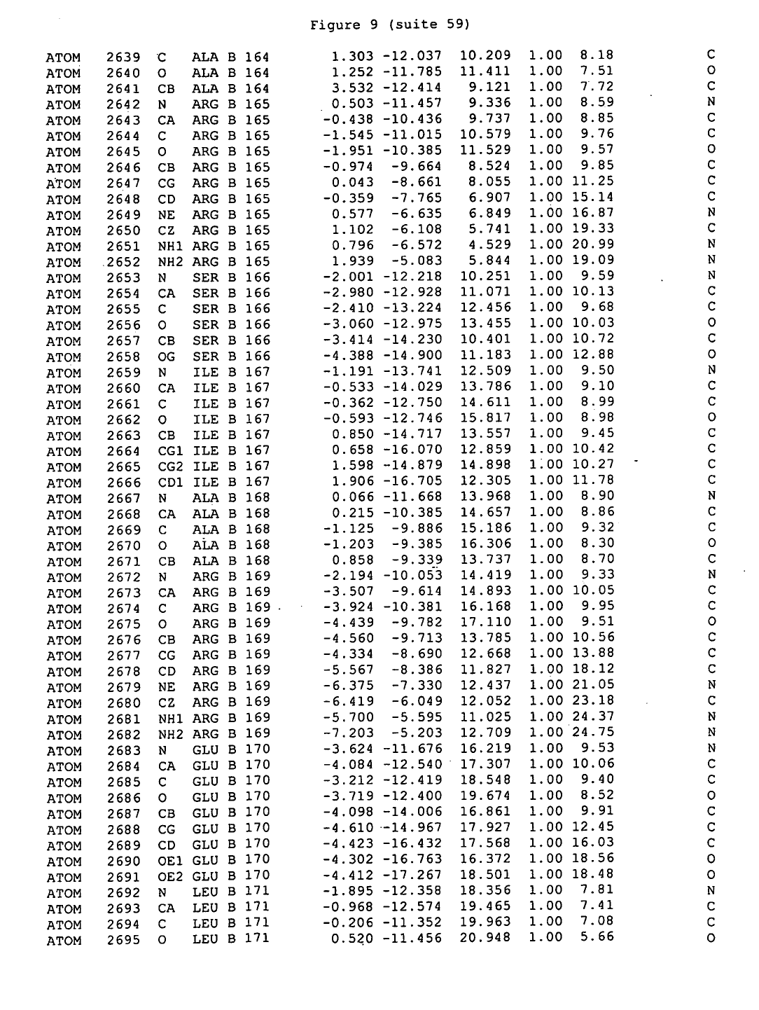

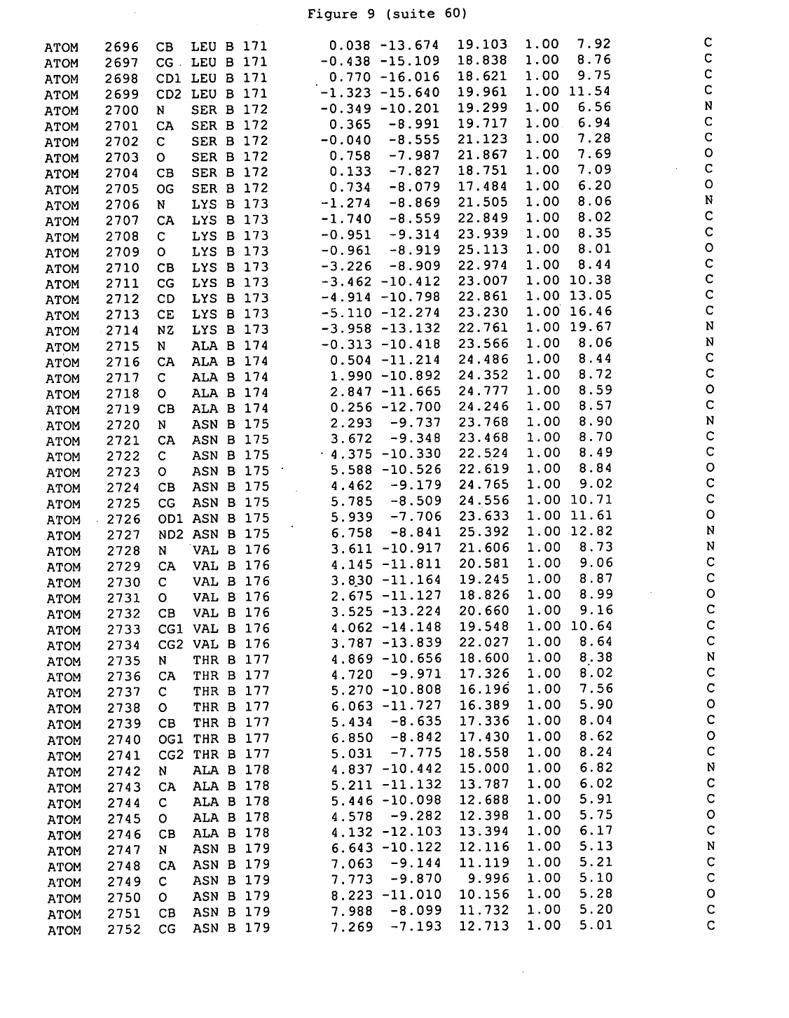

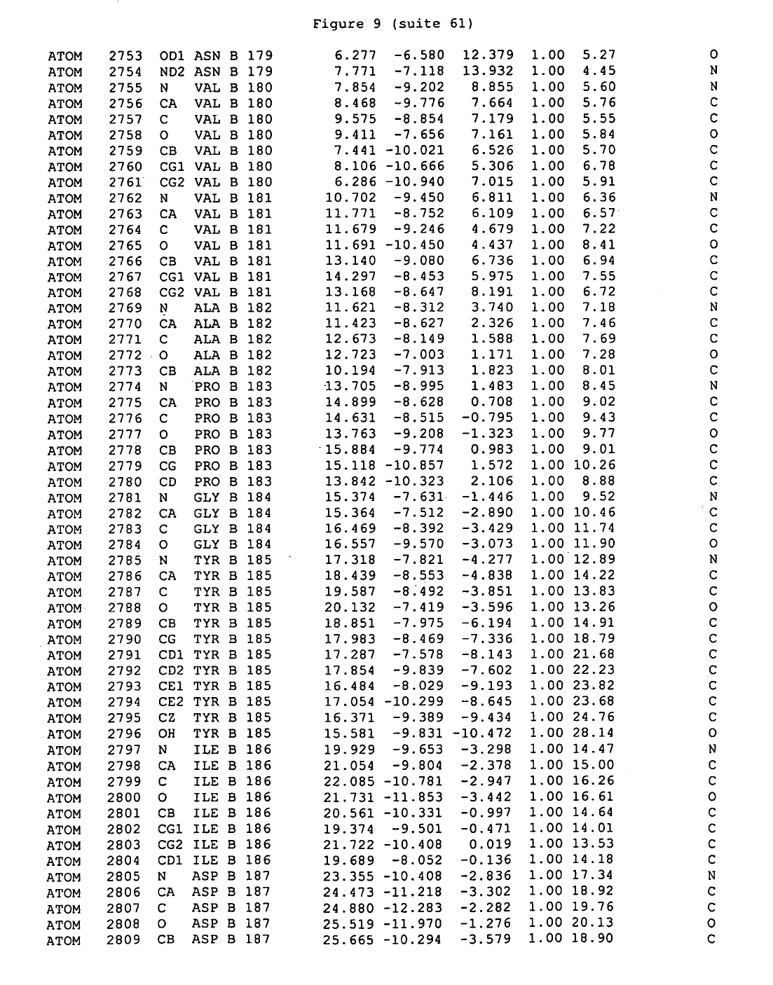

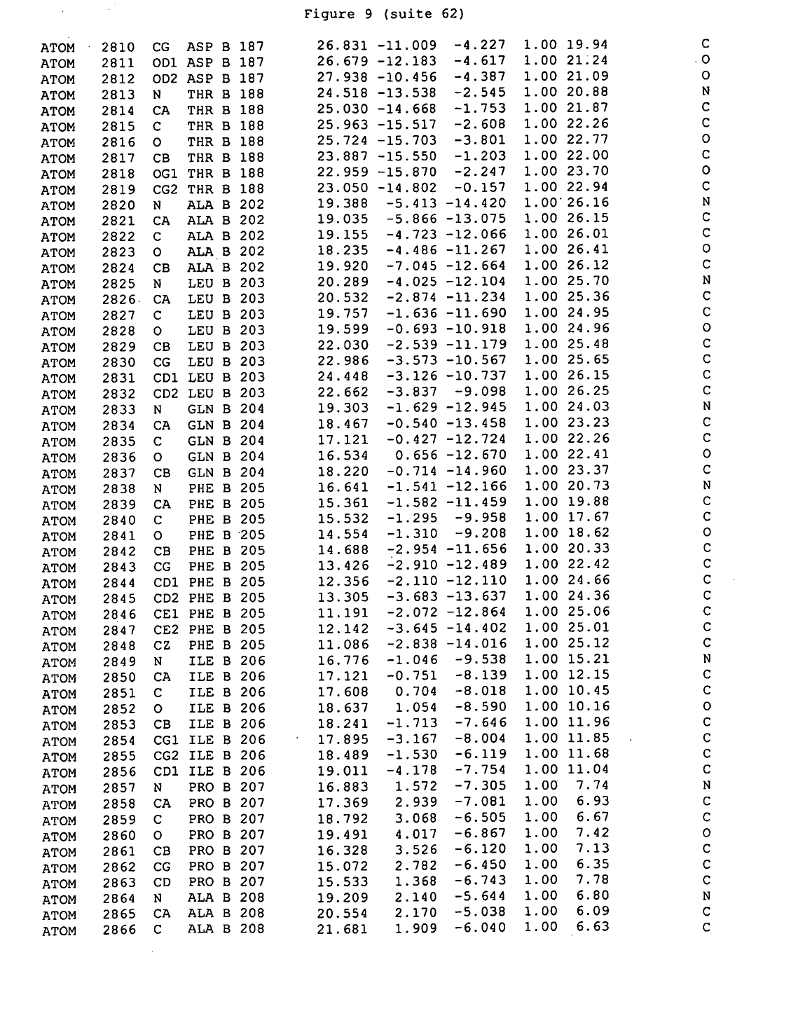

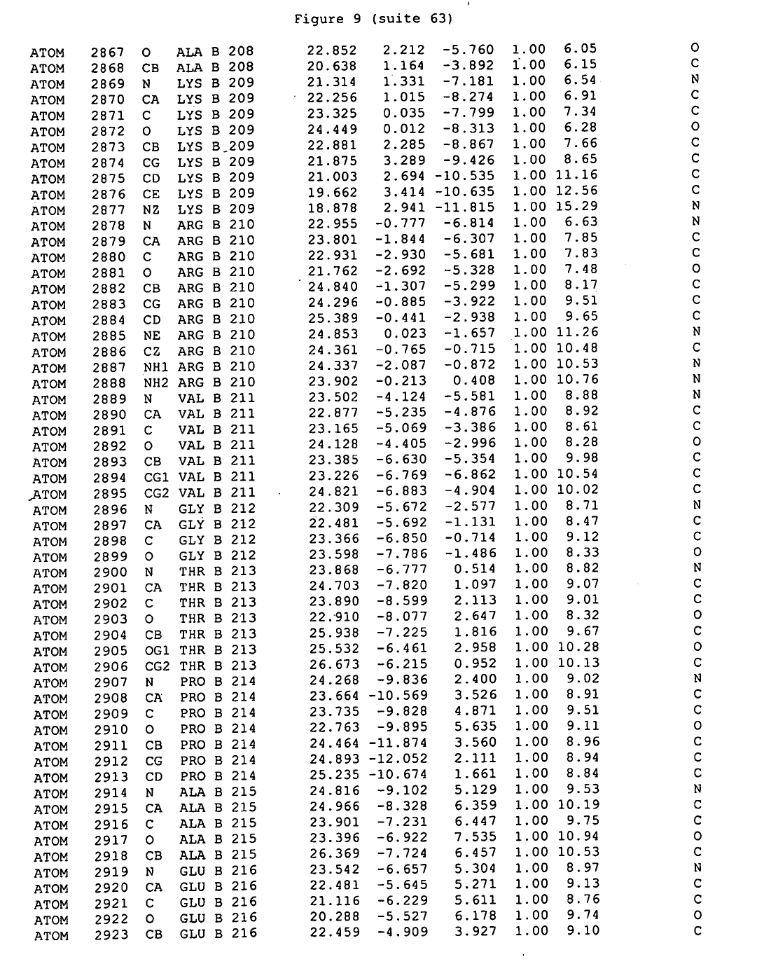

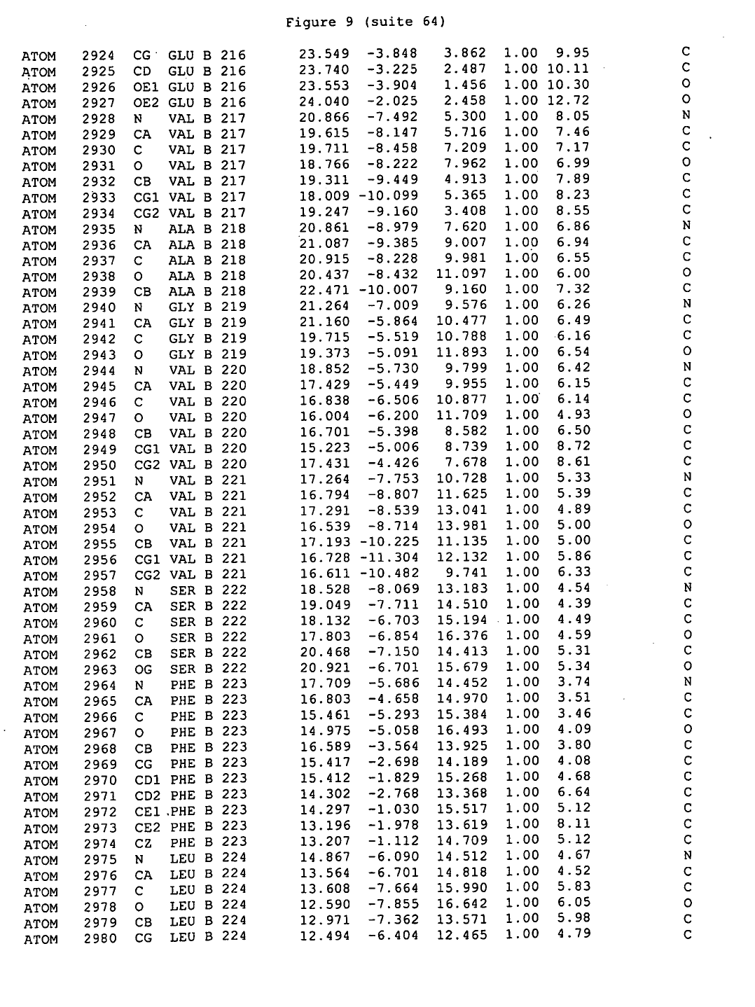

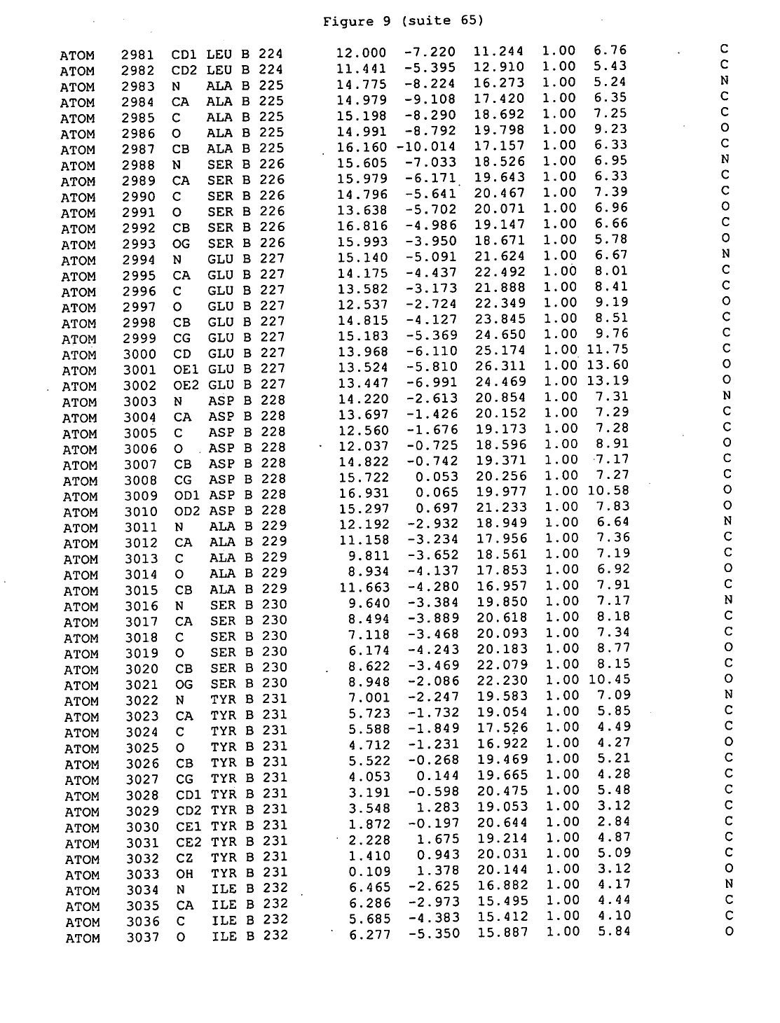

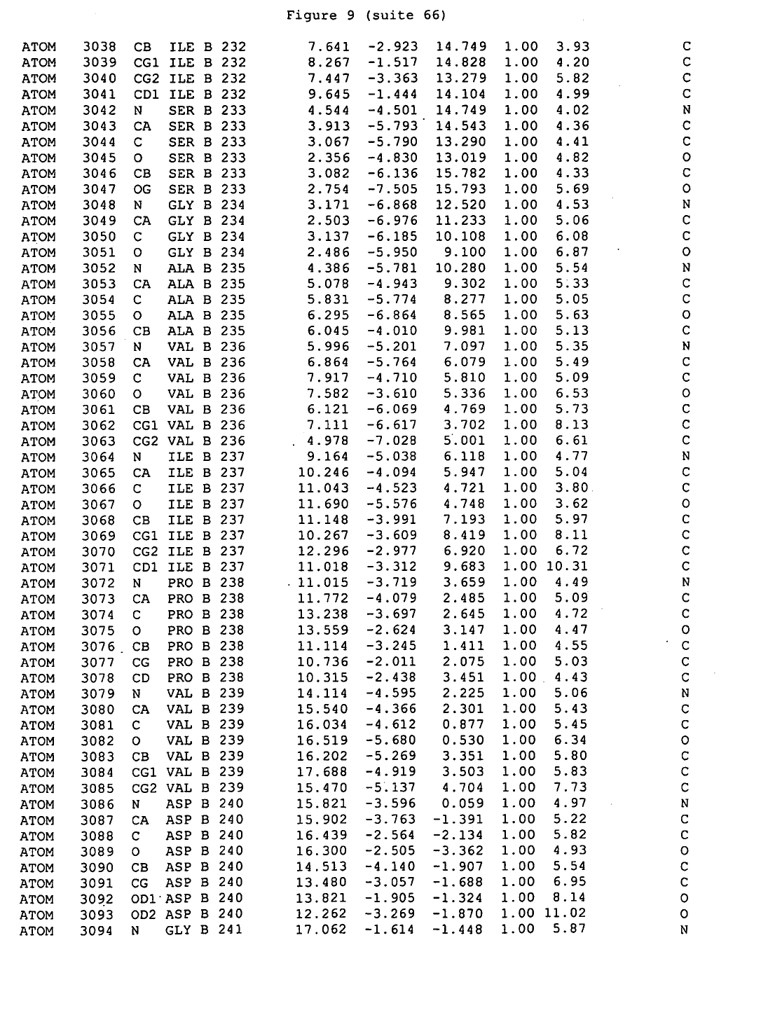

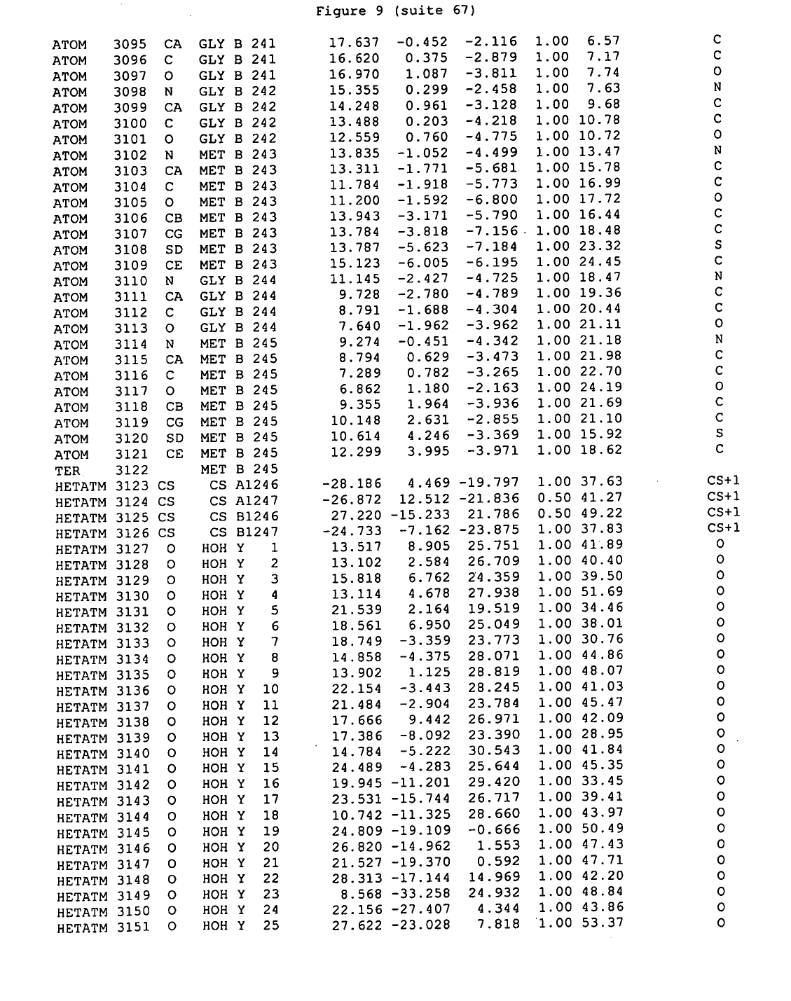

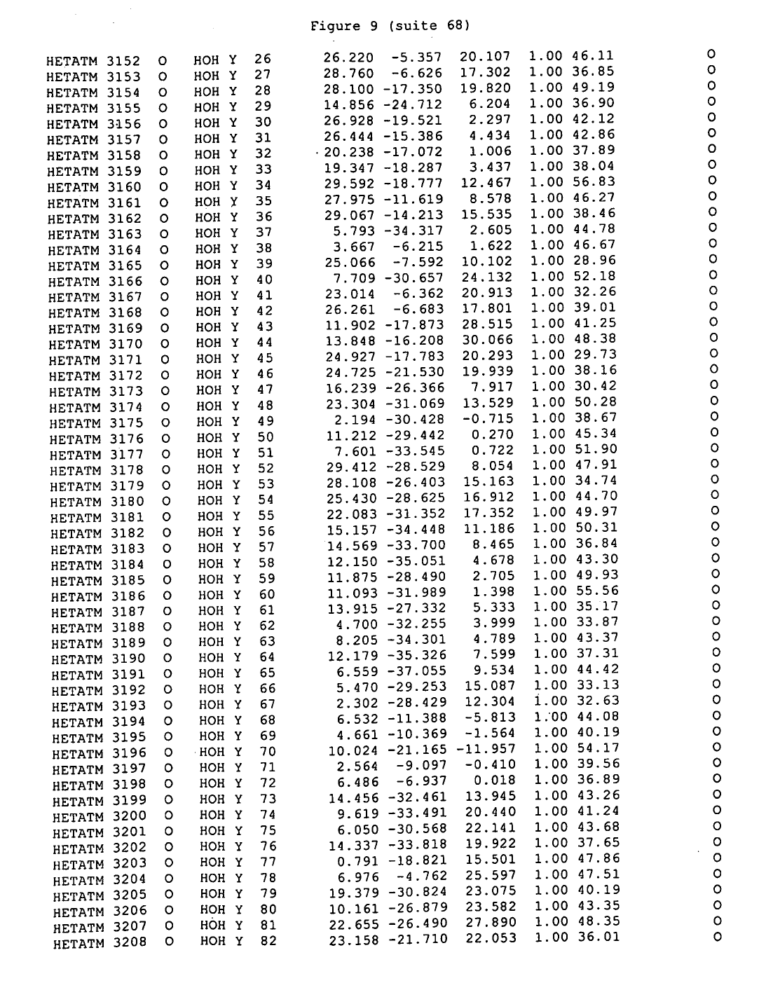

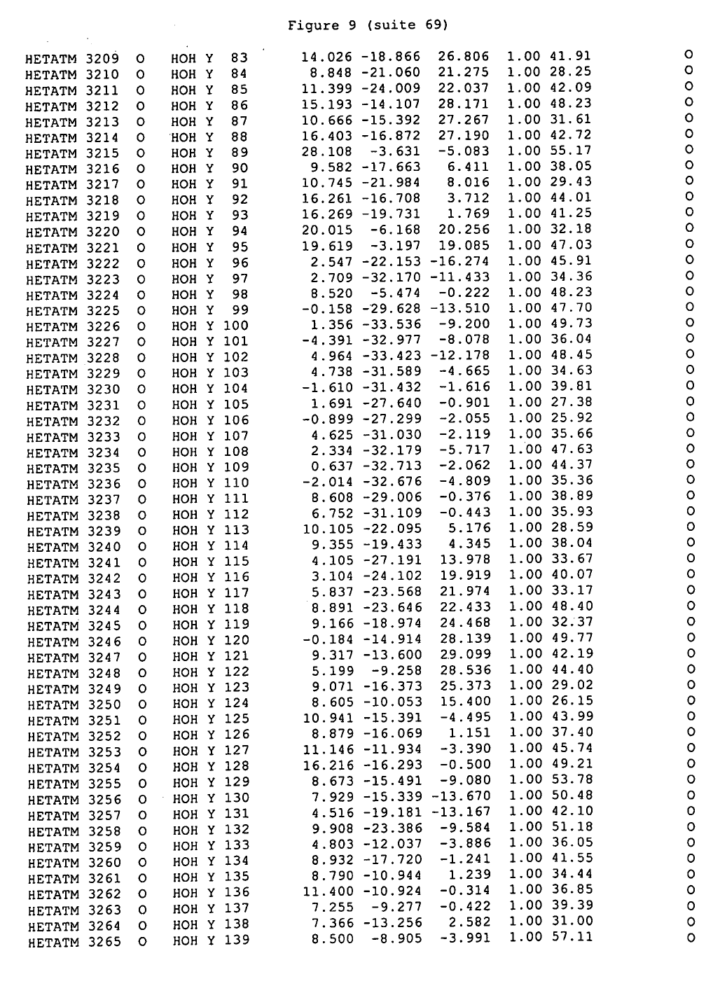

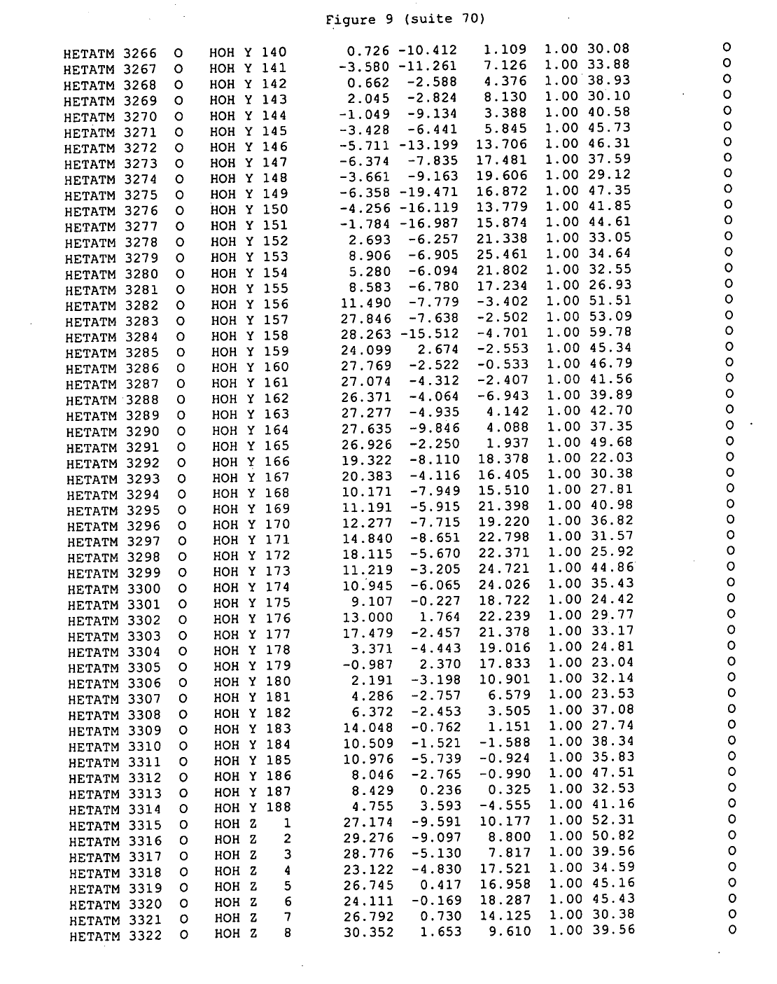

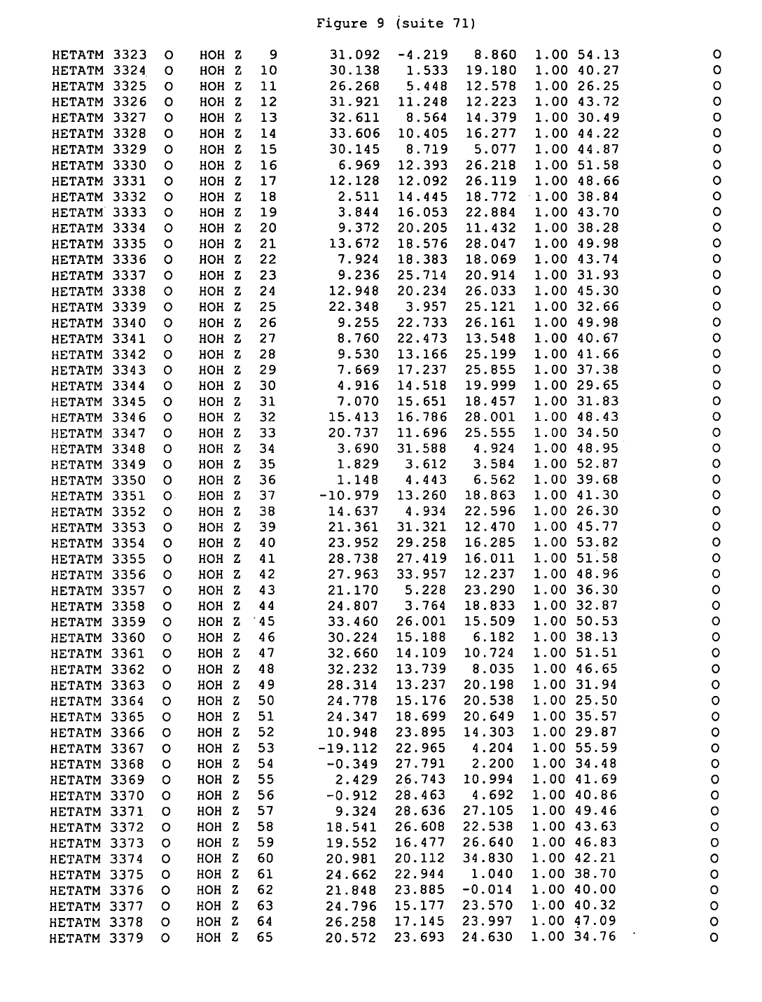

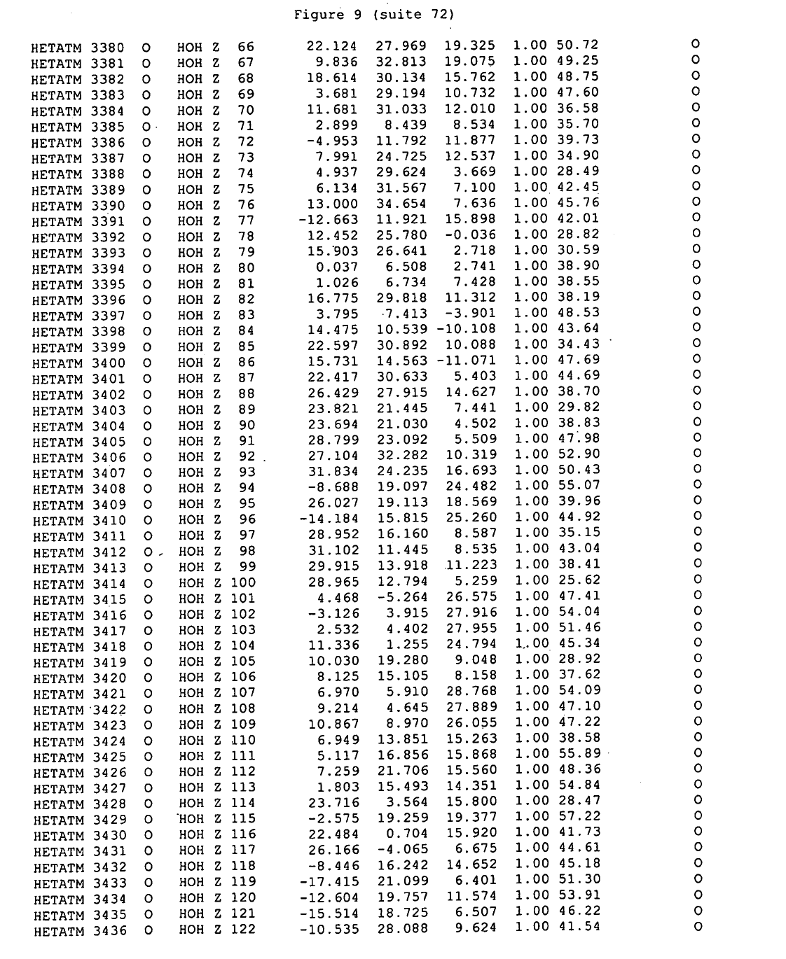

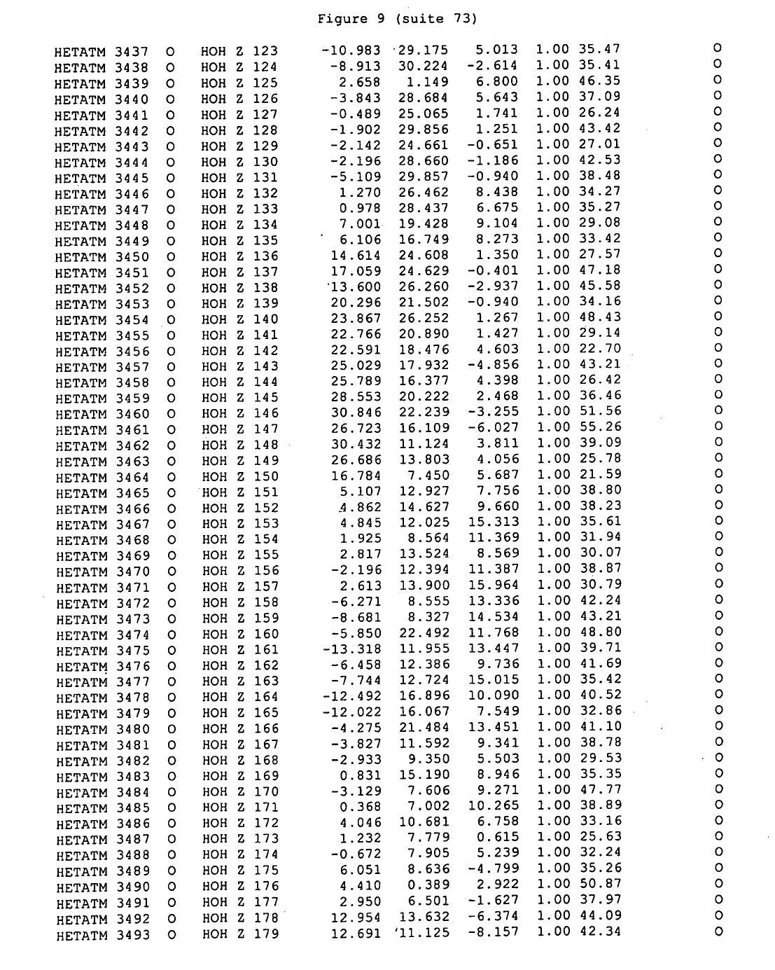

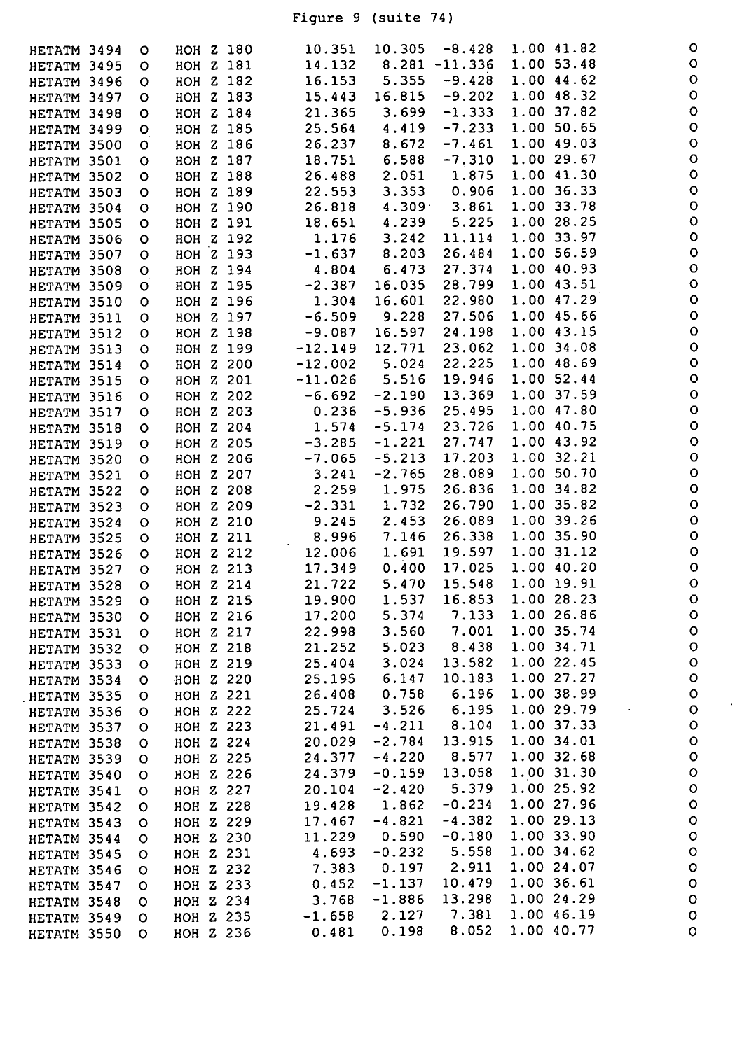

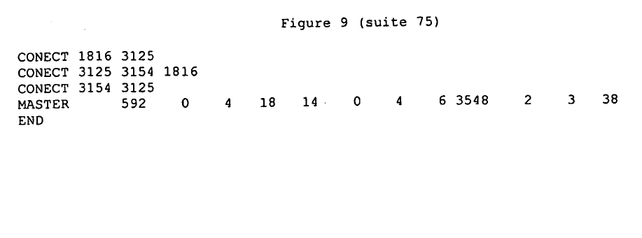

The docking of an acyl-CoA substrate appeared straightforwardly feasible in the modeled holo-form of MabA. By comparison with related NADPH-specific keto reductases and a mannitol debydrogenase, crystallized or modeled in complex with either a substrate or a competitive inhibitor (32-34), the orientation of the two substrate keto groups in MabA can be proposed to be similar to that observed for these ligands [FIG. 6(A)]. Importantly, a similar location and orientation of the substrate were observed in InhA (29) and have been postulated for KARbn (17). This location suggested the potential role of some hydrophilic residues [S92, D94, and QI50 in MabA; FIG. 6(C)]. They may form a “binding triad,” which was recently shown to also be conserved in the distantly related KR domains of polyketide synthases (35).

Our MabA model was compared with the crystal structure of the ternary complex InhA-NAD⁺-C16 substrate (29) and manual fitting of the C4, C8, C12, and C16 substrates was performed to identify the residues possibly involved in enzyme specificity. MabA substrate binding pocket contains numerous hydrophobie residues including W145, I147, Y185, I198, and F205 [FIG. 6(B)]. The interaction of the Y185 side chain with the substrate has been recently shown by directed mutagenesis (16). The residues W145 and I147 are specifically observed in mycobacterial FabG. In other KARs more polar residues substitute them. These substitutions suggested that MabA active site could accommodate the large and hydrophobic tail of a β-ketoacyl derivative with only local rearrangements. However, a significant difference in the conformation of the acyl chain of the substrate in MabA compared to that in InhA is predicted. Instead of a U shape, an L shape would be favored [FIG. 6(B)]. This is mainly due to the presence of hydrophilic residues [S92, D94, and Q150 in MabA; FIG. 6(C)] that are predicted to interact with two keto groups of the substrate (see above). For longer acyl chains (C18 and above) one might tentatively predict that the additional methylene groups are either to interacts with and cover the hydrophobic residues lying on the top of the substrate binding cleft (e.g., I198, F205) or potentially point into a second substrate binding site in the tetramer. However, this view might need to be revised in the in vivo complex FAS-II containing MabA. However, the current substrate docking is useful for a refined annotation of the MabA mycobacterial homologs.

Comparison with holo-KARbn and apo and holo-KARec suggested that the position occupied by isoleucine I147 in MabA (asparagine in KARec and KARbn) could be responsible for the recognition of substrates with long acyl chains [FIG. 6(B)]. The C4 substrate acyl chain should not contact the side chain of this residue according to our modeling study, while the longer chains could. The hydrophilic asparagine found at an equivalent position in holo-KARbn and apo-KARec would disfavor the binding of a long hydrophobic acyl chain. The observed conformation in holo-MabA is in agreement with the effect of the substitution of this isoleucine to an asparagine at the position 147 in MabA, like the decreased affinity of the mutant MabAI147N for the C12 substrate (15). The lower stability of the mutant protein observed during the purification step agreed with unfavorable contacts made by the hydrophilic asparagine side chain in the closed form, and the stabilizing contacts of I147 with the neighboring and hydrophobic residues such as W145 in the active conformation (“open form”) of the wild-type enzyme. These specific structural features might correlate with the observed distinct substrate specificity. Applying these rules to FabG2 and FabG4 of M. tuberculosis suggested that the latter is C4-specific (due to the presence of an asparagine) while the former (bearing a methionine) would share substrate specificity similar with MabA (FabG 1).

### CONCLUSION

This study provides a new structure of an “open” form of a bacterial KAR. It highlights a novel ligand-induced fit among the proteins of the so-called structural superfamily of SDRs. The active form of the protein was stabilized with a single mutation designed by comparative modeling. It also showed that the C-terminus, specific to mycobacterial MabA, adopts a particular conformation that locks the conformational changes.

The new structure of MabA was compared with those of the related KARs and of proteins of the SDR superfamily (27, 28), which also comprises InhA. The overall specificities of MabA make this enzyme a good candidate for rational antimycobacterial drug design. MabA shares only 20% sequence identity (over 200 aa) with InhA or the other ENRs, despite the similarity of their respective ligands (β-ketoacylCoA vs enoyl-CoA and NADPH vs NADH), despite their related functions. InhA has been crystallized in various forms including one complexed to the INH-NAD adduct (36) (PDB1ZID). The shape of the active site of MabA in the “open” form appeared similar to that of InhA. Comparative docking of the cofactor adduct or the substrate in MabA active site suggested that, in the observed “open” form, little adjustments of the catalytic residues is required for the enzyme to be able to bind the inhibitory adduct (16). In agreement with this result and the present structure of the active form, a point mutation (T21A) lying within the cofactor binding site of MabA was recently described in a M. tuberculosis clinical isolate resistant to isoniazid (37). According to the holo-form structure, the threonine T21 is hydrogen bonded to the strand β4 (through the backbone of residue N88; FIG. 2), which is involved in the conformational rearrangement described above. The mutation to alanine may at least destabilize active form, especially the interaction with the cofactor (mediated by R25 and N88 among other residues) and facilitate the release of the latter (a limiting step in SDR catalysis). Such a mechanism would limit the poisoning of the protein by the INH-NADP adduct. MabA inhibitors will have to be designed to fit the particular substrate binding site or prevent the conformational rearrangements. Several specific features of the protein observed in the “open” form can now be used for the design of new ligands like the EGCG and the related polyphenol recently described (38). Modeling studies of the closely related MabA from two other mycobacterial species, M. smegmatis and M. leprae, showed perfett conservation of the active site. Analysis of the overall structure suggests that they could be active and would share similar substrate specificity.

TABLE I
Data Collection and Refinement Statistics

| | | | |
|---|---|---|---|
| Protein | MabA-C60V/S144L (apo) | MabA-C60V/S144L (holo) | MabA-wt (mixed) |

Data collection

| | | | |
|---|---|---|---|
| Space group | C2 | C2 | C2 |
| Unit cell dimensions | α = 81.27 b = 116.99 c = 51.97 | α = 81.54 b = 117.11 c = 51.67 | α = 81.58 b = 117.16 c = 52.49 |
| | β = 122.05 | β = 122.62 | β = 122.65 |
| No of molecules per AU | 2 (named A and B) | 2 (named A and B) | 2 (named A and B) |
| Resolution range (Å) | 59.0-1.49 Å | 50.0-1.91 Å | 59.7-2.00 Å |
| Unique reflections | 60917 | 28001 | 24747 |
| R_{merge} (%)^{a,b} | 2.6 (41.1) | 5.2 (15.0) | 6.5 (31.8) |
| I/σ^{a} | 23.9 (0.9) | 17.0 (4.3) | 17.2 (1.8) |
| Completeness^{a} | 92.9 (70.9) | 93.1 (76.2) | 99.7 (99.9) |

Refinement

| | | | |
|---|---|---|---|
| R_{cryst}(%)^{c} | 19.9 | 18.5 | 18.2 |
| R_{free}(%)^{d} | 22.8 | 22.7 | 25.0 |

B values (Å²)

| | | | |
|---|---|---|---|
| Average | 15.6 | 24.4 | 20.0 |
| Main chain | 11.3/12.0 | 22.0/22.3 | 16.2/19.6 |
| Side chains | 12.8/13.4 | 23.5/23.3 | 17.9/20.1 |
| Waters | 40.1 | 44.2 | 39.1 |
| Cs | 41.5 | 53.7 | 45.7 |
| NADP | — | 32.7 | — |

R.M.S. deviations^{e}

| | | | |
|---|---|---|---|
| Bonds lengths (Å) | 0.010 | 0.010 | 0.015 |
| Bond angles (°) | 1.24 | 1.18 | 1.53 |
| Number of water molecule | 473 | 271 | 298 |
| Number of Cs | 4 | 4 | 4 |
| Number of NADP^{f} | 0 | 1 | 0 |
| Unseen residues | A: 1-8, 94-99, 144-148, 245-247 | A: 1-8 | A: 1-8 95-99 |
| | B: 1-8, 94-98, 143-149, 189- | B: 1-8, 190-201 | B: 1-8, 189-201 |
| | 201, 244-247 | | |

^{a}Values in parentheses refer to the outermost resolution shell, 1.53-1.49 Å for MabA-C60V/S144L(apo), 1.96-1.91 Å for MabA-C60V/S144L(holo), and 2.05-1.99 Å for MabA-wt.
^{b}R_{merge} = Σₕₖ, Σᵢ|I_{hkl, i} − I_{average, hkl}|/|ΣₕₖₗΣᵢ|I_{hkl, i}| × 100.
^{c}R_{cryst} = Σₕₖₗ|F_{obs} − F_{calc}|/Σₕₖₗ|F_{obs}| × 100.
^{d}R_{free} = Σ_{hk/LT}|F_{obs} − F_{calc}|/Σ_{hk/LT}|F_{obs} × 100 for 3626 reflections in MabA-C60VS144L(apo), 1462 reflections in MabA-C60VS144L(holo), and 1293 in MabA-wt.
^{e}Deviation from ideal values.
^{f}The nicotinamide moiety and its ribose group are not visible in the electronic density.

### REFERENCES

1. Barry C E 3rd, Mdluli K. Drug sensitivity and environmental adaptation of mycobacteria J cel wall components. Trends MicrobioI 1996;4:275-281.

2. Cole S T. Mycobacterium tuberculosis: drug-resistance mechanisms. Trends Microbiol 1994;2:411-415.

3. Iserman M. The WHO/IUATLD global project on anti-tuberculosis drug resistance surveillance 1994-1997. In: Anti-tuberculosis drug resistance in the world. Geneva: World Health Organisation; 1997.

4. Cole S T, Brosch R, Parkhill J, Gamier T, Churcher C, Harris D, Gordon S V, Eiglmeier K, Gas S, Barry C E 3rd, Tekaia F, Badcock K, Basham D, Brown D, Chillingworth T, Connor R, Davies R, Devlin K, Feltwell T, Gent]es S, Hamlin N, Holroyd S, Hornsby T, Jagels K, Barrell B G, et al. Deciphering the biology of Mycobacterium tuberculosis from the complete genome sequence. Nature 1998;393:537-544.

5. Daffe M, Draper P. The envelope layers of mycobacteria with reference to their pathogenicity. Adv Microb Physiol 1998;39:131-203.

6. Marrakchi H, Laneelle G, Quemard A. InhA, a target of the antituberculosis drug isoniazid, is involved in a mycobacterial fatty acid elongation system, FAS-II. Microbiology 2000;146:289-296.

7. Quemard A, Lacave C, Laneelle G. Isoniazid inhibition of mycolic acid synthesis by cell extracts of sensitive and resistant strains of Mycobacterium aurum. Antimicrob Agents Chemother 1991;35: 1035-1039.

8. Vilcheze C, Morbidoni H R, Weisbrod T R, Iwamoto H, Kuo M, Sacchettini J C, Jacobs W R Jr. Inactivation of the inhA-encoded fatty acid synthase II (FASII) enoyl-acyl carrier protein reductase induces accumulation of the FASII end products and cell lysis of Mycobacterium smegmatis. J Bacteriol 2000;182:4059-4067.

9. Odriozola J M, Ramos J A, B]och K. Fatty acid synthetase activity in Mycobacterium smegmatis. Characterization of the acyl carrier protein-dependent elongating system. Biochim Biophys Acta 1977; 488:207-217.

10. Bloch K. Control mechanisms for fatty acid synthesis in Mycobacterium smegmatis. Adv Enzymol Relat Areas Mol Biol 1977;45:1-84.

11. Banerjee A, Dubnau E, Quemard A, Balasubramanian V, Um K S, Wilson T, Collins D, de Lisle G, Jacobs W R Jr. inhA, a gene encoding a target for isoniazid and ethionamide in Mycobacterium tuberculosis. Science 1994; 263:227-230.

12. Quemard A, Sacchettini J C, Dessen A, Vilcheze C, Bittman R, Jacobs W R Jr, Blanchard J S. Enzymatic characterization of the target for isoniazid in Mycobacterium tuberculosis. Biochemistry 1995;34:8235-8241.

13. Parikh S L, Xiao G, Tonge P J. Inhibition of InhA, the enoyl reductase from Mycobacterium tuberculosis, by triclosan and isoniazid. Biochemistry 2000;39:7645-7650.

14. Marrakchi H, Ducasse S, Labesse G, Margeat E, Montrozier H, Emorine L, Charpentier X, Lanéelle G, Quémard A. Biochemical and structural studies of the Mycobacterium tuberculosis MabA (FabG1) protein involved in the fatty acid elongation system, FAS-II. Microbiology 2002;148:951-960.

15. Cohen-Gonsaud M, Ducasse S, Hoh F, Zerbib D, Labesse G, Quémard A. Crystal structure of MabA from Mycobacterium tuberculosis, a reductase involved in long-chain fatty acid biosynthesis. J Mol Biol 2002;320:249-261.

16. Ducasse-Cabanot S, Cohen-Gonsaud M, Marrakchi H, NGuyen M, Zerbib D, Bemadou J, Daffe M, Labesse G, Quémard A. In vitro inhibition of the beta-ketoacyl-ACP reductese MabA from Mycobacterium tuberculosis by isoniazid. Agents Chemother 2004;48:242-249.

17. Fisher M, Kroon J T, Martindale W, Stuitje A R, Slabas A R, Rafferty J B. The X-ray structure of Brassica napus beta-keto acyl carrier protein reductase and its implications for substrate binding and catalysis. Struct Fold Des 2000;8:339-347.

18. Price A C, Zhang Y M, Rock C O, White S W. Structure of beta ketoacyl-[acyl carrier protein] reductase from Escherichia coli: negative cooperativity and its structural basis. Biochemistry 2001;40:12772-12781.

19. Price A C, Zhang Y M, Rock C O, White S W. Cofactor-induced conformational rearrangements establish a catalytically competent active site and a proton relay conduit in FabG. Structure (Camb) 2004;12:417-428.

20. Jones T A, Zou J Y, Cowan S W, Kjeldgaard M. Improved methods for building protein models in electron density maps and the location of errors in these models Acta Crystallogr SectA 1991;47: 110-119.

21. Collaborative Computational Project 4. Acta Crystallogr Sect D Biol Crystallogr 1994;50:760-763.

22. Perrakis A, Morris R M, Lamzin Y S. Automated protein model building combined with iterative structure refinement. Nat Struct Biol 1999;6:458-463.

23. Winn M D, Isupov M N, Murshudov G N. Use of TLS parameters to model anisotropic displacements in macromolecular refinement. Acta Crystallogr 2001;D57:122-133.

24. Laskowski R A, MacArthur M W, Moss D S, Thornton J M. PRO-CHECK V2. Oxford, Eng]and: Oxford Mo]ecular Ltd.; 1992.

25. Berman H M, Westbrook J, Feng Z, GiUiland G, Bhat T N, Weissig H, Shindyalov I N, Bourne P E. The Protein Data Bank. Nucleic Acids Res 2000;28:235-242.

26. Altschul S F, Madden T L, Schaffer A A, Zhang J, Zhang Z, MiUer W, Lipman D J. Gapped BLAST and PSI-BLAST: a new generation of protein database search programs. Nucleic Acids Res 1997;25: 3389-3402.

27. Jornvall H, Persson B, Krook M, Atrian S, Gonzalez-Duarte R, Jeffery J, Ghosh D. Short-chain dehydrogenases/reductases (SDR). Biochemistry 1995;34:6003-6013.

28. Labesse G, Vidal-Cros A, Chomilier J, Gaudry M, Mornon J P. Structural comparisons lead to the definition of a new superfamily of NAD(P)(H)-accepting oxidoreductases: the single-domain reductases/epimerases/dehydrogenases (the “RED” family). Biochem J 1994;304:95-99.

29. Rozwarski D A, Vilcheze C, Sugantino M, Bittman R, Sacchettini J C. Crystal structure of the Mycobacterium tuberculosis enoyl ACP reductase, InhA, in complex with NAD⁺ and a C16 fatty acyl substrate. J Biol Chem 1999;274:15582-15589.

30. Sheldon P S, Kekwick R G, Smith C G, Sidebottom C, Slabas A R. 3-0xoacyl-[ACP] reductase from oilseed rape (Brassica napus). Biochim Biophys Acta 1992;1120:151-159.

31. Grimm C, Maser E, Mobus E, Klebe G, Reuter K, Ficner R. The crystal structure of 3alpha-hydroxysteroid dehydrogenase/carbonyl reductase from Comamonas testosteroni shows a novel oligomerization pattern within the short chain dehydrogenase/reductase family. J Biol Chem 2000;275:41333-41339.

32. Andersson A, Jordan D, Schneider G, Lindqvist Y. Crystal structure of the ternary complex of 1,3,8-trihydroxynaphthalene reductase from Magnaporthe grisea with NADPH and an active-site inhibitor. Structure 1996;4:1161-1170.

33. Horer S, Stoop J, Mooibroek H, Baumann U, Sassoon J. The crystallographic structure of the mannitol 2-dehydrogenase NADP⁺ binary complex from Agaricus bisporus. J Biol Chem 2001;276:27555-27561.

34. Nakajima K, Kato H, Oda J, Yamada Y, Hashimoto T. Site directed mutagenesis of putative substrate-binding residues reveals a mechanism controlling the different stereospecificities of two tropinone reductases. J Biol Chem 1999;274:16563-16568.

35. Reid R, Piagentini M, Rodriguez E, Ashley G, Viswanathan N, Carney J, Santi D V, Hutchinson C R, McDaniel R. A model of structure and cata]ysis for ketoreductase domains in modular polyketide synthases. Biochemistry 2003;42:72-79.

36. Rozwarski D A, Grant G A, Barton D H, Jacobs W R Jr, Sacchettini J C. Modification of the NADH of the isoniazid target (InhA) from Mycobacterium tuberculosis. Science 1998;279:98-102.

37. Ramaswamy S V, Reich R, Dou S-J, Jasperse L, Pan X, Wanger A, Quitugua T, Graviss E A. Single nucleotide polymorphisms in genes associated with isoniazid resistance in Mycobacterium tuberculosis. Antimicrob Agents Chemother 2003;47:1241-1250.

38. Zhang Y M, Rock C O. Evaluation of epigallocatechin gallate and related plant polyphenols as inhibitors of the FabG and FabI reductases of bacterial type II fatty-acid synthase. J Biol Chem 2004;30:30994-31001.

1. Complexes between the nicotinamide adenine dinucleotide phosphate of formula 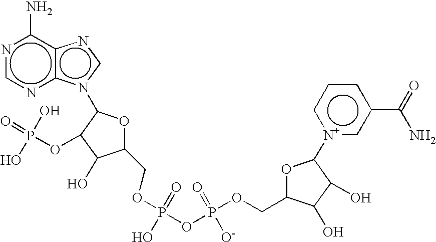
and:
the protein MabA of Mycobacterium tuberculosis, MabA having the following amino acid sequence SEQ ID NO: 1:
MTATATEGAK PPFVSRSVLV TGGNRGIGLA IAQRLAADGH
KVAVTHRGSG APKGLFGVEC DVTDSDAVDR AFTAVEEHQG
PVEVLVSNAG LSADAFLMRM TEEKFEKVIN ANLTGAFRVA
QRASRSMQRN KFGRMIFIGS VSGSWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH
or the proteins derived from the protein MabA mentioned above, and selected from the followings:
the MabA derived protein corresponding to the protein MabA in which the cysteine in position 60 is replaced by a valine residue, and the serine in position 144 is replaced by a leucine residue, said derived protein, also called C(60)V/S(144)L, corresponding to the following sequence SEQ ID NO 2:
MTATATEGAK PPFVSRSVLV TGGNRGIGLA IAQRLAADGH
KVAVTHRGSG APKGLFGVEV DVTDSDAVDR AFTAVEEHQG
PVEVLVSNAG LSADAFLMRM TEEKFEKVIN ANLTGAFRVA
QRASRSMQRN KFGRMIFIGS VSGLWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH
the MabA derived protein corresponding to the protein MabA in which the cysteine in position 60 is replaced by a valine residue, said derived protein, also called C(60)V, corresponding to the following sequence SEQ ID NO 3:
MTATATEGAK PPFVSRSVLV TGGNRGIGLA IAQRLAADGH
KVAVTHRGSG APKGLFGVEV DVTDSDAVDR AFTAVEEHQG
PVEVLVSNAG LSADAFLMRM TEEKFEKVIN ANLTGAFRVA
QRASRSMQRN KFGRMIFIGS VSGSWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH
the MabA derived protein corresponding to the protein MabA in which the to serine in position 144 is replaced by a leucine residue, said derived protein, also called S(144)L, corresponding to the following sequence SEQ ID NO 4:
MTATATEGAK PPFVSRSVLV TGGNRGIGLA IAQRLAADGH
KVAVTHRGSG APKGLFGVEC DVTDSDAVDR AFTAVEEHQG
PVEVLVSNAG LSADAFLMRM TEEKFEKVIN ANLTGAFRVA
QRASRSMQRN KFGRMIFIGS VSGLWGIGNQ ANYAASKAGV
IGMARSIARE LSKANVTANV VAPGYIDTDM TRALDERIQQ
GALQFIPAKR VGTPAEVAGV VSFLASEDAS YISGAVIPVD
GGMGMGH

2. Ternary complexes of a binary complex according to claim 1, and a ligand of the protein MabA, or of a recombinant protein derived from the protein MabA, and more particularly a molecule ligand capable of binding specifically at the level of the active site of the protein MabA, or proteins similar in structure to the protein MabA, and inhibiting the enzymatic activity of the latter.

3. Complexes according to claim 1 or 2, in crystallized form.

4. Crystals of protein complexes defined in claim 1 or 2, as obtained by the hanging-drop vapour diffusion method, by mixing said protein (1 μl of a 10 mg/ml solution) with a solution (1 μl) of NADP (50-100 mM), polyethylene glycol 3000 (6-12%), CsCl (150-450 mM), and optionally glycerol (10%), in PIPES buffer (50 mM) at pH 6.6.

5. Crystals of the complex between NADP and the recombinant protein MabA corresponding to the sequence SEQ ID NO: 1 according to claim 1, the atomic coordinates of the three-dimensional structure of protein MabA in said complex being represented in FIG. 7.

6. Crystals of the complex between NADP and the recombinant protein MabA C(60)V/S(144)L corresponding to the sequence SEQ ID NO: 2 according to claim 1, the atomic coordinates of the three-dimensional structure of protein MabA C(60)V/S(144)L in said complex being represented in FIG. 8.

7. Method for screening ligands of the protein MabA of the protein MabA, or of protein MabA C(60)V/S(144)L, or of protein MabA C(60)V, or of protein MabA S(144)L, in crystallo, said method comprising:
either the co-crystallization of the purified recombinant protein MabA, or MabA C(60)V/S(144)L, or MabA C(60)V, or MabA S(144)L, in the presence of NADP and of the potential ligand (or a mixture of potential ligands),
or the soaking of the crystals of the complexes as defined in claim 1 of NADP with MabA, or with MabA C(60)V/S(144)L, or with MabA C(60)V, or with MabA S(144)L, in a potential ligand solution (or a mixture of potential ligands),
and the determination by crystallography of the three-dimensional structure of the crystals of the ternary complexes of protein MabA, or MabA C(60)V/S(144)L, or MabA C(60)V, or MabA S(144)L, with a potential ligand and NADP.

8. Method for designing or screening ligands of the protein MabA, said method comprising the use of the coordinates of the three-dimensional structure of crystals of protein MabA, or of protein MabA C(60)V/S(144)L, or of protein MabA C(60)V, or of protein MabA S(144)L, in complexes of said proteins with NADP, and more particularly of the coordinates of the three-dimensional structure of crystals of protein MabA, or of protein MabA C(60)V/S(144)L, represented in FIGS. 7 and 8 respectively, for screening in silico of the virtual combinatorial libraries of potential ligands, advantageously using appropriate computer softwares, and the detection and rational structural optimization of the ligands capable of binding to said protein.

9. Method of rational design of ligands of the protein MabA, said method being carried out starting with known inhibitors of MabA for which the fine three-dimensional structure of the complex between said inhibitor and the recombinant protein MabA in purified form was determined, and rational structural optimization of said inhibitors by using an appropriate computer software in which the coordinates of the three-dimensional structure of protein MabA, or of protein MabA C(60)V/S(144)L, or of protein MabA C(60)V, or of protein MabA S(144)L, in crystals of complexes of said proteins with NADP, and more particularly the coordinates of the three-dimensional structure of protein MabA or of protein MabA C(60)V/S(144)L represented in FIGS. 7 and 8 respectively, have been entered.

10. Method according to any of claims 7 to 9, for designing or screening ligands of the protein MabA, or a recombinant protein derived from the protein MabA, and more particularly molecules capable of binding specifically at the level of the active site of the protein MabA, or proteins similar in structure to the protein MabA, and inhibiting the enzymatic activity of the latter.

11. Method according to claim 10, for designing or screening ligands acting as inhibitors of the protein MabA, or a recombinant protein derived from the protein MabA, these inhibitors being chosen in particular from:
the steroid derivatives,
the derivatives of the antituberculous antibiotic isoniazid (isonicotinic acid hydrazide), such as the derivatives of the isonicotinoyl-NAD(P) adduct,
the derivatives of N-acetyl cysteamine or other simplified types of derivatives of the coenzyme A, comprising a grafted fluorophore making it possible to use the fluorescence spectroscopy method, in particular time-resolved, for the detection of protein-ligand interactions,
the inhibiting derivatives of the protein InhA of Mycobacterium tuberculosis.

12. Method according to any of claims 7 to 11, for designing or screening ligands of the protein MabA, or a recombinant protein derived from the protein MabA, that can be used in pharmaceutical compositions, in particular within the framework of the treatment of pathologies linked to mycobacterial infections, such as tuberculosis due to infection by Mycobacterium tuberculosis, or by Mycobacterium africanium, or leprosy due to infection by Mycobacterium leprae, or mycobacteriosis due to infection by opportunist mycobacteria, such as Mycobacterium avium, Mycobacterium fortuitum, Mycobacterium kansasii, Mycobacterium chelonae.


SEQUENCE LISTING (5 sequences)

SEQ ID NO: 1 - protein, 247 aa, Mycobacterium tuberculosis
MTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 2 - protein, 247 aa, Mycobacterium tuberculosis
MTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 3 - protein, 247 aa, Mycobacterium tuberculosis
MTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 4 - protein, 247 aa, Mycobacterium tuberculosis
MTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 5 - protein, 20 aa, Artificial Sequence
  Synthetic Primer (Polyhistidine-tag)
MGSSHHHHHHSSGLVPRGSH